(19) United States
(12) Patent Application Publication
(10) Pub. No.: US 2024/0299352 A1
(43) Pub. Date: Sep. 12, 2024

(54) COMBINATION THERAPIES WITH SETD2 INHIBITORS

(71) Applicant: Epizyme, Inc., Cambridge, MA (US)

(72) Inventors: Maria Alejandra RAIMONDI, Jamaica Plain, MA (US); Jenniffer Anne TOTMAN, Berlin, MA (US); Vinny MOTWANI, Newton, MA (US); Katherine Louise COSMOPOULOS, Quechee, VT (US); Dorothy BRACH, Lynnfield, MA (US); Daniel T. DRANSFIELD, Hanson, MA (US)

(21) Appl. No.: 18/568,368

(22) PCT Filed: Jun. 8, 2022
(86) PCT No.: PCT/US2022/032718

§ 371 Date: Dec. 8, 2023

Related U.S. Application Data
(60) Provisional application No. 63/208,882, filed on Jun. 9, 2021.

(51) Int. Cl. A61K 31/404; A61K 45/06; A61P 35/00

(52) U.S. Cl. CPC A61K 31/404; A61K 45/06; A61P 35/00

(57) ABSTRACT

The present disclosure provides SETD2 protein inhibitors, and methods, uses, compositions, and kits for treating diseases, disorders, or conditions in a subject with a SETD2 protein inhibitor and a Second Therapeutic Agent, wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

### BACKGROUND OF THE INVENTION

### Field of the Invention

The present disclosure provides SETD2 protein inhibitors, and methods, uses, compositions, and kits for treating diseases, disorders, or conditions in a subject with a SETD2 protein inhibitor in combination with a Second Therapeutic Agent, wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

### Background

The selective addition of methyl groups to specific amino acid sites on histones is controlled by the action of a family of enzymes known as histone methyltransferases (HMTs). The level of expression of a particular gene is influenced by the presence or absence of one or more methyl groups at a relevant histone site. The specific effect of a methyl group at a particular histone site persists until the methyl group is removed by a histone demethylase, or until the modified histone is replaced through nucleosome turnover. In a like manner, other enzyme classes can decorate DNA and histones with other chemical species, and still other enzymes can remove these species to provide control of gene expression.

SETD2 is a human histone methyltransferase located at the cytogenic band p21.31 of chromosome 3 (3p21.31). The acronym “SETD2” stands for Suppressor of variegation, Enhancer of zeste, and Trithorax domain containing 2. The SETD2 protein comprises three conserved functional domains: (1) the triplicate AWS-SET-PostSET domain; (2) a WW domain; and (3) a Set2-Rbp1 interacting (“SRI”) domain. These three functional domains define the biological function of SETD2. See, Li, J. et al., Oncotarget 7:50719-50734 (2016). SETD2 is believed to be the single human gene responsible for the trimethylation of lysine 36 (Lys-36) of histone H3 (H3K36me3) using dimethylated Lys-36 (H3K36me2) as substrate. Edmunds, J. W. et al., The EMBO Journal 27:406-420 (2008).

Human SETD2 has been shown to have tumor suppressor functionality. Li, J. et al., Oncotarget 7:50719-50734 (2016). For example, inactivation of human SETD2 has been reported in renal cell carcinoma (RCC). Larkin, J., et al., Nature Reviews 9:147-155 (2012). Also, expression levels of SETD2 in breast cancer samples have been reported as significantly lower than in adjacent non-cancerous tissue (ANCT) samples. Newbold, R. F. and Mokbel, K., Anticancer Research 30: 3309-3311 (2010). Additionally, biallelic mutations and loss-of-function point mutations in SETD2 were reported in patients with acute leukemia. Zhu, X. et al., Nature Genetics 46: 287-293 (2014). Mutations in SETD2 have also been reported in pediatric high-grade gliomas. Fontebasso, A. M. et al., Acta Neuropathol. 125: 659-669 (2013).

### BRIEF SUMMARY OF THE INVENTION

The present disclosure generally provides SETD2 protein inhibitors, and methods, uses, compositions, and kits for treating diseases, disorders, or conditions in a subject with a SETD2 protein inhibitor and a Second Therapeutic Agent, wherein the second therapeutic agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

In one aspect, the present disclosure provides methods of treating diseases, disorders, or conditions, e.g., cancer, in a subject in need thereof with:
(1) a therapeutically effective amount of a substituted indole represented by any one of Formulae I, II, II-A, III, III-A, IV, IV-A, IV-B, IV-C, IV-D, V, V-A, V-B, VI, VII, VII-A, VII-B, VII-C, VII-D, VII-E, VII-F, VII-G, VII-H, VIII, VIII-A, or VIII-B, or a compound of Table 1, or a compound of Table 1B, below, and the pharmaceutically acceptable salts and solvates thereof, collectively referred to herein as a “Compounds of the Disclosure;” and
(2) a therapeutically effective amount of a Second Therapeutic Agent,
wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

In another aspect, the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, or one or more CARM1 inhibitors, or a combination thereof.

In another aspect, the present disclosure provides methods of treating diseases, disorders, or conditions, e.g., cancer, in a subject in need thereof with:
(1) a therapeutically effective amount of a Compound of the Disclosure;
(2) a therapeutically effective amount of a Second Therapeutic Agent; and
(3) a therapeutically effective amount of a Third Therapeutic Agent.
wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

In another aspect, the present disclosure provides a Compound of the Disclosure for use in treating cancer, e.g., multiple myeloma, in a subject in need thereof, wherein the Compound of the Disclosure is to be administered to the subject in combination with a Second Therapeutic Agent and, optionally, a Third Therapeutic Agent.

In another aspect, the present disclosure provides a Compound of the Disclosure for use in the manufacture of a medicament for treating cancer in a mammal, wherein the Compound of the Disclosure is to be administered to the subject in combination with a Second Therapeutic Agent and, optionally, a Third Therapeutic Agent.

In another aspect, the present disclosure provides a kit comprising a Compound of the Disclosure and a Second Therapeutic Agent and, optionally, a Third Therapeutic Agent

In another aspect, the present disclosure provides a pharmaceutical composition comprising a Compound of the Disclosure and a pharmaceutically acceptable carrier, for use in treating a disease, disorder, or condition in a subject in combination with a Second Therapeutic Agent and, optionally, a Third Therapeutic Agent.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

### I. Compounds of the Disclosure

Certain Compounds of the Disclosure are disclosed in WO 2020/037079 and WO 2021/168313 as SETD2 inhibitors. WO 2020/037079 and WO 2021/168313 are fully incorporated by reference herein in its entirety.

In one embodiment, Compounds of the Disclosure are compounds having Formula I:

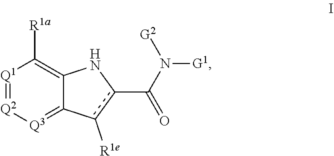

wherein:
R^{1a} is selected from the group consisting of halogen, alkyl, alkoxy, cycloalkyl, (hydroxy)alkyl, and (cycloalkyl)alkyl;
Q¹ is selected from the group consisting of —C(R^{1b})═ and —N═;
Q² is selected from the group consisting of —C(R^{1c})═ and —N═;
Q³ is selected from the group consisting of —C(R^{1d})═ and —N═;
provided that at least one of Q¹, Q², or Q³ is —C(R^{1b})═, —C(R^{1c})═, or —C(R^{1d})═, respectively;
R^{1b}, R^{1c}, and R^{1d} are each independently selected from the group consisting of hydrogen, halogen, alkyl, alkenyl, (hydroxy)alkyl, and alkoxy;
R^{1e} is selected from the group consisting of hydrogen, halogen, alkyl, cycloalkyl, (hydroxy)alkyl, and (cycloalkyl)alkyl;
[symbol] is a single or double bond;
G¹ is selected from the group consisting of optionally substituted aryl, optionally substituted heteroaryl, optionally substituted heterocyclo, optionally substituted cycloalkyl, (aryl)alkyl, (heteroaryl)alkyl, (heterocyclo)alkyl, (amino)(aryl)alkyl, (heteroaryl)(aryl)alkyl, (heteroaryl)(heterocyclo)alkyl, (heteroaryl)(carboxamido)alkyl, (heteroaryl)(cycloalkyl)alkyl, (aryl)(alkoxycarbonyl)alkyl, (cycloalkyl)alkyl, (heteroaryl)(amino)alkyl, (cycloalkyl)(alkoxycarbonyl)alkyl, (heteroaryl)(alkoxycarbonyl)alkyl, (heterocyclo)(cycloalkyl)alkyl, (aryl)(cycloalkyl)alkyl, (aryl)(hydroxy)alkyl, (cycloalkyl)(hydroxy)alkyl, (hydroxy)alkyl, optionally substituted alkyl, (aryl)(haloalkyl)alkyl, (cycloalkyl)(haloalkyl)alkyl, (hydroxy)(haloalkyl)alkyl, and (alkoxycarbonyl)(haloalkyl)alkyl; and
G² is selected from the group consisting of hydrogen and alkyl; or
G¹ and G² taken together with the nitrogen atom to which they are attached form an optionally substituted heterocyclo, or a pharmaceutically acceptable salt or solvate thereof.

In another embodiment, the compound having Formula I is not N-(1-(1-(L-alanyl)piperidin-4-yl)ethyl)-7-methyl-1H-indole-2-carboxamide, N-((1r,4r)-4-(3-aminopropanamido)cyclohexyl)-7-methyl-1H-indole-2-carboxamide, or N-((1r,4r)-4-aminocyclohexyl)-7-methyl-1H-indole-2-carboxamide.

In another embodiment, Compounds of the Disclosure are compounds having Formula I, wherein:
R^{1a} is selected from the group consisting of halogen, C₁-C₆ alkyl, C₁-C₆ alkoxy, C₃-C₈ cycloalkyl, (hydroxy)C₁₋₆ alkyl, and (C₃-C₆ cycloalkyl)C₁₋₆ alkyl;
R^{1b}, R^{1c}, and R^{1d} are each independently selected from the group consisting of hydrogen, halogen, C₁-C₆ alkyl, C₂-C₆ alkenyl, (hydroxy)C₁-C₆ alkyl, and C₁-C₆ alkoxy;
R^{1e} is selected from the group consisting of hydrogen and C₁-C₆ alkyl;
G¹ is selected from the group consisting of optionally substituted C₆-C₁₀ aryl, optionally substituted 5- to 10-membered heteroaryl, optionally substituted 3- to 10-membered heterocyclo, optionally substituted C₃-C₆ cycloalkyl, (C₆-C₁₀ aryl)C₁-C₆ alkyl, (5- to 10-membered heteroaryl)C₁-C₆ alkyl, (3- to 10-membered heterocyclo)C₁-C₆ alkyl, (amino)(C₆-C₁₀ aryl)C₁-C₆ alkyl, (5- to 14-membered heteroaryl)(C₆-C₁₀ aryl)C₁-C₆ alkyl, (5- to 10-membered heteroaryl)(3- to 10-membered heterocyclo)C₁-C₆ alkyl, (5- to 10-membered heteroaryl)(carboxamido)C₁-C₆ alkyl, (5- to 10-membered heteroaryl)(C₃-C₆ cycloalkyl)C₁-C₆ alkyl, (C₆-C₁₀ aryl)(alkoxycarbonyl)C₁-C₆ alkyl, (C₃-C₆ cycloalkyl)C₁-C₆ alkyl, (5- to 10-membered heteroaryl)(amino)C₁-C₆ alkyl, (C₃-C₆ cycloalkyl)(alkoxycarbonyl)C₁-C₆ alkyl, (5- to 14-membered heteroaryl)(alkoxycarbonyl)C₁-C₆ alkyl, (3- to 14-membered heterocyclo)(C₃-C₆ cycloalkyl)C₁-C₆ alkyl, (C₆₋₁₀ aryl)(C₃-C₆ cycloalkyl)C₁-C₆ alkyl, (C₆-C₁₀ aryl)(hydroxy)C₁-C₆ alkyl, (C₃-C₆ cycloalkyl)(hydroxy)C₁-C₆ alkyl, (hydroxy)C₁-C₆ alkyl, optionally substituted C₁-C₆ alkyl, (C₆-C₁₀ aryl)(C₁-C₆ haloalkyl)C₁-C₆ alkyl, (C₃-C₆cycloalkyl)(C₁-C₆ haloalkyl)C₁-C₆ alkyl, (hydroxy)(C₁-C₆ haloalkyl)C₁-C₆ alkyl; and (alkoxycarbonyl)(C₁-C₆ haloalkyl)C₁-C₆ alkyl; and
G² is selected from the group consisting of hydrogen and C₁-C₆ alkyl; or
G¹ and G² taken together with the nitrogen atom to which they are attached form a 5- to 10-membered optionally substituted heterocyclo, or a pharmaceutically acceptable salt or solvate thereof.

In another embodiment, Compounds of the Disclosure are compounds having Formula I, wherein:
R^{1a} is selected from the group consisting of halogen, C₁-C₃ alkyl, C₁-C₃ alkoxy, C₃-C₆ cycloalkyl, (hydroxy)C₁₋₄ alkyl, and (C₃-C₆ cycloalkyl)C₁₋₄ alkyl;
R^{1b}, R^{1c}, and R^{1d} are each independently selected from the group consisting of hydrogen, halogen, C₁-C₃ alkyl, C₂-C₄ alkenyl, (hydroxy)C₁-C₄ alkyl, and C₁-C₃ alkoxy;
R^{1e} is selected from the group consisting of hydrogen and C₁-C₃ alkyl;
G¹ is selected from the group consisting of optionally substituted C₆-C₁₀ aryl, optionally substituted 5- to 10-membered heteroaryl, optionally substituted 3- to 10-membered heterocyclo, optionally substituted C₃-C₈ cycloalkyl, (C₆-C₁₀ aryl)C₁-C₄ alkyl, (5- to 10-membered heteroaryl)C₁-C₆ alkyl, (3- to 10-membered heterocyclo)C₁-C₄ alkyl, (amino)(C₆-C₁₀ aryl)C₁-C₆ alkyl, (5- to 14-membered heteroaryl)(C₆-C₁₀ aryl)C₁-C₄ alkyl, (5- to 10-membered heteroaryl)(3- to 10-membered heterocyclo)C₁-C₄ alkyl, (5- to 10-membered heteroaryl)(carboxamido)C₁-C₄ alkyl, (5- to 10-membered heteroaryl)(C₃-C₆ cycloalkyl)C₁-C₄ alkyl, (C₆-C₁₀ aryl)(alkoxycarbonyl)C₁-C₄ alkyl, (C₃-C₆ cycloalkyl)C₁-C₄ alkyl, (5- to 10-membered heteroaryl)(amino)C₁-C₄ alkyl, (C₃-C₆ cycloalkyl)(alkoxycarbonyl)C₁-C₄ alkyl, (5- to 14-membered heteroaryl)(alkoxycarbonyl)C₁-C₄ alkyl, (3- to 14-membered heterocyclo)(C₃-C₆ cycloalkyl)C₁-C₄ alkyl, (C₆₋₁₀ aryl)(C₃-C₆ cycloalkyl)C₁-C₄ alkyl, (C₆-C₁₀ aryl)(hydroxy)C₁-C₄ alkyl, (C₃-C₆ cycloalkyl)(hydroxy)C₁-C₄ alkyl, (hydroxy)C₁-C₄ alkyl, optionally substituted C₁-C₄ alkyl, (C₆-C₁₀ aryl)(C₁-C₄ haloalkyl)C₁-C₄ alkyl, (C₃-C₆cycloalkyl)(C₁-C₄ haloalkyl)C₁-C₄ alkyl, (hydroxy)(C₁-C₄ haloalkyl)C₁-C₄ alkyl, and (alkoxycarbonyl)(C₁-C₄ haloalkyl)C₁-C₄ alkyl; and
G² is selected from the group consisting of hydrogen and C₁-C₄ alkyl; or
G¹ and G² taken together with the nitrogen atom to which they are attached form a 5- to 10-membered optionally substituted heterocyclo, or a pharmaceutically acceptable salt or solvate thereof.

In another embodiment, Compounds of the Disclosure are compounds having Formula I, wherein [symbol] is a double bond, or a pharmaceutically acceptable salt or solvate thereof.

In another embodiment, Compounds of the Disclosure are compounds having Formula I, wherein Q¹ and Q² are —C(H)═, or a pharmaceutically acceptable salt or solvate thereof.

In another embodiment, Compounds of the Disclosure are compounds having Formula I, wherein Q³ is —C(R^{1d})═; and R^{1d} is selected from the group consisting of hydrogen and halo, or a pharmaceutically acceptable salt or solvate thereof.

In another embodiment, Compounds of the Disclosure are compounds having Formula I, wherein R^{1e} is hydrogen, or a pharmaceutically acceptable salt or solvate thereof.

In another embodiment, Compounds of the Disclosure are compounds having Formula I, wherein R^{1a} is C₁-C₃ alkyl, or a pharmaceutically acceptable salt or solvate thereof.

In another embodiment, Compounds of the Disclosure are compounds having Formula I, wherein G² is hydrogen, or a pharmaceutically acceptable salt or solvate thereof.

In another embodiment, Compounds of the Disclosure are compounds having Formula II:

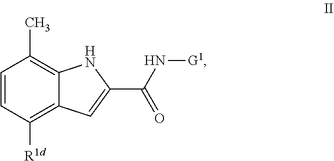

or a pharmaceutically acceptable salt or solvate thereof, wherein R^{1d} and G¹ are as defined in connection with Formula I.

In another embodiment, Compounds of the Disclosure are compounds having Formulae I or II, wherein R^{1d} is selected from the group consisting of hydrogen and fluoro, or a pharmaceutically acceptable salt or solvate thereof.

In another embodiment, Compounds of the Disclosure are compounds having Formula II-A:

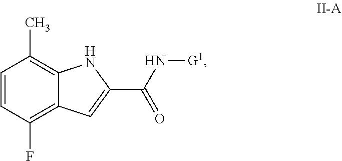

or a pharmaceutically acceptable salt or solvate thereof, wherein G¹ is as defined in connection with Formula II.

In another embodiment, Compounds of the Disclosure are compounds having Formulae I, II, or II-A, wherein G¹ is selected from the group consisting of optionally substituted C₆-C₁₀ aryl, optionally substituted 5- to 9-membered heteroaryl, optionally substituted 3- to 10-membered heterocyclo, optionally substituted C₆-C₈ cycloalkyl, (5- to 9-membered heteroaryl)C₁-C₆ alkyl, (5- to 9-membered heteroaryl)(C₆₋₁₀ aryl)C₁-C₄ alkyl, (5- to 9-membered heteroaryl heteroaryl)(C₃-C₆ cycloalkyl)C₁-C₄ alkyl, and (C₃-C₆ cycloalkyl)C₁-C₄ alkyl, or a pharmaceutically acceptable salt or solvate thereof.

In another embodiment, Compounds of the Disclosure are compounds having Formula III:

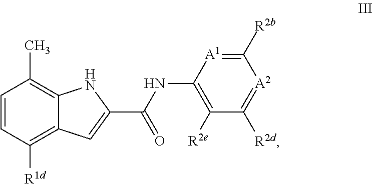

wherein:
A¹ is selected from the group consisting of —N═ and —C(R^{2a});
R^{2a} is selected from the group consisting of hydrogen, alkyl, halogen, and haloalkyl;
R^{2b} is selected from the group consisting of optionally substituted alkyl, optionally substituted heterocyclo, optionally substituted cycloalkyl, optionally substituted heteroaryl, optionally substituted aryl, (carboxamido)alkyl, —OR^{10c}, amino, (heterocyclo)alkyl, (amino)alkyl, (hydroxy)alkyl, carboxamido, (heteroaryl)alkyl, —S(═O)R^{9b}, —S(═O)₂R^{9b}, and —C(═O)R^{9c};
A² is selected from the group consisting of —N═ and —C(R^{2c})═;
R^{2c} is selected from the group consisting of hydrogen, alkyl, halogen, and haloalkyl;
R^{2d} is selected from the group consisting of hydrogen, alkyl, halogen, cyano, and haloalkyl;
R^{2e} is selected from the group consisting of hydrogen, alkyl, halogen, and haloalkyl;
R^{9b} is selected from the group consisting of amino, alkyl, optionally substituted cycloalkyl, optionally substituted aryl, optionally substituted heterocyclo, and optionally substituted heteroaryl;
R^{9c} is selected from the group consisting of amino, alkyl, optionally substituted cycloalkyl, optionally substituted aryl, optionally substituted heterocyclo, and optionally substituted heteroaryl; and
R^{10c} is selected from the group consisting of alkyl, (hydroxy)alkyl, and (amino)alkyl; and
R^{1d} is as defined in connection with Formula I, or a pharmaceutically acceptable salt or solvate thereof.

In another embodiment, Compounds of the Disclosure are compounds having Formula III-A:

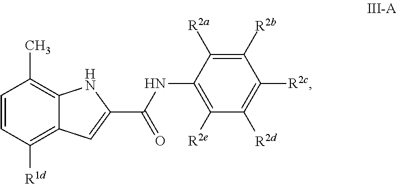

wherein R^{1d}, R^{2a}, R^{2b}, R^{2c}, R^{2d}, and R^{2e} are as defined in connection with Formula III, or a pharmaceutically acceptable salt or solvate thereof.

In another embodiment, Compounds of the Disclosure are compounds having Formula III or Formula III-A, wherein:
R^{2a} is selected from the group consisting of hydrogen, C₁-C₄ alkyl, halogen, and C₁-C₄ haloalkyl;
R^{2b} is selected from the group consisting of:
(A) unsubstituted 4- to 10-membered heterocyclo;
(B) substituted 4- to 10-membered heterocyclo having one, two, three, or four substituents independently selected from the group consisting of (i) —N(R^{3a})C(═O)R^{4a}; (ii) —NR^{5a}R^{5b}; (iii) unsubstituted 4- to 10-membered heterocyclo; (iv) substituted 4- to 10-membered heterocyclo having one, two, or three substituents independently selected from the group consisting of hydroxy, —NR^{5c}R^{5d}, C₁-C₄ alkyl, C₁-C₆ alkoxy, —C(R^{6a})(R^{6b})C(═O)NR^{5e}R^{5f}, —C(═O)R^{4b}, (hydroxy)C₁-C₄ alkyl, and halo; (v) unsubstituted C₃-C₆ cycloalkyl; (vi) (hydroxy)C₁-C₄ alkyl; (vii) C₁-C₆ alkyl; (viii) —C(═O)NR^{5g}R^{5h}; (ix) halo; (x) —C(═O)R^{4c}; (xi) C₁-C₆ haloalkyl; (xii) hydroxy; (xiii) (amino)C₁-C₄ alkyl; (xiv) (C₁-C₄ alkoxy)C₁-C₄ alkyl; (xv) —S(═O)₂R^{9a}; (xvi) (3- to 8-membered heterocyclo)C₁-C₄ alkyl; (xvii) C₁-C₆ alkoxy; (xviii) (C₃-C₆ cycloalkyl)C₁₋₄ alkyl; (xix) (C₆₋₁₀ aryl)C₁-C₄ alkyl; and (xxii) —OR^{10b};
(C) unsubstituted C₃-C₈ cycloalkyl;
(D) substituted C₃-C₈ cycloalkyl having one, two, three, or four substituents independently selected from the group consisting of (i) unsubstituted 4- to 10-membered heterocyclo; (ii) substituted 4- to 10-membered heterocyclo having one or two substituents, independently selected from the group consisting of amino and C₁-C₄ alkyl; (iii) unsubstituted 5- or 6-membered heteroaryl; (iv) substituted 5- or 6-membered heteroaryl having one, two, or three substituents independently selected from the group consisting of halo, C₁-C₄ alkyl, (3- to 8-membered heterocyclo)alkyl, hydroxy, and amino; (v) —NR⁵ⁱR^{5j}; (vi) cyano; (vii) —N(R^{3d})C(═O)R^{4f}; (viii) hydroxy; and (ix) C₁-C₄ alkyl;
(E) unsubstituted 5- to 10-membered heteroaryl;
(F) substituted 5- to 10-membered heteroaryl having one, two, three, or four substituents independently selected from the group consisting of (i) halo; (ii) C₁-C₄ alkyl; (C₁-C₄ alkoxy)C₁-C₄ alkyl; (hydroxy)C₁-C₄ alkyl; C₃-C₆ cycloalkyl; (amino)C₁-C₄ alkyl; unsubstituted C₃-C₆ cycloalkyl; substituted C₃-C₆ cycloalkyl having one, two, three, or four substituents independently selected from the group consisting of —NR^{5g}R^{5h}; unsubstituted 4- to 14-membered heterocyclo; substituted 4- to 14-membered heterocyclo having one or two substituents independently selected from the group consisting of hydroxy, amino, and C₁-C₄ alkyl; —NR^{5q}R^{5r}; and (ix) (3- to 8-membered heterocyclo)C₁-C₄ alkyl;
(G) unsubstituted C₆-C₁₀ aryl;
(H) substituted C₆-C₁₀ aryl, having one, two, three, or four substituents independently selected from the group consisting of (i) halo; (ii) C₁-C₄ alkyl; (iii) —CH—₂N(H)S(═O)₂R⁸; (iv) (5- to 9-membered heteroaryl)C₁-C₄ alkyl; (v) —OR^{10a}; (vi) —N(R^{3b})C(═O)R^{4b}; (vii) (amino)C₁-C₄ alkyl; and (viii) (hydroxy)C₁-C₄ alkyl;
(I) (carboxamido)C₁-C₄ alkyl;
(J) —OR^{10c};
(K) —NR^{5o}R^{5p};
(L) (3- to 8-membered heterocyclo)C₁-C₄ alkyl;
(M) (amino)C₁-C₄ alkyl;
(N) (hydroxy)C₁-C₄ alkyl;
(O) —C(═O)NR^{5s}R^{5t};
(P) (5- to 9-membered heteroaryl)C₁-C₄ alkyl; and
(Q) —S(═O)₂R^{9b};
R^{2c} is selected from the group consisting of hydrogen, C₁-C₄ alkyl, halogen, and C₁-C₄ haloalkyl;
R^{2d} is selected from the group consisting of hydrogen, C₁-C₄ alkyl, halogen, cyano, and C₁-C₄ haloalkyl;
R^{2e} is selected from the group consisting of hydrogen, C₁-C₄ alkyl, halogen, and C₁-C₄ haloalkyl;
R^{3a}, R^{3b}, R^{3c}, and R^{3d} are each independently selected from the group consisting of hydrogen, C₁-C₄ alkyl, optionally substituted C₃-C₆ cycloalkyl, and optionally substituted 4- to 14-membered heterocyclo;
R^{4a}, R^{4b}, R^{4c}, R^{4d}, R^{4e}, and R^{4f} are each independently selected from the group consisting of C₁-C₆ alkyl; C₁-C₆ haloalkyl; C₃-C₆ cycloalkyl; C₁-C₆ alkoxy; (C₁-C₄ alkoxy)C₁-C₄ alkyl; (C₆₋₁₀ aryl)C₁-C₄ alkyl; (5- to 9-membered heteroaryl)C₁-C₄ alkyl; (amino)C₁-C₄ alkyl; (hydroxy)C₁-C₄ alkyl; (cyano)C₁-C₄ alkyl; unsubstituted 4- to 14-membered heterocyclo; substituted 4- to 14-membered heterocyclo having one or two substituents independently selected from the group consisting of halo and C₁-C₄ alkyl; unsubstituted C₆-C₁₀ aryl; substituted C₆-C₁₀ aryl, having one, two, three, or four substituents independently selected from the group consisting of halo and C₁-C₄ alkyl; unsubstituted 5- or 6-membered heteroaryl; and substituted 5- or 6-membered heteroaryl having one, two, three, or four substituents independently selected from the group consisting of halo and C₁-C₄ alkyl;
R^{5a} and R^{5b} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;
R^{5c} and R^{5d} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R^{5c} and R^{5d} taken together with the nitrogen atom to which they are attached form an optionally substituted 4- to 14-membered heterocyclo;
R^{5e} and R^{5f} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R^{5e} and R^{5f} taken together with the nitrogen atom to which they are attached form an optionally substituted 4- to 14-membered heterocyclo;
R^{5g} and R^{5h} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R^{5g} and R^{5h} taken together with the nitrogen atom to which they are attached form an optionally substituted 4- to 14-membered heterocyclo;
R⁵ⁱ and R^{5j} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R⁵ⁱ and R^{5j} taken together with the nitrogen atom to which they are attached form an optionally substituted 4- to 14-membered heterocyclo;
R^{5k} and R^{5l} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R^{5k} and R^{5l} taken together with the nitrogen atom to which they are attached form an optionally substituted 4- to 14-membered heterocyclo;
R^{5m} and R⁵ⁿ are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R^{5m} and R⁵ⁿ taken together with the nitrogen atom to which they are attached form an optionally substituted 4- to 14-membered heterocyclo;
R^{5o} and R^{5p} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or
R^{5o} and R^{5p} taken together with the nitrogen atom to which they are attached form an optionally substituted 4- to 14-membered heterocyclo;
R^{5q} and R^{5r} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;
R^{5s} and R^{5t} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;
R^{6a}, R^{6b}, R^{6c}, and R^{6d} are each independently selected from the group consisting of hydrogen and C₁-C₄ alkyl;
R⁸ is C₁-C₆ alkyl;
R^{9a} is selected from the group consisting of C₁-C₆ alkyl; unsubstituted C₃-C₈ cycloalkyl; and substituted C₃-C₈ cycloalkyl having one or two substituents independently selected from the group consisting of halo, C₁-C₄ alkyl, amino, and (amino)C₁-C₄ alkyl;
R^{9b} is selected from the group consisting of C₁-C₆ alkyl and amino;
R^{10a} is selected from the group consisting of alkyl, (hydroxy)C₁-C₄ alkyl, and (amino)C₁-C₄ alkyl;
R^{10b} is (amino)C₁-C₄ alkyl; and
R^{10c} is (amino)C₁-C₄ alkyl, or a pharmaceutically acceptable salt or solvate thereof.

In another embodiment, Compounds of the Disclosure are compounds having Formula III or Formula III-A, wherein R^{2b} is an optionally substituted 3- to 10-membered heterocycle linked to the rest of the molecule through a nitrogen atom, e.g., R^{2b} is:

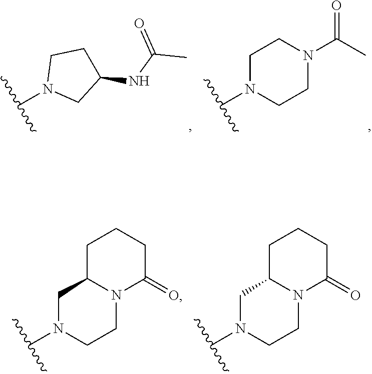

and the like.

In another embodiment, Compounds of the Disclosure are compounds having Formula III or Formula III-A, wherein:

R^{2b} is selected from the group consisting of

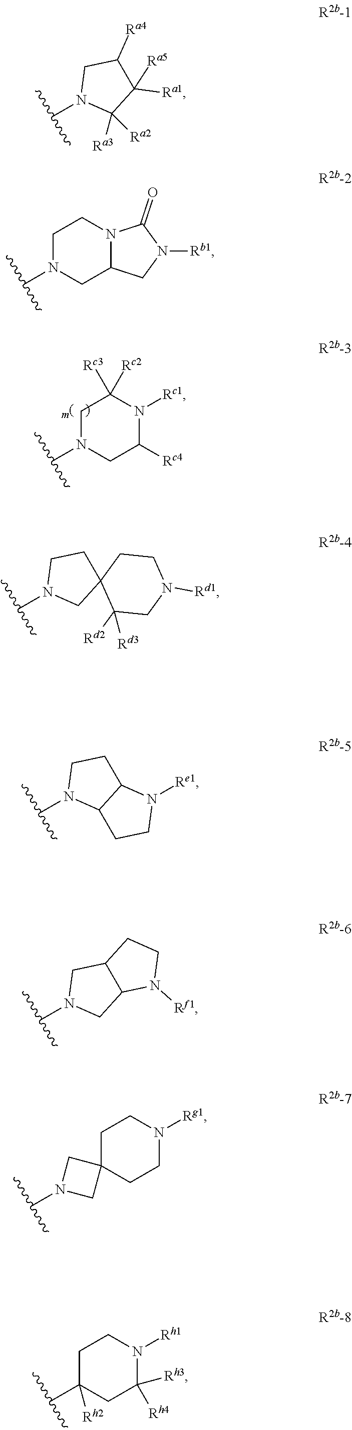

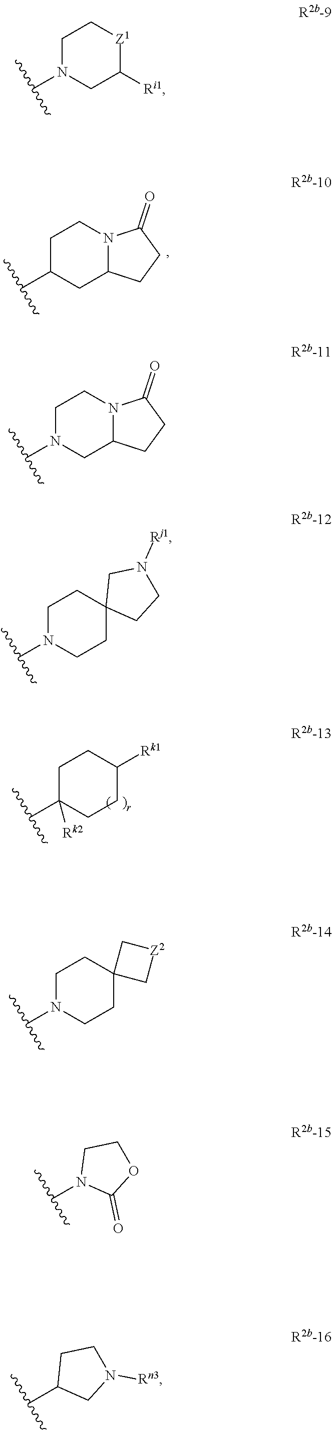

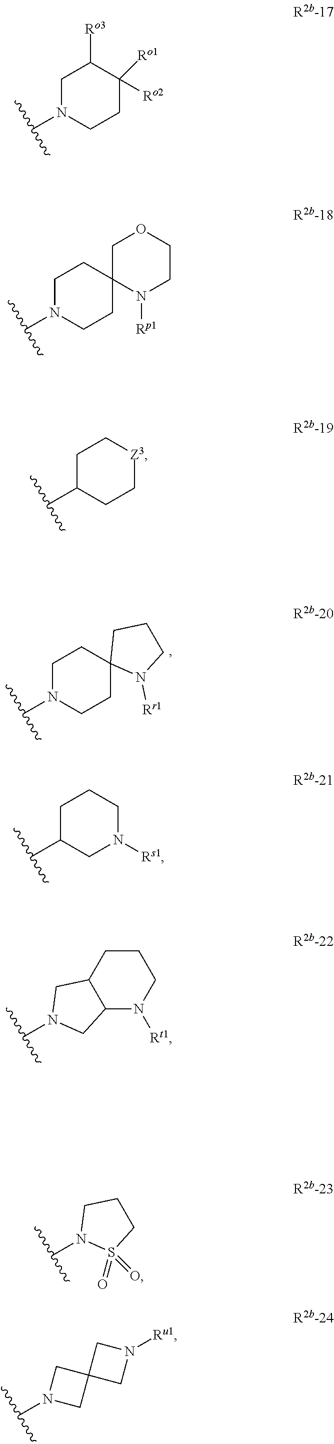

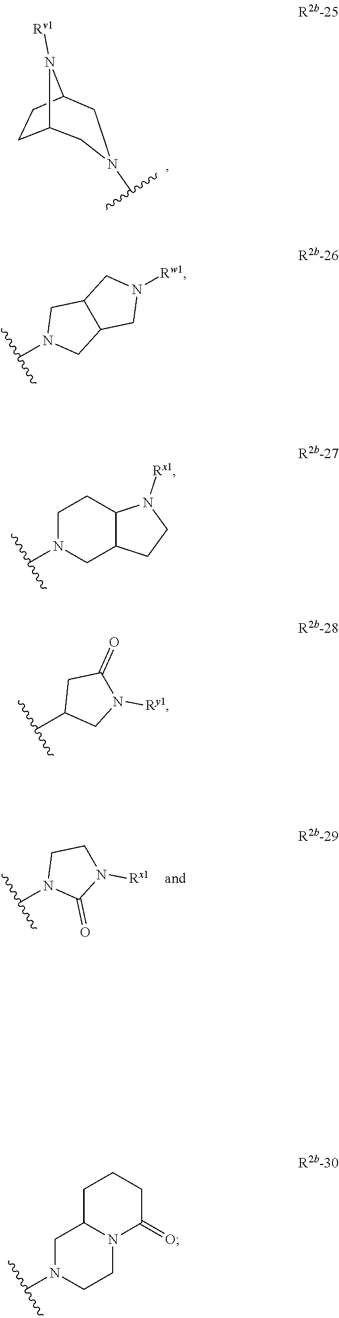

R^{a1} is selected from the group consisting of —N(R^{3a})C(═O)R^{4a}; —NR^{5a}R^{5b}; unsubstituted 4- to 10-membered heterocyclo; substituted 4- to 10-membered heterocyclo having one, two, or three substituents independently selected from the group consisting of hydroxy, —NR^{5c}R^{5d} C₁-C₄ alkyl, C₁-C₆ alkoxy, —C(R^{6a})(R^{6b})C(═O)NR^{5e}R^{5f}, C(═O)R^{4b}, (hydroxy)C₁-C₄ alkyl, and halo;
R^{a2} and R^{a3} are each hydrogen; or
R^{a2} and R^{a3} taken together with the carbon atom to which they are attached form a C(═O) group;
R^{a4} is selected from the group consisting of hydrogen, halo, and hydroxy;
R^{a5} is selected from the group consisting of hydrogen, C₁-C₄ alkyl, and C₃-C₆ cycloalkyl;
R^{b1} is selected from the group consisting of hydrogen, C₁-C₄ alkyl, and C₃-C₆ cycloalkyl;
R^{c1} is selected from the group consisting of hydrogen, C₁-C₄ alkyl, C₃-C₆ cycloalkyl, and —C(═O)R^{4c};
R^{c2} and R^{c3} are each independently selected from the group consisting of hydrogen, C₁-C₄ alkyl, and C₁-C₄ haloalkyl; or
R^{c2} and R^{c3} taken together with the carbon atom to which they are attached form a C(═O) group;
R^{c4} is selected from the group consisting of hydrogen and C₁-C₄ alkyl;
m is 1 or 2;
R^{d1} is selected from the group consisting of hydrogen, C₁-C₄ alkyl, and —C(═O)R^{4c},
R^{d2} and R^{d3} are each independently selected from the group consisting of hydrogen and fluoro;
R^{e1} is selected from the group consisting of hydrogen, C₁-C₄ alkyl, C₃-C₆ cycloalkyl, and —C(═O)R^{4c};
R¹ is selected from the group consisting of hydrogen, C₁-C₄ alkyl, C₃-C₆ cycloalkyl, and —C(═O)R^{4c},
R^{g1} is selected from the group consisting of hydrogen, C₁-C₄ alkyl, —C(═O)R^{4c}, C₁-C₄ haloalkyl, (C₁-C₄ alkoxy)C₁-C₄ alkyl
R^{h1} is selected from the group consisting of hydrogen, C₁-C₄ alkyl, C₃-C₆ cycloalkyl, and —C(═O)R^{4c};
R^{h2} is selected from the group consisting of hydrogen and C₁-C₄ alkyl;
R^{h3} and R^{h4} are each independently selected from the group consisting of hydrogen and C₁-C₄ alkyl; or
R^{h3} and R^{h4} taken together with the carbon atom to which they are attached form a C(═O) group;
R¹ is selected from the group consisting of hydrogen, C₁-C₄ alkyl, C₃-C₆ cycloalkyl, (hydroxy)C₁-C₄ alkyl, —N(R^{3a})C(═O)R^{4a}, and (amino)C₁-C₄ alkyl;
Z¹ is selected from the group consisting of —CH₂— and —O—;
R^{j1} is selected from the group consisting of hydrogen, C₁-C₄ alkyl, C₃-C₆ cycloalkyl, and —C(═O)R^{4c};
R^{k1} is selected from the group consisting of C₁-C₄ alkyl, unsubstituted 4- to 14-membered heterocyclo and —NR^{5a}R^{5b};
R^{k2} is selected from the group consisting of hydrogen, hydroxy, and C₁-C₄ alkyl;
r is 0, 1, or 2;
Z² is selected from the group consisting of —O— and —N(R^{m3})—;
R^{m3} is selected from the group consisting of hydrogen, C₁-C₄ alkyl, and C₁-C₄ haloalkyl;
Rⁿ³ is selected from the group consisting of hydrogen, C₁-C₄ alkyl, and —C(═O)R^{4c};
R^{o1} is selected from the group consisting of hydroxy, (hydroxy)C₁-C₄ alkyl, (amino)C₁-C₄ alkyl, (C₁-C₄ alkoxy)C₁-C₄ alkyl, C₁-C₄ alkoxy, —NR^{5a}R^{5b}, unsubstituted 4- to 14-membered heterocyclo, substituted 4- to 14-membered heterocyclo having one, two, or three substituents independently selected from the group consisting of halo, C₁-C₄ alkyl and C₁-C₄ alkoxy;
R^{o2} is selected from the group consisting of hydrogen, C₁-C₄ alkyl, and (C₁-C₄ alkoxy)C₁-C₄ alkyl
R^{o3} is selected from the group consisting of hydrogen, fluoro, and C₁-C₄ alkyl;
R^{p1} is selected from the group consisting of hydrogen, C₁-C₄ alkyl, and —C(═O)R^{4c};
Z³ is selected from the group consisting of —O— and —N(R^{g1})—;
R^{q1} is selected from the group consisting of hydrogen and C₁-C₄ alkyl;
R¹ is selected from the group consisting of hydrogen, C₁-C₄ alkyl, and —C(═O)R^{4c};
R^{s1} is selected from the group consisting of hydrogen, C₁-C₄ alkyl, and —C(═O)R^{4c};
R^{t1} is selected from the group consisting of hydrogen, C₁-C₄ alkyl, and —C(═O)R^{4c};
R^{u1} is selected from the group consisting of hydrogen, C₁-C₄ alkyl, and —C(═O)R^{4c};
R^{v1} is selected from the group consisting of hydrogen, C₁-C₄ alkyl, and —C(═O)R^{4c};
R^{w1} is selected from the group consisting of hydrogen, C₁-C₄ alkyl, and —C(═O)R^{4c};
R^{x1} is selected from the group consisting of hydrogen, C₁-C₄ alkyl, and —C(═O)R^{4c};
R^{y1} is selected from the group consisting of hydrogen and C₁-C₄ alkyl; and
R^{z1} is selected from the group consisting of hydrogen and C₁-C₄ alkyl, or a pharmaceutically acceptable salt or solvate thereof.

In another embodiment, Compounds of the Disclosure are compounds having Formula III or Formula III-A, wherein:
R^{2b} is selected from the group consisting of:

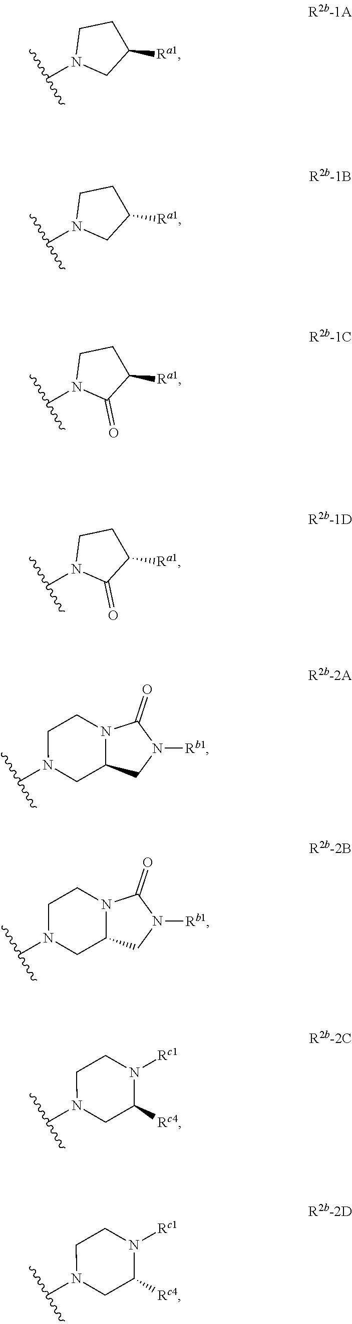

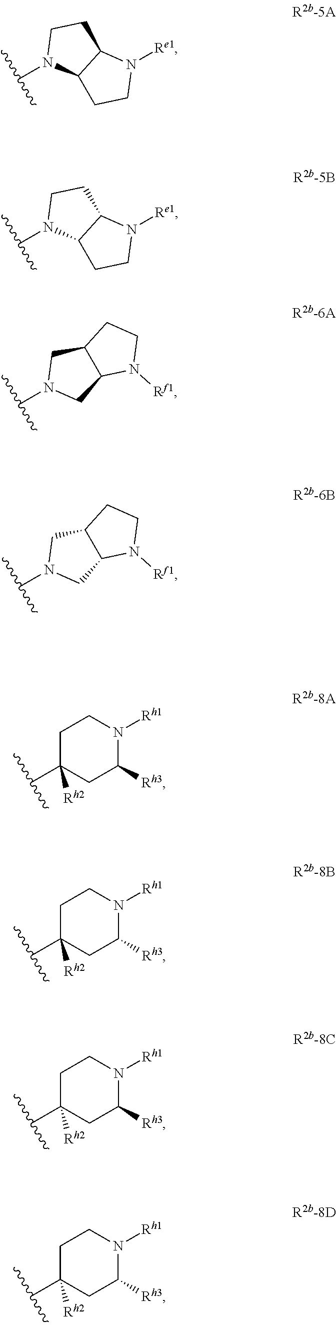

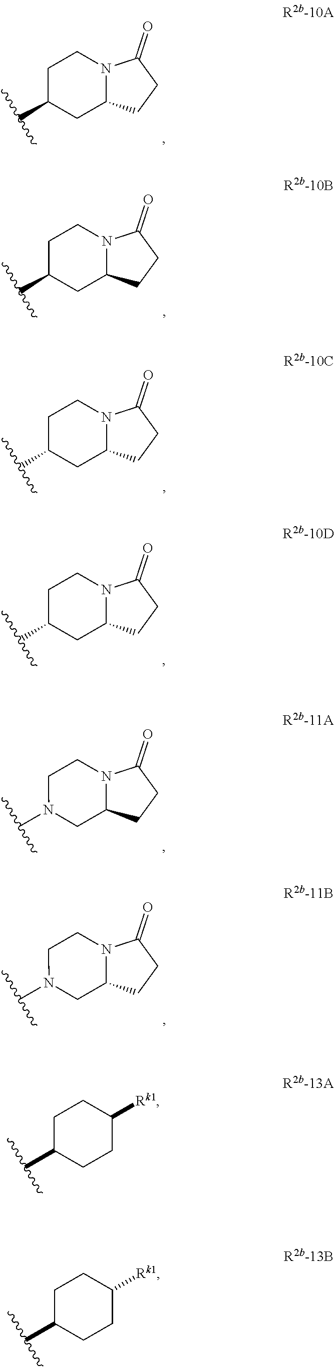

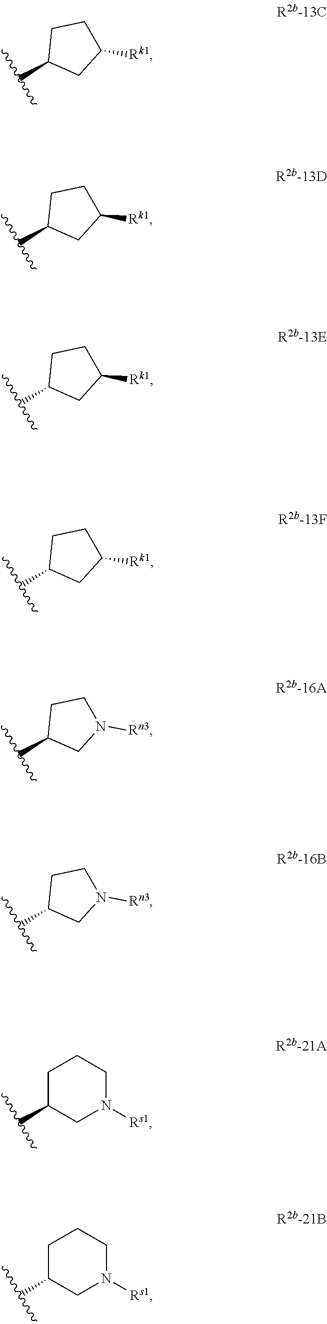

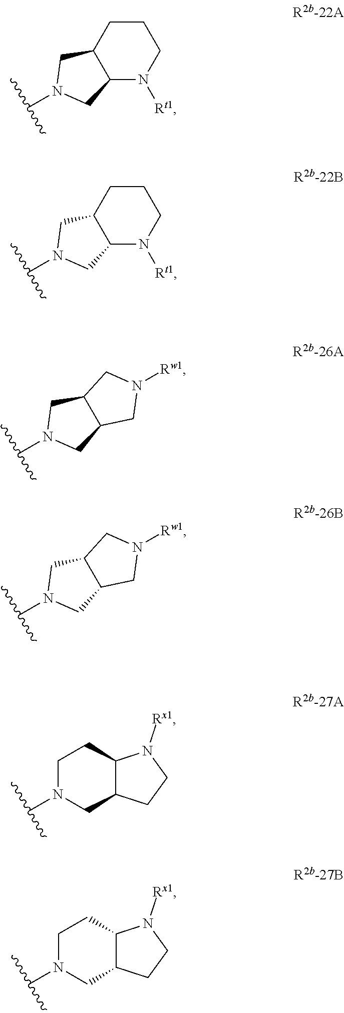

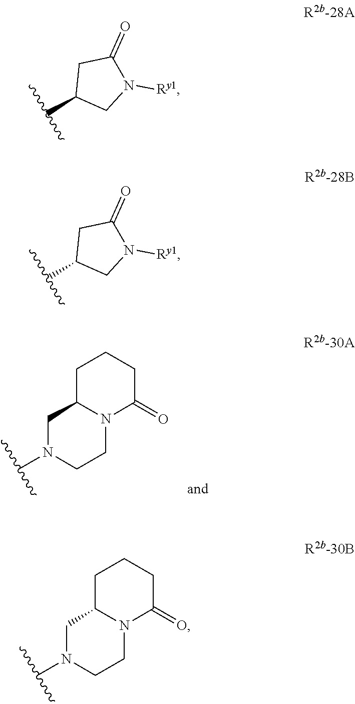

or a pharmaceutically acceptable salt or solvate thereof.

In another embodiment, Compounds of the Disclosure are compounds having Formula III or Formula III-A, wherein R^{2b} is R^{2b}-1, R^{2b}-1A, R^{2b}-1B, R^{2b}-1C, or R^{2b}-1D, or a pharmaceutically acceptable salt or solvate thereof. In another embodiment, R^{a1} is —N(R^{3a})C(═O)R^{4a}. In another embodiment, R^{a1} is —NR^{5a}R^{5b}. In another embodiment, R^{a1} is —NR^{5a}R^{5b} and R^{5a} and R^{5b} are independently selected from the group consisting of hydrogen and C₁-C₄ alkyl. In another embodiment, R^{a1} is optionally substituted 4- to 10-membered heterocyclo.

In another embodiment, Compounds of the Disclosure are compounds having Formula III or Formula III-A, wherein R^{2b} is R^{2b}-2, R^{2b}-2A, or R^{2b}-2B, or a pharmaceutically acceptable salt or solvate thereof. In another embodiment, R^{b1} is C₁-C₄ alkyl.

In another embodiment, Compounds of the Disclosure are compounds having Formula III or Formula III-A, wherein R^{2b} is R^{2b}-3, R^{2b}-3A, or R^{2b}-3B, or a pharmaceutically acceptable salt or solvate thereof. In another embodiment, R^{c1} is selected from the group consisting of C₁-C₄ alkyl, C₃-C₆ cycloalkyl, and —C(═O)R^{4c}. In another embodiment, R² and R^{c3} are each hydrogen. In another embodiment, R^{c2} and R^{c3} taken together with the carbon atom to which they are attached form a C(═O) group. In another embodiment, R^{c4} is hydrogen. In another embodiment, m is 1.

In another embodiment, Compounds of the Disclosure are compounds having Formula III or Formula III-A, wherein R^{2b} is R^{2b}-4, or a pharmaceutically acceptable salt or solvate thereof. In another embodiment, R^{d1} is C(═O)R^{4c}. In another embodiment, R^{d2} and R^{d3} are each hydrogen or fluoro.

In another embodiment, Compounds of the Disclosure are compounds having Formula III or Formula III-A, wherein R^{2b} is R^{2b}-5, R^{2b}-5A, or R^{2b}-5B, or a pharmaceutically acceptable salt or solvate thereof. In another embodiment, R^{e1} is —C(═O)R^{4c}.

In another embodiment, Compounds of the Disclosure are compounds having Formula III or Formula III-A, wherein R^{2b} is R^{2b}-6, R^{2b}-6A, or R^{2b}-6B, or a pharmaceutically acceptable salt or solvate thereof. In another embodiment, R^{f1} is C(═O)R^{4c}.

In another embodiment, Compounds of the Disclosure are compounds having Formula III or Formula III-A, wherein R^{2b} is R^{2b}-7, or a pharmaceutically acceptable salt or solvate thereof. In another embodiment, R^{g1} is C(═O)R^{4c}.

In another embodiment, Compounds of the Disclosure are compounds having Formula III or Formula III-A, wherein R^{2b} is R^{2b}-8, R^{2b}-8A, R^{2b}-8B, R^{2b}-8C, or R^{2b}-8D, or a pharmaceutically acceptable salt or solvate thereof. In another embodiment, R^{h1} is —C(═O)R^{4c}. In another embodiment, R^{h2} is selected from the group consisting of hydrogen and C₁-C₃ alkyl. In another embodiment, R^{h3} is hydrogen.

In another embodiment, Compounds of the Disclosure are compounds having Formula III or Formula III-A, wherein R^{2b} is R^{2b}-9, or a pharmaceutically acceptable salt or solvate thereof.

In another embodiment, Compounds of the Disclosure are compounds having Formula III or Formula III-A, wherein R^{2b} is selected from the group consisting of R^{2b}-10, R^{2b}-10A, R^{2b}-10B, R^{2b}-10C, and R^{2b}-10d, or a pharmaceutically acceptable salt or solvate thereof.

In another embodiment, Compounds of the Disclosure are compounds having Formula III or Formula III-A, wherein R^{2b} is selected from the group consisting of R^{2b}-11, R^{2b}-11A and R^{2b}-11B, or a pharmaceutically acceptable salt or solvate thereof.

In another embodiment, Compounds of the Disclosure are compounds having Formula III or Formula III-A, wherein R^{2b} is R^{2b}-12, or a pharmaceutically acceptable salt or solvate thereof. In another embodiment, R^{j1} is —C(═O)R^{4c}.

In another embodiment, Compounds of the Disclosure are compounds having Formula III or Formula III-A, wherein R^{2b} is selected from the group consisting of R^{2b}-13, R^{2b}-13A, R^{2b}-13B, R^{2b}-13C, R^{2b}-13D, R^{2b}-13E, and R^{2b}-13F, or a pharmaceutically acceptable salt or solvate thereof.

In another embodiment, Compounds of the Disclosure are compounds having Formula III or Formula III-A, wherein R^{2b} is R^{2b}-14, or a pharmaceutically acceptable salt or solvate thereof.

In another embodiment, Compounds of the Disclosure are compounds having Formula III or Formula III-A, wherein R^{2b} is R^{2b}-15, or a pharmaceutically acceptable salt or solvate thereof.

In another embodiment, Compounds of the Disclosure are compounds having Formula III or Formula III-A, wherein R^{2b} is selected from the group consisting of R^{2b}16, R^{2b}-16A and R^{2b}-16B, or a pharmaceutically acceptable salt or solvate thereof. In another embodiment, Rⁿ³ is —C(═O)R^{4c}.

In another embodiment, Compounds of the Disclosure are compounds having Formula III or Formula III-A, wherein R^{2b} is R^{2b}-17, or a pharmaceutically acceptable salt or solvate thereof.

In another embodiment, Compounds of the Disclosure are compounds having Formula III or Formula III-A, wherein R^{2b} is R^{2b}-18, or a pharmaceutically acceptable salt or solvate thereof.

In another embodiment, Compounds of the Disclosure are compounds having Formula III or Formula III-A, wherein R^{2b} is R^{2b}-19, or a pharmaceutically acceptable salt or solvate thereof.

In another embodiment, Compounds of the Disclosure are compounds having Formula III or Formula III-A, wherein R^{2b} is R^{2b}-20, or a pharmaceutically acceptable salt or solvate thereof.

In another embodiment, Compounds of the Disclosure are compounds having Formula III or Formula III-A, wherein R^{2b} is selected from the group consisting of R^{2b}21, R^{2b}-21A and R^{2b}-21B, or a pharmaceutically acceptable salt or solvate thereof.

In another embodiment, Compounds of the Disclosure are compounds having Formula III, wherein R^{2b} is selected from the group consisting of R^{2b}-22, R^{2b}-22A and R^{2b}-22B, or a pharmaceutically acceptable salt or solvate thereof.

In another embodiment, Compounds of the Disclosure are compounds having Formula III or Formula III-A, wherein R^{2b} is R^{2b}-23, or a pharmaceutically acceptable salt or solvate thereof.

In another embodiment, Compounds of the Disclosure are compounds having Formula III or Formula III-A, wherein R^{2b} is R^{2b}-24, or a pharmaceutically acceptable salt or solvate thereof.

In another embodiment, Compounds of the Disclosure are compounds having Formula III or Formula III-A, wherein R^{2b} is R^{2b}-25, or a pharmaceutically acceptable salt or solvate thereof.

In another embodiment, Compounds of the Disclosure are compounds having Formula III or Formula III-A, wherein R^{2b} is selected from the group consisting of R^{2b}-26, R^{2b}-26A and R^{2b}-26B, or a pharmaceutically acceptable salt or solvate thereof.

In another embodiment, Compounds of the Disclosure are compounds having Formula III or Formula III-A, wherein R^{2b} is selected from the group consisting of R^{2b}-27, R^{2b}-27A and R^{2b}-27B, or a pharmaceutically acceptable salt or solvate thereof.

In another embodiment, Compounds of the Disclosure are compounds having Formula III or Formula III-A, wherein R^{2b} is selected from the group consisting of R^{2b}28, R^{2b}-28A and R^{2b}-28B, or a pharmaceutically acceptable salt or solvate thereof.

In another embodiment, Compounds of the Disclosure are compounds having Formula III or Formula III-A, wherein R^{2b} is R^{2b}-29, or a pharmaceutically acceptable salt or solvate thereof.

In another embodiment, Compounds of the Disclosure are compounds having Formula III or Formula III-A, wherein R^{2b} is R^{2b}-30, R^{2b}-30A, or R^{2b}-30B, or a pharmaceutically acceptable salt or solvate thereof.

In another embodiment, Compounds of the Disclosure are compounds having Formula III or Formula III-A, wherein R^{2b} is any one or more of the R^{11a} groups provided in connection with Formula IV, see below, or a pharmaceutically acceptable salt or solvate thereof.

In another embodiment, Compounds of the Disclosure are compounds having Formula III or Formula III-A, wherein R⁴, is C₁-C₄ alkyl, or a pharmaceutically acceptable salt or solvate thereof.

In another embodiment, Compounds of the Disclosure are compounds having Formula III or Formula III-A, wherein R^{2d} is selected from the group consisting of hydrogen, fluoro, and chloro, or a pharmaceutically acceptable salt or solvate thereof.

In another embodiment, Compounds of the Disclosure are compounds having Formula III or Formula III-A, wherein R^{2d} is hydrogen, or a pharmaceutically acceptable salt or solvate thereof.

In another embodiment, Compounds of the Disclosure are compounds having Formula III in any of the above described embodiments, wherein A¹ and A² are —C(H)═; R^{2e} is hydrogen; and R^{2d} is selected from the group consisting of hydrogen and halogen, or a pharmaceutically acceptable salt or solvate thereof.

In another embodiment, Compounds of the Disclosure are compounds having Formula III or Formula III-A, wherein R^{2d} is fluoro, or a pharmaceutically acceptable salt or solvate thereof.

In another embodiment, Compounds of the Disclosure are compounds having Formula IV:

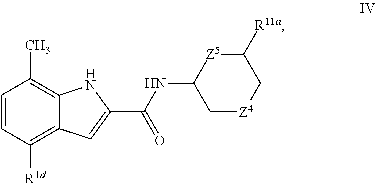

wherein:
Z⁴ is selected from the group consisting of —O—, —C(R^{28a})(R^{28b})—, and —N(R²³)—; or Z⁴ is absent;
Z⁵ is selected from the group consisting of —CH₂— and —CH₂CH₂—;
R^{11a} is selected from the group consisting of optionally substituted alkyl, optionally substituted heterocyclo, optionally substituted heteroaryl, and —N(R^{12b})C(═O)R^{13c};
R^{12b} is selected from the group consisting of hydrogen, alkyl, cycloalkyl, and heterocyclo;
R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, amino, (amino)alkyl, (C₃-C₆ cycloalkyl)oxy, and (4- to 8-membered heterocyclo)oxy;
R²³ is selected from the group consisting of hydrogen and C₁-C₄ alkyl;
R^{28a} and R^{28b} are independently selected from the group consisting of hydrogen, alkyl, and halo; and
R^{1d} is as defined in connection with Formula I, or a pharmaceutically acceptable salt or solvate thereof.

In another embodiment, Compounds of the Disclosure are compounds having Formula IV, wherein Z⁴ is selected from the group consisting of —O— and —CH₂—; or Z⁴ is absent, or a pharmaceutically acceptable salt or solvate thereof.

In another embodiment, Compounds of the Disclosure are compounds having Formula IV, wherein:
Z⁴ is selected from the group consisting of —O— and —CH₂—; or Z⁴ is absent;
Z⁵ is selected from the group consisting of —CH₂— and —CH₂CH₂—;
R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, and
R^{1d} is as defined in connection with Formula I, or a pharmaceutically acceptable salt or solvate thereof.

In another embodiment, Compounds of the Disclosure are compounds having Formula IV-A:

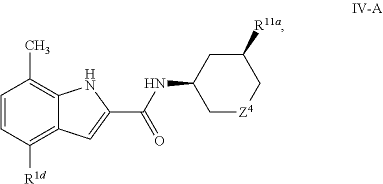

or a pharmaceutically acceptable salt or solvate thereof, wherein R^{1d}, R^{11a}, and Z⁴ are as defined in connection with Formula IV.

In another embodiment, Compounds of the Disclosure are compounds having Formula IV-B:

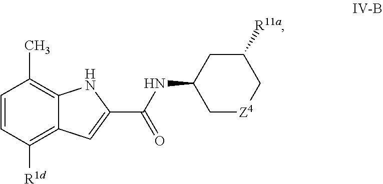

or a pharmaceutically acceptable salt or solvate thereof, wherein R^{1d}, R^{11a}, and Z⁴ are as defined in connection with Formula IV.

In another embodiment, Compounds of the Disclosure are compounds having Formula IV-C:

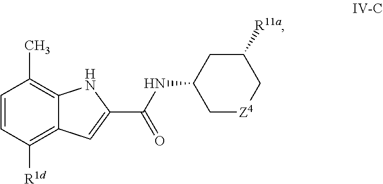

or a pharmaceutically acceptable salt or solvate thereof, wherein R^{1d}, R^{11a}, and Z⁴ are as defined in connection with Formula IV.

In another embodiment, Compounds of the Disclosure are compounds having Formula IV-D:

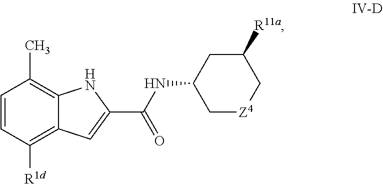

or a pharmaceutically acceptable salt or solvate thereof, wherein R^{1d}, R^{11a}, and Z⁴ are as defined in connection with Formula IV.

In another embodiment, Compounds of the Disclosure are compounds having any one of Formulae IV, IV-A, IV-B, IV-C, or IV-D, wherein:
R^{11a} is selected from the group consisting of: (A) unsubstituted 4- to 14-membered heterocyclo; (B) substituted 4- to 14-membered heterocyclo having one, two or three substituents independently selected from the group consisting of —N(R^{12a})C(═O)R^{13a}; —C(═O)R^{13b}; C₁-C₄ alkyl; (C₁-C₄ alkoxy)C₁-C₄ alkyl; (hydroxy)C₁-C₄ alkyl; C₁-C₄ haloalkyl; amino; hydroxy; —N(R^{12a})S(═O)₂R²⁴; —S(═O)₂R²⁴; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; (C) unsubstituted 5- to 10-membered heteroaryl; (D) substituted 5- or 6-membered heteroaryl having one, two, three, or four substituents independently selected from the group consisting of halo, C₁-C₄ alkyl, and (amino)alkyl; (E) C₁-C₆ alkyl; and (F) —N(R^{12b})C(═O)R^{13c};
R^{12a} and R^{12b} are each independently selected from the group consisting of hydrogen, C₁-C₄ alkyl, (C₁-C₄ alkoxy)C₁-C₄ alkyl, and (hydroxy)C₁-C₄ alkyl;
R^{13a}, R^{13b}, and R^{13c} are each independently selected from the group consisting of C₁-C₆ alkyl; C₁-C₆ haloalkyl; unsubstituted C₃-C₆ cycloalkyl; C₁-C₆ alkoxy; (C₁-C₄ alkoxy)C₁-C₄ alkyl; (hydroxy)C₁-C₄ alkyl; (cyano)alkyl; unsubstituted C₆-C₁₀ aryl; substituted C₆-C₁₀ aryl, having one, two, three, or four substituents independently selected from the group consisting of halo, amino, hydroxy, and C₁-C₄ alkyl; unsubstituted 5- or 6-membered heteroaryl; substituted 5- or 6-membered heteroaryl having one, two, three, or four substituents independently selected from the group consisting of halo, amino, hydroxy, and C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; amino; (amino)alkyl; (C₃-C₆ cycloalkyl)oxy; and (4- to 8-membered heterocyclo)oxy; and

R²⁴ is selected from the group consisting of C₁-C₄ alkyl and (hydroxy)C₁-C₄ alkyl.

In another embodiment, Compounds of the Disclosure are compounds having Formula IV, IV-A, IV-B, IV-C, or IV-D, wherein Z⁴ is —C(R^{28a})(R^{28b})—; and R^{28a} and R^{28b} are independently selected from the group consisting of hydrogen, C₁-C₄ alkyl, and fluoro, or a pharmaceutically acceptable salt or solvate thereof.

In another embodiment, Compounds of the Disclosure are compounds having Formula IV, IV-A, IV-B, IV-C, or IV-D, wherein Z⁴ is —C(R^{28a})(R^{28b})—; R^{28a} is hydrogen; and R^{28b} is selected from the group consisting of C₁-C₄ alkyl and fluoro, or a pharmaceutically acceptable salt or solvate thereof.

In another embodiment, Compounds of the Disclosure are compounds having Formula IV, IV-A, IV-B, IV-C, or IV-D, wherein Z⁴ is —C(R^{28a})(R^{28b})—; and R^{28a} and R^{28b} are independently C₁-C₄ alkyl, or a pharmaceutically acceptable salt or solvate thereof.

In another embodiment, Compounds of the Disclosure are compounds having Formula IV, IV-A, IV-B, IV-C, or IV-D, wherein Z⁴ is selected from the group consisting of —O—, —CH₂—, and —N(R²³), or Z⁴ is absent, or a pharmaceutically acceptable salt or solvate thereof.

In another embodiment, Compounds of the Disclosure are compounds having any one of Formulae IV, IV-A, IV-B, IV-C, or IV-D, wherein Z⁴ is —CH₂—, or a pharmaceutically acceptable salt or solvate thereof.

In another embodiment, Compounds of the Disclosure are compounds having any one of Formulae IV, IV-A, IV-B, IV-C, or IV-D, wherein R^{11a} is an optionally substituted 3- to 10-membered heterocycle linked to the rest of the molecule through a nitrogen atom, e.g., R^{11a} is

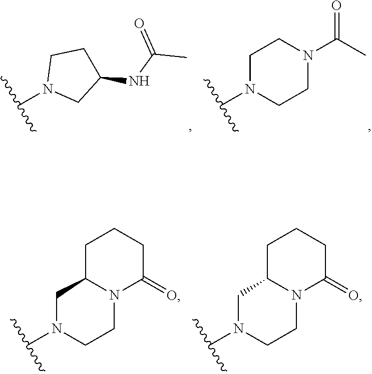

and the like.

In another embodiment, Compounds of the Disclosure are compounds having any one of Formulae IV, IV-A, IV-B, IV-C, or IV-D, wherein R^{11a} is a substituted 4- to 14-membered heterocyclo selected from the group consisting of:

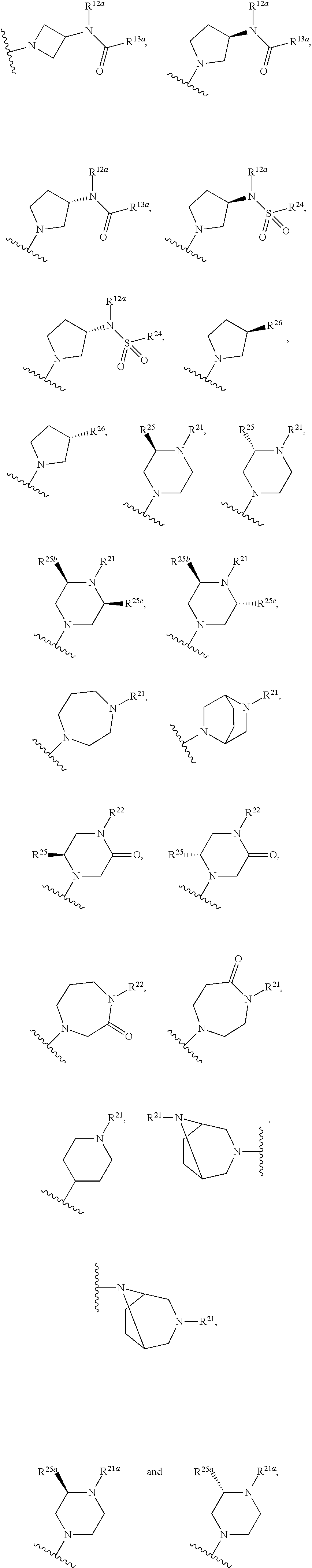

R^{12a} is selected from the group consisting of hydrogen, C₁-C₃ alkyl, (C₁-C₄ alkoxy)C₁-C₄ alkyl; and (hydroxy)C₁-C₄ alkyl;
R^{13a} is selected from the group consisting of C₁-C₄ alkyl; amino; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; (C₁-C₄ alkoxy)C₁-C₄ alkyl; (hydroxy)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;
R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;
R²¹ is selected from the group consisting of hydrogen, —C(═O)R^{13b}, C₁-C₄ alkyl, C₁-C₄ haloalkyl, unsubstituted 4- to 14-membered heterocyclo, and —S(═O)₂R²⁴;
R²² is C₁-C₄ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;
R²⁴ is selected from the group consisting of C₁-C₄ alkyl and (hydroxy)C₁-C₄ alkyl;
R²⁵ is selected from the group consisting of hydrogen, C₁-C₄ alkyl, and C₁-C₄ haloalkyl;
R^{25b} and R^{25c} are independently selected from the group consisting of C₁-C₄ alkyl and C₁-C₄ haloalkyl;
R²⁶ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl; and
R^{21a} and R^{25a} taken together with the atoms to which they are attached form an optionally substituted 4- to 8-membered heterocyclo, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of:

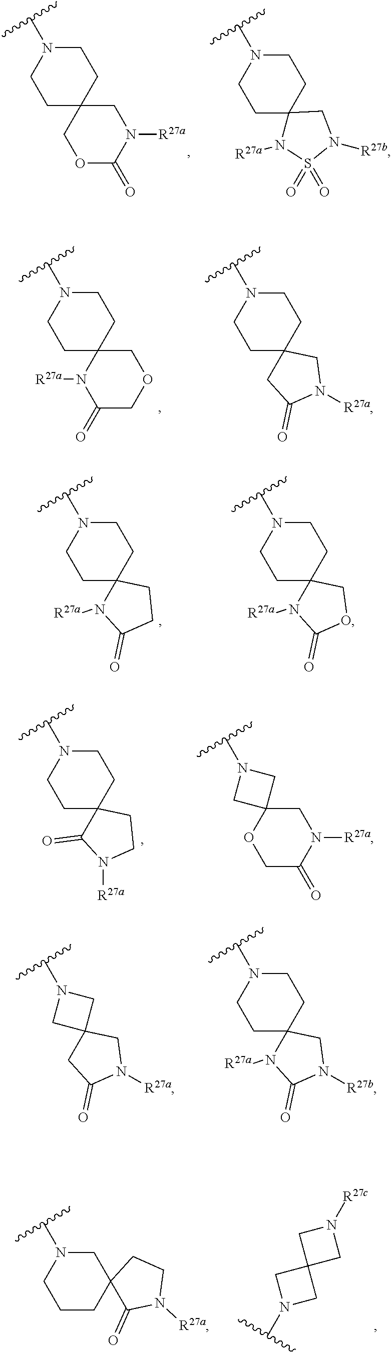

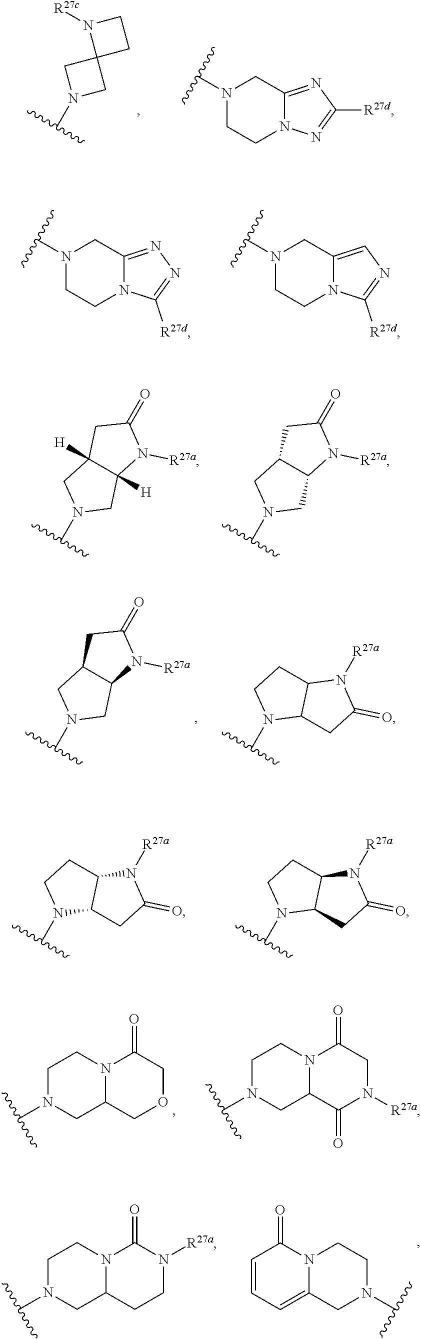

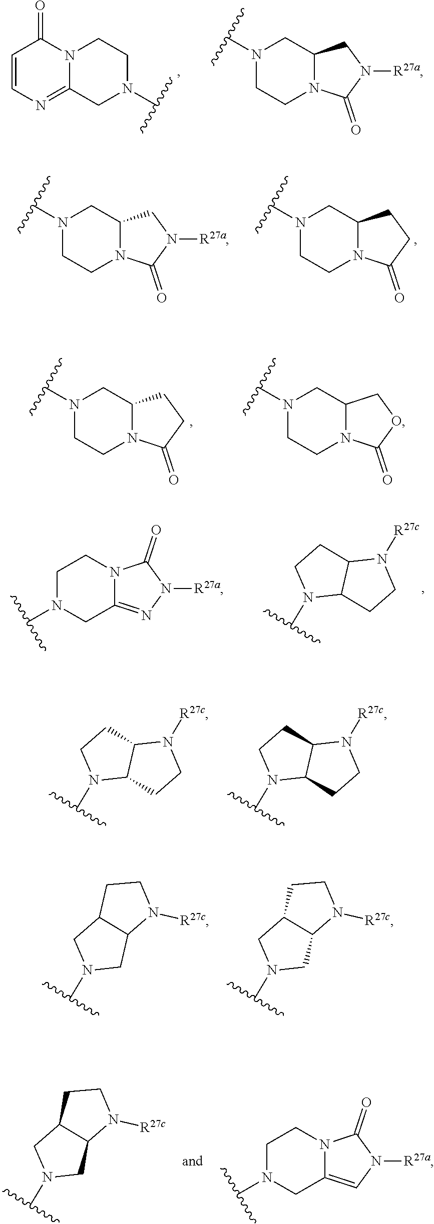

wherein:
R^{27a} and R^{27b} are each independently selected from the group consisting of hydrogen, C₁-C₄ alkyl, C₁-C₄ haloalkyl, (C₁-C₄ alkoxy)C₁-C₄ alkyl; and (hydroxy)C₁-C₄ alkyl;
R^{27c} is selected from the group consisting of hydrogen; —C(═O)R^{13b}; C₁-C₄ alkyl; C₁-C₄ haloalkyl; unsubstituted 4- to 14-membered heterocyclo; and substituted 4- to 14-membered heterocyclo having one or two substituents independently selected from the group consisting of amino, hydroxy, and C₁-C₄ alkyl; and —S(═O)₂R²⁴;
R^{27d} is selected from the group consisting of hydrogen; C₁-C₄ alkyl; and C₁-C₄ haloalkyl;
R^{13b} is selected from the group consisting of C₁-C₄ alkyl; aminoC₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy; and
R²⁴ is selected from the group consisting of C₁-C₄ alkyl and (hydroxy)C₁-C₄ alkyl, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of

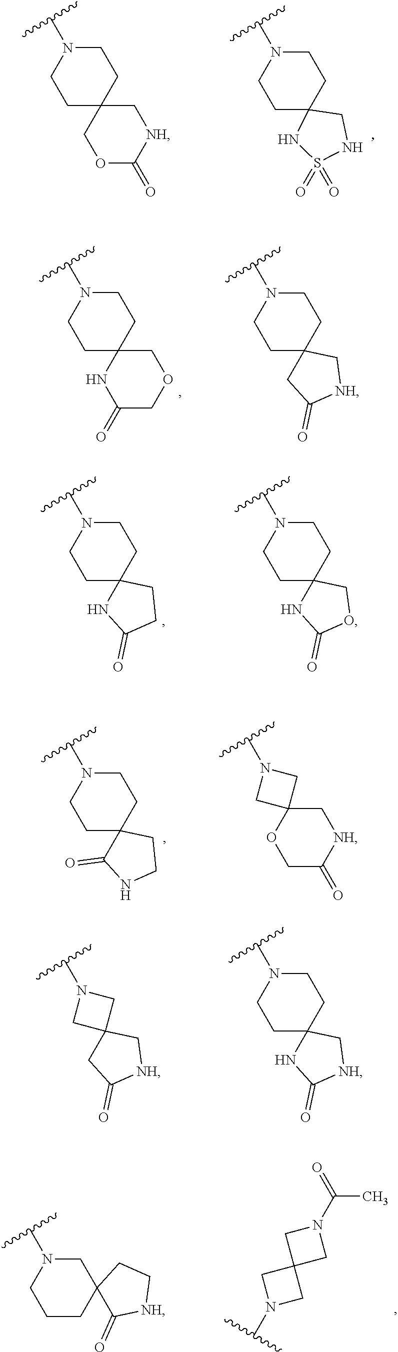

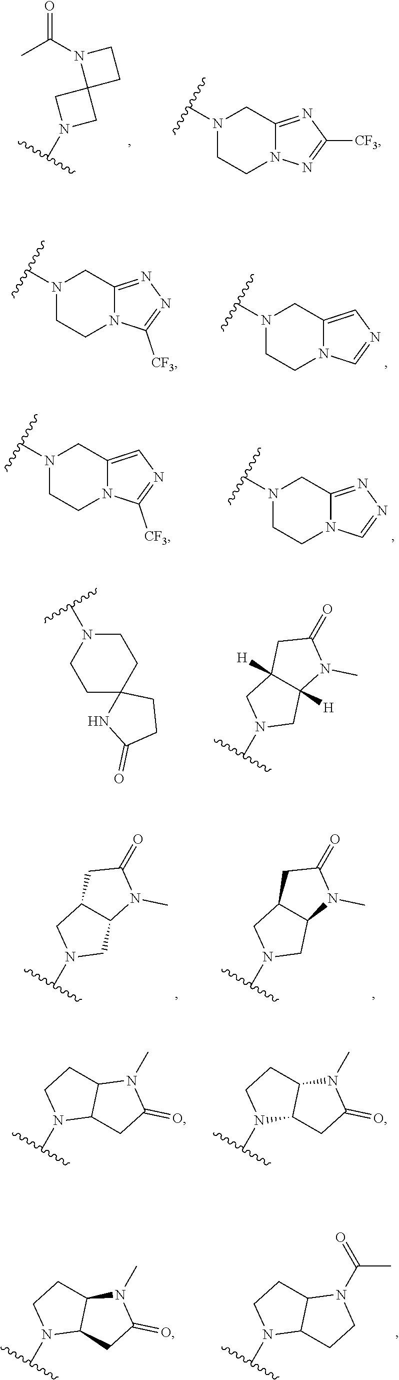

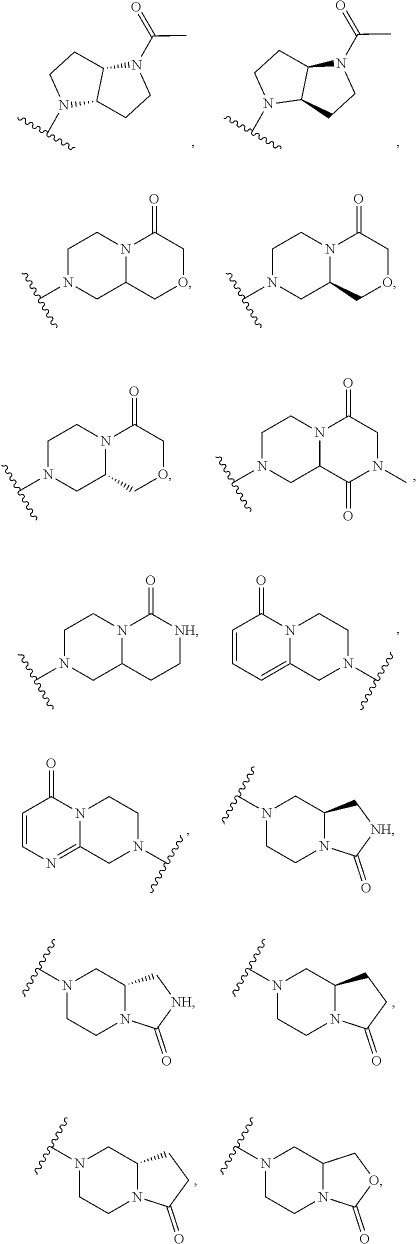

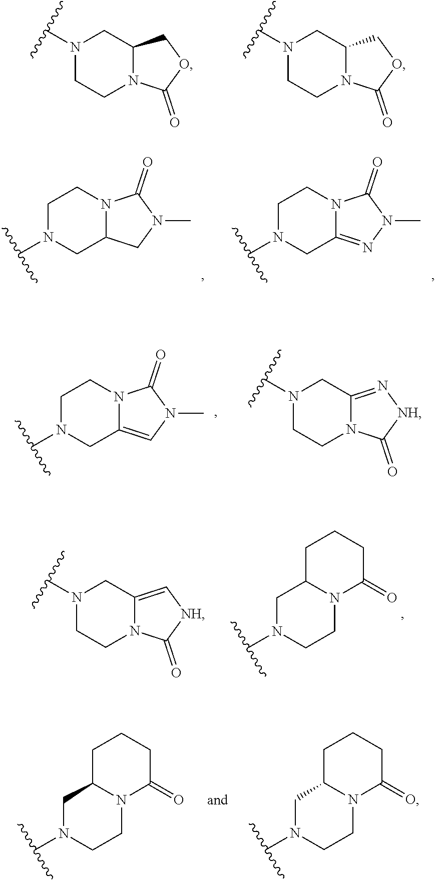

or a pharmaceutically acceptable salt or solvate thereof

In another embodiment, Compounds of the Disclosure are compounds having any one of Formulae IV, IV-A, IV-B, IV-C, or IV-D, wherein R^{11a} is selected from the group consisting of unsubstituted 4- to 14-membered heterocyclo; substituted 4- to 14-membered heterocyclo having one or two substituents independently selected from the group consisting of —N(R^{12a})C(═O)R^{13a}, —C(═O)R^{13b}, and C₁-C₄ alkyl; unsubstituted 5- to 10-membered heteroaryl; and substituted 5- or 6-membered heteroaryl having one or two substituents independently selected from the group consisting of halo and C₁-C₄ alkyl, or a pharmaceutically acceptable salt or solvate thereof.

In another embodiment, Compounds of the Disclosure are compounds having any one of Formulae IV, IV-A, IV-B, IV-C, or IV-D, wherein R^{11a} is a substituted 4- to 14-membered heterocyclo is selected from the group consisting of:

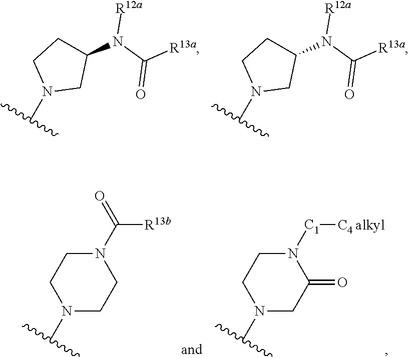

or a pharmaceutically acceptable salt or solvate thereof. In another embodiment, R^{12a} is selected from the group consisting of hydrogen and C₁-C₃ alkyl; R^{13a} is C₁-C₄ alkyl; and R^{13b} is C₁-C₄ alkyl, or a pharmaceutically acceptable salt or solvate thereof. In another embodiment, R^{12a} is selected from the group consisting of hydrogen and methyl; R^{13a} is methyl; and R^{13b} is methyl, or a pharmaceutically acceptable salt or solvate thereof.

In another embodiment, Compounds of the Disclosure are compounds having any one of Formulae IV, IV-A, IV-B, IV-C, or IV-D, wherein R^{11a} is any one or more of the R^{2b} groups provided in connection with Formula III, see above, or a pharmaceutically acceptable salt or solvate thereof.

In another embodiment, Compounds of the Disclosure are compounds having any one of Formulae IV, IV-A, IV-B, IV-C, or IV-D, wherein:
R^{11a} is selected from the group consisting of:

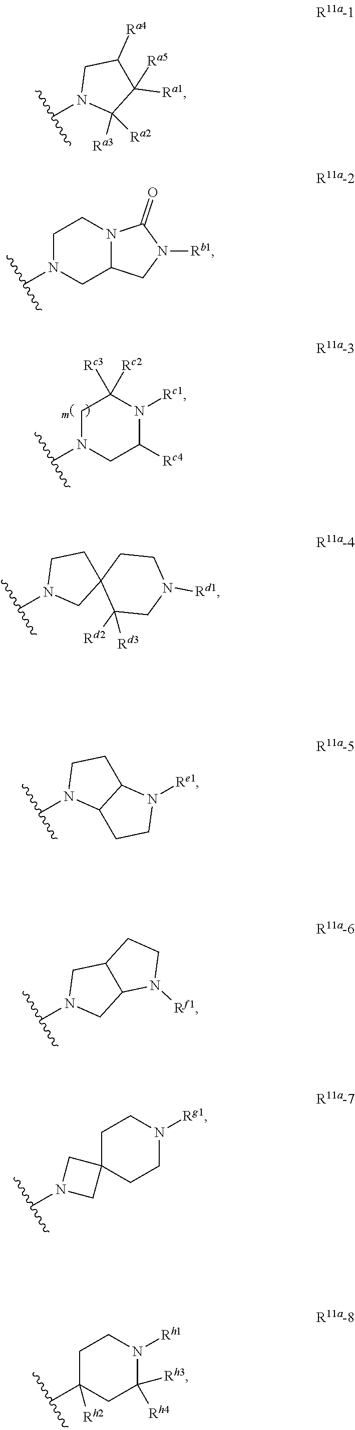

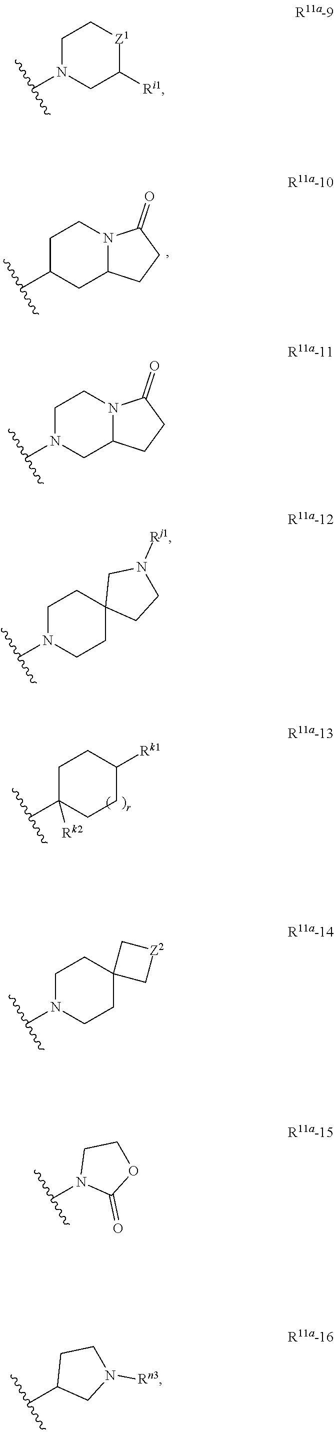

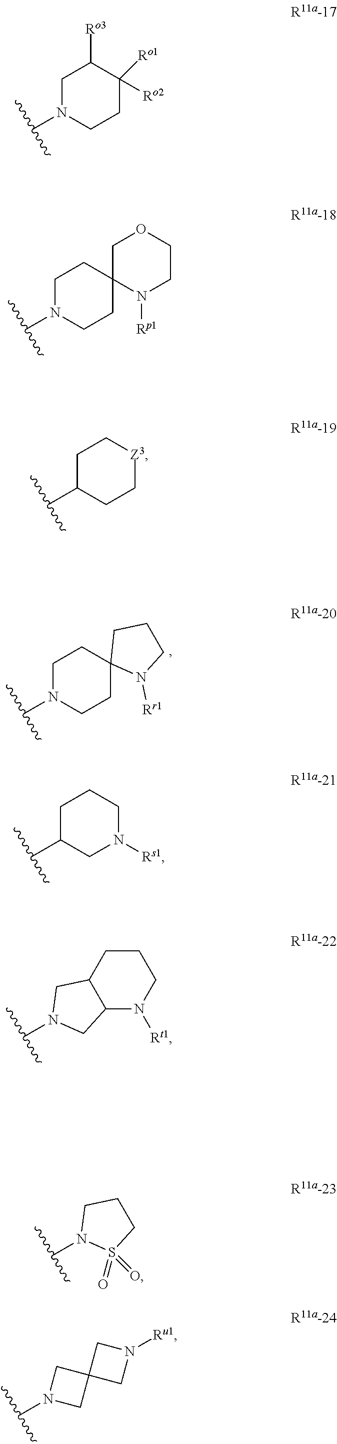

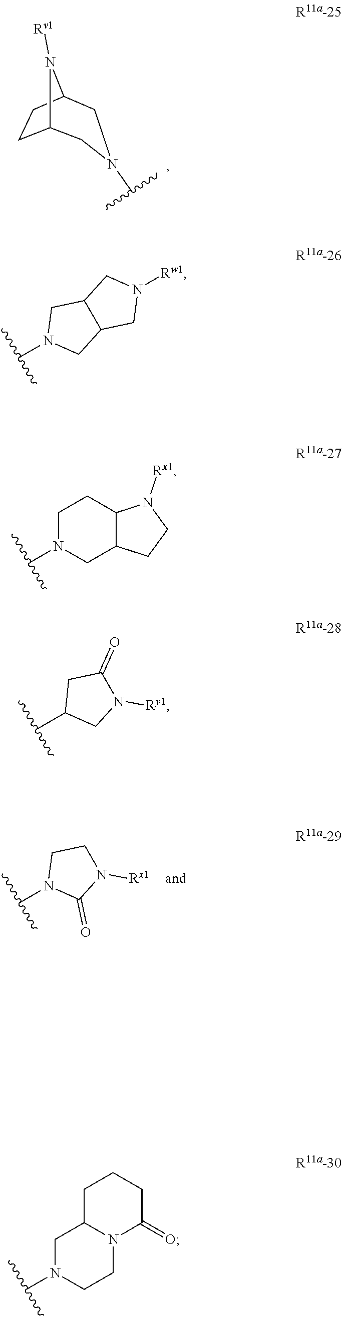

and R^{a1}, R^{a2}, R^{a3}, R^{a4}, R^{a5}, R^{b1}, R^{c1}, R^{c2}, R^{c3}, R^{c4}, m, R^{d1}, R^{d2}, R^{d3}, R^{e1}, R^{f1}, R^{g1}, R^{h1}, R^{h2}, R^{h3}, R^{h4}, Rⁱ¹, Z¹, R^{j1}, R^{k1}, R^{k2}, r, Z², Rⁿ³, R^{o1}, R^{o2}, R^{o3}, R^{p1}, Z³, R^{r1}, R^{s1}, R^{t1}, R^{u1}, R^{v1}, R^{w1}, R^{x1}, R^{y1}, and R^{z1} are as defined in connection with Formula III; or a pharmaceutically acceptable salt or solvate thereof.

In another embodiment, Compounds of the Disclosure are compounds having any one of Formulae IV, IV-A, IV-B, IV-C, or IV-D, wherein:
R^{11a} is selected from the group consisting of:

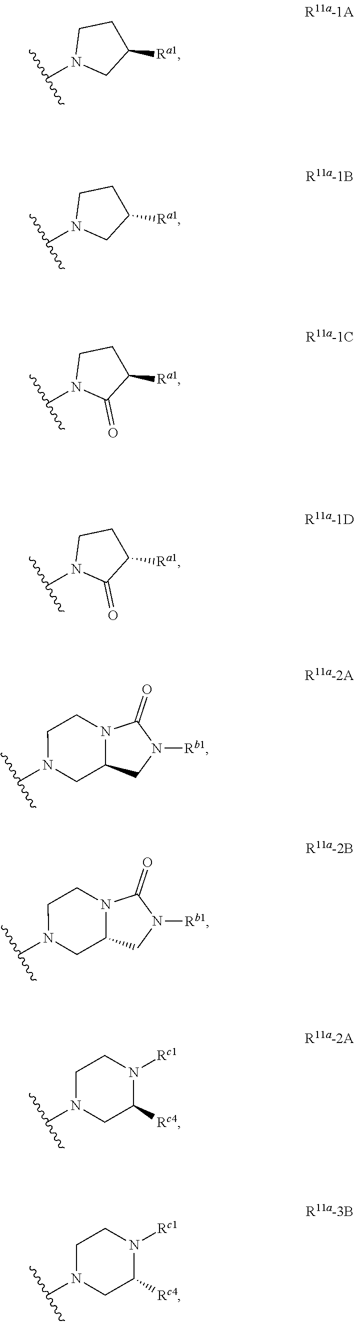

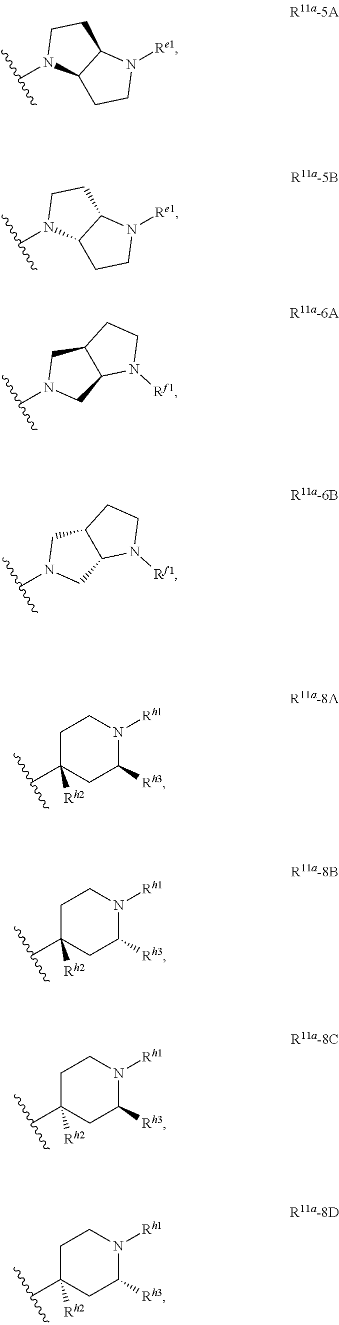

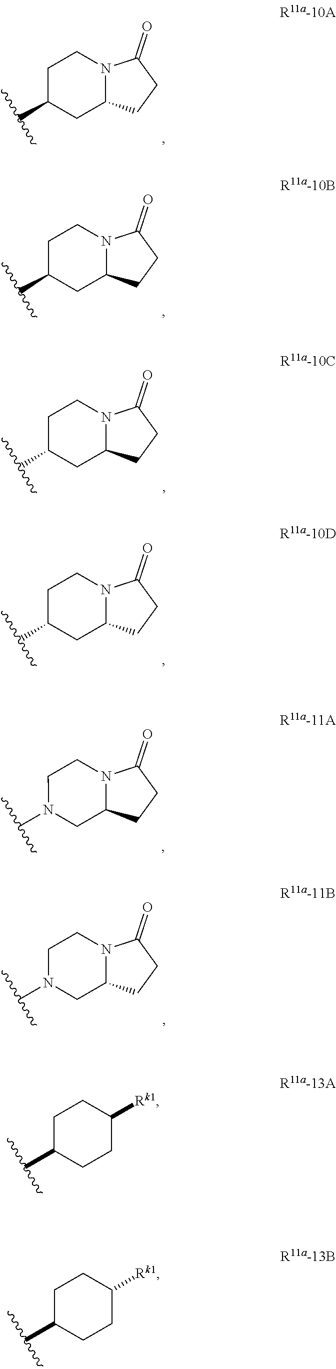

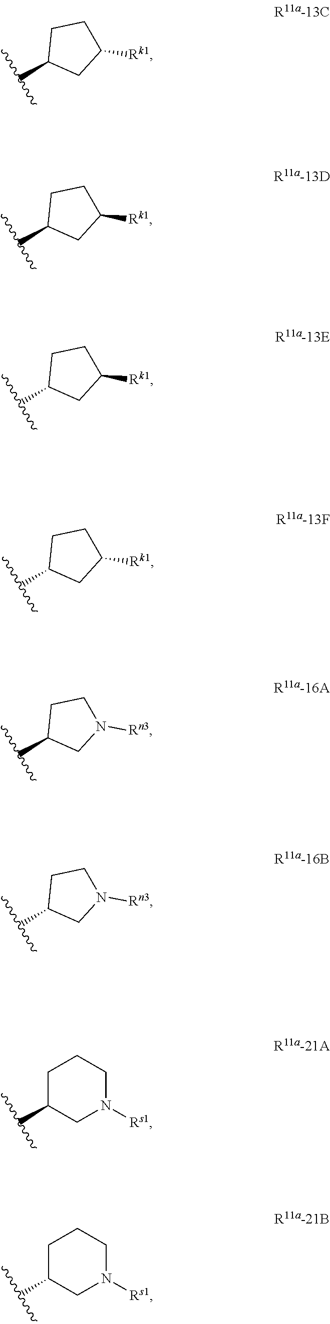

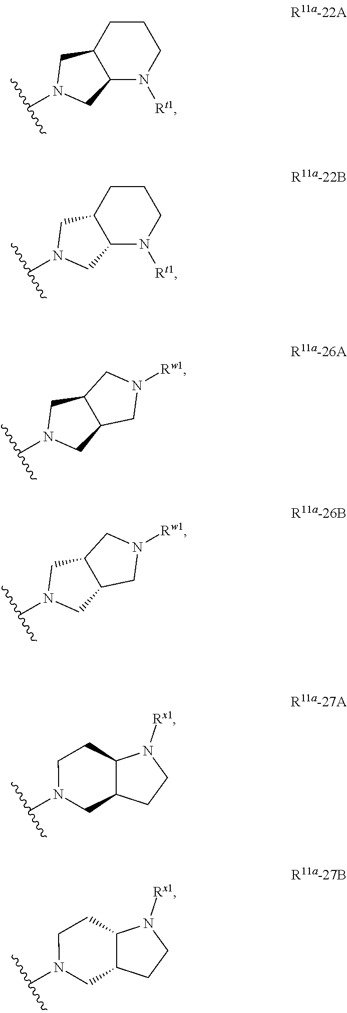

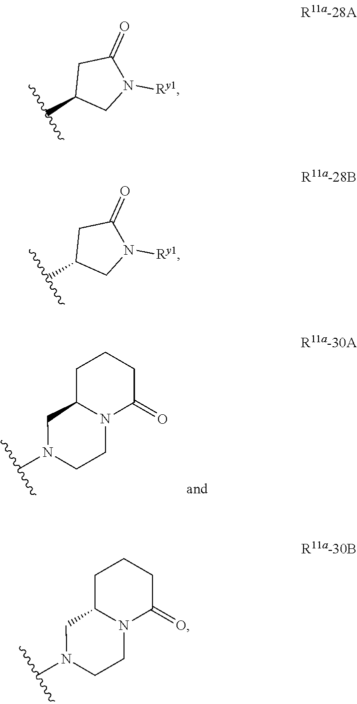

and R^{a1}, R^{a5}, R^{b1}, R^{e1}, R^{f1}, R^{h1}, R^{h2}, R^{h3}, R^{k1}, Rⁿ³, R^{s1}, R^{t1}, R^{w1}, R^{x1}, and R^{y1} are as defined in connection with Formula III; or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-1, R^{11a}-1A, R^{11a}-1B, R^{11a}-1C, or R^{11a}-1D, or a pharmaceutically acceptable salt or solvate thereof. In another embodiment, R^{a1} is —N(R^{3a})C(═O)R^{4a}. In another embodiment, R^{a1} is —NR^{5a}R^{5b}In another embodiment, R^{a1} is —NR^{5a}R^{5b} and R^{5a} and R^{5b} are independently selected from the group consisting of hydrogen and C₁-C₄ alkyl. In another embodiment, R^{a1} is optionally substituted 4- to 10-membered heterocyclo.

In another embodiment, Compounds of the Disclosure are compounds having any one of Formulae IV, IV-A, IV-B, IV-C, or IV-D, wherein R^{11a} is R^{11a}-2, R^{11a}-2A, or R^{11a}-2B, or a pharmaceutically acceptable salt or solvate thereof. In another embodiment, R^{b1} is C₁-C₄ alkyl.

In another embodiment, Compounds of the Disclosure are compounds having any one of Formulae IV, IV-A, IV-B, IV-C, or IV-D, wherein R^{11a} is R^{11a}-3, R^{11a}-3A, or R^{11a}-3B, or a pharmaceutically acceptable salt or solvate thereof. In another embodiment, R^{c1} is selected from the group consisting of C₁-C₄ alkyl, C₃-C₆ cycloalkyl, and —C(═O)R^{4c}. In another embodiment, R^{c2} and R^{c3} are each hydrogen. In another embodiment, R^{c2} and R^{c3} taken together with the carbon atom to which they are attached form a C(═O) group. In another embodiment, R^{c4} is hydrogen. In another embodiment, m is 1.

In another embodiment, Compounds of the Disclosure are compounds having any one of Formulae IV, IV-A, IV-B, IV-C, or IV-D, wherein R^{11a} is R^{11a}-4, or a pharmaceutically acceptable salt or solvate thereof. In another embodiment, R^{d1} is C(═O)R^{4c}. In another embodiment, R^{d2} and R^{d3} are each hydrogen or fluoro.

In another embodiment, Compounds of the Disclosure are compounds having any one of Formulae IV, IV-A, IV-B, IV-C, or IV-D, wherein R^{11a} is R^{11a}-5, R^{11a}-5A, or R^{11a}-5B, or a pharmaceutically acceptable salt or solvate thereof. In another embodiment, R^{e1} is —C(═O)R^{4c}.

In another embodiment, Compounds of the Disclosure are compounds having any one of Formulae IV, IV-A, IV-B, IV-C, or IV-D, wherein R^{11a} is R^{11a}-6, R^{11a}-6A, or R^{11a}-6B, or a pharmaceutically acceptable salt or solvate thereof. In another embodiment, R¹ is C(═O)R^{4c}.

In another embodiment, Compounds of the Disclosure are compounds having any one of Formulae IV, IV-A, IV-B, IV-C, or IV-D, wherein R^{11a} is R^{11a}-7, or a pharmaceutically acceptable salt or solvate thereof. In another embodiment, R^{g1} is C(═O)R^{4c}.

In another embodiment, Compounds of the Disclosure are compounds having any one of Formulae IV, IV-A, IV-B, IV-C, or IV-D, wherein R^{11a} is R^{11a}-8, R^{11a}-8A, R^{11a}-8B, R^{11a}-8C, or R^{11a}-8D, or a pharmaceutically acceptable salt or solvate thereof. In another embodiment, R^{h1} is —C(═O)R^{4c}. In another embodiment, R^{h2} is selected from the group consisting of hydrogen and C₁-C₃ alkyl. In another embodiment, R^{h3} is hydrogen.

In another embodiment, Compounds of the Disclosure are compounds any one of Formulae IV, IV-A, IV-B, IV-C, or IV-D, wherein R^{11a} is R^{11a}-9, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-10, R^{11a}-10A, R^{11a}-10B, R^{11a}-10C, and R^{11a}-10D, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-11, R^{11a}-11A and R^{11a}-11B, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-12, or a pharmaceutically acceptable salt or solvate thereof. In another embodiment, R^{j1} is —C(═O)R^{4c}.

In another embodiment, Compounds of the Disclosure are compounds having any one of Formulae IV, IV-A, IV-B, IV-C, or IV-D, wherein R^{11a} is selected from the group consisting of R^{11a}-13, R^{11a}-13A, R^{11a}-13B, R^{11a}-13C, R^{11a}-13D, R^{11a}-13E, and R^{11a}-13F, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-14, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-15, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-16, R^{11a}-16A and R^{11a}-16B, or a pharmaceutically acceptable salt or solvate thereof. In another embodiment, Rⁿ³ is —C(═O)R^{4c}.

In another embodiment, Compounds of the Disclosure are compounds having any one of Formulae IV, IV-A, IV-B, IV-C, or IV-D, wherein R^{11a} is R^{11a}-17, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-18, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-19, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-20, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-21, R^{11a}-21A and R^{11a}-21B, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-22, R^{11a}-22A and R^{11a}-22B, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-23, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-24, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-25, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-26, R^{11a}-26A and R^{11a}-26B, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-27, R^{11a}-27A and R^{11a}-27B, or a pharmaceutically acceptable salt or solvate thereof.

In another embodiment, Compounds of the Disclosure are compounds having any one of Formulae IV, IV-A, IV-B, IV-C, or IV-D, wherein R^{11a} is selected from the group consisting of R^{11a}-28, R^{11a}-28A and R^{11a}-28B, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-29, or a pharmaceutically acceptable salt or solvate thereof.

In another embodiment, Compounds of the Disclosure are compounds having any one of Formulae IV, IV-A, IV-B, IV-C, or IV-D, wherein R^{11a} is R^{11a}-30, R^{11a}-30A, or R^{11a}-30B, or a pharmaceutically acceptable salt or solvate thereof.

In another embodiment, Compounds of the Disclosure are compounds having any one of Formulae IV-A, IV-B, IV-C, or IV-D, wherein:
Z⁴ is —CH₂—;
R^{11a} is selected from the group consisting of:

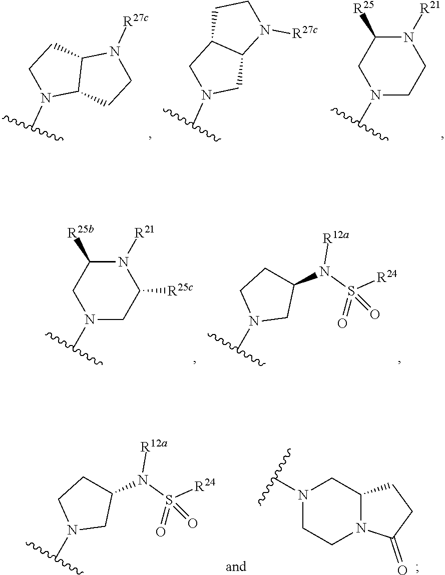

R^{12a} is selected from the group consisting of hydrogen and C₁-C₃ alkyl;
R²¹ is —C(═O)R^{13b};
R^{27c} is —C(═O)R^{13b};
R^{13b} is selected from the group consisting of C₁-C₄ alkyl and (hydroxy)C₁-C₄ alkyl;
R²⁴ is C₁-C₄ alkyl;
R²⁵ is selected from the group consisting of hydrogen, C₁-C₄ alkyl, and C₁-C₄ haloalkyl; and
R^{25b} and R^{25c} are independently selected from the group consisting of C₁-C₄ alkyl and C₁-C₄ haloalkyl, or a pharmaceutically acceptable salt or solvate thereof.

In another embodiment, Compounds of the Disclosure are compounds having any one of Formulae IV-A, IV-B, IV-C, or IV-D, wherein:
Z⁴ is —CH₂—; and
R^{11a} is selected from the group consisting of:

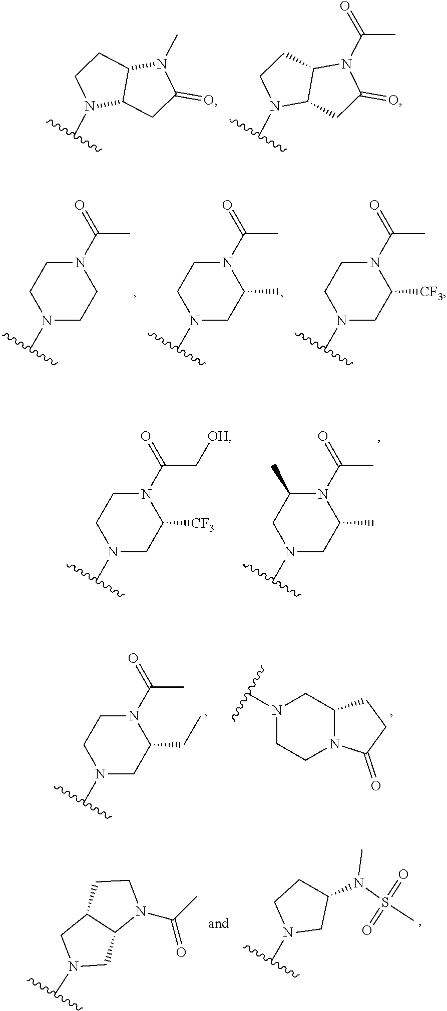

or a pharmaceutically acceptable salt or solvate thereof.

In another embodiment, Compounds of the Disclosure are compounds having any one of Formulae IV-A, IV-B, IV-C, or IV-D, wherein:
Z⁴ is —CH₂—; and
R^{11a} is selected from the group consisting of:

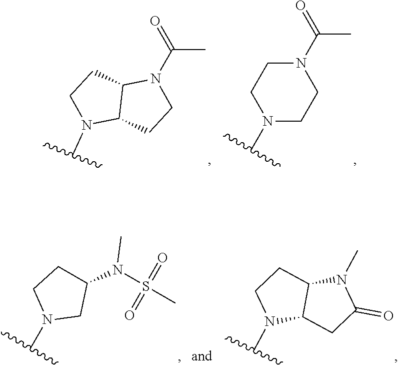

or a pharmaceutically acceptable salt or solvate thereof.

In another embodiment, Compounds of the Disclosure are compounds having any one of Formulae IV-A, IV-B, IV-C, or IV-D, wherein:
Z⁴ is —CH₂—;
R^{11a} is:

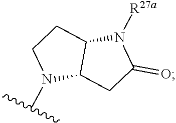

and
R^{27a} is selected from the group consisting of hydrogen, C₁-C₄ alkyl, C₁-C₄ haloalkyl, (C₁-C₄ alkoxy)C₁-C₄ alkyl, and (hydroxy)C₁-C₄ alkyl, or a pharmaceutically acceptable salt or solvate thereof. In another embodiment, R^{27a} is methyl.

In another embodiment, Compounds of the Disclosure are compounds Formula V:

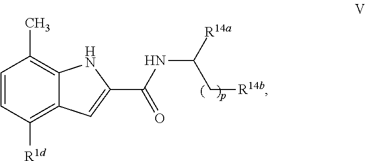

wherein:
R^{14a} is selected from the group consisting of optionally substituted alkyl and optionally substituted heteroaryl;
R^{14b} is selected from the group consisting of optionally substituted alkyl, optionally substituted heteroaryl, optionally substituted aryl, optionally substituted heterocyclo, optionally substituted cycloalkyl, and carboxamido; and
p is 0, 1, 2, or 3; or a pharmaceutically acceptable salt or solvate thereof.

In another embodiment, Compounds of the Disclosure are compounds having Formula V-A:

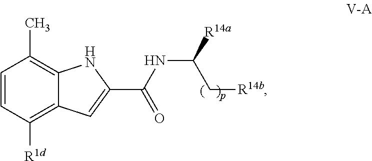

wherein R^{1d}, R^{14a}, R^{14d}, and p are as defined in connection with Formula V, or a pharmaceutically acceptable salt or solvate thereof.

In another embodiment, Compounds of the Disclosure are compounds having Formula V-B:

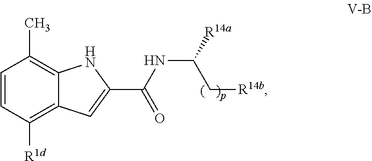

wherein R^{1d}, R^{14a}, R^{14d}, and p are as defined in connection with Formula V, or a pharmaceutically acceptable salt or solvate thereof.

In another embodiment, Compounds of the Disclosure are compounds having any one of Formulae V, V-A, or V-B, wherein:
R^{14a} is selected from the group consisting of (A) unsubstituted 5- to 10-membered heteroaryl; (B) substituted 5- or 10-membered heteroaryl having one, two, three, or four substituents independently selected from the group consisting of (i) halo; (ii) C₁-C₄ alkyl; (iii) C₁-C₄ alkoxy; (iv) (3- to 8-membered heterocyclo)C₁-C₄ alkyl; (v) (5- to 9-membered heteroaryl)C₁-C₄ alkyl; (vi) —C(═O)NR^{15a}R^{15b}; (vii) unsubstituted 5- to 10-membered heteroaryl; (viii) substituted 5- or 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; (ix) —OR¹⁶ (x) unsubstituted C₃-C₆ cycloalkyl; (xi) substituted C₃-C₆ cycloalkyl having one, two, three, or four substituents independently selected from the group consisting of C₁-C₄ alkyl and —N(R^{17a})C(═O)R^{18a}; (xii) cyano; (xiii) unsubstituted 4- to 14-membered heterocyclo; (xiv) substituted 4- to 14-membered heterocyclo having one or two substituents independently selected from the group consisting of C₁-C₄ alkyl, (5- to 9-membered heteroaryl)C₁-C₄ alkyl; (xv) (carboxy)C₁-C₄ alkyl; (xvi) (carboxamido)C₁-C₄ alkyl; and (xvii) carboxy; and (C) C₁-C₆ alkyl;
R^{14b} is selected from the group consisting of: (A) unsubstituted 5- to 10-membered heteroaryl; (B) substituted 5- or 10-membered heteroaryl having one, two, three, or four substituents independently selected from the group consisting of halo, C₁-C₄ alkyl, and (C₃-C₆ cycloalkyl)C₁-C₄ alkyl; (C) unsubstituted C₆-C₁₀ aryl; (D) substituted C₆-C₁₀ aryl, having one, two, three, or four substituents independently selected from the group consisting of halo, C₁-C₄ alkyl, and (3- to 8-membered heterocyclo)C₁-C₄ alkyl; (E) unsubstituted 4- to 14-membered heterocyclo; (F) substituted 4- to 14-membered heterocyclo having one, two, three, or four substituents independently selected from the group consisting of hydroxy, amino, and C₁-C₄ alkyl; (G) —C(═O)NR^{15c}R^{15d}; (H) unsubstituted C₃-C₆ cycloalkyl; and (I) C₁-C₆ alkyl;
p is 0, 1, 2, or 3;
R^{15a} and R^{15b} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{g5g}R^{15h}; or
R^{15a} and R^{15b} taken together with the nitrogen atom to which they are attached form an optionally substituted 4- to 14-membered heterocyclo;
R^{15c} and R^{15d} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or
R^{15c} and R^{15d} taken together with the nitrogen atom to which they are attached form an optionally substituted 4- to 14-membered heterocyclo;
R^{15e} and R^{15f} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or
R^{15e} and R^{15f} taken together with the nitrogen atom to which they are attached form an optionally substituted 4- to 14-membered heterocyclo;
R^{15g} and R^{15h} are independently selected from the group consisting of: (A) hydrogen; (B) C₁-C₆ alkyl; (C) C₁-C₆ haloalkyl; (D) C₁-C₆ alkoxy; (E) (C₁-C₄ alkoxy)C₁-C₄ alkyl; (F) (hydroxy)C₁-C₄ alkyl; (G) (cyano)alkyl; (H) unsubstituted C₆-C₁₀ aryl; (I) substituted C₆-C₁₀ aryl, having one, two, three, or four substituents independently selected from the group consisting of halo, amino, hydroxy, and C₁-C₄ alkyl; (J) unsubstituted 5- or 6-membered heteroaryl; (K) substituted 5- or 6-membered heteroaryl having one, two, three, or four substituents independently selected from the group consisting of halo, amino, hydroxy, and C₁-C₄ alkyl; (L) unsubstituted 4- to 14-membered heterocyclo; (M) substituted 4- to 14-membered heterocyclo having one or two substituents independently selected from the group consisting of amino, hydroxy, and C₁-C₄ alkyl; (N) unsubstituted C₃-C₈ cycloalkyl; and (O) substituted C₃-C₈ cycloalkyl having one, two, three, or four substituents independently selected from the group consisting of C₁-C₆ alkyl and —NR^{15g}R^{15h}; or
R^{15g} and R^{15g} taken together with the nitrogen atom to which they are attached form an optionally substituted 4- to 14-membered heterocyclo;
R¹⁶ is (amino)(hydroxy)C₁-C₄ alkyl;
R^{17a} is selected from the group consisting of hydrogen and C₁-C₄ alkyl;
R^{18a} is selected from the group consisting of: (A) C₁-C₆ alkyl; (B) C₁-C₆ haloalkyl; (C) C₁-C₆ alkoxy; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of amino, hydroxy, and C₁-C₄ alkyl,
or a pharmaceutically acceptable salt or solvate thereof.

In another embodiment, Compounds of the Disclosure are compounds having any one of Formulae V, V-A, or V-B, wherein R^{14a} is selected from the group consisting of unsubstituted 5- to 10-membered heteroaryl; and substituted 5- or 10-membered heteroaryl having one, two, or three substituents independently selected from the group consisting of C₁-C₄ alkyl; C₁-C₄ alkoxy; (3- to 8-membered heterocyclo)C₁-C₄ alkyl; (5- to 9-membered heteroaryl)C₁-C₄ alkyl; —C(═O)NR^{15a}R^{15b}; unsubstituted 5- to 10-membered heteroaryl; substituted 5- or 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; unsubstituted C₃-C₆ cycloalkyl; and substituted C₃-C₆ cycloalkyl having one, two, or three substituents independently selected from the group consisting of C₁-C₄ alkyl and —N(R^{17a})C(═O)R^{18a}, or a pharmaceutically acceptable salt or solvate thereof.

In another embodiment, Compounds of the Disclosure are compounds having any one of Formulae V, V-A, or V-B, wherein R^{14a} is a substituted pyridyl having one, two, or three substituents independently selected from the group consisting of C₁-C₄ alkyl; C₁-C₄ alkoxy; (3- to 8-membered heterocyclo)C₁-C₄ alkyl; (5- to 9-membered heteroaryl)C₁-C₄ alkyl; —C(═O)NR^{15a}R^{15b}; unsubstituted 5- to 10-membered heteroaryl; substituted 5- to 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; unsubstituted C₃-C₆ cycloalkyl; and substituted C₃-C₆ cycloalkyl having one, two, or three substituents independently selected from the group consisting of C₁-C₄ alkyl and —N(R^{17a})C(═O)R^{18a}, or a pharmaceutically acceptable salt or solvate thereof.

In another embodiment, Compounds of the Disclosure are compounds having any one of Formulae V, V-A, or V-B, wherein R^{14b} is selected from the group consisting of unsubstituted 5- to 10-membered heteroaryl; substituted 5- to 10-membered heteroaryl having one or two substituents independently selected from the group consisting of C₁-C₄ alkyl and (C₃-C₆ cycloalkyl)C₁-C₄ alkyl; unsubstituted C₆-C₁₀ aryl; substituted C₆-C₁₀ aryl, having one or two substituents independently selected from the group consisting of C₁-C₄ alkyl and (3- to 8-membered heterocyclo)C₁-C₄ alkyl; unsubstituted 4- to 14-membered heterocyclo; substituted 4- to 14-membered heterocyclo having one or two substituents independently selected from the group consisting of hydroxy, amino, and C₁—C₄ alkyl; and unsubstituted C₃-C₆ cycloalkyl, or a pharmaceutically acceptable salt or solvate thereof.

In another embodiment, Compounds of the Disclosure are compounds having any one of Formulae V, V-A, or V-B, wherein R^{14b} is selected from the group consisting of unsubstituted 5- or 6-membered heteroaryl; substituted 5- or 6-membered heteroaryl having one or two substituents independently selected from the group consisting of C₁-C₄ alkyl and (C₃-C₆ cycloalkyl)C₁-C₄ alkyl; unsubstituted phenyl; substituted phenyl, having one or two substituents independently selected from the group consisting of C₁-C₄ alkyl and (3- to 8-membered heterocyclo)C₁-C₄ alkyl; and unsubstituted C₃-C₆ cycloalkyl, or a pharmaceutically acceptable salt or solvate thereof.

In another embodiment, Compounds of the Disclosure are compounds having any one of Formulae V, V-A, or V-B, wherein p is 0, or a pharmaceutically acceptable salt or solvate thereof.

In another embodiment, Compounds of the Disclosure are compounds having any one of Formulae V, V-A, or V-B, wherein p is 1, or a pharmaceutically acceptable salt or solvate thereof.

In another embodiment, Compounds of the Disclosure are compounds having Formula VI:

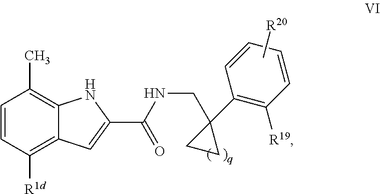

wherein:
R¹⁹ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl;
R²⁰ is selected from the group consisting of hydrogen, halo, and C₁-C₄ alkyl; and
q is 1, 2, or 3, or a pharmaceutically acceptable salt or solvate thereof.

In another embodiment, Compounds of the Disclosure are compounds having Formula VI, wherein q is 1.

In another embodiment, Compounds of the Disclosure are compounds having Formula VII:

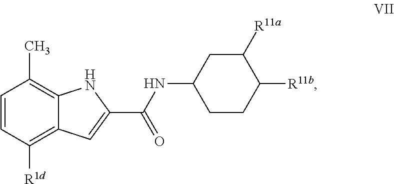

wherein:
R^{11b} is selected from the group consisting of C₁-C₄ alkyl, halo, and C₁-C₄ haloalkyl; and
R^{1d} and R^{11a} are as defined in connection with Formula IV, or a pharmaceutically acceptable salt or solvate thereof.

In another embodiment, Compounds of the Disclosure are compounds having Formula VII-A:

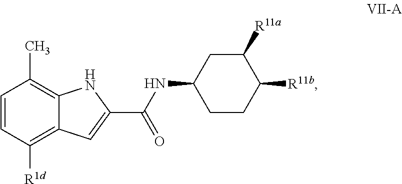

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-B:

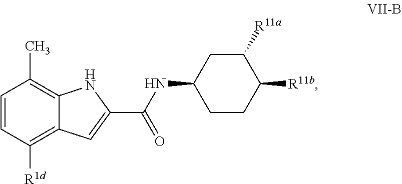

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-C:

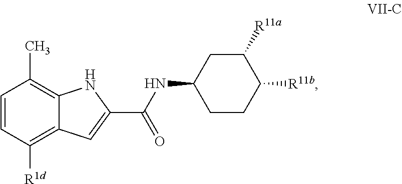

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-D:

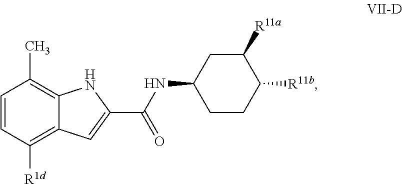

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-E:

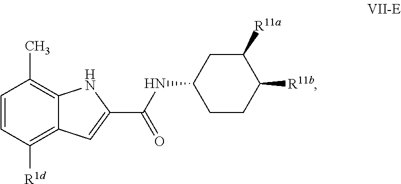

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-F:

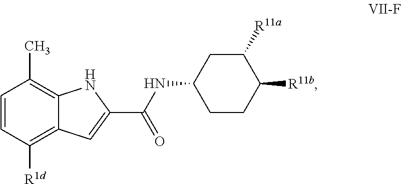

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-G:

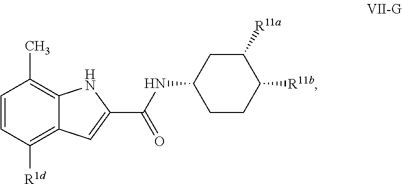

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VII-H:

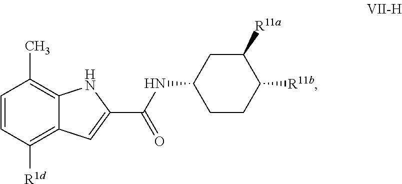

wherein R^{1d}, R^{11a}, and R^{11b} are as defined in connection with Formula VII, or a pharmaceutically acceptable salt or solvate thereof.

In another embodiment, Compounds of the Disclosure are compounds having Formula VIII:

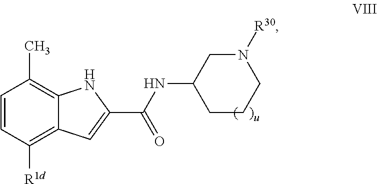

wherein:
R³⁰ is selected from the group consisting of hydrogen; C₁-C₆ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; —C(═O)R^{13b}, and —S(═O)₂R²⁴;
R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;
R²⁴ is selected from the group consisting of C₁-C₄ alkyl and (hydroxy)C₁-C₄ alkyl;
u is 0, 1, 2, or 3; and
R^{1d} is as defined in connection with Formula I, or a pharmaceutically acceptable salt or solvate thereof.

In another embodiment, Compounds of the Disclosure are compounds having Formula VIII-A:

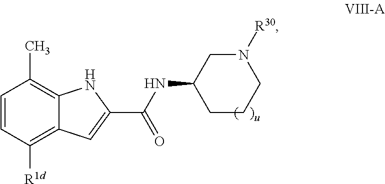

wherein R^{1d}, R³⁰, and u are as defined in connection with Formula VIII, or a pharmaceutically acceptable salt or solvate thereof.

In another embodiment, Compounds of the Disclosure are compounds having Formula VIII-B:

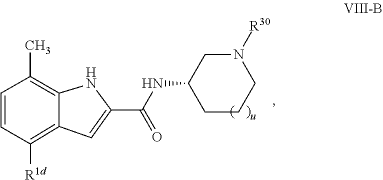

wherein R^{1d}, R³⁰, and u are as defined in connection with Formula VIII, or a pharmaceutically acceptable salt or solvate thereof.

In another embodiment, Compounds of the Disclosure are compounds of Table 1, and the pharmaceutically acceptable salts or solvates thereof. The chemical names of the compounds of Table 1 were generated by Chemdraw® Professional version 17.0.0.206. Mass spectroscopy and biological data of representative Compounds of the Disclosure are provided in Table 1B and/or WO 2020/037079 and/or WO 2021/168313. In another embodiment, Compounds of the Disclosure are compounds of Table 1B, and the pharmaceutically acceptable salts or solvates thereof. The biological data in Table 1B were generated following the protocols described in EXAMPLES 11 and 12 of WO 2020/037079.

In another embodiment, Compounds of the Disclosure are selected from the group consisting of Cpd. Nos. 1, 2, 3, 4, 5, 6, 7, 8, 9, 10, 11, 12, 13, 14, 15, 16, 17, 18, 824, 828, 839, 870, 922, 930, 942, 995, 1007, 1025, 1043, 1044, 1045, 1048, 1051, 1055, 1070, 1078, 1083, 1097, 1117, 1138, 1180, 1184, and 1192, and the pharmaceutically acceptable salts or solvates thereof. In another embodiment, Compounds of the Disclosure are selected from the group consisting of Cpd. Nos. 15, 922, 930, 942, 1055, 1070, 1117, 1180, 1184, and 1192, and the pharmaceutically acceptable salts or solvates thereof. In another embodiment, Compounds of the Disclosure are selected from the group consisting of Cpd. Nos. 1228, 1229, 1230, 1231, 1232, 1233, 1234 and 1235, and the pharmaceutically acceptable salts or solvates thereof.

In another embodiment, Compounds of the Disclosure are selected from the group consisting of Cpd. Nos. 15, 942, 1184, and 1232, and the pharmaceutically acceptable salts or solvates thereof.

In a non-limiting embodiment, the Compound of the Disclosure is Cpd. No. 15. In a non-limiting embodiment, the Compound of the Disclosure is Cpd. No. 1228. In a non-limiting embodiment, the Compound of the Disclosure is Cpd. No. 1229. In a non-limiting embodiment, the Compound of the Disclosure is Cpd. No. 1230. In a non-limiting embodiment, the Compound of the Disclosure is Cpd. No. 1231. In a non-limiting embodiment, the Compound of the Disclosure is Cpd. No. 1232. In a non-limiting embodiment, the Compound of the Disclosure is Cpd. No. 1233. In a non-limiting embodiment, the Compound of the Disclosure is Cpd. No. 1234. In a non-limiting embodiment, the Compound of the Disclosure is Cpd. No. 1235. In a non-limiting embodiment, the Compound of the Disclosure is a pharmaceutically acceptable salt or solvate of Cpd. No. 15. In a non-limiting embodiment, the Compound of the Disclosure is a pharmaceutically acceptable salt or solvate of Cpd. No. 1228. In a non-limiting embodiment, the Compound of the Disclosure is a pharmaceutically acceptable salt or solvate of Cpd. No. 1229. In a non-limiting embodiment, the Compound of the Disclosure is a pharmaceutically acceptable salt or solvate of Cpd. No. 1230. In a non-limiting embodiment, the Compound of the Disclosure is a pharmaceutically acceptable salt or solvate of Cpd. No. 1231. In a non-limiting embodiment, the Compound of the Disclosure is a pharmaceutically acceptable salt or solvate of Cpd. No. 1232. In a non-limiting embodiment, the Compound of the Disclosure is a pharmaceutically acceptable salt or solvate of Cpd. No. 1233. In a non-limiting embodiment, the Compound of the Disclosure is a pharmaceutically acceptable salt or solvate of Cpd. No. 1234. In a non-limiting embodiment, the Compound of the Disclosure is a pharmaceutically acceptable salt or solvate of Cpd. No. 1235.

TABLE 1

| Cpd. | |
|---|---|
| No. | Chemical Name |
| 1 | 4-fluoro-N-(3-fluoro-5-(3-(N-methylacetamido)pyrrolidin-1-yl)phenyl)-7-methyl- |
| | 1H-indole-2-carboxamide |
| 2 | 4-fluoro-N-(3-fluoro-5-(3-(2-methyl-3-oxohexahydroimidazo[1,5-a]pyrazin- |
| | 7(1H)-yl)pyrrolidin-1-yl)phenyl)-7-methyl-1H-indole-2-carboxamide |
| 3 | N-(3-(3-(dimethylamino)-2-oxopyrrolidin-1-yl)-5-fluorophenyl)-4-fluoro-7- |
| | methyl-1H-indole-2-carboxamide |
| 4 | N-(3-(3-(4-(2-(dimethylamino)-2-oxoethyl)piperazin-1-yl)pyrrolidin-1-yl)-5- |
| | fluorophenyl)-4-fluoro-7-methyl-1H-indole-2-carboxamide |
| 5 | N-(3-(8-acetyl-2,8-diazaspiro[4.5]decan-2-yl)-5-fluorophenyl)-4-fluoro-7-methyl- |
| | 1H-indole-2-carboxamide |
| 6 | N-(3-(3-(4-(2-amino-2-oxoethyl)piperazin-1-yl)pyrrolidin-1-yl)-5-fluorophenyl)- |
| | 4-fluoro-7-methyl-1H-indole-2-carboxamide |
| 7 | N-(3-(4-acetylpiperazin-1-yl)-5-fluorophenyl)-4-fluoro-7-methyl-1H-indole-2- |
| | carboxamide |
| 8 | N-(3-(3-(1,1-dioxidothiomorpholino)pyrrolidin-1-yl)-5-fluorophenyl)-4-fluoro-7- |
| | methyl-1H-indole-2-carboxamide |
| 9 | N-(3-(3-(4-acetylpiperazin-1-yl)pyrrolidin-1-yl)-5-fluorophenyl)-4-fluoro-7- |
| | methyl-1H-indole-2-carboxamide |
| 10 | N-(3-(4-cyclopropyl-3-oxopiperazin-1-yl)-5-fluorophenyl)-4-fluoro-7-methyl-1H- |
| | indole-2-carboxamide |
| 11 | 4-fluoro-N-(3-fluoro-5-(3-morpholinopyrrolidin-1-yl)phenyl)-7-methyl-1H- |
| | indole-2-carboxamide |
| 12 | 4-fluoro-N-(3-fluoro-5-(4-(2-methoxyacetyl)piperazin-1-yl)phenyl)-7-methyl-1H- |
| | indole-2-carboxamide |
| 13 | N-(3-(4-acetylhexahydropyrrolo[3,2-b]pyrrol-1(2H)-yl)-5-fluorophenyl)-4-fluoro- |
| | 7-methyl-1H-indole-2-carboxamide |
| 14 | N-(3-(1-acetylhexahydropyrrolo[3,4-b]pyrrol-5(1H)-yl)-5-fluorophenyl)-4-fluoro- |
| | 7-methyl-1H-indole-2-carboxamide |
| 15 | N-((1R,3S)-3-(4-acetylpiperazin-1-yl)cyclohexyl)-4-fluoro-7-methyl-1H-indole- |
| | 2-carboxamide |
| 16 | 4-fluoro-N-(3-fluoro-5-(3-(2-(hydroxymethyl)morpholino)pyrrolidin-1- |
| | yl)phenyl)-7-methyl-1H-indole-2-carboxamide |
| 17 | 4-fluoro-N-(3-fluoro-5-(3-(4-methyl-3-oxopiperazin-1-yl)pyrrolidin-1-yl)phenyl)- |
| | 7-methyl-1H-indole-2-carboxamide |
| 18 | 4-fluoro-N-(3-fluoro-5-(2-oxo-[1,3′-bipyrrolidin]-1′-yl)phenyl)-7-methyl-1H- |
| | indole-2-carboxamide |
| 19 | N-(3-(7-acetyl-2,7-diazaspiro[3.5]nonan-2-yl)-5-fluorophenyl)-4-fluoro-7- |
| | methyl-1H-indole-2-carboxamide |
| 20 | 4-fluoro-N-(3-fluoro-5-(4-methyl-3-oxopiperazin-1-yl)phenyl)-7-methyl-1H- |
| | indole-2-carboxamide |
| 21 | 4-fluoro-N-(3-fluoro-5-(4-methyl-5-oxo-1,4-diazepan-1-yl)phenyl)-7-methyl-1H- |
| | indole-2-carboxamide |
| 22 | N-(3-chloro-5-(3-morpholinopyrrolidin-1-yl)phenyl)-4-fluoro-7-methyl-1H- |
| | indole-2-carboxamide |
| 23 | N-(3-(1-acetyl-4-methylpiperidin-4-yl)-5-fluorophenyl)-4-fluoro-7-methyl-1H- |
| | indole-2-carboxamide |
| 24 | 4-fluoro-N-(3-fluoro-5-(2-(2-hydroxyethyl)morpholino)phenyl)-7-methyl-1H- |
| | indole-2-carboxamide |
| 25 | N-(3-(4-(dimethylcarbamoyl)piperazin-1-yl)-5-fluorophenyl)-4-fluoro-7-methyl- |
| | 1H-indole-2-carboxamide |
| 26 | 4-fluoro-N-(3-fluoro-5-((7S,8aS)-3-oxooctahydroindolizin-7-yl)phenyl)-7- |
| | methyl-1H-indole-2-carboxamide |
| 27 | 4-fluoro-7-methyl-N-(3-(6-oxohexahydropyrrolo[1,2-a]pyrazin-2(1H)-yl)phenyl)- |
| | 1H-indole-2-carboxamide |
| 28 | N-(3-(6,6-difluoro-2,8-diazaspiro[4.5]decan-2-yl)-5-fluorophenyl)-4-fluoro-7- |
| | methyl-1H-indole-2-carboxamide |
| 29 | N-(3-(3-(5,6-dihydroimidazo[1,2-a]pyrazin-7(8H)-yl)pyrrolidin-1-yl)-5- |
| | fluorophenyl)-4-fluoro-7-methyl-1H-indole-2-carboxamide |
| 30 | N-(3-(2-acetyl-2,8-diazaspiro[4.5]decan-8-yl)-5-fluorophenyl)-4-fluoro-7-methyl- |
| | 1H-indole-2-carboxamide |
| 31 | N-(3-(4-acetylpiperazin-1-yl)-5-chlorophenyl)-4-fluoro-7-methyl-1H-indole-2- |
| | carboxamide |
| 32 | N-(3-(4-acetyl-3-methylpiperazin-1-yl)phenyl)-4-fluoro-7-methyl-1H-indole-2- |
| | carboxamide |
| 34 | N-(3-(4-acetylpiperazin-1-yl)phenyl)-4-fluoro-7-methyl-1H-indole-2- |
| | carboxamide |
| 35 | 4-fluoro-N-(3-fluoro-5-((1s,4s)-4-morpholinocyclohexyl)phenyl)-7-methyl-1H- |
| | indole-2-carboxamide |
| 36 | 4-fluoro-7-methyl-N-(3-(4-propionylpiperazin-1-yl)phenyl)-1H-indole-2- |
| | carboxamide |
| 37 | 4-fluoro-7-methyl-N-(3-(3-oxooctahydroindolizin-7-yl)phenyl)-1H-indole-2- |
| | carboxamide |
| 38 | 4-fluoro-7-methyl-N-(3-(3-(N-methylacetamido)pyrrolidin-1-yl)phenyl)-1H- |
| | indole-2-carboxamide |
| 39 | N-(3-(3-(4-(dimethylglycyl)piperazin-1-yl)pyrrolidin-1-yl)-5-fluorophenyl)-4- |
| | fluoro-7-methyl-1H-indole-2-carboxamide |
| 40 | N-(3-(7-(2,2-difluoroethyl)-2,7-diazaspiro[3.5]nonan-2-yl)-5-fluorophenyl)-4- |
| | fluoro-7-methyl-1H-indole-2-carboxamide |
| 41 | N-(3-(7-(2,2-difluoroethyl)-2,7-diazaspiro[3.5]nonan-2-yl)phenyl)-4-fluoro-7- |
| | methyl-1H-indole-2-carboxamide |
| 42 | 4-fluoro-7-methyl-N-(3-(3-(N-methylacetamido)piperidin-1-yl)phenyl)-1H- |
| | indole-2-carboxamide |
| 43 | N-(3-(4-(dimethylalanyl)piperazin-1-yl)-5-fluorophenyl)-4-fluoro-7-methyl-1H- |
| | indole-2-carboxamide |
| 44 | 4-fluoro-N-(3-(4-(2-methoxyacetyl)piperazin-1-yl)phenyl)-7-methyl-1H-indole-2- |
| | carboxamide |
| 45 | N-(3-(1-acetylpiperidin-4-yl)phenyl)-4-fluoro-7-methyl-1H-indole-2- |
| | carboxamide |
| 46 | 4-fluoro-7-methyl-N-((1R,3S)-3-(3-(N-methylacetamido)pyrrolidin-1- |
| | yl)cyclohexyl)-1H-indole-2-carboxamide |
| 47 | 4-fluoro-N-(3-fluoro-5-(3-((2-fluoroethyl)(methyl)amino)pyrrolidin-1-yl)phenyl)- |
| | 7-methyl-1H-indole-2-carboxamide |
| 48 | 4-fluoro-N-(3-fluoro-5-(2-(hydroxymethyl)morpholino)phenyl)-7-methyl-1H- |
| | indole-2-carboxamide |
| 49 | 4-fluoro-7-methyl-N-(3-(4-nicotinoylpiperazin-1-yl)phenyl)-1H-indole-2- |
| | carboxamide |
| 50 | 4-fluoro-N-(3-(2-(2-hydroxyethyl)morpholino)phenyl)-7-methyl-1H-indole-2- |
| | carboxamide |
| 51 | 4-fluoro-7-methyl-N-(3-((7R,8aR)-3-oxooctahydroindolizin-7-yl)phenyl)-1H- |
| | indole-2-carboxamide |
| 52 | N-(3-(4-acetylpiperazin-1-yl)-2-fluorophenyl)-4-fluoro-7-methyl-1H-indole-2- |
| | carboxamide |
| 53 | N-(3-(3-(dimethylamino)pyrrolidin-1-yl)-5-fluorophenyl)-4-fluoro-7-methyl-1H- |
| | indole-2-carboxamide |
| 54 | 4-fluoro-7-methyl-N-(3-(2-methyl-3-oxohexahydroimidazo[1,5-a]pyrazin-7(1H)- |
| | yl)phenyl)-1H-indole-2-carboxamide |
| 55 | N-(3-(4-(dimethylcarbamoyl)piperazin-1-yl)phenyl)-4-fluoro-7-methyl-1H- |
| | indole-2-carboxamide |
| 56 | N-(3-(1-acetylpyrrolidin-3-yl)-5-fluorophenyl)-4-fluoro-7-methyl-1H-indole-2- |
| | carboxamide |
| 57 | N-(3-(2-oxa-7-azaspiro[3.5]nonan-7-yl)phenyl)-4-fluoro-7-methyl-1H-indole-2- |
| | carboxamide |
| 58 | 4-fluoro-7-methyl-N-(3-(4-methyl-3-oxopiperazin-1-yl)phenyl)-1H-indole-2- |
| | carboxamide |
| 59 | N-(3-(3-(dimethylamino)pyrrolidin-1-yl)phenyl)-4-fluoro-7-methyl-1H-indole-2- |
| | carboxamide |
| 60 | N-(3-(1,3,4-oxadiazol-2-yl)phenyl)-4-fluoro-7-methyl-1H-indole-2-carboxamide |
| 61 | N-(3-((3S,4S)-3-(dimethylamino)-4-hydroxypyrrolidin-1-yl)phenyl)-4-fluoro-7- |
| | methyl-1H-indole-2-carboxamide |
| 62 | N-(3-(4-(dimethylglycyl)piperazin-1-yl)-5-fluorophenyl)-4-fluoro-7-methyl-1H- |
| | indole-2-carboxamide |
| 63 | N-(3-(3-(dimethylamino)-4-fluoropyrrolidin-1-yl)phenyl)-4-fluoro-7-methyl-1H- |
| | indole-2-carboxamide |
| 64 | 4-fluoro-7-methyl-N-(3-(pyrimidin-5-yl)phenyl)-1H-indole-2-carboxamide |
| 65 | 4-fluoro-7-methyl-N-(3-(5-methyl-1,3,4-thiadiazol-2-yl)phenyl)-1H-indole-2- |
| | carboxamide |
| 66 | 4-fluoro-7-methyl-N-(3-(2-oxooxazolidin-3-yl)phenyl)-1H-indole-2-carboxamide |
| 68 | N-(3-((1r,4r)-4-(dimethylamino)cyclohexyl)phenyl)-4-fluoro-7-methyl-1H- |
| | indole-2-carboxamide |
| 69 | N-(3-((1s,4s)-4-(dimethylamino)cyclohexyl)-5-fluorophenyl)-4-fluoro-7-methyl- |
| | 1H-indole-2-carboxamide |
| 70 | N-(3-(4-(dimethylglycyl)-3-methylpiperazin-1-yl)-5-fluorophenyl)-4-fluoro-7- |
| | methyl-1H-indole-2-carboxamide |
| 71 | N-(3-(4-acetyl-3-ethylpiperazin-1-yl)phenyl)-4-fluoro-7-methyl-1H-indole-2- |
| | carboxamide |
| 72 | 4-fluoro-N-(3-(4-(2-hydroxyacetyl)piperazin-1-yl)phenyl)-7-methyl-1H-indole-2- |
| | carboxamide |
| 73 | N-(3-(1-acetylpyrrolidin-3-yl)phenyl)-4-fluoro-7-methyl-1H-indole-2- |
| | carboxamide |
| 74 | 4-fluoro-7-methyl-N-(3-(3-morpholinopyrrolidin-1-yl)phenyl)-1H-indole-2- |
| | carboxamide |
| 75 | 4-fluoro-N-(3-(4-(3-methoxypropanoyl)piperazin-1-yl)phenyl)-7-methyl-1H- |
| | indole-2-carboxamide |
| 76 | 4-fluoro-7-methyl-N-(3-(4-morpholinopiperidin-1-yl)phenyl)-1H-indole-2- |
| | carboxamide |
| 77 | N-(3-(4-acetyl-3-(trifluoromethyl)piperazin-1-yl)phenyl)-4-fluoro-7-methyl-1H- |
| | indole-2-carboxamide |
| 78 | 4-fluoro-7-methyl-N-(3-(4-(N-methylacetamido)piperidin-1-yl)phenyl)-1H- |
| | indole-2-carboxamide |
| 79 | N-(3-(4-acetylpiperazin-1-yl)-4-fluorophenyl)-4-fluoro-7-methyl-1H-indole-2- |
| | carboxamide |
| 80 | N-(3-(4-(dimethylamino)piperidin-1-yl)-5-fluorophenyl)-4-fluoro-7-methyl-1H- |
| | indole-2-carboxamide |
| 81 | 4-fluoro-N-(3-(5-(methoxymethyl)-1,3,4-thiadiazol-2-yl)phenyl)-7-methyl-1H- |
| | indole-2-carboxamide |
| 82 | 4-fluoro-N-(7-fluoroisoquinolin-5-yl)-7-methyl-1H-indole-2-carboxamide |
| 83 | methyl 2-(3-(4-fluoro-7-methyl-1H-indole-2-carboxamido)phenyl)-2,8- |
| | diazaspiro[4.5]decane-8-carboxylate |
| 84 | N-(3-(4-acetylpiperazin-1-yl)phenyl)-7-bromo-4-fluoro-1H-indole-2-carboxamide |
| 85 | 4-fluoro-7-methyl-N-(2-(1-methyl-1H-pyrazol-4-yl)-1-(3-methylpyridin-2- |
| | yl)ethyl)-1H-indole-2-carboxamide |
| 86 | N-(3-(1,8-diazaspiro[4.5]decan-8-yl)phenyl)-4-fluoro-7-methyl-1H-indole-2- |
| | carboxamide |
| 87 | 4-fluoro-N-(3-(4-hydroxy-4-methylpiperidin-1-yl)phenyl)-7-methyl-1H-indole-2- |
| | carboxamide |
| 88 | N-(3-(4-ethyl-3-oxopiperazin-1-yl)phenyl)-4-fluoro-7-methyl-1H-indole-2- |
| | carboxamide |
| 89 | 4-fluoro-N-(3-fluoro-5-(1′-methyl-5′-oxo-[1,3′-bipyrrolidin]-3-yl)phenyl)-7- |
| | methyl-1H-indole-2-carboxamide |
| 90 | 4-fluoro-7-methyl-N-(3-((7S,8aS)-3-oxooctahydroindolizin-7-yl)phenyl)-1H- |
| | indole-2-carboxamide |
| 91 | N-(3-chloro-5-(4-methylpiperazin-1-yl)phenyl)-4-fluoro-7-methyl-1H-indole-2- |
| | carboxamide |
| 92 | 4-fluoro-N-(3-(4-(2-fluoroacetyl)piperazin-1-yl)phenyl)-7-methyl-1H-indole-2- |
| | carboxamide |
| 93 | N-(3-(4-(cyclopropanecarbonyl)piperazin-1-yl)phenyl)-4-fluoro-7-methyl-1H- |
| | indole-2-carboxamide |
| 94 | (R)-N-(3-(3-(dimethylamino)pyrrolidin-1-yl)phenyl)-4-fluoro-7-methyl-1H- |
| | indole-2-carboxamide |
| 95 | N-(3-(4-oxa-1,9-diazaspiro[5.5]undecan-9-yl)phenyl)-4-fluoro-7-methyl-1H- |
| | indole-2-carboxamide |
| 96 | 4-fluoro-N-(3-fluoro-5-((7R,8aS)-3-oxooctahydroindolizin-7-yl)phenyl)-7- |
| | methyl-1H-indole-2-carboxamide |
| 97 | N-(3-((3R,5S)-4-acetyl-3,5-dimethylpiperazin-1-yl)phenyl)-4-fluoro-7-methyl- |
| | 1H-indole-2-carboxamide |
| 98 | 4-fluoro-7-methyl-N-(3-(2-methyl-2,7-diazaspiro[3.5]nonan-7-yl)phenyl)-1H- |
| | indole-2-carboxamide |
| 99 | N-(3-(4-(dimethylamino)-3-methylpiperidin-1-yl)phenyl)-4-fluoro-7-methyl-1H- |
| | indole-2-carboxamide |
| 100 | N-(3-(4-cyclopropyl-3-oxopiperazin-1-yl)phenyl)-4-fluoro-7-methyl-1H-indole- |
| | 2-carboxamide |
| 101 | 4-fluoro-7-methyl-N-(3-(tetrahydro-2H-pyran-4-yl)phenyl)-1H-indole-2- |
| | carboxamide |
| 102 | (S)-N-(3-(3-(dimethylamino)pyrrolidin-1-yl)phenyl)-4-fluoro-7-methyl-1H- |
| | indole-2-carboxamide |
| 103 | N-(3-(4,4-bis(hydroxymethyl)piperidin-1-yl)phenyl)-4-fluoro-7-methyl-1H- |
| | indole-2-carboxamide |
| 104 | N-(3-(1-acetyl-1,8-diazaspiro[4.5]decan-8-yl)-5-fluorophenyl)-4-fluoro-7-methyl- |
| | 1H-indole-2-carboxamide |
| 105 | 4-fluoro-N-(3-fluoro-5-((3aS,6aS)-1-methylhexahydropyrrolo[3,4-b]pyrrol- |
| | 5(1H)-yl)phenyl)-7-methyl-1H-indole-2-carboxamide |
| 106 | N-(3-chloro-5-((1s,4s)-4-(dimethylamino)cyclohexyl)phenyl)-4-fluoro-7-methyl- |
| | 1H-indole-2-carboxamide |
| 107 | 4-fluoro-N-(3-(5-(hydroxymethyl)-1,3,4-thiadiazol-2-yl)phenyl)-7-methyl-1H- |
| | indole-2-carboxamide |
| 108 | 4-fluoro-7-methyl-N-(3-(oxazol-5-yl)phenyl)-1H-indole-2-carboxamide |
| 109 | 4-fluoro-N-(3-(7-(2-methoxyethyl)-2,7-diazaspiro[3.5]nonan-2-yl)phenyl)-7- |
| | methyl-1H-indole-2-carboxamide |
| 110 | 4-fluoro-7-methyl-N-(3-(2-(2,2,2-trifluoroethyl)-2,7-diazaspiro[3.5]nonan-7- |
| | yl)phenyl)-1H-indole-2-carboxamide |
| 111 | N-(3-((3R,4S)-4-(dimethylamino)-3-methylpiperidin-1-yl)phenyl)-4-fluoro-7- |
| | methyl-1H-indole-2-carboxamide |
| 112 | methyl 2-(3-(4-fluoro-7-methyl-1H-indole-2-carboxamido)phenyl)-2,7- |
| | diazaspiro[3.5]nonane-7-carboxylate |
| 113 | N-(3-(4-(dimethylamino)piperidin-1-yl)phenyl)-4-fluoro-7-methyl-1H-indole-2- |
| | carboxamide |
| 114 | 4-fluoro-N-(3-fluoro-5-((8aR)-3-oxooctahydroindolizin-7-yl)phenyl)-7-methyl- |
| | 1H-indole-2-carboxamide |
| 115 | 4-fluoro-N-(3-(2-(hydroxymethyl)morpholino)phenyl)-7-methyl-1H-indole-2- |
| | carboxamide |
| 116 | 4-fluoro-7-methyl-N-(3-(pyridin-4-yl)phenyl)-1H-indole-2-carboxamide |
| 117 | N-(3-(4-((dimethylamino)methyl)piperidin-1-yl)phenyl)-4-fluoro-7-methyl-1H- |
| | indole-2-carboxamide |
| 118 | 4-fluoro-7-methyl-N-(3-(2-oxopyrrolidin-1-yl)phenyl)-1H-indole-2-carboxamide |
| 119 | N-(3-(5-cyclopropyl-1,3,4-oxadiazol-2-yl)phenyl)-4-fluoro-7-methyl-1H-indole- |
| | 2-carboxamide |
| 120 | N-(3-chloro-5-(3-(dimethylamino)pyrrolidin-1-yl)phenyl)-4-fluoro-7-methyl-1H- |
| | indole-2-carboxamide |
| 121 | methyl 4-(3-(4-fluoro-7-methyl-1H-indole-2-carboxamido)phenyl)piperazine-1- |
| | carboxylate |
| 122 | 4-fluoro-N-(3-fluoro-5-((3aR,6aR)-1-methylhexahydropyrrolo[3,4-b]pyrrol- |
| | 5(1H)-yl)phenyl)-7-methyl-1H-indole-2-carboxamide |
| 123 | N-(3-(1-acetylpiperidin-3-yl)phenyl)-4-fluoro-7-methyl-1H-indole-2- |
| | carboxamide |
| 124 | 4-fluoro-7-methyl-N-(3-(octahydro-6H-pyrrolo[3,4-b]pyridin-6-yl)phenyl)-1H- |
| | indole-2-carboxamide |
| 125 | N-(3-(1,1-dioxidoisothiazolidin-2-yl)phenyl)-4-fluoro-7-methyl-1H-indole-2- |
| | carboxamide |
| 126 | 4-fluoro-N-(3-(4-hydroxypiperidin-1-yl)phenyl)-7-methyl-1H-indole-2- |
| | carboxamide |
| 127 | methyl 8-(3-(4-fluoro-7-methyl-1H-indole-2-carboxamido)phenyl)-2,8- |
| | diazaspiro[4.5]decane-2-carboxylate |
| 128 | 4-fluoro-7-methyl-N-(3-(3-(methylamino)pyrrolidin-1-yl)phenyl)-1H-indole-2- |
| | carboxamide |
| 129 | 4-fluoro-7-methyl-N-(3-(2-methylmorpholino)phenyl)-1H-indole-2-carboxamide |
| 130 | 4-fluoro-N-(3-(3-((2-fluoroethyl)(methyl)amino)pyrrolidin-1-yl)phenyl)-7- |
| | methyl-1H-indole-2-carboxamide |
| 131 | 4-fluoro-7-methyl-N-((1S,3R)-3-(pyrimidin-5-yl)cyclohexyl)-1H-indole-2- |
| | carboxamide |
| 132 | N-(3-(5-((dimethylamino)methyl)-1,3,4-thiadiazol-2-yl)phenyl)-4-fluoro-7- |
| | methyl-1H-indole-2-carboxamide |
| 133 | 4-fluoro-N-(3-(2-(methoxymethyl)morpholino)phenyl)-7-methyl-1H-indole-2- |
| | carboxamide |
| 134 | 4-fluoro-N-(isoquinolin-5-yl)-7-methyl-1H-indole-2-carboxamide |
| 135 | 4-fluoro-7-methyl-N-(3-(6-methyl-2,6-diazaspiro[3.3]heptan-2-yl)phenyl)-1H- |
| | indole-2-carboxamide |
| 136 | 4-fluoro-7-methyl-N-(4′-(methylsulfonamidomethyl)-[1,1′-biphenyl]-3-yl)-1H- |
| | indole-2-carboxamide |
| 137 | 4-fluoro-7-methyl-N-(3-(pyridin-3-yl)phenyl)-1H-indole-2-carboxamide |
| 138 | 4-fluoro-7-methyl-N-(3-(4-(1-methylpiperidine-3-carbonyl)piperazin-1- |
| | yl)phenyl)-1H-indole-2-carboxamide |
| 139 | 4-fluoro-7-methyl-N-(3-(4-(methylcarbamoyl)piperazin-1-yl)phenyl)-1H-indole- |
| | 2-carboxamide |
| 140 | N-(3-(1-(dimethylglycyl)pyrrolidin-3-yl)-5-fluorophenyl)-4-fluoro-7-methyl-1H- |
| | indole-2-carboxamide |
| 141 | N-(2-(1-(cyclopropylmethyl)-1H-pyrazol-4-yl)-1-(3-methylpyridin-2-yl)ethyl)-4- |
| | fluoro-7-methyl-1H-indole-2-carboxamide |
| 142 | N-(3-(1-(2,2-difluoroethyl)-1,8-diazaspiro[4.5]decan-8-yl)phenyl)-4-fluoro-7- |
| | methyl-1H-indole-2-carboxamide |
| 143 | N-(3-(2-cyclopropylmorpholino)phenyl)-4-fluoro-7-methyl-1H-indole-2- |
| | carboxamide |
| 144 | 4-fluoro-7-methyl-N-(3-(4-(methylamino)piperidin-1-yl)phenyl)-1H-indole-2- |
| | carboxamide |
| 145 | N-(3-chloro-5-(pyrimidin-5-yl)phenyl)-4-fluoro-7-methyl-1H-indole-2- |
| | carboxamide |
| 146 | 4-fluoro-7-methyl-N-(3-(4-(tetrahydrofuran-3-yl)piperazin-1-yl)phenyl)-1H- |
| | indole-2-carboxamide |
| 147 | 4-fluoro-7-methyl-N-(3-(3-oxopiperazin-1-yl)phenyl)-1H-indole-2-carboxamide |
| 148 | 4-fluoro-N-(3-(3-((2-hydroxyethyl)(methyl)amino)pyrrolidin-1-yl)phenyl)-7- |
| | methyl-1H-indole-2-carboxamide |
| 149 | 4-fluoro-N-(3-(4-(3-hydroxypropanoyl)piperazin-1-yl)phenyl)-7-methyl-1H- |
| | indole-2-carboxamide |
| 150 | N-(3-(4-(3-aminopropyl)piperidin-1-yl)phenyl)-4-fluoro-7-methyl-1H-indole-2- |
| | carboxamide |
| 151 | 4-fluoro-7-methyl-N-(3-(1-methyl-1H-pyrazol-4-yl)phenyl)-1H-indole-2- |
| | carboxamide |
| 152 | 4-fluoro-7-methyl-N-(3-(4-(4-methylpiperazin-1-yl)piperidin-1-yl)phenyl)-1H- |
| | indole-2-carboxamide |
| 153 | N-(3-(3-(4,4-difluoropiperidin-1-yl)pyrrolidin-1-yl)phenyl)-4-fluoro-7-methyl- |
| | 1H-indole-2-carboxamide |
| 154 | N-(3-((3R,4R)-3-(dimethylamino)-4-hydroxypyrrolidin-1-yl)phenyl)-4-fluoro-7- |
| | methyl-1H-indole-2-carboxamide |
| 155 | 4-fluoro-N-(3-(4-(3-methoxyazetidin-1-yl)piperidin-1-yl)phenyl)-7-methyl-1H- |
| | indole-2-carboxamide |
| 156 | 4-fluoro-N-(3-(3-((2-methoxyethyl)(methyl)amino)pyrrolidin-1-yl)phenyl)-7- |
| | methyl-1H-indole-2-carboxamide |
| 157 | 4-fluoro-7-methyl-N-(3-(4-(pyridin-3-ylamino)piperidin-1-yl)phenyl)-1H-indole- |
| | 2-carboxamide |
| 159 | 4-fluoro-N-(3-fluoro-5-(3-methyl-3-morpholinopyrrolidin-1-yl)phenyl)-7-methyl- |
| | 1H-indole-2-carboxamide |
| 160 | 4-fluoro-7-methyl-N-(3-(1-methyl-2-oxopiperidin-4-yl)phenyl)-1H-indole-2- |
| | carboxamide |
| 161 | N-((5-((1H-imidazol-1-yl)methyl)-3-methylpyridin-2-yl)(cyclopropyl)methyl)-4- |
| | fluoro-7-methyl-1H-indole-2-carboxamide |
| 162 | N-(3-(1H-1,2,4-triazol-1-yl)phenyl)-4-fluoro-7-methyl-1H-indole-2-carboxamide |
| 163 | N-(3-(8-acetyl-3,8-diazabicyclo[3.2.1]octan-3-yl)phenyl)-4-fluoro-7-methyl-1H- |
| | indole-2-carboxamide |
| 164 | 4-fluoro-7-methyl-N-(3-(1-(2-morpholinoethyl)-1H-pyrazol-4-yl)phenyl)-1H- |
| | indole-2-carboxamide |
| 165 | 4-fluoro-7-methyl-N-(3-(5-methylhexahydropyrrolo[3,4-c]pyrrol-2(1H)- |
| | yl)phenyl)-1H-indole-2-carboxamide |
| 166 | 4-fluoro-7-methyl-N-(3-((7S,8aR)-3-oxooctahydroindolizin-7-yl)phenyl)-1H- |
| | indole-2-carboxamide |
| 167 | N-(3-(2-((dimethylamino)methyl)morpholino)phenyl)-4-fluoro-7-methyl-1H- |
| | indole-2-carboxamide |
| 168 | 4-fluoro-7-methyl-N-(3-(1-methylpiperidin-4-yl)phenyl)-1H-indole-2- |
| | carboxamide |
| 169 | 4-fluoro-7-methyl-N-((1R,3S)-3-(4-methyl-3-oxopiperazin-1-yl)cyclohexyl)-1H- |
| | indole-2-carboxamide |
| 170 | N-(3-(4-(2-cyanoacetyl)piperazin-1-yl)phenyl)-4-fluoro-7-methyl-1H-indole-2- |
| | carboxamide |
| 171 | 4-fluoro-7-methyl-N-(3-(1-methyl-1H-imidazol-2-yl)phenyl)-1H-indole-2- |
| | carboxamide |
| 172 | 4-fluoro-7-methyl-N-(3-(1-methyloctahydro-5H-pyrrolo[3,2-c]pyridin-5- |
| | yl)phenyl)-1H-indole-2-carboxamide |
| 173 | N-(3-(4,4-bis(methoxymethyl)piperidin-1-yl)phenyl)-4-fluoro-7-methyl-1H- |
| | indole-2-carboxamide |
| 174 | 4-fluoro-N-(3-(3-(3-hydroxyazetidin-1-yl)pyrrolidin-1-yl)phenyl)-7-methyl-1H- |
| | indole-2-carboxamide |
| 175 | N-(2-(1-(cyclopropylmethyl)-1H-pyrazol-4-yl)-1-(3-methylpyridin-2-yl)ethyl)-7- |
| | methyl-1H-indole-2-carboxamide |
| 176 | 4-fluoro-7-methyl-N-(3-(4-(methylsulfonyl)piperazin-1-yl)phenyl)-1H-indole-2- |
| | carboxamide |
| 177 | 4-fluoro-7-methyl-N-(3-(5-oxopyrrolidin-3-yl)phenyl)-1H-indole-2-carboxamide |
| 178 | N-(3-(3-(azetidin-1-yl)pyrrolidin-1-yl)phenyl)-4-fluoro-7-methyl-1H-indole-2- |
| | carboxamide |
| 179 | N-(3-(2,7-diazaspiro[3.5]nonan-7-yl)phenyl)-4-fluoro-7-methyl-1H-indole-2- |
| | carboxamide |
| 180 | 4-fluoro-7-methyl-N-(3-(4-(pyrrolidin-1-yl)piperidin-1-yl)phenyl)-1H-indole-2- |
| | carboxamide |
| 181 | 4-fluoro-7-methyl-N-(3-(3-oxohexahydroimidazo[1,5-a]pyrazin-7(1H)- |
| | yl)phenyl)-1H-indole-2-carboxamide |
| 182 | 4-fluoro-7-methyl-N-(3-(1-methyloctahydro-6H-pyrrolo[3,4-b]pyridin-6- |
| | yl)phenyl)-1H-indole-2-carboxamide |
| 183 | 4-fluoro-N-(3-fluoro-5-(4-methyl-3-oxo-1,4-diazepan-1-yl)phenyl)-7-methyl-1H- |
| | indole-2-carboxamide |
| 184 | N-(3-(2,7-diazaspiro[3.5]nonan-2-yl)phenyl)-4-fluoro-7-methyl-1H-indole-2- |
| | carboxamide |
| 185 | N-(5-(4-acetylpiperazin-1-yl)-2-fluorophenyl)-4-fluoro-7-methyl-1H-indole-2- |
| | carboxamide |
| 186 | N-(3-((3R,4S)-4-(dimethylamino)-3-fluoropiperidin-1-yl)phenyl)-4-fluoro-7- |
| | methyl-1H-indole-2-carboxamide |
| 187 | N-(3-(4-(dimethylamino)piperidin-1-yl)-5-methylphenyl)-4-fluoro-7-methyl-1H- |
| | indole-2-carboxamide |
| 188 | N-(3-chloro-5-(4-(3-morpholinopropyl)piperidin-1-yl)phenyl)-4-fluoro-7-methyl- |
| | 1H-indole-2-carboxamide |
| 189 | N-(3-(2,8-diazaspiro[4.5]decan-2-yl)phenyl)-4-fluoro-7-methyl-1H-indole-2- |
| | carboxamide |
| 190 | N-(3-chloro-5-(4-(dimethylamino)piperidin-1-yl)phenyl)-4-fluoro-7-methyl-1H- |
| | indole-2-carboxamide |
| 191 | 4-fluoro-7-methyl-N-(3-(3-methyl-2-oxoimidazolidin-1-yl)phenyl)-1H-indole-2- |
| | carboxamide |
| 192 | N-(3-(4-aminopiperidin-1-yl)phenyl)-4-fluoro-7-methyl-1H-indole-2- |
| | carboxamide |
| 193 | N-(3-(4-acetyl-3-isobutylpiperazin-1-yl)phenyl)-4-fluoro-7-methyl-1H-indole-2- |
| | carboxamide |
| 194 | 4-fluoro-7-methyl-N-(3-morpholinophenyl)-1H-indole-2-carboxamide |
| 195 | N-(3-(4-(dimethylamino)-1-hydroxycyclohexyl)phenyl)-4-fluoro-7-methyl-1H- |
| | indole-2-carboxamide |
| 196 | 4-fluoro-N-(3-(isoxazol-5-yl)phenyl)-7-methyl-1H-indole-2-carboxamide |
| 197 | 4-fluoro-7-methyl-N-(3-(1-methyl-1H-imidazol-5-yl)phenyl)-1H-indole-2- |
| | carboxamide |
| 198 | 4-fluoro-N-(3-(4-methoxypiperidin-1-yl)phenyl)-7-methyl-1H-indole-2- |
| | carboxamide |
| 199 | N-(3-(4-(cyclopropylmethyl)piperazin-1-yl)phenyl)-4-fluoro-7-methyl-1H-indole- |
| | 2-carboxamide |
| 200 | 4-fluoro-7-methyl-N-(3-(5-oxo-1,4-diazepan-1-yl)phenyl)-1H-indole-2- |
| | carboxamide |
| 201 | 4-fluoro-N-(3-(4-isopropyl-3-oxopiperazin-1-yl)phenyl)-7-methyl-1H-indole-2- |
| | carboxamide |
| 202 | N-(3-((3S,4R)-4-(dimethylamino)-3-methylpiperidin-1-yl)phenyl)-4-fluoro-7- |
| | methyl-1H-indole-2-carboxamide |
| 203 | N-(3-(2-((ethylamino)methyl)morpholino)phenyl)-4-fluoro-7-methyl-1H-indole- |
| | 2-carboxamide |
| 204 | 4-fluoro-N-(3-(4-(2-hydroxyethyl)piperazin-1-yl)phenyl)-7-methyl-1H-indole-2- |
| | carboxamide |
| 205 | 4-fluoro-N-(3-(4-isopropyl-3-methylpiperazin-1-yl)phenyl)-7-methyl-1H-indole- |
| | 2-carboxamide |
| 206 | N-(3-(2-(2,2-difluoroethyl)-2,8-diazaspiro[4.5]decan-8-yl)phenyl)-4-fluoro-7- |
| | methyl-1H-indole-2-carboxamide |
| 207 | 4-fluoro-7-methyl-N-(3-(4-(tetrahydro-2H-pyran-4-yl)piperazin-1-yl)phenyl)-1H- |
| | indole-2-carboxamide |
| 208 | N-(3-(4-(dimethylglycyl)piperazin-1-yl)phenyl)-4-fluoro-7-methyl-1H-indole-2- |
| | carboxamide |
| 209 | 4-fluoro-7-methyl-N-(3-(3-oxomorpholino)phenyl)-1H-indole-2-carboxamide |
| 210 | 4-fluoro-N-(3-(4-((2-hydroxyethyl)amino)piperidin-1-yl)phenyl)-7-methyl-1H- |
| | indole-2-carboxamide |
| 211 | 4-fluoro-7-methyl-N-((1R,3S)-3-morpholinocyclohexyl)-1H-indole-2- |
| | carboxamide |
| 212 | 4-fluoro-7-methyl-N-(3-(1-methyl-1,8-diazaspiro[4.5]decan-8-yl)phenyl)-1H- |
| | indole-2-carboxamide |
| 213 | N-(1-((1r,4r)-4-(dimethylamino)cyclohexyl)-1H-indazol-4-yl)-4-fluoro-7-methyl- |
| | 1H-indole-2-carboxamide |
| 214 | N-(3-((3R,5R)-4-acetyl-3,5-dimethylpiperazin-1-yl)phenyl)-4-fluoro-7-methyl- |
| | 1H-indole-2-carboxamide |
| 215 | 4-fluoro-7-methyl-N-(3-(pyrrolo[1,2-a]pyrazin-7-yl)phenyl)-1H-indole-2- |
| | carboxamide |
| 216 | 4-fluoro-7-methyl-N-(3-(3-methylmorpholino)phenyl)-1H-indole-2-carboxamide |
| 217 | N-(3-((1R,3R)-3-(dimethylamino)cyclopentyl)phenyl)-4-fluoro-7-methyl-1H- |
| | indole-2-carboxamide |
| 218 | N-(3-(6-oxa-3-azabicyclo[3.1.1]heptan-3-yl)phenyl)-4-fluoro-7-methyl-1H- |
| | indole-2-carboxamide |
| 219 | 4-fluoro-N-(3-(1-(2-hydroxyethyl)-1H-pyrazol-4-yl)phenyl)-7-methyl-1H-indole- |
| | 2-carboxamide |
| 220 | 4-fluoro-N-(3-(hexahydropyrrolo[1,2-a]pyrazin-2(1H)-yl)phenyl)-7-methyl-1H- |
| | indole-2-carboxamide |
| 221 | N-(3-(1H-imidazol-1-yl)phenyl)-4-fluoro-7-methyl-1H-indole-2-carboxamide |
| 222 | N-(3-(4-(dimethylamino)-4-methylpiperidin-1-yl)phenyl)-4-fluoro-7-methyl-1H- |
| | indole-2-carboxamide |
| 223 | 4-fluoro-N-(3-(4-(2-methoxyethyl)piperazin-1-yl)phenyl)-7-methyl-1H-indole-2- |
| | carboxamide |
| 224 | 4-fluoro-7-methyl-N-(3-(oxetan-3-yl)phenyl)-1H-indole-2-carboxamide |
| 225 | 4-fluoro-7-methyl-N-(3-(4-(propylamino)piperidin-1-yl)phenyl)-1H-indole-2- |
| | carboxamide |
| 226 | N-(3-(1-(dimethylglycyl)pyrrolidin-3-yl)phenyl)-4-fluoro-7-methyl-1H-indole-2- |
| | carboxamide |
| 227 | N-(3-(4-(bis(2-hydroxyethyl)amino)piperidin-1-yl)phenyl)-4-fluoro-7-methyl-1H- |
| | indole-2-carboxamide |
| 228 | N-(4′-((1H-imidazol-1-yl)methyl)-[1,1′-biphenyl]-3-yl)-4-fluoro-7-methyl-1H- |
| | indole-2-carboxamide |
| 229 | 4-fluoro-7-methyl-N-(3-(4-methyl-3-(trifluoromethyl)piperazin-1-yl)phenyl)-1H- |
| | indole-2-carboxamide |
| 230 | 4-fluoro-N-(3-fluoro-5-(1′-methyl-2′-oxo-[1,3′-bipyrrolidin]-3-yl)phenyl)-7- |
| | methyl-1H-indole-2-carboxamide |
| 231 | 4-fluoro-N-(4′-(2-hydroxyethoxy)-[1,1′-biphenyl]-3-yl)-7-methyl-1H-indole-2- |
| | carboxamide |
| 232 | 4-fluoro-7-methyl-N-(5-(4-methylpiperazin-1-yl)imidazo[1,5-a]pyridin-7-yl)-1H- |
| | indole-2-carboxamide |
| 233 | N-(3-(5,6-dihydroimidazo[1,2-a]pyrazin-7(8H)-yl)phenyl)-4-fluoro-7-methyl-1H- |
| | indole-2-carboxamide |
| 234 | 4-fluoro-7-methyl-N-(3-(4-methylpiperazin-1-yl)phenyl)-1H-indole-2- |
| | carboxamide |
| 235 | 4-fluoro-7-methyl-N-(3-(pyrazolo[1,5-a]pyridin-4-yl)phenyl)-1H-indole-2- |
| | carboxamide |
| 236 | N-(3-(4-cyclopropylpiperazin-1-yl)phenyl)-4-fluoro-7-methyl-1H-indole-2- |
| | carboxamide |
| 237 | 4-fluoro-7-methyl-N-(3-(4-methyl-4H-1,2,4-triazol-3-yl)phenyl)-1H-indole-2- |
| | carboxamide |
| 238 | 4-fluoro-7-methyl-N-(quinolin-5-yl)-1H-indole-2-carboxamide |
| 239 | 4-fluoro-7-methyl-N-(3-(1-methyl-1H-pyrazol-5-yl)phenyl)-1H-indole-2- |
| | carboxamide |
| 240 | N-(3-(4-(2-aminoethoxy)piperidin-1-yl)phenyl)-4-fluoro-7-methyl-1H-indole-2- |
| | carboxamide |
| 241 | N-(3-(2,8-diazaspiro[4.5]decan-8-yl)phenyl)-4-fluoro-7-methyl-1H-indole-2- |
| | carboxamide |
| 242 | N-(3-(4-acetylpiperazin-1-yl)-5-(trifluoromethyl)phenyl)-4-fluoro-7-methyl-1H- |
| | indole-2-carboxamide |
| 243 | 4-fluoro-N-(3-(4-isopropylpiperazin-1-yl)phenyl)-7-methyl-1H-indole-2- |
| | carboxamide |
| 244 | 4-fluoro-7-methyl-N-(3-(2-(4-methylpiperazin-1-yl)ethyl)phenyl)-1H-indole-2- |
| | carboxamide |
| 245 | 4-fluoro-7-methyl-N-(3-(1-methylhexahydropyrrolo[3,4-b]pyrrol-5(1H)- |
| | yl)phenyl)-1H-indole-2-carboxamide |
| 246 | N-(3-(4-(3-aminopropyl)piperidin-1-yl)-5-chlorophenyl)-4-fluoro-7-methyl-1H- |
| | indole-2-carboxamide |
| 247 | 4-fluoro-7-methyl-N-(1-(1-methylpiperidin-4-yl)-1H-indazol-4-yl)-1H-indole-2- |
| | carboxamide |
| 248 | N-(3-(4-acetyl-1,4-diazepan-1-yl)phenyl)-4-fluoro-7-methyl-1H-indole-2- |
| | carboxamide |
| 249 | N-(3-(1,2,4-oxadiazol-3-yl)phenyl)-4-fluoro-7-methyl-1H-indole-2-carboxamide |
| 250 | N-(3-(8-oxa-3-azabicyclo[3.2.1]octan-3-yl)phenyl)-4-fluoro-7-methyl-1H-indole- |
| | 2-carboxamide |
| 251 | N-(3-(4-(dimethylamino)bicyclo[4.1.0]heptan-1-yl)phenyl)-4-fluoro-7-methyl- |
| | 1H-indole-2-carboxamide |
| 252 | N-(3-(1H-pyrazol-4-yl)phenyl)-4-fluoro-7-methyl-1H-indole-2-carboxamide |
| 253 | N-(3-(4-(dimethylamino)-4-(methoxymethyl)piperidin-1-yl)phenyl)-4-fluoro-7- |
| | methyl-1H-indole-2-carboxamide |
| 254 | N-(3-(4-((dimethylamino)methyl)-4-hydroxypiperidin-1-yl)phenyl)-4-fluoro-7- |
| | methyl-1H-indole-2-carboxamide |
| 255 | N-(3-(1-cyano-4-(dimethylamino)cyclohexyl)phenyl)-4-fluoro-7-methyl-1H- |
| | indole-2-carboxamide |
| 256 | N-(3-(2-(dimethylamino)-2-oxoethyl)phenyl)-4-fluoro-7-methyl-1H-indole-2- |
| | carboxamide |
| 257 | N-(3-(6-acetyl-3,6-diazabicyclo[3.1.1]heptan-3-yl)phenyl)-4-fluoro-7-methyl-1H- |
| | indole-2-carboxamide |
| 258 | 4-fluoro-7-methyl-N-(1-(3-methyl-5-(2-(4-methylpiperazin-1-yl)ethyl)pyridin-2- |
| | yl)-2-phenylethyl)-1H-indole-2-carboxamide |
| 259 | N-(3-(8-(2,2-difluoroethyl)-2,8-diazaspiro[4.5]decan-2-yl)phenyl)-4-fluoro-7- |
| | methyl-1H-indole-2-carboxamide |
| 260 | tert-butyl 4-(3-(4-fluoro-7-methyl-1H-indole-2-carboxamido)phenyl)piperazine- |
| | 1-carboxylate |
| 261 | 4-fluoro-7-methyl-N-(3-(oxazol-2-yl)phenyl)-1H-indole-2-carboxamide |
| 262 | 4-fluoro-N-(3-(4-methoxy-4-methylpiperidin-1-yl)phenyl)-7-methyl-1H-indole-2- |
| | carboxamide |
| 263 | N-(3′-acetamido-[1,1′-biphenyl]-3-yl)-4-fluoro-7-methyl-1H-indole-2- |
| | carboxamide |
| 264 | N-(3-((1S,3S)-3-(dimethylamino)cyclopentyl)phenyl)-4-fluoro-7-methyl-1H- |
| | indole-2-carboxamide |
| 265 | 4-fluoro-N-(3-(3-(hydroxymethyl)morpholino)phenyl)-7-methyl-1H-indole-2- |
| | carboxamide |
| 266 | N-(3′-((dimethylamino)methyl)-[1,1′-biphenyl]-3-yl)-4-fluoro-7-methyl-1H- |
| | indole-2-carboxamide |
| 267 | N-(3-(1-acetyl-4-carbamoylpiperidin-4-yl)-5-fluorophenyl)-4-fluoro-7-methyl- |
| | 1H-indole-2-carboxamide |
| 268 | 4-fluoro-N-(3-(4-hydroxy-1′-methyl-[4,4′-bipiperidin]-1-yl)phenyl)-7-methyl-1H- |
| | indole-2-carboxamide |
| 269 | N-(cyclopropyl(4-methoxy-3-methylpyridin-2-yl)methyl)-4-fluoro-7-methyl-1H- |
| | indole-2-carboxamide |
| 270 | 4-fluoro-7-methyl-N-(4-methyl-3-(4-methylpiperazin-1-yl)phenyl)-1H-indole-2- |
| | carboxamide |
| 271 | N-(3-(4-acetylpiperazin-1-yl)-5-fluorophenyl)-4-fluoro-1H-indole-2-carboxamide |
| 272 | 4-fluoro-N-(3-(imidazo[1,2-a]pyridin-5-yl)phenyl)-7-methyl-1H-indole-2- |
| | carboxamide |
| 273 | N-(cyclopropyl(3-methyl-5-((2-oxo-2,3-dihydro-1H-imidazol-1- |
| | yl)methyl)pyridin-2-yl)methyl)-4-fluoro-7-methyl-1H-indole-2-carboxamide |
| 274 | N-(3-(4-(dimethylamino)piperidin-1-yl)-5-(fluoromethyl)phenyl)-4-fluoro-7- |
| | methyl-1H-indole-2-carboxamide |
| 275 | N-(3-chloro-5-(4-(3-morpholinopropyl)piperidin-1-yl)phenyl)-4-fluoro-7-methyl- |
| | 1H-indole-2-carboxamide |
| 276 | N-(3-(3-((dimethylamino)methyl)-1H-1,2,4-triazol-1-yl)phenyl)-4-fluoro-7- |
| | methyl-1H-indole-2-carboxamide |
| 277 | 4-fluoro-N-(3-(2-(hydroxymethyl)-4-methylpiperazin-1-yl)phenyl)-7-methyl-1H- |
| | indole-2-carboxamide |
| 278 | 4-fluoro-N-(3-(imidazo[1,5-a]pyridin-6-yl)phenyl)-7-methyl-1H-indole-2- |
| | carboxamide |
| 279 | 4-fluoro-7-methyl-N-(3-(4-methyl-1,4-diazepan-1-yl)phenyl)-1H-indole-2- |
| | carboxamide |
| 280 | 4-fluoro-N-(3-(4-isopropyl-2-methylpiperazin-1-yl)phenyl)-7-methyl-1H-indole- |
| | 2-carboxamide |
| 281 | N-(3-(4H-1,2,4-triazol-4-yl)phenyl)-4-fluoro-7-methyl-1H-indole-2-carboxamide |
| 282 | N-(3-(3-(dimethylamino)cyclopentyl)phenyl)-4-fluoro-7-methyl-1H-indole-2- |
| | carboxamide |
| 283 | 4-fluoro-7-methyl-N-(3-(pyrrolidin-3-yl)phenyl)-1H-indole-2-carboxamide |
| 284 | N-(3-(2-oxa-5-azabicyclo[2.2.1]heptan-5-yl)phenyl)-4-fluoro-7-methyl-1H- |
| | indole-2-carboxamide |
| 285 | 4-fluoro-7-methyl-N-(3-(pyridin-2-yl)phenyl)-1H-indole-2-carboxamide |
| 286 | 4-fluoro-N-(3-(imidazo[1,2-a]pyridin-2-yl)phenyl)-7-methyl-1H-indole-2- |
| | carboxamide |
| 287 | N-(3-(4-oxa-7-azaspiro[2.5]octan-7-yl)phenyl)-4-fluoro-7-methyl-1H-indole-2- |
| | carboxamide |
| 288 | 4-fluoro-7-methyl-N-(3-(4-(methylamino)-4-oxobutyl)phenyl)-1H-indole-2- |
| | carboxamide |
| 289 | N-(3-(2,5-dimethylmorpholino)phenyl)-4-fluoro-7-methyl-1H-indole-2- |
| | carboxamide |
| 290 | 4-fluoro-7-methyl-N-(3-(pyrazolo[1,5-a]pyridin-3-yl)phenyl)-1H-indole-2- |
| | carboxamide |
| 291 | N-(3-(3-oxa-8-azabicyclo[3.2.1]octan-8-yl)phenyl)-4-fluoro-7-methyl-1H-indole- |
| | 2-carboxamide |
| 292 | N-(3-(5-acetyl-2,5-diazabicyclo[2.2.1]heptan-2-yl)phenyl)-4-fluoro-7-methyl-1H- |
| | indole-2-carboxamide |
| 293 | 4-fluoro-7-methyl-N-(3-(3-(methylamino)-3-oxopropyl)phenyl)-1H-indole-2- |
| | carboxamide |
| 294 | 4-fluoro-7-methyl-N-(3-(1-methyloctahydro-1H-indol-5-yl)phenyl)-1H-indole-2- |
| | carboxamide |
| 295 | N-(2-(4-acetylpiperazin-1-yl)pyrimidin-4-yl)-4-fluoro-7-methyl-1H-indole-2- |
| | carboxamide |
| 296 | N-(3-(1,2,4-oxadiazol-5-yl)phenyl)-4-fluoro-7-methyl-1H-indole-2-carboxamide |
| 297 | 4-fluoro-7-methyl-N-(3-(piperidin-1-yl)phenyl)-1H-indole-2-carboxamide |
| 298 | N-(3-((3S,5S)-4-acetyl-3,5-dimethylpiperazin-1-yl)phenyl)-4-fluoro-7-methyl- |
| | 1H-indole-2-carboxamide |
| 299 | 4-fluoro-N-(3-(imidazo[1,5-a]pyridin-1-yl)phenyl)-7-methyl-1H-indole-2- |
| | carboxamide |
| 300 | N-(3-(2-amino-2-oxoethyl)phenyl)-4-fluoro-7-methyl-1H-indole-2-carboxamide |
| 301 | N-(3-((3S,4R)-4-(dimethylamino)-3-fluoropiperidin-1-yl)phenyl)-4-fluoro-7- |
| | methyl-1H-indole-2-carboxamide |
| 302 | N-(3-((1R,3S)-3-(dimethylamino)cyclopentyl)phenyl)-4-fluoro-7-methyl-1H- |
| | indole-2-carboxamide |
| 303 | N-(3-((2S,4R)-4-(dimethylamino)-2-methylpiperidin-1-yl)phenyl)-4-fluoro-7- |
| | methyl-1H-indole-2-carboxamide |
| 304 | N-(3-(4H-1,2,4-triazol-3-yl)phenyl)-4-fluoro-7-methyl-1H-indole-2-carboxamide |
| 305 | 4-fluoro-7-methyl-N-(5-(3-(N-methylacetamido)piperidin-1-yl)tetrahydro-2H- |
| | pyran-3-yl)-1H-indole-2-carboxamide |
| 306 | N-(3-(3-cyclopropylmorpholino)phenyl)-4-fluoro-7-methyl-1H-indole-2- |
| | carboxamide |
| 307 | 4-fluoro-7-methyl-N-(3-(morpholin-3-yl)phenyl)-1H-indole-2-carboxamide |
| 308 | N-(3-((2R,4R)-4-(dimethylamino)-2-methylpiperidin-1-yl)phenyl)-4-fluoro-7- |
| | methyl-1H-indole-2-carboxamide |
| 309 | N-((5-((1H-imidazol-1-yl)methyl)-3-methylpyridin-2-yl)(cyclopropyl)methyl)-7- |
| | methyl-1H-indole-2-carboxamide |
| 310 | N-(3-(3-aminopiperidin-1-yl)phenyl)-4-fluoro-7-methyl-1H-indole-2- |
| | carboxamide |
| 311 | N-(3-(difluoromethyl)-5-(4-(dimethylamino)piperidin-1-yl)phenyl)-4-fluoro-7- |
| | methyl-1H-indole-2-carboxamide |
| 312 | 4-fluoro-7-methyl-N-(3-(4-phenylpiperazin-1-yl)phenyl)-1H-indole-2- |
| | carboxamide |
| 313 | N-(3-((1r,4r)-4-(dimethylamino)cyclohexyl)-5-fluorophenyl)-4-fluoro-7-methyl- |
| | 1H-indole-2-carboxamide |
| 314 | 4-fluoro-7-methyl-N-(3-(pyrrolidine-2-carboxamido)phenyl)-1H-indole-2- |
| | carboxamide |
| 315 | ″″″4-fluoro-N-(3-(2-hydroxypropan-2-yl)phenyl)-7-methyl-1H-indole-2- |
| | carboxamide |
| 316 | 4-fluoro-7-methyl-N-(3-(pyrimidin-2-yl)phenyl)-1H-indole-2-carboxamide |
| 317 | N-(cyclopropyl(5-((2,4-dimethyl-1H-imidazol-1-yl)methyl)-3-methylpyridin-2- |
| | yl)methyl)-4-fluoro-7-methyl-1H-indole-2-carboxamide |
| 318 | N-(3-(2,4-dimethylpiperazin-1-yl)phenyl)-4-fluoro-7-methyl-1H-indole-2- |
| | carboxamide |
| 319 | N-(3-(5-amino-1,3,4-oxadiazol-2-yl)phenyl)-4-fluoro-7-methyl-1H-indole-2- |
| | carboxamide |
| 320 | 4-fluoro-7-methyl-N-(2-methyl-3-(4-methylpiperazin-1-yl)phenyl)-1H-indole-2- |
| | carboxamide |
| 321 | 4-fluoro-7-methyl-N-(3-(2-oxopiperazin-1-yl)phenyl)-1H-indole-2-carboxamide |
| 322 | N-(3-(1H-pyrazol-5-yl)phenyl)-4-fluoro-7-methyl-1H-indole-2-carboxamide |
| 323 | N-(3-(2-(dimethylamino)ethoxy)phenyl)-4-fluoro-7-methyl-1H-indole-2- |
| | carboxamide |
| 324 | N-(3-((1S,3R)-3-(dimethylamino)cyclopentyl)phenyl)-4-fluoro-7-methyl-1H- |
| | indole-2-carboxamide |
| 325 | 4-fluoro-7-methyl-N-(3-(methyl(piperidin-4-yl)amino)phenyl)-1H-indole-2- |
| | carboxamide |
| 326 | 4-fluoro-7-methyl-N-(3-(6-(methylamino)pyridin-2-yl)phenyl)-1H-indole-2- |
| | carboxamide |
| 327 | 4-fluoro-7-methyl-N-(2-oxo-2,3,4,5-tetrahydro-1H-benzo[d]azepin-1-yl)-1H- |
| | indole-2-carboxamide |
| 328 | N-(3-(5,6-dihydro-[1,2,4]triazolo[4,3-a]pyrazin-7(8H)-yl)phenyl)-4-fluoro-7- |
| | methyl-1H-indole-2-carboxamide |
| 329 | N-(3-(dimethylcarbamoyl)phenyl)-4-fluoro-7-methyl-1H-indole-2-carboxamide |
| 330 | 4-fluoro-7-methyl-N-(3-((1-methylpyrrolidin-3-yl)amino)phenyl)-1H-indole-2- |
| | carboxamide |
| 331 | 4-fluoro-N-(3-(3-(2-hydroxyethyl)morpholino)phenyl)-7-methyl-1H-indole-2- |
| | carboxamide |
| 332 | 4-fluoro-7-methyl-N-(3-(pyrrolidin-1-yl)phenyl)-1H-indole-2-carboxamide |
| 333 | N-(3-chloro-5-(morpholinomethyl)phenyl)-4-fluoro-7-methyl-1H-indole-2- |
| | carboxamide |
| 334 | 4-fluoro-7-methyl-N-(1-(3-methylpyridin-2-yl)-4-morpholino-4-oxobutyl)-1H- |
| | indole-2-carboxamide |
| 335 | N-(3-(3-(dimethylamino)piperidin-1-yl)phenyl)-4-fluoro-7-methyl-1H-indole-2- |
| | carboxamide |
| 336 | 4-fluoro-N-(3-(4-isopropylpiperidin-1-yl)phenyl)-7-methyl-1H-indole-2- |
| | carboxamide |
| 337 | N-(3-(1H-imidazol-4-yl)phenyl)-4-fluoro-7-methyl-1H-indole-2-carboxamide |
| 338 | 4-fluoro-7-methyl-N-(8-(4-methylpiperazin-1-yl)imidazo[1,2-a]pyridin-6-yl)-1H- |
| | indole-2-carboxamide |
| 339 | N-(3-chloro-5-((1r,4r)-4-(dimethylamino)cyclohexyl)phenyl)-4-fluoro-7-methyl- |
| | 1H-indole-2-carboxamide |
| 340 | 4-fluoro-7-methyl-N-(2-(4-methylpiperazin-1-yl)pyridin-4-yl)-1H-indole-2- |
| | carboxamide |
| 341 | 4-fluoro-N-(3-(2-hydroxyethyl)phenyl)-7-methyl-1H-indole-2-carboxamide |
| 342 | 4-fluoro-7-methyl-N-(3-(methyl(1-methylpyrrolidin-3-yl)amino)phenyl)-1H- |
| | indole-2-carboxamide |
| 343 | N-(3-((1s,4s)-4-(dimethylamino)cyclohexyl)phenyl)-4-fluoro-7-methyl-1H- |
| | indole-2-carboxamide |
| 344 | 4-fluoro-7-methyl-N-(6-(4-methylpiperazin-1-yl)pyridin-2-yl)-1H-indole-2- |
| | carboxamide |
| 345 | N-(3-cyano-5-(4-(dimethylamino)piperidin-1-yl)phenyl)-4-fluoro-7-methyl-1H- |
| | indole-2-carboxamide |
| 346 | 4-fluoro-N-(4′-(hydroxymethyl)-[1,1′-biphenyl]-3-yl)-7-methyl-1H-indole-2- |
| | carboxamide |
| 347 | N-(3-(3-(4-aminopiperidin-1-yl)pyrrolidin-1-yl)phenyl)-4-fluoro-7-methyl-1H- |
| | indole-2-carboxamide |
| 348 | 4-fluoro-7-methyl-N-(3-(5-methyl-2,5-diazabicyclo[2.2.1]heptan-2-yl)phenyl)- |
| | 1H-indole-2-carboxamide |
| 349 | 4-fluoro-7-methyl-N-(3-(3-methyl-3,8-diazabicyclo[3.2.1]octan-8-yl)phenyl)-1H- |
| | indole-2-carboxamide |
| 350 | 4-fluoro-7-methyl-N-(8-(4-methylpiperazin-1-yl)quinolin-6-yl)-1H-indole-2- |
| | carboxamide |
| 351 | 4-fluoro-7-methyl-N-(3-(methyl(1-methylpiperidin-4-yl)amino)phenyl)-1H- |
| | indole-2-carboxamide |
| 352 | 4-fluoro-7-methyl-N-(3-((1-methylpiperidin-3-yl)methyl)phenyl)-1H-indole-2- |
| | carboxamide |
| 353 | 4-fluoro-N-(3-fluoro-5-((1r,4r)-4-morpholinocyclohexyl)phenyl)-7-methyl-1H- |
| | indole-2-carboxamide |
| 354 | 4-fluoro-7-methyl-N-(3-(pyrazolo[1,5-a]pyridin-2-yl)phenyl)-1H-indole-2- |
| | carboxamide |
| 355 | 4-fluoro-7-methyl-N-(5-(4-methylpiperazin-1-yl)imidazo[1,2-a]pyridin-7-yl)-1H- |
| | indole-2-carboxamide |
| 356 | N-(2-(1-(cyclopropylmethyl)-1H-imidazol-4-yl)-1-(3-methylpyridin-2-yl)ethyl)- |
| | 4-fluoro-7-methyl-1H-indole-2-carboxamide |
| 357 | N-(3-((1H-imidazol-2-yl)methyl)phenyl)-4-fluoro-7-methyl-1H-indole-2- |
| | carboxamide |
| 358 | 4-fluoro-7-methyl-N-(1-(3-methylpyridin-2-yl)-2-(4- |
| | (morpholinomethyl)phenyl)ethyl)-1H-indole-2-carboxamide |
| 359 | N-((1S,3S)-3-(4-acetylpiperazin-1-yl)cyclohexyl)-4-fluoro-7-methyl-1H-indole-2- |
| | carboxamide |
| 360 | N-(5-(4-acetylpiperazin-1-yl)-1-methylpiperidin-3-yl)-4-fluoro-7-methyl-1H- |
| | indole-2-carboxamide |
| 361 | 4-fluoro-7-methyl-N-(8-oxo-6,7,8,9-tetrahydro-5H-pyrido[2,3-d]azepin-9-yl)-1H- |
| | indole-2-carboxamide |
| 362 | N-((1S,3S)-3-(1,4-oxazepan-4-yl)cyclohexyl)-4-fluoro-7-methyl-1H-indole-2- |
| | carboxamide |
| 363 | N-(5-(4-acetylpiperazin-1-yl)tetrahydro-2H-pyran-3-yl)-4-fluoro-7-methyl-1H- |
| | indole-2-carboxamide |
| 364 | 4-fluoro-7-methyl-N-(2,3,4,5-tetrahydro-1H-benzo[d]azepin-1-yl)-1H-indole-2- |
| | carboxamide |
| 365 | N-(3-(3-acetyl-3,6-diazabicyclo[3.1.1]heptan-6-yl)phenyl)-4-fluoro-7-methyl-1H- |
| | indole-2-carboxamide |
| 366 | N-(1′-acetyl-[1,4′-bipiperidin]-3-yl)-4-fluoro-7-methyl-1H-indole-2-carboxamide |
| 367 | 4-fluoro-7-methyl-N-(1′-methyl-2′-oxo-[1,4′-bipiperidin]-3-yl)-1H-indole-2- |
| | carboxamide |
| 368 | 4-fluoro-7-methyl-N-(5-(pyrimidin-5-yl)tetrahydro-2H-pyran-3-yl)-1H-indole-2- |
| | carboxamide |
| 369 | 4-fluoro-7-methyl-N-((1S,3S)-3-(pyrimidin-5-yl)cyclohexyl)-1H-indole-2- |
| | carboxamide |
| 370 | 4-fluoro-7-methyl-N-((1S,3S)-3-(4-methyl-3-oxo-1,4-diazepan-1-yl)cyclohexyl)- |
| | 1H-indole-2-carboxamide |
| 371 | 4-fluoro-7-methyl-N-(5-(1-methylpiperidin-4-yl)-1H-imidazol-2-yl)-1H-indole-2- |
| | carboxamide |
| 372 | N-((1R,3R)-3-(4-acetylpiperazin-1-yl)cyclohexyl)-4-fluoro-7-methyl-1H-indole- |
| | 2-carboxamide |
| 373 | N-((1S,3R)-3-(4-acetylpiperazin-1-yl)cyclohexyl)-4-fluoro-7-methyl-1H-indole- |
| | 2-carboxamide |
| 374 | 4-fluoro-7-methyl-N-(3-morpholinocyclohexyl)-1H-indole-2-carboxamide |
| 375 | N-(3-(4-(dimethylamino)-4-(trifluoromethyl)piperidin-1-yl)phenyl)-4-fluoro-7- |
| | methyl-1H-indole-2-carboxamide |
| 376 | N-(3-(4-acetylpiperazin-1-yl)cyclopentyl)-4-fluoro-7-methyl-1H-indole-2- |
| | carboxamide |
| 377 | N-(1-(2-(dimethylamino)-2-oxoethyl)piperidin-3-yl)-4-fluoro-7-methyl-1H- |
| | indole-2-carboxamide |
| 378 | N-(1-(3-(dimethylamino)-3-oxopropyl)piperidin-3-yl)-4-fluoro-7-methyl-1H- |
| | indole-2-carboxamide |
| 379 | 4-fluoro-7-methyl-N-((1R,3R)-3-(pyrimidin-5-yl)cyclohexyl)-1H-indole-2- |
| | carboxamide |
| 380 | 4-fluoro-7-methyl-N-((1R,3S)-3-(pyrimidin-5-yl)cyclohexyl)-1H-indole-2- |
| | carboxamide |
| 381 | 4-fluoro-7-methyl-N-((1R,3R)-3-morpholinocyclohexyl)-1H-indole-2- |
| | carboxamide |
| 382 | 4-fluoro-7-methyl-N-((1R,3R)-3-(4-methyl-3-oxopiperazin-1-yl)cyclohexyl)-1H- |
| | indole-2-carboxamide |
| 383 | N-(4-(4-acetylpiperazin-1-yl)cyclohexyl)-4-fluoro-7-methyl-1H-indole-2- |
| | carboxamide |
| 384 | 4-fluoro-7-methyl-N-(1-(2-(methylamino)-2-oxoethyl)piperidin-3-yl)-1H-indole- |
| | 2-carboxamide |
| 385 | 4-fluoro-7-methyl-N-((1R,3R)-3-(3-(N-methylacetamido)pyrrolidin-1- |
| | yl)cyclohexyl)-1H-indole-2-carboxamide |
| 386 | 4-fluoro-7-methyl-N-(1-(3-(methylamino)-3-oxopropyl)piperidin-3-yl)-1H- |
| | indole-2-carboxamide |
| 387 | 4-fluoro-7-methyl-N-((1R,3R)-3-(4-methyl-3-oxo-1,4-diazepan-1-yl)cyclohexyl)- |
| | 1H-indole-2-carboxamide |
| 388 | N-((1R,3R)-3-(1,4-oxazepan-4-yl)cyclohexyl)-4-fluoro-7-methyl-1H-indole-2- |
| | carboxamide |
| 389 | 4-fluoro-7-methyl-N-(1-(3-(N-methylacetamido)propyl)piperidin-3-yl)-1H- |
| | indole-2-carboxamide |
| 390 | 4-fluoro-7-methyl-N-(3-methyl-2-oxo-2,3,4,5-tetrahydro-1H-benzo[d]azepin-1- |
| | yl)-1H-indole-2-carboxamide |
| 391 | 4-fluoro-7-methyl-N-(3-(4-methylpiperazin-1-yl)-2H-indazol-7-yl)-1H-indole-2- |
| | carboxamide |
| 392 | 4-fluoro-7-methyl-N-((1-(2-(4-methylpiperazin-1-yl)phenyl)cyclopropyl)methyl)- |
| | 1H-indole-2-carboxamide |
| 393 | 7-methyl-N-(3-morpholinophenyl)-1H-indole-2-carboxamide |
| 394 | 4-fluoro-7-methyl-N-(3-(1-methyl-1H-pyrazol-3-yl)phenyl)-1H-indole-2- |
| | carboxamide |
| 395 | N-(3-(1-aminocyclopropyl)phenyl)-4-fluoro-7-methyl-1H-indole-2-carboxamide |
| 396 | N-(3-(4-glycylpiperazin-1-yl)phenyl)-7-methyl-1H-indole-2-carboxamide |
| 397 | N-(3-(5-amino-1H-pyrazol-4-yl)phenyl)-4-fluoro-7-methyl-1H-indole-2- |
| | carboxamide |
| 398 | N-(3-(4-(dimethylamino)piperidin-1-yl)-5-ethylphenyl)-4-fluoro-7-methyl-1H- |
| | indole-2-carboxamide |
| 399 | 4-fluoro-7-methyl-N-(3-(methylsulfonyl)phenyl)-1H-indole-2-carboxamide |
| 400 | N-(cyclopropyl(3-methylimidazo[1,2-a]pyridin-2-yl)methyl)-4-fluoro-7-methyl- |
| | 1H-indole-2-carboxamide |
| 401 | 4-fluoro-7-methyl-N-(8-(4-methylpiperazin-1-yl)isoquinolin-6-yl)-1H-indole-2- |
| | carboxamide |
| 402 | N-(cyclopropyl(5-methyl-6′-(morpholinomethyl)-[3,3′-bipyridin]-6-yl)methyl)-7- |
| | methyl-1H-indole-2-carboxamide |
| 403 | 4-fluoro-7-methyl-N-(5,6,7,8-tetrahydroisoquinolin-5-yl)-1H-indole-2- |
| | carboxamide |
| 404 | N-(cyclopropyl(3-methyl-5-(pyrimidin-2-yl)pyridin-2-yl)methyl)-7-methyl-1H- |
| | indole-2-carboxamide |
| 405 | N-(3-(4-((2-(diethylamino)ethyl)sulfonyl)piperazin-1-yl)phenyl)-7-methyl-1H- |
| | indole-2-carboxamide |
| 406 | N-(cyclopropyl(5-methyl-[3,3′-bipyridin]-6-yl)methyl)-7-methyl-1H-indole-2- |
| | carboxamide |
| 407 | 4-fluoro-7-methyl-N-(2-methyl-5-(4-methylpiperazin-1-yl)phenyl)-1H-indole-2- |
| | carboxamide |
| 408 | 7-methyl-N-(1-(3-methylpyridin-2-yl)-2-phenylethyl)-1H-indole-2-carboxamide |
| 409 | 4-fluoro-7-methyl-N-(3-((4-methylpiperazin-1-yl)methyl)isoquinolin-6-yl)-1H- |
| | indole-2-carboxamide |
| 410 | 4-fluoro-7-methyl-N-(5-(4-methylpiperazin-1-yl)pyridin-3-yl)-1H-indole-2- |
| | carboxamide |
| 411 | 4-fluoro-7-methyl-N-(3-(3-methyl-2-oxo-3,8-diazabicyclo[3.2.1]octan-8- |
| | yl)phenyl)-1H-indole-2-carboxamide |
| 412 | N-(cyclopropyl(3-methyl-5-((1-methylpiperidin-4-yl)carbamoyl)pyridin-2- |
| | yl)methyl)-4-fluoro-7-methyl-1H-indole-2-carboxamide |
| 413 | N-(3-(4-((2-aminoethyl)amino)piperidin-1-yl)phenyl)-4-fluoro-7-methyl-1H- |
| | indole-2-carboxamide |
| 414 | 4-fluoro-7-methyl-N-(1-(3-methyl-5-(2-(4-methylpiperazin-1-yl)ethyl)pyridin-2- |
| | yl)-3-phenylpropyl)-1H-indole-2-carboxamide |
| 415 | N-(3-(4-(dimethylamino)piperidin-1-yl)-2H-indazol-7-yl)-4-fluoro-7-methyl-1H- |
| | indole-2-carboxamide |
| 416 | 4-fluoro-7-methyl-N-(2-(piperidin-4-ylamino)quinazolin-6-yl)-1H-indole-2- |
| | carboxamide |
| 417 | 4-fluoro-7-methyl-N-(3-(4-(1-phenylethyl)piperazin-1-yl)phenyl)-1H-indole-2- |
| | carboxamide |
| 418 | N-(3-(2-aminoethyl)phenyl)-4-fluoro-7-methyl-1H-indole-2-carboxamide |
| 419 | N-((3-chloropyridin-2-yl)(cyclopropyl)methyl)-7-methyl-1H-indole-2- |
| | carboxamide |
| 420 | 7-methyl-N-(1-(3-methylpyridin-2-yl)-4-morpholino-4-oxobutyl)-1H-indole-2- |
| | carboxamide |
| 421 | 4-fluoro-7-methyl-N-(3-((4-methylpiperazin-1-yl)methyl)phenyl)-1H-indole-2- |
| | carboxamide |
| 422 | N-(3-(3-oxa-6-azabicyclo[3.1.1]heptan-6-yl)phenyl)-4-fluoro-7-methyl-1H- |
| | indole-2-carboxamide |
| 423 | N-(cyclopropyl(3-methyl-5-(4-(2-(pyridin-3-yl)acetamido)cyclohexyl)pyridin-2- |
| | yl)methyl)-7-methyl-1H-indole-2-carboxamide |
| 424 | N-(cyclopropyl(3-methylpyrazolo[1,5-a]pyridin-2-yl)methyl)-4-fluoro-7-methyl- |
| | 1H-indole-2-carboxamide |
| 425 | 4-fluoro-7-methyl-N-(3-(pyrrolidin-2-yl)phenyl)-1H-indole-2-carboxamide |
| 426 | N-((6′-amino-5-methyl-[3,3′-bipyridin]-6-yl)(cyclopropyl)methyl)-7-methyl-1H- |
| | indole-2-carboxamide |
| 427 | N-(3-((dimethylamino)methyl)imidazo[1,2-a]pyridin-6-yl)-7-methyl-1H-indole-2- |
| | carboxamide |
| 428 | N-(3-(1H-imidazol-2-yl)phenyl)-4-fluoro-7-methyl-1H-indole-2-carboxamide |
| 429 | N-(3-(4-((4-aminocyclohexyl)sulfonyl)piperazin-1-yl)phenyl)-7-methyl-1H- |
| | indole-2-carboxamide |
| 430 | N-(cyclopropyl(5-(2-hydroxy-3-((pyridin-3-ylmethyl)amino)propoxy)-3- |
| | methylpyridin-2-yl)methyl)-7-methyl-1H-indole-2-carboxamide |
| 431 | 7-methyl-N-(1-(3-methylpyridin-2-yl)-4-morpholino-4-oxobutyl)-4-vinyl-1H- |
| | indole-2-carboxamide |
| 432 | N-((4-cyano-3-methylpyridin-2-yl)(cyclopropyl)methyl)-4-fluoro-7-methyl-1H- |
| | indole-2-carboxamide |
| 433 | N-(3-(4-(((1H-imidazol-5-yl)methyl)amino)piperidin-1-yl)phenyl)-4-fluoro-7- |
| | methyl-1H-indole-2-carboxamide |
| 434 | N-(4-(4-hydroxypiperidin-1-yl)-1-(3-methylpyridin-2-yl)-4-oxobutyl)-7-methyl- |
| | 1H-indole-2-carboxamide |
| 435 | N-(cyclopropyl(3-methyl-5-(2-(4-methylpiperazin-1-yl)ethyl)pyridin-2- |
| | yl)methyl)-7-methyl-1H-indole-2-carboxamide |
| 436 | N-(3-(8-(dimethylamino)-3-azabicyclo[3.2.1]octan-3-yl)phenyl)-4-fluoro-7- |
| | methyl-1H-indole-2-carboxamide |
| 437 | N-(isoquinolin-6-yl)-7-methyl-1H-indole-2-carboxamide |
| 438 | 4-fluoro-7-methyl-N-(3-(piperidin-2-yl)phenyl)-1H-indole-2-carboxamide |
| 439 | N-(3-(4-(2-aminoacetamido)piperidin-1-yl)phenyl)-7-methyl-1H-indole-2- |
| | carboxamide |
| 440 | N-(cyclopropyl(3-methyl-5-(4-(3-(pyridin-3-yl)propyl)piperazin-1-yl)pyridin-2- |
| | yl)methyl)-7-methyl-1H-indole-2-carboxamide |
| 441 | N-(cyclopropyl(3-methyl-5-(2-(4-methylpiperazin-1-yl)-2-oxoethyl)pyridin-2- |
| | yl)methyl)-7-methyl-1H-indole-2-carboxamide |
| 442 | 7-methyl-N-(2-methyl-5-(morpholinomethyl)phenyl)-1H-indole-2-carboxamide |
| 443 | N-(6-(4-((2-aminoethyl)sulfonyl)piperazin-1-yl)pyridin-2-yl)-7-methyl-1H- |
| | indole-2-carboxamide |
| 444 | N-(3-(5-amino-1H-pyrazol-3-yl)phenyl)-4-fluoro-7-methyl-1H-indole-2- |
| | carboxamide |
| 445 | N-((5-(1H-imidazol-2-yl)-3-methylpyridin-2-yl)(cyclopropyl)methyl)-7-methyl- |
| | 1H-indole-2-carboxamide |
| 446 | N-(4-(dimethylamino)-1-(3-methylpyridin-2-yl)-4-oxobutyl)-7-methyl-1H-indole- |
| | 2-carboxamide |
| 447 | N-(cyclopropyl(3-methyl-5-(2-(2-(pyridin-3-yl)acetamido)ethyl)pyridin-2- |
| | yl)methyl)-7-methyl-1H-indole-2-carboxamide |
| 448 | 7-methyl-N-(7-(4-methylpiperazine-1-carbonyl)naphthalen-2-yl)-1H-indole-2- |
| | carboxamide |
| 449 | N-(3-(4-((2-aminoethyl)sulfonyl)piperazin-1-yl)phenyl)-7-methyl-1H-indole-2- |
| | carboxamide |
| 450 | N-(cyclopropyl(4-methylpyridazin-3-yl)methyl)-4-fluoro-7-methyl-1H-indole-2- |
| | carboxamide |
| 451 | N-((5-(((1S,2R)-2-aminocyclobutyl)carbamoyl)-3-methylpyridin-2- |
| | yl)(cyclopropyl)methyl)-7-methyl-1H-indole-2-carboxamide |
| 452 | N-(3-(4-((2-aminoethyl)amino)piperidin-1-yl)phenyl)-7-methyl-1H-indole-2- |
| | carboxamide |
| 453 | 7-methyl-N-(7-((4-methylpiperazin-1-yl)methyl)naphthalen-2-yl)-1H-indole-2- |
| | carboxamide |
| 454 | 4-fluoro-7-methyl-N-(3-sulfamoylphenyl)-1H-indole-2-carboxamide |
| 455 | N-((5-(((1R,2R)-2-aminocyclobutyl)carbamoyl)-3-methylpyridin-2- |
| | yl)(cyclopropyl)methyl)-7-methyl-1H-indole-2-carboxamide |
| 456 | 7-methyl-N-(4-methyl-3-(morpholinomethyl)phenyl)-1H-indole-2-carboxamide |
| 457 | 7-methyl-N-((1-(2-morpholinophenyl)cyclopropyl)methyl)-1H-indole-2- |
| | carboxamide |
| 458 | 4-fluoro-7-methyl-N-(3-oxo-1,2,3,4-tetrahydroisoquinolin-4-yl)-1H-indole-2- |
| | carboxamide |
| 459 | N-(imidazo[1,2-a]pyridin-6-yl)-7-methyl-1H-indole-2-carboxamide |
| 460 | N-(4-(5,6-dihydroimidazo[1,5-a]pyrazin-7(8H)-yl)-1-(3-methylpyridin-2-yl)-4- |
| | oxobutyl)-7-methyl-1H-indole-2-carboxamide |
| 461 | N-(cyclopropyl(5-(2-hydroxy-3-((3-methoxybenzyl)amino)propoxy)-3- |
| | methylpyridin-2-yl)methyl)-7-methyl-1H-indole-2-carboxamide |
| 462 | N-(cyclopropyl(3-methylpyridin-2-yl)methyl)-4-fluoro-7-methyl-1H-indole-2- |
| | carboxamide |
| 463 | (R)-N-(cyclopropyl(3-methylpyridin-2-yl)methyl)-7-methyl-1H-indole-2- |
| | carboxamide |
| 464 | 7-ethyl-N-(1-(3-methylpyridin-2-yl)-4-morpholino-4-oxobutyl)-1H-indole-2- |
| | carboxamide |
| 465 | 7-methyl-N-(3-((4-methylpiperazin-1-yl)methyl)isoquinolin-6-yl)-1H-indole-2- |
| | carboxamide |
| 466 | 7-methyl-N-(quinazolin-7-yl)-1H-indole-2-carboxamide |
| 467 | N-(3-(4-(2-aminoethyl)piperazin-1-yl)phenyl)-7-methyl-1H-indole-2- |
| | carboxamide |
| 468 | N-(3-(3-((dimethylamino)methyl)morpholino)phenyl)-4-fluoro-7-methyl-1H- |
| | indole-2-carboxamide |
| 469 | 7-methyl-N-(2-methyl-5-(4-methylpiperazin-1-yl)phenyl)-1H-indole-2- |
| | carboxamide |
| 470 | N-(cyclopropyl(3-methyl-5-((1-methylpiperidin-4-yl)carbamoyl)pyridin-2- |
| | yl)methyl)-7-methyl-1H-indole-2-carboxamide |
| 471 | 7-methyl-N-(1-(3-methylpyridin-2-yl)propyl)-1H-indole-2-carboxamide |
| 472 | N-(cyclopropyl(3-methylquinolin-2-yl)methyl)-7-methyl-1H-indole-2- |
| | carboxamide |
| 473 | 7-methyl-N-(2-(piperidin-4-ylamino)quinazolin-6-yl)-1H-indole-2-carboxamide |
| 474 | 7-methyl-N-(2-methyl-1-(3-methylpyridin-2-yl)propyl)-1H-indole-2-carboxamide |
| 475 | N-(cyclopropyl(3-methylpyridin-2-yl)methyl)-7-methyl-1H-indole-2- |
| | carboxamide |
| 476 | N-(5-(4-((2-aminoethyl)sulfonyl)piperazin-1-yl)-2-methylphenyl)-7-methyl-1H- |
| | indole-2-carboxamide |
| 477 | N-(cyclopropyl(3-methyl-5-((4-methylpiperazin-1-yl)methyl)pyridin-2- |
| | yl)methyl)-7-methyl-1H-indole-2-carboxamide |
| 478 | 7-methyl-N-(3-(morpholinomethyl)phenyl)-1H-indole-2-carboxamide |
| 479 | 7-methyl-N-phenyl-1H-indole-2-carboxamide |
| 480 | 7-methyl-N-(1-(3-methylpyridin-2-yl)ethyl)-1H-indole-2-carboxamide |
| 481 | N-(cyclopropyl(7-methylimidazo[1,2-b]pyridazin-6-yl)methyl)-4-fluoro-7- |
| | methyl-1H-indole-2-carboxamide |
| 482 | 7-methyl-N-(2-oxo-2,3,4,5-tetrahydro-1H-benzo[d]azepin-1-yl)-1H-indole-2- |
| | carboxamide |
| 483 | 7-methyl-N-(2-(2-morpholinophenyl)propyl)-1H-indole-2-carboxamide |
| 484 | N-(imidazo[1,2-a]pyrimidin-6-yl)-7-methyl-1H-indole-2-carboxamide |
| 485 | 4-ethyl-7-methyl-N-(1-(3-methylpyridin-2-yl)-4-morpholino-4-oxobutyl)-1H- |
| | indole-2-carboxamide |
| 486 | N-(3-((diethylamino)methyl)phenyl)-7-methyl-1H-indole-2-carboxamide |
| 487 | 7-methyl-N-(7-(morpholinomethyl)naphthalen-2-yl)-1H-indole-2-carboxamide |
| 488 | 7-methyl-N-(3-((4-methylpiperazin-1-yl)methyl)phenyl)-1H-indole-2- |
| | carboxamide |
| 489 | 7-methyl-N-(1-(3-methylpyridin-2-yl)-2-morpholinoethyl)-1H-indole-2- |
| | carboxamide |
| 490 | 7-methyl-N-(2-methyl-5-morpholinophenyl)-1H-indole-2-carboxamide |
| 491 | N-(2-(dimethylamino)-2-oxo-1-(pyridin-2-yl)ethyl)-4-fluoro-7-methyl-1H-indole- |
| | 2-carboxamide |
| 492 | N-(cyclopropyl(2-hydroxy-6-methylphenyl)methyl)-7-methyl-1H-indole-2- |
| | carboxamide |
| 493 | N-(cyclopropyl(5-methylpyrimidin-4-yl)methyl)-7-methyl-1H-indole-2- |
| | carboxamide |
| 494 | 7-methyl-N-(1-(3-methylpyridin-2-yl)-5-morpholino-5-oxopentyl)-1H-indole-2- |
| | carboxamide |
| 495 | N-(3-((dimethylamino)methyl)-5-methylimidazo[1,2-a]pyridin-6-yl)-7-methyl- |
| | 1H-indole-2-carboxamide |
| 496 | N-((5-(((1r,4r)-4-aminocyclohexyl)carbamoyl)-3-methylpyridin-2- |
| | yl)(cyclopropyl)methyl)-7-methyl-1H-indole-2-carboxamide |
| 497 | N-(cyclopropyl(3-methylpyridin-2-yl)methyl)-5-fluoro-7-methyl-1H-indole-2- |
| | carboxamide |
| 498 | 4-fluoro-7-methyl-N-(5,6,7,8-tetrahydroquinolin-8-yl)-1H-indole-2-carboxamide |
| 499 | N-(2-(4-acetylpiperazin-1-yl)benzyl)-7-methyl-1H-indole-2-carboxamide |
| 500 | N-(2-cyclopropyl-1-(3-methylpyridin-2-yl)ethyl)-7-methyl-1H-indole-2- |
| | carboxamide |
| 501 | N-(3-(2-amino-1H-imidazol-4-yl)phenyl)-4-fluoro-7-methyl-1H-indole-2- |
| | carboxamide |
| 502 | N-(3-(5-aminoisoxazol-3-yl)phenyl)-4-fluoro-7-methyl-1H-indole-2-carboxamide |
| 503 | N-(2-amino-2-oxo-1-(pyridin-2-yl)ethyl)-4-fluoro-7-methyl-1H-indole-2- |
| | carboxamide |
| 504 | 7-methyl-N-((1r,4r)-4-methylcyclohexyl)-1H-indole-2-carboxamide |
| 505 | 7-methyl-N-(tetrahydro-2H-pyran-4-yl)-1H-indole-2-carboxamide |
| 506 | N-(cyclopropyl(3-methyl-6-oxo-1,6-dihydropyridin-2-yl)methyl)-7-methyl-1H- |
| | indole-2-carboxamide |
| 507 | N-(cyclopropyl(isoquinolin-1-yl)methyl)-4-fluoro-7-methyl-1H-indole-2- |
| | carboxamide |
| 508 | 7-methyl-N-((2-morpholinocyclohexyl)methyl)-1H-indole-2-carboxamide |
| 509 | N-(3-(4-acetylpiperazin-1-yl)phenyl)-7-cyclopropyl-1H-indole-2-carboxamide |
| 510 | N-(cyclopropyl(3-methyl-5-(pyrrolidin-3-ylcarbamoyl)pyridin-2-yl)methyl)-7- |
| | methyl-1H-indole-2-carboxamide |
| 511 | 7-methyl-N-(2-morpholinophenethyl)-1H-indole-2-carboxamide |
| 512 | N-((1r,4r)-4-acetamidocyclohexyl)-4-fluoro-7-methyl-1H-indole-2-carboxamide |
| 513 | N-(cyclobutyl(3-methylpyridin-2-yl)methyl)-7-methyl-1H-indole-2-carboxamide |
| 514 | 4-methoxy-7-methyl-N-(1-(3-methylpyridin-2-yl)-4-morpholino-4-oxobutyl)-1H- |
| | indole-2-carboxamide |
| 515 | 7-methoxy-N-(1-(3-methylpyridin-2-yl)-4-morpholino-4-oxobutyl)-1H-indole-2- |
| | carboxamide |
| 516 | N-((5-(((1R,3R)-3-aminocyclopentyl)carbamoyl)-3-methylpyridin-2- |
| | yl)(cyclopropyl)methyl)-7-methyl-1H-indole-2-carboxamide |
| 517 | 4-fluoro-7-methyl-N-(2-oxo-2-(phenylamino)-1-(pyridin-2-yl)ethyl)-1H-indole-2- |
| | carboxamide |
| 518 | N-(cyclopropyl(3-methylpyrazin-2-yl)methyl)-7-methyl-1H-indole-2- |
| | carboxamide |
| 519 | 4-fluoro-7-methyl-N-(5-(1-methylpiperidin-4-yl)-1,3,4-thiadiazol-2-yl)-1H- |
| | indole-2-carboxamide |
| 520 | 7-methyl-N-(2-oxo-2,3,4,5-tetrahydro-1H-benzo[b]azepin-4-yl)-1H-indole-2- |
| | carboxamide |
| 521 | N-(cyclopropyl(3-ethylpyridin-2-yl)methyl)-7-methyl-1H-indole-2-carboxamide |
| 522 | N-cyclohexyl-7-methyl-1H-indole-2-carboxamide |
| 523 | 7-methyl-N-(1-(3-methylpyridin-2-yl)-4-oxo-4-(pyridin-3-ylamino)butyl)-1H- |
| | indole-2-carboxamide |
| 524 | N-(3-(5-amino-1H-1,2,4-triazol-3-yl)phenyl)-4-fluoro-7-methyl-1H-indole-2- |
| | carboxamide |
| 525 | methyl (R)-3-(4-bromophenyl)-3-(7-methyl-1H-indole-2- |
| | carboxamido)propanoate |
| 526 | 7-methyl-N-(naphthalen-2-yl)-1H-indole-2-carboxamide |
| 527 | methyl (R)-3-(4-cyanophenyl)-3-(7-methyl-1H-indole-2-carboxamido)propanoate |
| 528 | 7-methyl-N-(2-(pyrrolidin-1-yl)benzyl)-1H-indole-2-carboxamide |
| 529 | N-(cyclopropyl(3,6-dimethyl-5-((1-methylpiperidin-4-yl)carbamoyl)pyridin-2- |
| | yl)methyl)-7-methyl-1H-indole-2-carboxamide |
| 530 | N-(3-(4-amino-[1,4′-bipiperidin]-1′-yl)phenyl)-4-fluoro-7-methyl-1H-indole-2- |
| | carboxamide |
| 531 | N-(6-(4-((2-aminoethyl)amino)piperidin-1-yl)pyridin-2-yl)-7-methyl-1H-indole- |
| | 2-carboxamide |
| 532 | 4-fluoro-7-methyl-N-(3-(5-oxo-4,5-dihydro-1H-1,2,4-triazol-3-yl)phenyl)-1H- |
| | indole-2-carboxamide |
| 533 | 7-methyl-N-(quinazolin-6-yl)-1H-indole-2-carboxamide |
| 534 | 4-fluoro-7-methyl-N-(quinazolin-8-yl)-1H-indole-2-carboxamide |
| 535 | N-((5-(azetidin-3-ylcarbamoyl)-3-methylpyridin-2-yl)(cyclopropyl)methyl)-7- |
| | methyl-1H-indole-2-carboxamide |
| 536 | N-(3-((dimethylamino)methyl)imidazo[1,2-a]pyridin-6-yl)-4-fluoro-7-methyl-1H- |
| | indole-2-carboxamide |
| 537 | N-(6-(diethylamino)-5,6,7,8-tetrahydronaphthalen-2-yl)-7-methyl-1H-indole-2- |
| | carboxamide |
| 538 | 7-methyl-N-(4-oxo-3,4-dihydroquinazolin-6-yl)-1H-indole-2-carboxamide |
| 539 | 4-fluoro-7-methyl-N-(3-(4-methyl-2-phenylpiperazin-1-yl)phenyl)-1H-indole-2- |
| | carboxamide |
| 540 | 4-fluoro-7-methyl-N-(2-(4-methylpiperazin-1-yl)phenethyl)-1H-indole-2- |
| | carboxamide |
| 541 | 7-methyl-N-(2-(piperazin-1-ylmethyl)quinazolin-6-yl)-1H-indole-2-carboxamide |
| 542 | 2-(6-(cyclopropyl(4-fluoro-7-methyl-1H-indole-2-carboxamido)methyl)-5- |
| | methylpyridin-3-yl)acetic acid |
| 543 | N-((3R,4S)-1-(4-aminobutanoyl)-3-methylpiperidin-4-yl)-7-methyl-1H-indole-2- |
| | carboxamide |
| 544 | 6-(cyclopropyl(4-fluoro-7-methyl-1H-indole-2-carboxamido)methyl)-5- |
| | methylnicotinic acid |
| 545 | 4-fluoro-7-methyl-N-(3-(5-oxo-4,5-dihydro-1,2,4-oxadiazol-3-yl)phenyl)-1H- |
| | indole-2-carboxamide |
| 546 | N-(2-((dimethylamino)methyl)imidazo[1,2-a]pyridin-6-yl)-7-methyl-1H-indole-2- |
| | carboxamide |
| 547 | N-(3-(2,3-dihydro-4H-benzo[b][1,4]oxazin-4-yl)phenyl)-4-fluoro-7-methyl-1H- |
| | indole-2-carboxamide |
| 548 | 4-fluoro-7-methyl-N-(1-(1-methylpiperidin-4-yl)-1H-pyrazol-3-yl)-1H-indole-2- |
| | carboxamide |
| 549 | N-(2-(4-(2-(dimethylamino)ethyl)piperidin-1-yl)phenethyl)-7-methyl-1H-indole- |
| | 2-carboxamide |
| 550 | N-(2-(benzylamino)-2-oxo-1-(pyridin-2-yl)ethyl)-4-fluoro-7-methyl-1H-indole-2- |
| | carboxamide |
| 551 | N-(3-((dimethylamino)methyl)-7-methylimidazo[1,2-a]pyridin-6-yl)-7-methyl- |
| | 1H-indole-2-carboxamide |
| 552 | N-((1R,3s,5S)-8-(4-aminobutanoyl)-8-azabicyclo[3.2.1]octan-3-yl)-7-methyl-1H- |
| | indole-2-carboxamide |
| 553 | N-((1r,4r)-4-(3-aminopropanamido)cyclohexyl)-7-bromo-1H-indole-2- |
| | carboxamide |
| 554 | 7-methyl-N-(2-(4-methylpiperazin-1-yl)benzyl)-1H-indole-2-carboxamide |
| 555 | N-(3-(4-cyclopropyl-3,4-dihydroquinoxalin-1(2H)-yl)phenyl)-4-fluoro-7-methyl- |
| | 1H-indole-2-carboxamide |
| 556 | methyl 3-(2-fluorophenyl)-3-(7-methyl-1H-indole-2-carboxamido)propanoate |
| 557 | 4-fluoro-7-methyl-N-(5-(1-methylpiperidin-4-yl)-1,3,4-oxadiazol-2-yl)-1H- |
| | indole-2-carboxamide |
| 558 | 4-fluoro-7-methyl-N-(5-(1-methylpiperidin-4-yl)-1H-pyrazol-3-yl)-1H-indole-2- |
| | carboxamide |
| 559 | 4-fluoro-7-methyl-N-(5-(1-methylpiperidin-4-yl)-4H-1,2,4-triazol-3-yl)-1H- |
| | indole-2-carboxamide |
| 560 | 4-fluoro-7-methyl-N-(2-morpholino-2-oxo-1-(pyridin-2-yl)ethyl)-1H-indole-2- |
| | carboxamide |
| 561 | 4-fluoro-7-methyl-N-(1-(1-methylpiperidin-4-yl)-1H-imidazol-4-yl)-1H-indole-2- |
| | carboxamide |
| 562 | 4-fluoro-7-methyl-N-(2-(1-methylpiperidin-4-yl)-1H-imidazol-5-yl)-1H-indole-2- |
| | carboxamide |
| 563 | N-(cyclopentyl(3-methylpyridin-2-yl)methyl)-7-methyl-1H-indole-2-carboxamide |
| 564 | N-(cyclopropyl(2-oxoindolin-7-yl)methyl)-7-methyl-1H-indole-2-carboxamide |
| 565 | N-(1-(4-(dimethylamino)cyclohexyl)-1H-pyrazol-4-yl)-4-fluoro-7-methyl-1H- |
| | indole-2-carboxamide |
| 566 | N-(2-(2-((dimethylamino)methyl)morpholino)phenethyl)-7-methyl-1H-indole-2- |
| | carboxamide |
| 567 | 7-methyl-N-(1-(3-methylpyridin-2-yl)-4-(piperazin-1-yl)butyl)-1H-indole-2- |
| | carboxamide |
| 568 | N-(2,2-dimethyl-1-(3-methylpyridin-2-yl)propyl)-7-methyl-1H-indole-2- |
| | carboxamide |
| 569 | 4-fluoro-7-methyl-N-(1-(1-methylpiperidin-4-yl)-1H-pyrazol-4-yl)-1H-indole-2- |
| | carboxamide |
| 570 | 7-methyl-N-(pyrazolo[1,5-a]pyridin-7-ylmethyl)-1H-indole-2-carboxamide |
| 571 | 7-methyl-N-(1-(3-methylpyridin-2-yl)-4-oxo-4-((tetrahydrofuran-3- |
| | yl)amino)butyl)-1H-indole-2-carboxamide |
| 572 | 7-methyl-N-(1-(3-methylpyridin-2-yl)-4-oxo-4-(piperazin-1-yl)butyl)-1H-indole- |
| | 2-carboxamide |
| 573 | N-((5-(((1S,2S)-2-aminocyclobutyl)carbamoyl)-3-methylpyridin-2- |
| | yl)(cyclopropyl)methyl)-7-methyl-1H-indole-2-carboxamide |
| 574 | N-(cyclopropyl(3-methylpyridin-2-yl)methyl)-7-(hydroxymethyl)-1H-indole-2- |
| | carboxamide |
| 575 | 7-methyl-N-(2-morpholinobenzyl)-1H-indole-2-carboxamide |
| 576 | 2-(cyclopropyl(7-methyl-1H-indole-2-carboxamido)methyl)-3-methylpyridine 1- |
| | oxide |
| 577 | N-(cyclopropyl(2-methyl-4-(piperazine-1-carbonyl)phenyl)methyl)-7-methyl-1H- |
| | indole-2-carboxamide |
| 578 | N-(imidazo[1,2-a]pyridin-7-yl)-7-methyl-1H-indole-2-carboxamide |
| 579 | N-(4-((4-aminocyclohexyl)amino)-1-(3-methylpyridin-2-yl)-4-oxobutyl)-7- |
| | methyl-1H-indole-2-carboxamide |
| 580 | N-(2-(1H-imidazol-1-yl)phenethyl)-7-methyl-1H-indole-2-carboxamide |
| 581 | N-((1r,4r)-4-((3-aminopropyl)sulfonamido)cyclohexyl)-7-methyl-1H-indole-2- |
| | carboxamide |
| 582 | N-(4-(diethylamino)-1-(3-methylpyridin-2-yl)butyl)-7-methyl-1H-indole-2- |
| | carboxamide |
| 583 | N-(cyclopropyl(2-methyl-5-(piperazine-1-carbonyl)phenyl)methyl)-7-methyl-1H- |
| | indole-2-carboxamide |
| 584 | N-((5-(((1R,2S)-2-aminocyclobutyl)carbamoyl)-3-methylpyridin-2- |
| | yl)(cyclopropyl)methyl)-7-methyl-1H-indole-2-carboxamide |
| 585 | 4-fluoro-7-methyl-N-(2-(methylamino)-2-oxo-1-(pyridin-2-yl)ethyl)-1H-indole-2- |
| | carboxamide |
| 586 | ethyl 3-(7-methyl-1H-indole-2-carboxamido)-3-phenylpropanoate |
| 587 | 7-methyl-N-((1r,4r)-4-(3-(piperidin-1-yl)propanamido)cyclohexyl)-1H-indole-2- |
| | carboxamide |
| 588 | N-((3S,4R)-1-(4-aminobutanoyl)-3-methylpiperidin-4-yl)-7-methyl-1H-indole-2- |
| | carboxamide |
| 589 | N-((1r,4r)-4-(3-(diethylamino)propanamido)cyclohexyl)-7-methyl-1H-indole-2- |
| | carboxamide |
| 590 | N-((3R,4R)-1-(4-aminobutanoyl)-3-methylpiperidin-4-yl)-7-methyl-1H-indole-2- |
| | carboxamide |
| 591 | 7-methyl-N-(3-(3-methylpyridin-2-yl)azetidin-3-yl)-1H-indole-2-carboxamide |
| 592 | N-((1s,4s)-4-(3-aminopropanamido)cyclohexyl)-7-methyl-1H-indole-2- |
| | carboxamide |
| 593 | N-(1-(4-aminobutanoyl)pyrrolidin-3-yl)-7-methyl-1H-indole-2-carboxamide |
| 594 | N-(1-(4-aminobutanoyl)azetidin-3-yl)-7-methyl-1H-indole-2-carboxamide |
| 595 | 7-methyl-N-(2-(3-methylpyridin-2-yl)propan-2-yl)-1H-indole-2-carboxamide |
| 596 | N-((1s,3s)-3-(3-aminopropanamido)cyclobutyl)-7-methyl-1H-indole-2- |
| | carboxamide |
| 597 | N-((1r,3r)-3-(3-aminopropanamido)cyclobutyl)-7-methyl-1H-indole-2- |
| | carboxamide |
| 598 | N-(cyclopropyl(3-methylpyridin-2-yl)methyl)-7-methyl-1H-pyrrolo[2,3- |
| | c]pyridine-2-carboxamide |
| 599 | N-(cyclopropyl(3-methylpyridin-2-yl)methyl)-7-methyl-1H-pyrrolo[3,2- |
| | b]pyridine-2-carboxamide |
| 600 | N-(3-(3-aminopropanamido)cyclopentyl)-7-methyl-1H-indole-2-carboxamide |
| 601 | N,7-dimethyl-N-(1-(3-methylpyridin-2-yl)-2-morpholinoethyl)-1H-indole-2- |
| | carboxamide |
| 602 | N-((5-(aminomethyl)-3-methylisoxazol-4-yl)(cyclopropyl)methyl)-7-methyl-1H- |
| | indole-2-carboxamide |
| 603 | N-(2-(dimethylamino)-1-(pyridin-3-yl)ethyl)-7-methyl-1H-indole-2-carboxamide |
| 604 | N-(cyclopropyl(3-methylpyridin-2-yl)methyl)pyrazolo[1,5-a]pyrimidine-2- |
| | carboxamide |
| 605 | 7-methyl-N-((1r,4r)-4-(2-(pyridin-3-yl)acetamido)cyclohexyl)-1H-indole-2- |
| | carboxamide |
| 606 | N-(cyclopropyl(3-methylpyridin-2-yl)methyl)-4-(hydroxymethyl)-7-methyl-1H- |
| | indole-2-carboxamide |
| 607 | N-(cyclopropyl(3-methylpyridin-2-yl)methyl)-7-methylindoline-2-carboxamide |
| 608 | N-(cyclopropyl(3-methylpyridin-2-yl)methyl)-7-methyl-1H-pyrrolo[3,2- |
| | c]pyridine-2-carboxamide |
| 609 | 4-fluoro-7-methyl-N-(quinolin-8-yl)-1H-indole-2-carboxamide |
| 612 | 4-fluoro-7-methyl-N-(4-(4-methylpiperazin-1-yl)pyridin-2-yl)-1H-indole-2- |
| | carboxamide |
| 613 | (S)-N-(cyclopropyl(3-methylpyridin-2-yl)methyl)-7-methyl-1H-indole-2- |
| | carboxamide |
| 614 | N-((3S,4S)-1-(4-aminobutanoyl)-3-methylpiperidin-4-yl)-7-methyl-1H-indole-2- |
| | carboxamide |
| 615 | N-(cyclopropyl(6-((dimethylamino)methyl)pyridin-2-yl)methyl)-7-methyl-1H- |
| | indole-2-carboxamide |
| 616 | (R)-N-(2-hydroxy-1-phenylethyl)-7-methyl-1H-indole-2-carboxamide |
| 617 | 7-methyl-N-(2-oxo-6-phenylazepan-4-yl)-1H-indole-2-carboxamide |
| 618 | 7-((1H-pyrazol-5-yl)methyl)-N-(cyclopropyl(3-methylpyridin-2-yl)methyl)-1H- |
| | indole-2-carboxamide |
| 619 | (2,3-dihydrospiro[indene-1,2′-pyrrolidin]-1′-yl)(7-methyl-1H-indol-2- |
| | yl)methanone |
| 620 | 7-methyl-N-(1-oxo-2,3,4,5-tetrahydro-1H-benzo[c]azepin-5-yl)-1H-indole-2- |
| | carboxamide |
| 622 | N-(5-chloro-2-morpholinophenethyl)-7-methyl-1H-indole-2-carboxamide |
| 623 | N-(imidazo[1,2-a]pyrazin-6-yl)-7-methyl-1H-indole-2-carboxamide |
| 624 | 7-methyl-N-(2-methyl-3-(morpholinomethyl)phenyl)-1H-indole-2-carboxamide |
| 625 | N-((1S,4S,5S)-2-(4-aminobutanoyl)-2-azabicyclo[2.2.2]octan-5-yl)-7-methyl-1H- |
| | indole-2-carboxamide |
| 626 | 7-methyl-N-(1-(pyrimidin-2-yl)piperidin-3-yl)-1H-indole-2-carboxamide |
| 627 | N-(2-(dimethylamino)-1-(3-methylpyridin-2-yl)ethyl)-7-methyl-1H-indole-2- |
| | carboxamide |
| 628 | ethyl 4-(7-methyl-1H-indole-2-carboxamido)piperidine-1-carboxylate |
| 629 | N-((1r,4r)-4-(3-aminopropanamido)-4-methylcyclohexyl)-7-methyl-1H-indole-2- |
| | carboxamide |
| 630 | ethyl 3-cyclobutyl-2-(7-methyl-1H-indole-2-carboxamido)propanoate |
| 631 | N-((1R,4R,5S)-2-(3-aminopropanoyl)-2-azabicyclo[2.2.2]octan-5-yl)-7-methyl- |
| | 1H-indole-2-carboxamide |
| 632 | 7-methyl-N-(pyridin-4-yl)-1H-indole-2-carboxamide |
| 633 | 7-methyl-N-(6-(morpholinomethyl)pyridin-2-yl)-1H-indole-2-carboxamide |
| 634 | methyl (R)-3-(3-chlorophenyl)-3-(7-methyl-1H-indole-2-carboxamido)propanoate |
| 635 | N-(2-(2′-(aminomethyl)-[1,1′-biphenyl]-2-yl)ethyl)-7-methyl-1H-indole-2- |
| | carboxamide |
| 636 | 7-methyl-N-(2-(piperazin-1-yl)benzyl)-1H-indole-2-carboxamide |
| 637 | N-(di(pyridin-2-yl)methyl)-7-methyl-1H-indole-2-carboxamide |
| 638 | N-(2-(dimethylamino)benzyl)-7-methyl-1H-indole-2-carboxamide |
| 639 | N-(1-(4-aminobutanoyl)piperidin-4-yl)-7-methyl-1H-indole-2-carboxamide |
| 640 | N-(8-(3-aminopropanamido)bicyclo[3.2.1]octan-3-yl)-7-methyl-1H-indole-2- |
| | carboxamide |
| 641 | N-((1s,4s)-4-(3-aminopropanamido)-4-methylcyclohexyl)-7-methyl-1H-indole-2- |
| | carboxamide |
| 642 | 7-methyl-N-(2,2,2-trifluoro-1-(3-fluorophenyl)ethyl)-1H-indole-2-carboxamide |
| 643 | 7-methyl-N-(1-(pyridin-3-yl)cyclopropyl)-1H-indole-2-carboxamide |
| 644 | N-((1r,4r)-4-(3-aminopropanamido)cyclohexyl)-4,7-dimethyl-1H-indole-2- |
| | carboxamide |
| 645 | N-((1r,4r)-4-(3-aminopropanamido)cyclohexyl)-N,7-dimethyl-1H-indole-2- |
| | carboxamide |
| 646 | N-(1-((3-aminopropyl)sulfonyl)piperidin-3-yl)-7-methyl-1H-indole-2- |
| | carboxamide |
| 648 | methyl 3-(furan-2-yl)-3-(7-methyl-1H-indole-2-carboxamido)propanoate |
| 649 | methyl 3-(7-methyl-1H-indole-2-carboxamido)-3-(thiophen-3-yl)propanoate |
| 650 | methyl 3-(7-methyl-1H-indole-2-carboxamido)-3-(thiophen-2-yl)propanoate |
| 652 | N-(1-(4-(1H-pyrazol-1-yl)phenyl)ethyl)-7-methyl-1H-indole-2-carboxamide |
| 653 | N-(4-chloro-2-morpholinophenethyl)-7-methyl-1H-indole-2-carboxamide |
| 654 | N-((1S,4R)-bicyclo[2.2.1]heptan-2-yl)-7-methyl-1H-indole-2-carboxamide |
| 655 | N-((1r,4r)-4-(3-aminopropanamido)cyclohexyl)-3,7-dimethyl-1H-indole-2- |
| | carboxamide |
| 656 | N-((1R,3r,5S)-8-(4-aminobutanoyl)-8-azabicyclo[3.2.1]octan-3-yl)-7-methyl- |
| | 3a,7a-dihydro-1H-indole-2-carboxamide |
| 657 | N-((1S,4S,5R)-2-(4-aminobutanoyl)-2-azabicyclo[2.2.2]octan-5-yl)-7-methyl-1H- |
| | indole-2-carboxamide |
| 658 | methyl (R)-3-(7-methyl-1H-indole-2-carboxamido)-3-(pyridin-3-yl)propanoate |
| 659 | methyl 3-(3-methoxyphenyl)-3-(7-methyl-1H-indole-2-carboxamido)propanoate |
| 660 | N-(cyclopropyl(pyridin-2-yl)methyl)-7-methyl-1H-indole-2-carboxamide |
| 661 | N-((1r,4r)-4-((2-aminoethyl)sulfonamido)cyclohexyl)-7-methyl-1H-indole-2- |
| | carboxamide |
| 662 | N-((1s,4s)-4-(hydroxymethyl)cyclohexyl)-7-methyl-1H-indole-2-carboxamide |
| 663 | N-(1-((3-aminopropyl)sulfonyl)piperidin-4-yl)-7-methyl-1H-indole-2- |
| | carboxamide |
| 664 | N-(1-(4-aminobutanoyl)piperidin-3-yl)-7-methyl-1H-indole-2-carboxamide |
| 665 | 7-methyl-N-(pyrimidin-5-yl)-1H-indole-2-carboxamide |
| 666 | methyl (R)-3-(7-methyl-1H-indole-2-carboxamido)-3-phenylpropanoate |
| 667 | N-((6-(2-amino-2-oxoethyl)pyridin-2-yl)(cyclopropyl)methyl)-7-methyl-1H- |
| | indole-2-carboxamide |
| 668 | 7-methyl-N-(1-(3-methyl-1,2,4-oxadiazol-5-yl)propyl)-1H-indole-2-carboxamide |
| 669 | methyl 3-(3-bromophenyl)-3-(7-methyl-1H-indole-2-carboxamido)propanoate |
| 670 | N-(1-acetylpiperidin-4-yl)-7-methyl-1H-indole-2-carboxamide |
| 671 | methyl 3-(7-methyl-1H-indole-2-carboxamido)-3-(pyridin-3-yl)propanoate |
| 672 | N-((1R,4R,5R)-2-(3-aminopropanoyl)-2-azabicyclo[2.2.2]octan-5-yl)-7-methyl- |
| | 1H-indole-2-carboxamide |
| 673 | 7-methyl-N-(pyridin-2-yl)-1H-indole-2-carboxamide |
| 674 | 7-methyl-N-(3-(pyrrolidin-1-ylmethyl)benzyl)-1H-indole-2-carboxamide |
| 675 | ethyl 3,3,3-trifluoro-2-(7-methyl-1H-indole-2-carboxamido)propanoate |
| 676 | methyl (S)-3-(7-methyl-1H-indole-2-carboxamido)-3-(pyridin-3-yl)propanoate |
| 677 | methyl (S)-3-(7-methyl-1H-indole-2-carboxamido)-3-(o-tolyl)propanoate |
| 678 | 7-methyl-N-(3,3,3-trifluoro-2-hydroxypropyl)-1H-indole-2-carboxamide |
| 679 | 7-methyl-N-(2-(5-oxopyrrolidin-2-yl)phenyl)-1H-indole-2-carboxamide |
| 680 | N-(cyclopropyl(pyrimidin-2-yl)methyl)-7-methyl-1H-indole-2-carboxamide |
| 681 | N-(2-(3,5-dimethoxyphenyl)-2-hydroxyethyl)-7-methyl-1H-indole-2-carboxamide |
| 682 | 7-methyl-N-(3-(methylamino)-1-(3-methylpyridin-2-yl)-3-oxopropyl)-1H-indole- |
| | 2-carboxamide |
| 683 | methyl (S)-3-(4-chlorophenyl)-3-(7-methyl-1H-indole-2-carboxamido)propanoate |
| 684 | (3,4-dihydroisoquinolin-2(1H)-yl)(7-methyl-1H-indol-2-yl)methanone |
| 685 | N-(3-(3-aminopropanamido)bicyclo[3.2.1]octan-8-yl)-7-methyl-1H-indole-2- |
| | carboxamide |
| 686 | N-(2-hydroxy-2-(o-tolyl)ethyl)-7-methyl-1H-indole-2-carboxamide |
| 687 | (S)-7-methyl-N-(2,2,2-trifluoro-1-phenylethyl)-1H-indole-2-carboxamide |
| 688 | 7-methyl-N-(1-(4-methylthiazol-2-yl)ethyl)-1H-indole-2-carboxamide |
| 689 | N-(cyclopropyl(pyridin-3-yl)methyl)-7-methyl-1H-indole-2-carboxamide |
| 690 | methyl (S)-3-(7-methyl-1H-indole-2-carboxamido)-3-phenylpropanoate |
| 691 | 7-methyl-N-(4-(morpholinomethyl)phenyl)-1H-indole-2-carboxamide |
| 692 | (R)-N-(1-hydroxy-3-phenylpropan-2-yl)-7-methyl-1H-indole-2-carboxamide |
| 693 | 7-methyl-N-((1-methyl-1H-pyrazol-5-yl)methyl)-1H-indole-2-carboxamide |
| 694 | dimethyl (7-methyl-1H-indole-2-carbonyl)-D-aspartate |
| 695 | N-(2-hydroxy-2-(3-methoxyphenyl)ethyl)-7-methyl-1H-indole-2-carboxamide |
| 697 | N-((1r,4r)-4-(3-aminopropanamido)cyclohexyl)-7-chloro-1H-indole-2- |
| | carboxamide |
| 698 | N-((1r,4r)-4-acetamidocyclohexyl)-7-methyl-1H-indole-2-carboxamide |
| 699 | 7-methyl-N-(2-oxo-1,2,3,4-tetrahydroquinolin-3-yl)-1H-indole-2-carboxamide |
| 700 | ethyl 3-(7-methyl-1H-indole-2-carboxamido)-3-(pyridin-4-yl)propanoate |
| 701 | methyl 2-(7-methyl-1H-indole-2-carboxamido)-2-phenylacetate |
| 702 | 7-methyl-N-((5-methylisoxazol-4-yl)methyl)-1H-indole-2-carboxamide |
| 703 | (S)-N-(2-hydroxy-1-phenylethyl)-7-methyl-1H-indole-2-carboxamide |
| 704 | 7-methyl-N-(2-oxo-2,3,4,5-tetrahydro-1H-benzo[b]azepin-5-yl)-1H-indole-2- |
| | carboxamide |
| 705 | N-(2-hydroxy-1-phenylethyl)-7-methyl-1H-indole-2-carboxamide |
| 706 | N-((1r,4r)-4-aminocyclohexyl)-7-bromo-1H-indole-2-carboxamide |
| 707 | methyl 3-(3-fluorophenyl)-3-(7-methyl-1H-indole-2-carboxamido)propanoate |
| 708 | dimethyl (7-methyl-1H-indole-2-carbonyl)-L-aspartate |
| 709 | 7-methyl-N-(3-(morpholinomethyl)benzyl)-1H-indole-2-carboxamide |
| 710 | N-(imidazo[1,2-alpyrazin-6-ylmethyl)-7-methyl-1H-indole-2-carboxamide |
| 711 | N-((1r,4r)-4-(3-aminopropanamido)cyclohexyl)-3-ethyl-7-methyl-1H-indole-2- |
| | carboxamide |
| 712 | 7-methyl-N-(1-(1-methyl-1H-pyrazol-3-yl)ethyl)-1H-indole-2-carboxamide |
| 713 | (R)-7-methyl-N-(1-(m-tolyl)ethyl)-1H-indole-2-carboxamide |
| 714 | 7-methyl-N-(1-(naphthalen-1-yl)ethyl)-1H-indole-2-carboxamide |
| 715 | 7-methyl-N-(piperidin-4-yl)-1H-indole-2-carboxamide |
| 716 | 7-methyl-N-(2-(piperidin-1-yl)benzyl)-1H-indole-2-carboxamide |
| 717 | N-(cyclopropyl(3-methylpyridin-2-yl)methyl)-7-(trifluoromethyl)-1H-indole-2- |
| | carboxamide |
| 718 | (7-methyl-1H-indol-2-yl)(1,3,4,5-tetrahydro-2H-benzo[c]azepin-2-yl)methanone |
| 719 | 7-methyl-N-((5-methylisoxazol-3-yl)methyl)-1H-indole-2-carboxamide |
| 720 | N-(2-(2,6-difluorophenyl)-2-hydroxyethyl)-7-methyl-1H-indole-2-carboxamide |
| 721 | N-((2,5-dimethyloxazol-4-yl)methyl)-7-methyl-1H-indole-2-carboxamide |
| 722 | 7-methyl-N-((3-methylpyridin-2-yl)(pyrrolidin-3-yl)methyl)-1H-indole-2- |
| | carboxamide |
| 723 | 7-methyl-N-((5-methylpyrazin-2-yl)methyl)-1H-indole-2-carboxamide |
| 724 | 7-methyl-N-(4-(morpholinomethyl)pyridin-2-yl)-1H-indole-2-carboxamide |
| 725 | N-(3-(dimethylamino)-1-(pyridin-3-yl)propyl)-7-methyl-1H-indole-2- |
| | carboxamide |
| 726 | (R)-7-methyl-N-(2,2,2-trifluoro-1-phenylethyl)-1H-indole-2-carboxamide |
| 727 | N-(cyclopropyl(5-((dimethylamino)methyl)pyridin-3-yl)methyl)-7-methyl-1H- |
| | indole-2-carboxamide |
| 728 | N-(3-(dimethylamino)-1-(3-methylpyridin-2-yl)propyl)-7-methyl-1H-indole-2- |
| | carboxamide |
| 729 | N-((1H-imidazol-5-yl)methyl)-7-methyl-1H-indole-2-carboxamide |
| 730 | N-((1r,4r)-4-aminocyclohexyl)-4,7-dimethyl-1H-indole-2-carboxamide |
| 731 | N-(2-cyclopropyl-2-hydroxyethyl)-7-methyl-1H-indole-2-carboxamide |
| 732 | 7-methyl-N-((4-methyl-1,2,5-oxadiazol-3-yl)methyl)-1H-indole-2-carboxamide |
| 733 | 7-methyl-N-(oxazol-4-ylmethyl)-1H-indole-2-carboxamide |
| 735 | 7-methyl-N-(1-(3-methyl-1,2,4-oxadiazol-5-yl)ethyl)-1H-indole-2-carboxamide |
| 736 | N-((1r,4r)-4-aminocyclohexyl)-3,7-dimethyl-1H-indole-2-carboxamide |
| 738 | N-((1r,4r)-4-(3-aminopropanamido)cyclohexyl)-5,7-dimethyl-1H-indole-2- |
| | carboxamide |
| 746 | 7-methyl-N-(2-morpholinoethyl)-1H-indole-2-carboxamide |
| 747 | 7-methyl-N-((tetrahydrofuran-2-yl)methyl)-1H-indole-2-carboxamide |
| 748 | N-(2,3-dihydro-1H-inden-1-yl)-7-methyl-1H-indole-2-carboxamide |
| 749 | 7-methyl-N-(1-phenylethyl)-1H-indole-2-carboxamide |
| 750 | 7-methyl-N-(3-(piperidin-1-yl)benzyl)-1H-indole-2-carboxamide |
| 751 | 7-methyl-N-(3-(piperidin-1-ylsulfonyl)benzyl)-1H-indole-2-carboxamide |
| 752 | N-(1-benzylpiperidin-4-yl)-7-methyl-1H-indole-2-carboxamide |
| 753 | 7-methyl-N-(1,2,3,4-tetrahydronaphthalen-1-yl)-1H-indole-2-carboxamide |
| 754 | N-(2-hydroxy-2-(4-methoxyphenyl)ethyl)-7-methyl-1H-indole-2-carboxamide |
| 755 | 7-methyl-N-(4-(morpholinomethyl)benzyl)-1H-indole-2-carboxamide |
| 756 | N-((1r,4r)-4-aminocyclohexyl)-5,7-dimethyl-1H-indole-2-carboxamide |
| 757 | 2-(4-(7-methyl-1H-indole-2-carbonyl)piperazin-1-yl)nicotinamide |
| 758 | 7-methyl-N-(4-(piperidin-1-ylsulfonyl)benzyl)-1H-indole-2-carboxamide |
| 759 | N-((1s,3s)-3-(hydroxymethyl)cyclobutyl)-7-methyl-1H-indole-2-carboxamide |
| 761 | N-((1r,4r)-4-aminocyclohexyl)-N,7-dimethyl-1H-indole-2-carboxamide |
| 763 | (S)-N-(1-hydroxy-3-methylbutan-2-yl)-7-methyl-1H-indole-2-carboxamide |
| 764 | methyl 3-(4-chlorophenyl)-3-(7-methyl-1H-indole-2-carboxamido)propanoate |
| 765 | (S)-N-(1-hydroxy-3-phenylpropan-2-yl)-7-methyl-1H-indole-2-carboxamide |
| 766 | tert-butyl (2-(7-methyl-1H-indole-2-carboxamido)propyl)carbamate |
| 767 | N-(2-(cyclopropylmethoxy)benzyl)-7-methyl-1H-indole-2-carboxamide |
| 768 | 7-methyl-N-((1-methyl-1H-1,2,4-triazol-5-yl)methyl)-1H-indole-2-carboxamide |
| 769 | N-(1-cyclopropyl-2,2,2-trifluoroethyl)-7-methyl-1H-indole-2-carboxamide |
| 771 | 1-(7-methyl-1H-indole-2-carbonyl)piperidine-4-carboxamide |
| 773 | methyl 3-(4-isopropylphenyl)-3-(7-methyl-1H-indole-2-carboxamido)propanoate |
| 774 | methyl 3-(2-bromophenyl)-3-(7-methyl-1H-indole-2-carboxamido)propanoate |
| 775 | methyl 3-(7-methyl-1H-indole-2-carboxamido)-3-phenylpropanoate |
| 776 | methyl 3-(4-fluorophenyl)-3-(7-methyl-1H-indole-2-carboxamido)propanoate |
| 777 | methyl 3-(4-methoxyphenyl)-3-(7-methyl-1H-indole-2-carboxamido)propanoate |
| 778 | methyl 3-(4-bromophenyl)-3-(7-methyl-1H-indole-2-carboxamido)propanoate |
| 779 | N-(benzo[d]oxazol-2-ylmethyl)-7-methyl-1H-indole-2-carboxamide |
| 780 | 7-methyl-N-((1r,4r)-4-(trifluoromethyl)cyclohexyl)-1H-indole-2-carboxamide |
| 781 | 2-(4-(7-methyl-1H-indole-2-carbonyl)piperazin-1-yl)acetamide |
| 782 | 7-methyl-N-(2-(5-methyl-1,2,4-oxadiazol-3-yl)ethyl)-1H-indole-2-carboxamide |
| 783 | methyl 3-(7-methyl-1H-indole-2-carboxamido)-3-(p-tolyl)propanoate |
| 784 | N-(3,3-difluoro-2-hydroxypropyl)-7-methyl-1H-indole-2-carboxamide |
| 785 | 7-methyl-N-(1-(5-methyl-1,2,4-oxadiazol-3-yl)ethyl)-1H-indole-2-carboxamide |
| 786 | 1-(7-methyl-1H-indole-2-carbonyl)pyrrolidine-2-carboxamide |
| 787 | 4-(7-methyl-1H-indole-2-carbonyl)piperazine-1-carboxamide |
| 788 | 7-methyl-N-(1-(2-(trifluoromethyl)phenyl)ethyl)-1H-indole-2-carboxamide |
| 790 | 7-methyl-N-(1-(3-(trifluoromethyl)phenyl)ethyl)-1H-indole-2-carboxamide |
| 791 | 7-methyl-N-((1r,4r)-4-(2,2,2-trifluoroacetamido)cyclohexyl)-1H-indole-2- |
| | carboxamide |
| 793 | 7-methyl-N-(2-morpholinophenyl)-1H-indole-2-carboxamide |
| 794 | (3-aminopiperidin-1-yl)(7-methyl-1H-indol-2-yl)methanone |
| 795 | 7-methyl-N-(pyridin-3-yl)-1H-indole-2-carboxamide |
| 796 | N-(imidazo[1,2-a]pyridin-2-yl)-7-methyl-1H-indole-2-carboxamide |
| 797 | ethyl (1R,2R)-2-(7-methyl-1H-indole-2-carboxamido)cyclohexane-1-carboxylate |
| 798 | 3-amino-N-(1-(7-methyl-1H-indole-2-carbonyl)piperidin-3-yl)propanamide |
| 799 | (3-(dimethylamino)piperidin-1-yl)(7-methyl-1H-indol-2-yl)methanone |
| 800 | (7-methyl-1H-indol-2-yl)(3-(methylamino)piperidin-1-yl)methanone |
| 801 | N-(cyclopropyl(o-tolyl)methyl)-7-methyl-1H-indole-2-carboxamide |
| 802 | N-(cyclopropyl(3-methylpyridin-2-yl)methyl)-7-isopropyl-1H-indole-2- |
| | carboxamide |
| 803 | N-(cyclopropyl(pyridin-2-yl)methyl)-7-ethyl-1H-indole-2-carboxamide |
| 804 | 7-methyl-N-(quinazolin-2-yl)-1H-indole-2-carboxamide |
| 805 | 7-cyclopropyl-N-(cyclopropyl(3-methylpyridin-2-yl)methyl)-1H-indole-2- |
| | carboxamide |
| 808 | N-(cyclopropyl(3-methylpyridin-2-yl)methyl)-6-fluoro-7-methyl-1H-indole-2- |
| | carboxamide |
| 809 | 7-methyl-N-(3-(methylamino)-3-oxo-1-(pyridin-3-yl)propyl)-1H-indole-2- |
| | carboxamide |
| 810 | (5,6-dihydroimidazo[1,5-a]pyrazin-7(8H)-yl)(7-methyl-1H-indol-2-yl)methanone |
| 811 | (5,6-dihydro-[1,2,4]triazolo[4,3-a]pyrazin-7(8H)-yl)(7-methyl-1H-indol-2- |
| | yl)methanone |
| 812 | (5,6-dihydro-[1,2,4]triazolo[1,5-a]pyrazin-7(8H)-yl)(7-methyl-1H-indol-2- |
| | yl)methanone |
| 814 | 2-(7-methyl-1H-indole-2-carbonyl)-1,2,3,4-tetrahydro-5H-benzo[c]azepin-5-one |
| 815 | N-(3-(dimethylamino)-2-phenylpropyl)-7-methyl-1H-indole-2-carboxamide |
| 816 | 7-methyl-N-(2-oxo-7-phenylazepan-4-yl)-1H-indole-2-carboxamide |
| 817 | N-((3-benzylpyridin-2-yl)(cyclopropyl)methyl)-7-methyl-1H-indole-2- |
| | carboxamide |
| 818 | N-(2-([1,1′-biphenyl]-2-yl)ethyl)-7-methyl-1H-indole-2-carboxamide |
| 819 | N-(cyclopropyl(3-methylpyridin-2-yl)methyl)-7-propyl-1H-indole-2-carboxamide |
| 820 | (2,3-dihydrospiro[indene-1,3′-pyrrolidin]-1′-yl)(7-methyl-1H-indol-2- |
| | yl)methanone |
| 821 | 7-(cyclobutylmethyl)-N-(cyclopropyl(3-methylpyridin-2-yl)methyl)-1H-indole-2- |
| | carboxamide |
| 822 | 4-fluoro-7-methyl-N-((1R,3R)-3-(3-oxo-1,4-diazepan-1-yl)cyclohexyl)-1H- |
| | indole-2-carboxamide |
| 823 | 4-fluoro-7-methyl-N-((1R,3S)-3-(3-oxo-1,4-diazepan-1-yl)cyclohexyl)-1H- |
| | indole-2-carboxamide |
| 824 | ethyl 4-((1S,3R)-3-(4-fluoro-7-methyl-1H-indole-2- |
| | carboxamido)cyclohexyl)piperazine-1-carboxylate |
| 825 | ethyl 4-((1R,3R)-3-(4-fluoro-7-methyl-1H-indole-2- |
| | carboxamido)cyclohexyl)piperazine-1-carboxylate |
| 826 | 4-fluoro-7-methyl-N-((1R,3S)-3-(4-(2,2,2-trifluoroethyl)piperazin-1- |
| | yl)cyclohexyl)-1H-indole-2-carboxamide |
| 827 | 4-fluoro-7-methyl-N-((1R,3R)-3-(4-(2,2,2-trifluoroethyl)piperazin-1- |
| | yl)cyclohexyl)-1H-indole-2-carboxamide |
| 828 | 4-fluoro-7-methyl-N-((1R,3S)-3-(4-(morpholine-4-carbonyl)piperazin-1- |
| | yl)cyclohexyl)-1H-indole-2-carboxamide |
| 829 | 4-fluoro-7-methyl-N-((1R,3R)-3-(4-(morpholine-4-carbonyl)piperazin-1- |
| | yl)cyclohexyl)-1H-indole-2-carboxamide |
| 830 | N-((1R,3S)-3-(4-(cyclopropanecarbonyl)piperazin-1-yl)cyclohexyl)-4-fluoro-7- |
| | methyl-1H-indole-2-carboxamide |
| 831 | N-((1R,3R)-3-(4-(cyclopropanecarbonyl)piperazin-1-yl)cyclohexyl)-4-fluoro-7- |
| | methyl-1H-indole-2-carboxamide |
| 832 | 4-fluoro-7-methyl-N-((1R)-3-(4-methyl-5-oxo-1,4-diazepan-1-yl)cyclohexyl)-1H- |
| | indole-2-carboxamide |
| 833 | N-((1R,3S)-3-(3-acetamidopyrrolidin-1-yl)cyclohexyl)-4-fluoro-7-methyl-1H- |
| | indole-2-carboxamide |
| 834 | N-((1R,3R)-3-(3-acetamidopyrrolidin-1-yl)cyclohexyl)-4-fluoro-7-methyl-1H- |
| | indole-2-carboxamide |
| 835 | N-((1R,3S)-3-(4-ethyl-3-oxopiperazin-1-yl)cyclohexyl)-4-fluoro-7-methyl-1H- |
| | indole-2-carboxamide |
| 836 | N-((1R,3R)-3-(4-ethyl-3-oxopiperazin-1-yl)cyclohexyl)-4-fluoro-7-methyl-1H- |
| | indole-2-carboxamide |
| 837 | 4-fluoro-N-((1R,3S)-3-(4-isobutyrylpiperazin-1-yl)cyclohexyl)-7-methyl-1H- |
| | indole-2-carboxamide |
| 838 | 4-fluoro-N-((1R,3R)-3-(4-isobutyrylpiperazin-1-yl)cyclohexyl)-7-methyl-1H- |
| | indole-2-carboxamide |
| 839 | 4-fluoro-7-methyl-N-((1R,3S)-3-(4-propionylpiperazin-1-yl)cyclohexyl)-1H- |
| | indole-2-carboxamide |
| 840 | 4-fluoro-7-methyl-N-((1R,3R)-3-(4-propionylpiperazin-1-yl)cyclohexyl)-1H- |
| | indole-2-carboxamide |
| 841 | 4-fluoro-7-methyl-N-((1R,3S)-3-((S)-3-(N-methylacetamido)pyrrolidin-1- |
| | yl)cyclohexyl)-1H-indole-2-carboxamide |
| 842 | 4-fluoro-7-methyl-N-((1R,3R)-3-((S)-3-(N-methylacetamido)pyrrolidin-1- |
| | yl)cyclohexyl)-1H-indole-2-carboxamide |
| 843 | 4-fluoro-N-((1R,3S)-3-(3-(N-(2-methoxyethyl)acetamido)pyrrolidin-1- |
| | yl)cyclohexyl)-7-methyl-1H-indole-2-carboxamide |
| 844 | 4-fluoro-N-((1R,3R)-3-(3-(N-(2-methoxyethyl)acetamido)pyrrolidin-1- |
| | yl)cyclohexyl)-7-methyl-1H-indole-2-carboxamide |
| 845 | 4-fluoro-N-((1R,3R)-3-(3-(N-(2-methoxyethyl)acetamido)pyrrolidin-1- |
| | yl)cyclohexyl)-7-methyl-1H-indole-2-carboxamide |
| 846 | 4-fluoro-N-((1R,3S)-3-(4-(2-methoxyacetyl)piperazin-1-yl)cyclohexyl)-7-methyl- |
| | 1H-indole-2-carboxamide |
| 847 | 4-fluoro-N-((1R,3R)-3-(4-(2-methoxyacetyl)piperazin-1-yl)cyclohexyl)-7-methyl- |
| | 1H-indole-2-carboxamide |
| 848 | 4-fluoro-7-methyl-N-((1R,3S)-3-((R)-3-(N-methylacetamido)pyrrolidin-1- |
| | yl)cyclohexyl)-1H-indole-2-carboxamide |
| 849 | 4-fluoro-7-methyl-N-((1R,3R)-3-((R)-3-(N-methylacetamido)pyrrolidin-1- |
| | yl)cyclohexyl)-1H-indole-2-carboxamide |
| 850 | 4-fluoro-7-methyl-N-((1R,3S)-3-(4-(methylsulfonyl)piperazin-1-yl)cyclohexyl)- |
| | 1H-indole-2-carboxamide |
| 851 | 4-fluoro-7-methyl-N-((1R,3R)-3-(4-(methylsulfonyl)piperazin-1-yl)cyclohexyl)- |
| | 1H-indole-2-carboxamide |
| 852 | N-((1R,3S)-3-(5,6-dihydroimidazo[1,2-a]pyrazin-7(8H)-yl)cyclohexyl)-4-fluoro- |
| | 7-methyl-1H-indole-2-carboxamide |
| 853 | N-((1R,3R)-3-(5,6-dihydroimidazo[1,2-a]pyrazin-7(8H)-yl)cyclohexyl)-4-fluoro- |
| | 7-methyl-1H-indole-2-carboxamide |
| 854 | N-((1R,3S)-3-(4-(dimethylglycyl)piperazin-1-yl)cyclohexyl)-4-fluoro-7-methyl- |
| | 1H-indole-2-carboxamide |
| 855 | N-((1R,3R)-3-(4-(dimethylglycyl)piperazin-1-yl)cyclohexyl)-4-fluoro-7-methyl- |
| | 1H-indole-2-carboxamide |
| 856 | N-((1R,3S)-3-(4-acetylpiperazin-1-yl)cyclohexyl)-7-methyl-1H-indole-2- |
| | carboxamide |
| 857 | N-((1R,3R)-3-(4-acetylpiperazin-1-yl)cyclohexyl)-7-methyl-1H-indole-2- |
| | carboxamide |
| 858 | 4-fluoro-7-methyl-N-((1R)-3-(4-(2-(methylamino)-2-oxoethyl)piperazin-1- |
| | yl)cyclohexyl)-1H-indole-2-carboxamide |
| 859 | N-((1R)-3-(4-(2-(dimethylamino)-2-oxoethyl)piperazin-1-yl)cyclohexyl)-4- |
| | fluoro-7-methyl-1H-indole-2-carboxamide |
| 860 | N-((1R,3S)-3-(5,6-dihydro-[1,2,4]triazolo[4,3-a]pyrazin-7(8H)-yl)cyclohexyl)-4- |
| | fluoro-7-methyl-1H-indole-2-carboxamide |
| 861 | N-((1R,3R)-3-(5,6-dihydro-[1,2,4]triazolo[4,3-a]pyrazin-7(8H)-yl)cyclohexyl)-4- |
| | fluoro-7-methyl-1H-indole-2-carboxamide |
| 862 | 4-fluoro-N-((1R)-3-(3-(N-isopropylacetamido)pyrrolidin-1-yl)cyclohexyl)-7- |
| | methyl-1H-indole-2-carboxamide |
| 863 | 4-fluoro-7-methyl-N-((1R,3S)-3-(6-oxohexahydropyrrolo[1,2-a]pyrazin-2(1H)- |
| | yl)cyclohexyl)-1H-indole-2-carboxamide |
| 864 | 4-fluoro-7-methyl-N-((1R,3R)-3-(6-oxohexahydropyrrolo[1,2-a]pyrazin-2(1H)- |
| | yl)cyclohexyl)-1H-indole-2-carboxamide |
| 865 | 4-fluoro-7-methyl-N-((1R,3S)-3-(3-oxotetrahydro-3H-oxazolo[3,4-a]pyrazin- |
| | 7(1H)-yl)cyclohexyl)-1H-indole-2-carboxamide |
| 866 | 4-fluoro-7-methyl-N-((1R,3R)-3-(3-oxotetrahydro-3H-oxazolo[3,4-a]pyrazin- |
| | 7(1H)-yl)cyclohexyl)-1H-indole-2-carboxamide |
| 867 | N-(1-(1-acetylpiperidin-4-yl)azepan-3-yl)-4-fluoro-7-methyl-1H-indole-2- |
| | carboxamide |
| 868 | N-((1R,3S)-3-(5,6-dihydro-[1,2,4]triazolo[1,5-a]pyrazin-7(8H)-yl)cyclohexyl)-4- |
| | fluoro-7-methyl-1H-indole-2-carboxamide |
| 869 | N-((1R,3R)-3-(5,6-dihydro-[1,2,4]triazolo[1,5-a]pyrazin-7(8H)-yl)cyclohexyl)-4- |
| | fluoro-7-methyl-1H-indole-2-carboxamide |
| 870 | 4-fluoro-N-((1R,3S)-3-(4-(2-hydroxyacetyl)piperazin-1-yl)cyclohexyl)-7-methyl- |
| | 1H-indole-2-carboxamide |
| 871 | 4-fluoro-N-((1R,3R)-3-(4-(2-hydroxyacetyl)piperazin-1-yl)cyclohexyl)-7-methyl- |
| | 1H-indole-2-carboxamide |
| 872 | 4-fluoro-N-((1R,3S)-3-(3-(N-(2-hydroxyethyl)acetamido)pyrrolidin-1- |
| | yl)cyclohexyl)-7-methyl-1H-indole-2-carboxamide |
| 873 | 4-fluoro-N-((1R,3R)-3-(3-(N-(2-hydroxyethyl)acetamido)pyrrolidin-1- |
| | yl)cyclohexyl)-7-methyl-1H-indole-2-carboxamide |
| 874 | 4-fluoro-7-methyl-N-((1R)-3-(1-oxo-2,8-diazaspiro[4.5]decan-8-yl)cyclohexyl)- |
| | 1H-indole-2-carboxamide |
| 875 | ethyl (R)-3-(4-fluoro-7-methyl-1H-indole-2-carboxamido)-[1,4′-bipiperidine]-1′- |
| | carboxylate |
| 876 | ethyl (S)-3-(4-fluoro-7-methyl-1H-indole-2-carboxamido)-[1,4′-bipiperidine]-1′- |
| | carboxylate |
| 877 | 1-(4-((1-(4-fluoro-7-methyl-1H-indole-2-carbonyl)piperidin-3- |
| | yl)methyl)piperazin-1-yl)ethan-1-one |
| 878 | N-((1R,3S)-3-(4-(2,2-difluoroacetyl)piperazin-1-yl)cyclohexyl)-4-fluoro-7- |
| | methyl-1H-indole-2-carboxamide |
| 879 | N-((1R,3R)-3-(4-(2,2-difluoroacetyl)piperazin-1-yl)cyclohexyl)-4-fluoro-7- |
| | methyl-1H-indole-2-carboxamide |
| 880 | 4-fluoro-N-((1R,3S)-3-(3-(2-methoxy-N-methylacetamido)pyrrolidin-1- |
| | yl)cyclohexyl)-7-methyl-1H-indole-2-carboxamide |
| 881 | 4-fluoro-N-((1R,3R)-3-(3-(2-methoxy-N-methylacetamido)pyrrolidin-1- |
| | yl)cyclohexyl)-7-methyl-1H-indole-2-carboxamide |
| 882 | 4-fluoro-7-methyl-N-((1R,3S)-3-(1-oxo-2,7-diazaspiro[4.5]decan-7- |
| | yl)cyclohexyl)-1H-indole-2-carboxamide |
| 883 | 4-fluoro-7-methyl-N-((1R,3R)-3-(1-oxo-2,7-diazaspiro[4.5]decan-7- |
| | yl)cyclohexyl)-1H-indole-2-carboxamide |
| 884 | N-((1R,3S)-3-(2,4-dimethyl-3-oxopiperazin-1-yl)cyclohexyl)-4-fluoro-7-methyl- |
| | 1H-indole-2-carboxamide |
| 885 | N-((1R,3R)-3-(2,4-dimethyl-3-oxopiperazin-1-yl)cyclohexyl)-4-fluoro-7-methyl- |
| | 1H-indole-2-carboxamide |
| 886 | 1-(4-(1-(4-fluoro-7-methyl-1H-indole-2-carbonyl)piperidine-3- |
| | carbonyl)piperazin-1-yl)ethan-1-one |
| 887 | 4-fluoro-7-methyl-N-((1R,3S)-3-(4-methyl-3-oxo-1,4-diazepan-1-yl)cyclohexyl)- |
| | 1H-indole-2-carboxamide |
| 888 | N-((1R,3S)-3-(6-acetyl-2,6-diazaspiro[3.3]heptan-2-yl)cyclohexyl)-4-fluoro-7- |
| | methyl-1H-indole-2-carboxamide |
| 889 | N-((1R,3R)-3-(6-acetyl-2,6-diazaspiro[3.3]heptan-2-yl)cyclohexyl)-4-fluoro-7- |
| | methyl-1H-indole-2-carboxamide |
| 890 | 4-fluoro-7-methyl-N-((1R)-3-(2-oxo-1,3,8-triazaspiro[4.5]decan-8- |
| | yl)cyclohexyl)-1H-indole-2-carboxamide |
| 891 | (R)-N-(1′-(dimethylglycyl)-[1,4′-bipiperidin]-3-yl)-4-fluoro-7-methyl-1H-indole- |
| | 2-carboxamide |
| 892 | (S)-N-(1′-(dimethylglycyl)-[1,4′-bipiperidin]-3-yl)-4-fluoro-7-methyl-1H-indole- |
| | 2-carboxamide |
| 893 | 4-fluoro-7-methyl-N-((1R)-3-(5-oxo-1,4-diazepan-1-yl)cyclohexyl)-1H-indole-2- |
| | carboxamide |
| 894 | 4-fluoro-7-methyl-N-((1R)-3-(2-oxo-1,8-diazaspiro[4.5]decan-8-yl)cyclohexyl)- |
| | 1H-indole-2-carboxamide |
| 895 | 4-fluoro-7-methyl-N-(3-(2-oxo-3-oxa-1,8-diazaspiro[4.5]decan-8-yl)cyclohexyl)- |
| | 1H-indole-2-carboxamide |
| 896 | 4-fluoro-7-methyl-N-((1R)-3-(3-oxo-2,8-diazaspiro[4.5]decan-8-yl)cyclohexyl)- |
| | 1H-indole-2-carboxamide |
| 897 | 4-fluoro-7-methyl-N-((1R,3S)-3-(7-oxo-2,6-diazaspiro[3.4]octan-2- |
| | yl)cyclohexyl)-1H-indole-2-carboxamide |
| 898 | 4-fluoro-7-methyl-N-((1R,3R)-3-(7-oxo-2,6-diazaspiro[3.4]octan-2- |
| | yl)cyclohexyl)-1H-indole-2-carboxamide |
| 899 | 4-fluoro-7-methyl-N-((1R,3S)-3-(3-(N-methylmethylsulfonamido)pyrrolidin-1- |
| | yl)cyclohexyl)-1H-indole-2-carboxamide |
| 900 | 4-fluoro-7-methyl-N-((1R,3R)-3-((R)-3-(N-methylmethylsulfonamido)pyrrolidin- |
| | 1-yl)cyclohexyl)-1H-indole-2-carboxamide |
| 901 | 4-fluoro-7-methyl-N-((1R,3R)-3-((S)-3-(N-methylmethylsulfonamido)pyrrolidin- |
| | 1-yl)cyclohexyl)-1H-indole-2-carboxamide |
| 902 | 4-fluoro-7-methyl-N-((1R,3R)-3-(pyridin-3-yl)cyclohexyl)-1H-indole-2- |
| | carboxamide |
| 903 | 4-fluoro-7-methyl-N-((1R,3S)-3-(pyridin-3-yl)cyclohexyl)-1H-indole-2- |
| | carboxamide |
| 904 | 4-fluoro-7-methyl-N-((1S,3S)-3-(pyridin-3-yl)cyclohexyl)-1H-indole-2- |
| | carboxamide |
| 905 | 4-fluoro-7-methyl-N-((1S,3R)-3-(pyridin-3-yl)cyclohexyl)-1H-indole-2- |
| | carboxamide |
| 906 | N-((1R,3S)-3-(4-acetyl-3-methylpiperazin-1-yl)cyclohexyl)-4-fluoro-7-methyl- |
| | 1H-indole-2-carboxamide |
| 907 | N-((1R,3R)-3-(4-acetyl-3-methylpiperazin-1-yl)cyclohexyl)-4-fluoro-7-methyl- |
| | 1H-indole-2-carboxamide |
| 908 | N-((1R,3S)-3-(3-(2-(dimethylamino)-N-methylacetamido)pyrrolidin-1- |
| | yl)cyclohexyl)-4-fluoro-7-methyl-1H-indole-2-carboxamide |
| 909 | N-((1R,3R)-3-(3-(2-(dimethylamino)-N-methylacetamido)pyrrolidin-1- |
| | yl)cyclohexyl)-4-fluoro-7-methyl-1H-indole-2-carboxamide |
| 910 | 4-fluoro-7-methyl-N-((1R,3S)-3-(2-oxo-[1,3′-bipyrrolidin]-1′-yl)cyclohexyl)-1H- |
| | indole-2-carboxamide |
| 911 | 4-fluoro-7-methyl-N-((1R,3R)-3-(2-oxo-[1,3′-bipyrrolidin]-1′-yl)cyclohexyl)-1H- |
| | indole-2-carboxamide |
| 912 | 4-fluoro-7-methyl-N-((1R,3R)-3-(pyridin-4-yl)cyclohexyl)-1H-indole-2- |
| | carboxamide |
| 913 | 4-fluoro-7-methyl-N-((1R,3S)-3-(pyridin-4-yl)cyclohexyl)-1H-indole-2- |
| | carboxamide |
| 914 | 4-fluoro-7-methyl-N-((1S,3R)-3-(pyridin-4-yl)cyclohexyl)-1H-indole-2- |
| | carboxamide |
| 915 | 4-fluoro-7-methyl-N-((1S,3S)-3-(pyridin-4-yl)cyclohexyl)-1H-indole-2- |
| | carboxamide |
| 916 | 4-fluoro-7-methyl-N-((1R,3S)-3-(4-methyl-2-oxopiperazin-1-yl)cyclohexyl)-1H- |
| | indole-2-carboxamide |
| 917 | 4-fluoro-7-methyl-N-((1R,3R)-3-(4-methyl-2-oxopiperazin-1-yl)cyclohexyl)-1H- |
| | indole-2-carboxamide |
| 918 | 4-fluoro-7-methyl-N-((1R,3S)-3-(4-(oxetane-3-carbonyl)piperazin-1- |
| | yl)cyclohexyl)-1H-indole-2-carboxamide |
| 919 | 4-fluoro-7-methyl-N-((1R,3R)-3-(4-(oxetane-3-carbonyl)piperazin-1- |
| | yl)cyclohexyl)-1H-indole-2-carboxamide |
| 920 | 4-fluoro-7-methyl-N-((1R,3S)-3-((R)-6-oxohexahydropyrrolo[1,2-a]pyrazin- |
| | 2(1H)-yl)cyclohexyl)-1H-indole-2-carboxamide |
| 921 | 4-fluoro-7-methyl-N-((1R,3R)-3-((R)-6-oxohexahydropyrrolo[1,2-a]pyrazin- |
| | 2(1H)-yl)cyclohexyl)-1H-indole-2-carboxamide |
| 922 | 4-fluoro-7-methyl-N-((1R,3S)-3-((S)-6-oxohexahydropyrrolo[1,2-a]pyrazin- |
| | 2(1H)-yl)cyclohexyl)-1H-indole-2-carboxamide |
| 923 | 4-fluoro-7-methyl-N-((1R,3R)-3-((S)-6-oxohexahydropyrrolo[1,2-a]pyrazin- |
| | 2(1H)-yl)cyclohexyl)-1H-indole-2-carboxamide |
| 924 | 4-fluoro-7-methyl-N-((1R,3S)-3-(4-(tetrahydrofuran-3-yl)piperazin-1- |
| | yl)cyclohexyl)-1H-indole-2-carboxamide |
| 925 | 4-fluoro-7-methyl-N-((1R,3R)-3-(4-(tetrahydrofuran-3-yl)piperazin-1- |
| | yl)cyclohexyl)-1H-indole-2-carboxamide |
| 926 | N-((1R,3S)-3-(8-acetyl-3,8-diazabicyclo[3.2.1]octan-3-yl)cyclohexyl)-4-fluoro-7- |
| | methyl-1H-indole-2-carboxamide |
| 927 | N-((1R,3R)-3-(8-acetyl-3,8-diazabicyclo[3.2.1]octan-3-yl)cyclohexyl)-4-fluoro-7- |
| | methyl-1H-indole-2-carboxamide |
| 928 | 4-fluoro-7-methyl-N-((1R,3S)-3-(3-oxo-2,8-diazaspiro[4.5]decan-8- |
| | yl)cyclohexyl)-1H-indole-2-carboxamide |
| 929 | 4-fluoro-7-methyl-N-((1R,3R)-3-(3-oxo-2,8-diazaspiro[4.5]decan-8- |
| | yl)cyclohexyl)-1H-indole-2-carboxamide |
| 930 | N-((1R,3S)-3-((R)-4-acetyl-3-methylpiperazin-1-yl)cyclohexyl)-4-fluoro-7- |
| | methyl-1H-indole-2-carboxamide |
| 931 | N-((1R,3R)-3-((R)-4-acetyl-3-methylpiperazin-1-yl)cyclohexyl)-4-fluoro-7- |
| | methyl-1H-indole-2-carboxamide |
| 932 | 4-fluoro-7-methyl-N-((1R,3S)-3-(5-oxo-1,4-diazepan-1-yl)cyclohexyl)-1H- |
| | indole-2-carboxamide |
| 933 | 4-fluoro-7-methyl-N-((1R,3R)-3-(5-oxo-1,4-diazepan-1-yl)cyclohexyl)-1H- |
| | indole-2-carboxamide |
| 934 | 4-fluoro-N-((1R,3S)-3-(3-(2-hydroxy-N-methylacetamido)pyrrolidin-1- |
| | yl)cyclohexyl)-7-methyl-1H-indole-2-carboxamide |
| 935 | 4-fluoro-N-((1R,3R)-3-(3-(2-hydroxy-N-methylacetamido)pyrrolidin-1- |
| | yl)cyclohexyl)-7-methyl-1H-indole-2-carboxamide |
| 936 | N-((1R,3S)-3-(5-acetyl-2,5-diazabicyclo[2.2.1]heptan-2-yl)cyclohexyl)-4-fluoro- |
| | 7-methyl-1H-indole-2-carboxamide |
| 937 | N-((1R,3R)-3-(5-acetyl-2,5-diazabicyclo[2.2.1]heptan-2-yl)cyclohexyl)-4-fluoro- |
| | 7-methyl-1H-indole-2-carboxamide |
| 938 | 4-fluoro-7-methyl-N-((1R,3S)-3-(2-oxo-1,8-diazaspiro[4.5]decan-8- |
| | yl)cyclohexyl)-1H-indole-2-carboxamide |
| 939 | 4-fluoro-7-methyl-N-((1R,3R)-3-(2-oxo-1,8-diazaspiro[4.5]decan-8- |
| | yl)cyclohexyl)-1H-indole-2-carboxamide |
| 940 | 4-fluoro-7-methyl-N-((1R,3S)-3-(2-oxo-1,3,8-triazaspiro[4.5]decan-8- |
| | yl)cyclohexyl)-1H-indole-2-carboxamide |
| 941 | 4-fluoro-7-methyl-N-((1R,3R)-3-(2-oxo-1,3,8-triazaspiro[4.5]decan-8- |
| | yl)cyclohexyl)-1H-indole-2-carboxamide |
| 942 | 4-fluoro-7-methyl-N-((1R,3S)-3-((S)-3-(N-methylmethylsulfonamido)pyrrolidin- |
| | 1-yl)cyclohexyl)-1H-indole-2-carboxamide |
| 943 | 4-fluoro-7-methyl-N-((1R,3S)-3-((R)-3-(N-methylmethylsulfonamido)pyrrolidin- |
| | 1-yl)cyclohexyl)-1H-indole-2-carboxamide |
| 944 | 4-fluoro-7-methyl-N-((1R,3S)-3-(4-methyl-5-oxo-1,4-diazepan-1-yl)cyclohexyl)- |
| | 1H-indole-2-carboxamide |
| 945 | 4-fluoro-7-methyl-N-((1R,3R)-3-(4-methyl-5-oxo-1,4-diazepan-1-yl)cyclohexyl)- |
| | 1H-indole-2-carboxamide |
| 946 | 4-fluoro-7-methyl-N-((1R,3S)-3-(3-(N-methyloxetane-3-carboxamido)pyrrolidin- |
| | 1-yl)cyclohexyl)-1H-indole-2-carboxamide |
| 947 | 4-fluoro-7-methyl-N-((1R,3R)-3-(3-(N-methyloxetane-3-carboxamido)pyrrolidin- |
| | 1-yl)cyclohexyl)-1H-indole-2-carboxamide |
| 948 | N-((1R,3S)-3-(4-(ethylcarbamoyl)piperazin-1-yl)cyclohexyl)-4-fluoro-7-methyl- |
| | 1H-indole-2-carboxamide |
| 949 | N-((1R,3R)-3-(4-(ethylcarbamoyl)piperazin-1-yl)cyclohexyl)-4-fluoro-7-methyl- |
| | 1H-indole-2-carboxamide |
| 950 | N-((1R,3S)-3-(1-acetyl-1,6-diazaspiro[3.3]heptan-6-yl)cyclohexyl)-4-fluoro-7- |
| | methyl-1H-indole-2-carboxamide |
| 951 | N-((1R,3R)-3-(1-acetyl-1,6-diazaspiro[3.3]heptan-6-yl)cyclohexyl)-4-fluoro-7- |
| | methyl-1H-indole-2-carboxamide |
| 952 | N-((1R,3S)-3-(3-acetyl-3,8-diazabicyclo[3.2.1]octan-8-yl)cyclohexyl)-4-fluoro-7- |
| | methyl-1H-indole-2-carboxamide |
| 953 | N-((1R,3R)-3-(3-acetyl-3,8-diazabicyclo[3.2.1]octan-8-yl)cyclohexyl)-4-fluoro-7- |
| | methyl-1H-indole-2-carboxamide |
| 954 | N-((1R,3S)-3-(5-((dimethylamino)methyl)-1,3,4-oxadiazol-2-yl)cyclohexyl)-4- |
| | fluoro-7-methyl-1H-indole-2-carboxamide |
| 955 | N-((1S,3R)-3-(5-((dimethylamino)methyl)-1,3,4-oxadiazol-2-yl)cyclohexyl)-4- |
| | fluoro-7-methyl-1H-indole-2-carboxamide |
| 956 | 4-fluoro-7-methyl-N-((1R,3R)-3-(piperazin-1-yl)cyclohexyl)-1H-indole-2- |
| | carboxamide |
| 957 | 4-fluoro-7-methyl-N-((1R,3R)-3-(piperazin-1-yl)cyclohexyl)-1H-indole-2- |
| | carboxamide |
| 958 | 4-fluoro-7-methyl-N-((1R,3S)-3-(3-(2-oxooxazolidin-3-yl)pyrrolidin-1- |
| | yl)cyclohexyl)-1H-indole-2-carboxamide |
| 959 | 4-fluoro-7-methyl-N-((1R,3R)-3-(3-(2-oxooxazolidin-3-yl)pyrrolidin-1- |
| | yl)cyclohexyl)-1H-indole-2-carboxamide |
| 960 | 4-fluoro-7-methyl-N-((1R,3S)-3-(3-(N-methylacetamido)azetidin-1- |
| | yl)cyclohexyl)-1H-indole-2-carboxamide |
| 961 | N-((1R,3S)-3-(5-acetyl-2,5-diazabicyclo[2.2.2]octan-2-yl)cyclohexyl)-4-fluoro-7- |
| | methyl-1H-indole-2-carboxamide |
| 962 | N-((1R,3R)-3-(5-acetyl-2,5-diazabicyclo[2.2.2]octan-2-yl)cyclohexyl)-4-fluoro-7- |
| | methyl-1H-indole-2-carboxamide |
| 963 | 4-fluoro-7-methyl-N-((1R,3S)-3-(2-oxo-3-oxa-1,8-diazaspiro[4.5]decan-8- |
| | yl)cyclohexyl)-1H-indole-2-carboxamide |
| 964 | 4-fluoro-7-methyl-N-((1R,3R)-3-(2-oxo-3-oxa-1,8-diazaspiro[4.5]decan-8- |
| | yl)cyclohexyl)-1H-indole-2-carboxamide |
| 965 | 4-fluoro-7-methyl-N-((1R,3S)-3-(2-oxo-1-(2,2,2-trifluoroethyl)-1,8- |
| | diazaspiro[4.5]decan-8-yl)cyclohexyl)-1H-indole-2-carboxamide |
| 966 | 4-fluoro-7-methyl-N-((1R,3R)-3-(2-oxo-1-(2,2,2-trifluoroethyl)-1,8- |
| | diazaspiro[4.5]decan-8-yl)cyclohexyl)-1H-indole-2-carboxamide |
| 967 | N-((1R,3S)-3-((S)-4-acetyl-3-methylpiperazin-1-yl)cyclohexyl)-4-fluoro-7- |
| | methyl-1H-indole-2-carboxamide |
| 968 | N-((1R,3R)-3-((S)-4-acetyl-3-methylpiperazin-1-yl)cyclohexyl)-4-fluoro-7- |
| | methyl-1H-indole-2-carboxamide |
| 969 | 4-fluoro-7-methyl-N-((1R,3S)-3-(3-(N-methylisobutyramido)pyrrolidin-1- |
| | yl)cyclohexyl)-1H-indole-2-carboxamide |
| 970 | 4-fluoro-7-methyl-N-((1R,3R)-3-(3-(N-methylisobutyramido)pyrrolidin-1- |
| | yl)cyclohexyl)-1H-indole-2-carboxamide |
| 971 | 4-fluoro-7-methyl-N-((1R,3S)-3-(3-(N- |
| | methylcyclopropanecarboxamido)pyrrolidin-1-yl)cyclohexyl)-1H-indole-2- |
| | carboxamide |
| 972 | 4-fluoro-7-methyl-N-((1R,3R)-3-(3-(N- |
| | methylcyclopropanecarboxamido)pyrrolidin-1-yl)cyclohexyl)-1H-indole-2- |
| | carboxamide |
| 973 | 4-fluoro-7-methyl-N-((1R,3S)-3-(3-oxo-4-(tetrahydrofuran-3-yl)piperazin-1- |
| | yl)cyclohexyl)-1H-indole-2-carboxamide |
| 974 | 4-fluoro-7-methyl-N-((1R,3R)-3-(3-oxo-4-(tetrahydrofuran-3-yl)piperazin-1- |
| | yl)cyclohexyl)-1H-indole-2-carboxamide |
| 975 | 4-fluoro-7-methyl-N-((1R,3S)-3-(5-methyl-1,3,4-oxadiazol-2-yl)cyclohexyl)-1H- |
| | indole-2-carboxamide |
| 976 | 4-fluoro-7-methyl-N-((1S,3R)-3-(5-methyl-1,3,4-oxadiazol-2-yl)cyclohexyl)-1H- |
| | indole-2-carboxamide |
| 977 | N-((1R,3S)-3-(4-cyclopropyl-3-oxopiperazin-1-yl)cyclohexyl)-4-fluoro-7-methyl- |
| | 1H-indole-2-carboxamide |
| 978 | N-((1R,3R)-3-(4-cyclopropyl-3-oxopiperazin-1-yl)cyclohexyl)-4-fluoro-7- |
| | methyl-1H-indole-2-carboxamide |
| 979 | N-((1R,3S)-3-(4-acetyl-1,4-diazepan-1-yl)cyclohexyl)-4-fluoro-7-methyl-1H- |
| | indole-2-carboxamide |
| 980 | N-((1R,3R)-3-(4-acetyl-1,4-diazepan-1-yl)cyclohexyl)-4-fluoro-7-methyl-1H- |
| | indole-2-carboxamide |
| 981 | 4-fluoro-7-methyl-N-((1R,3S)-3-(4-(methylsulfonyl)-1,4-diazepan-1- |
| | yl)cyclohexyl)-1H-indole-2-carboxamide |
| 982 | 4-fluoro-7-methyl-N-((1R,3R)-3-(4-(methylsulfonyl)-1,4-diazepan-1- |
| | yl)cyclohexyl)-1H-indole-2-carboxamide |
| 983 | 4-fluoro-N-((1R,3S)-3-((R)-3-(2-hydroxy-N-methylacetamido)pyrrolidin-1- |
| | yl)cyclohexyl)-7-methyl-1H-indole-2-carboxamide |
| 984 | 4-fluoro-N-((1R,3R)-3-((R)-3-(2-hydroxy-N-methylacetamido)pyrrolidin-1- |
| | yl)cyclohexyl)-7-methyl-1H-indole-2-carboxamide |
| 985 | N-((3S,5S)-5-(4-acetylpiperazin-1-yl)tetrahydro-2H-pyran-3-yl)-4-fluoro-7- |
| | methyl-1H-indole-2-carboxamide |
| 986 | N-((3S,5R)-5-(4-acetylpiperazin-1-yl)tetrahydro-2H-pyran-3-yl)-4-fluoro-7- |
| | methyl-1H-indole-2-carboxamide |
| 987 | N-((3R,5R)-5-(4-acetylpiperazin-1-yl)tetrahydro-2H-pyran-3-yl)-4-fluoro-7- |
| | methyl-1H-indole-2-carboxamide |
| 988 | N-((3R,5S)-5-(4-acetylpiperazin-1-yl)tetrahydro-2H-pyran-3-yl)-4-fluoro-7- |
| | methyl-1H-indole-2-carboxamide |
| 989 | 4-fluoro-7-methyl-N-((1R,3S)-3-(3-(trifluoromethyl)-5,6-dihydro- |
| | [1,2,4]triazolo[4,3-a]pyrazin-7(8H)-yl)cyclohexyl)-1H-indole-2-carboxamide |
| 990 | 4-fluoro-7-methyl-N-((1R,3R)-3-(3-(trifluoromethyl)-5,6-dihydro- |
| | [1,2,4]triazolo[4,3-a]pyrazin-7(8H)-yl)cyclohexyl)-1H-indole-2-carboxamide |
| 991 | N-((1R,3S)-3-(5,6-dihydroimidazo[1,5-a]pyrazin-7(8H)-yl)cyclohexyl)-4-fluoro- |
| | 7-methyl-1H-indole-2-carboxamide |
| 992 | N-((1R,3R)-3-(5,6-dihydroimidazo[1,5-a]pyrazin-7(8H)-yl)cyclohexyl)-4-fluoro- |
| | 7-methyl-1H-indole-2-carboxamide |
| 993 | 4-fluoro-7-methyl-N-((1R,3S)-3-(3-(trifluoromethyl)-5,6-dihydroimidazo[1,5- |
| | a]pyrazin-7(8H)-yl)cyclohexyl)-1H-indole-2-carboxamide |
| 994 | 4-fluoro-7-methyl-N-((1R,3R)-3-(3-(trifluoromethyl)-5,6-dihydroimidazo[1,5- |
| | a]pyrazin-7(8H)-yl)cyclohexyl)-1H-indole-2-carboxamide |
| 995 | 4-fluoro-N-((1R,3S)-3-((S)-3-(2-hydroxy-N-methylacetamido)pyrrolidin-1- |
| | yl)cyclohexyl)-7-methyl-1H-indole-2-carboxamide |
| 996 | 4-fluoro-N-((1R,3R)-3-((S)-3-(2-hydroxy-N-methylacetamido)pyrrolidin-1- |
| | yl)cyclohexyl)-7-methyl-1H-indole-2-carboxamide |
| 997 | isopropyl 4-((1S,3R)-3-(4-fluoro-7-methyl-1H-indole-2- |
| | carboxamido)cyclohexyl)piperazine-1-carboxylate |
| 998 | isopropyl 4-((1R,3R)-3-(4-fluoro-7-methyl-1H-indole-2- |
| | carboxamido)cyclohexyl)piperazine-1-carboxylate |
| 999 | tetrahydro-2H-pyran-4-yl 4-((1S,3R)-3-(4-fluoro-7-methyl-1H-indole-2- |
| | carboxamido)cyclohexyl)piperazine-1-carboxylate |
| 1000 | tetrahydro-2H-pyran-4-yl 4-((1R,3R)-3-(4-fluoro-7-methyl-1H-indole-2- |
| | carboxamido)cyclohexyl)piperazine-1-carboxylate |
| 1001 | 4-fluoro-7-methyl-N-((1R,3S)-3-(3-(trifluoromethyl)-5,6-dihydroimidazo[1,5- |
| | a]pyrazin-7(8H)-yl)cyclohexyl)-1H-indole-2-carboxamide |
| 1002 | N-((1R,3S)-3-(3-(dimethylamino)-2-oxopyrrolidin-1-yl)cyclohexyl)-4-fluoro-7- |
| | methyl-1H-indole-2-carboxamide |
| 1003 | 4-fluoro-7-methyl-N-((1R,3S)-3-(pyrimidin-4-yl)cyclohexyl)-1H-indole-2- |
| | carboxamide |
| 1004 | 4-fluoro-7-methyl-N-((1R,3S)-3-(2-oxo-4-(tetrahydrofuran-3-yl)piperazin-1- |
| | yl)cyclohexyl)-1H-indole-2-carboxamide |
| 1005 | N-((1R,3S)-3-(4-ethyl-2-oxopiperazin-1-yl)cyclohexyl)-4-fluoro-7-methyl-1H- |
| | indole-2-carboxamide |
| 1006 | N-((3S,5R)-5-(4-acetylpiperazin-1-yl)-1-methylpiperidin-3-yl)-4-fluoro-7-methyl- |
| | 1H-indole-2-carboxamide |
| 1007 | N-((1R,3S)-3-(1-acetylpiperidin-4-yl)cyclohexyl)-4-fluoro-7-methyl-1H-indole-2- |
| | carboxamide |
| 1008 | 4-fluoro-7-methyl-N-((1R,3S)-3-(3-oxo-2-oxa-4,9-diazaspiro[5.5]undecan-9- |
| | yl)cyclohexyl)-1H-indole-2-carboxamide |
| 1009 | N-((1R,3S)-3-(2,2-dioxido-2-thia-1,3,8-triazaspiro[4.5]decan-8-yl)cyclohexyl)-4- |
| | fluoro-7-methyl-1H-indole-2-carboxamide |
| 1010 | 4-fluoro-7-methyl-N-((1R,3S)-3-(2-oxo-4-oxa-1,9-diazaspiro[5.5]undecan-9- |
| | yl)cyclohexyl)-1H-indole-2-carboxamide |
| 1011 | 4-fluoro-7-methyl-N-((1R,3S)-3-(5-methyl-1,2,4-oxadiazol-3-yl)cyclohexyl)-1H- |
| | indole-2-carboxamide |
| 1012 | 4-fluoro-7-methyl-N-((1R,3S)-3-(6-oxo-1,6-dihydro-1,2,4-triazin-3- |
| | yl)cyclohexyl)-1H-indole-2-carboxamide |
| 1013 | 4-fluoro-7-methyl-N-((1R,3S)-3-(6-oxo-1,6-dihydropyrimidin-2-yl)cyclohexyl)- |
| | 1H-indole-2-carboxamide |
| 1014 | N-((1R,3S)-3-(5-((dimethylamino)methyl)-1,2,4-oxadiazol-3-yl)cyclohexyl)-4- |
| | fluoro-7-methyl-1H-indole-2-carboxamide |
| 1015 | N-((1R,3R)-3-(4-acetylpiperazin-1-yl)-4-fluorocyclohexyl)-4-fluoro-7-methyl- |
| | 1H-indole-2-carboxamide |
| 1016 | N-((1R,3R)-3-(4-acetylpiperazin-1-yl)-4-(trifluoromethyl)cyclohexyl)-4-fluoro-7- |
| | methyl-1H-indole-2-carboxamide |
| 1017 | N-((1R,3S)-3-(4-acetyl-2-(trifluoromethyl)piperazin-1-yl)cyclohexyl)-4-fluoro-7- |
| | methyl-1H-indole-2-carboxamide |
| 1018 | N-((1R,3S)-3-(4-acetyl-3-(trifluoromethyl)piperazin-1-yl)cyclohexyl)-4-fluoro-7- |
| | methyl-1H-indole-2-carboxamide |
| 1019 | 4-fluoro-7-methyl-N-((1R,3S)-3-(3-oxo-2,8-diazaspiro[4.5]decan-8- |
| | yl)cyclohexyl)-1H-indole-2-carboxamide |
| 1020 | 4-fluoro-7-methyl-N-((1R,3S)-3-(7-oxo-5-oxa-2,8-diazaspiro[3.5]nonan-2- |
| | yl)cyclohexyl)-1H-indole-2-carboxamide |
| 1021 | N-((1R,3S)-3-(4-acetyl-2-methylpiperazin-1-yl)cyclohexyl)-4-fluoro-7-methyl- |
| | 1H-indole-2-carboxamide |
| 1022 | N-((1R,3S)-3-(4-acetyl-3-(hydroxymethyl)piperazin-1-yl)cyclohexyl)-4-fluoro-7- |
| | methyl-1H-indole-2-carboxamide |
| 1023 | N-((1R,3S)-3-(4-acetyl-2-(hydroxymethyl)piperazin-1-yl)cyclohexyl)-4-fluoro-7- |
| | methyl-1H-indole-2-carboxamide |
| 1024 | N-((1R,3S)-3-(7-acetyl-4,7-diazaspiro[2.5]octan-4-yl)cyclohexyl)-4-fluoro-7- |
| | methyl-1H-indole-2-carboxamide |
| 1025 | N-((1R,3S)-3-(4-acetyl-4,7-diazaspiro[2.5]octan-7-yl)cyclohexyl)-4-fluoro-7- |
| | methyl-1H-indole-2-carboxamide |
| 1026 | N-((1R,3S)-3-(3,4-dimethyl-2-oxopiperazin-1-yl)cyclohexyl)-4-fluoro-7-methyl- |
| | 1H-indole-2-carboxamide |
| 1027 | 4-fluoro-7-methyl-N-(1-(pyridin-4-yl)piperidin-3-yl)-1H-indole-2-carboxamide |
| 1028 | 4-fluoro-7-methyl-N-(1′-methyl-2′-oxo-[1,4′-bipiperidin]-3-yl)-1H-indole-2- |
| | carboxamide |
| 1029 | isopropyl 4-((1S,3R)-3-(4-fluoro-7-methyl-1H-indole-2- |
| | carboxamido)cyclohexyl)piperazine-1-carboxylate |
| 1030 | tetrahydro-2H-pyran-4-yl 4-((1S,3R)-3-(4-fluoro-7-methyl-1H-indole-2- |
| | carboxamido)cyclohexyl)piperazine-1-carboxylate |
| 1031 | 4-fluoro-7-methyl-N-((1R,3S)-3-(2-(trifluoromethyl)-5,6-dihydro- |
| | [1,2,4]triazolo[1,5-a]pyrazin-7(8H)-yl)cyclohexyl)-1H-indole-2-carboxamide |
| 1032 | N-((1R,3S)-3-((4-acetylpiperazin-1-yl)methyl)cyclohexyl)-4-fluoro-7-methyl-1H- |
| | indole-2-carboxamide |
| 1033 | N-((1R,3S)-3-(5,6-dihydroimidazo[1,5-a]pyrazin-7(8H)-yl)cyclohexyl)-4-fluoro- |
| | 7-methyl-1H-indole-2-carboxamide |
| 1034 | 4-fluoro-7-methyl-N-((1R,3S)-3-(3-(trifluoromethyl)-5,6-dihydroimidazo[1,5- |
| | a]pyrazin-7(8H)-yl)cyclohexyl)-1H-indole-2-carboxamide |
| 1035 | 4-fluoro-N-((1R,3R,4R)-4-fluoro-3-(2-oxo-1,8-diazaspiro[4.5]decan-8- |
| | yl)cyclohexyl)-7-methyl-1H-indole-2-carboxamide |
| 1036 | 4-fluoro-N-((1R,3R,4R)-4-fluoro-3-((S)-3-(N-methylacetamido)pyrrolidin-1- |
| | yl)cyclohexyl)-7-methyl-1H-indole-2-carboxamide |
| 1037 | 4-fluoro-7-methyl-N-((1R,3S)-3-(4-((tetrahydro-2H-pyran-4- |
| | yl)carbamoyl)piperazin-1-yl)cyclohexyl)-1H-indole-2-carboxamide |
| 1038 | 4-fluoro-7-methyl-N-((1R,3S)-3-((3aS,6aS)-1-methyl-2- |
| | oxohexahydropyrrolo[3,4-b]pyrrol-5(1H)-yl)cyclohexyl)-1H-indole-2- |
| | carboxamide |
| 1039 | 4-fluoro-7-methyl-N-((1R,3S)-3-((3aR,6aR)-1-methyl-2- |
| | oxohexahydropyrrolo[3,4-b]pyrrol-5(1H)-yl)cyclohexyl)-1H-indole-2- |
| | carboxamide |
| 1040 | 4-fluoro-7-methyl-N-((1R,3S)-3-(4-methyl-5-oxohexahydropyrrolo[3,2-b]pyrrol- |
| | 1(2H)-yl)cyclohexyl)-1H-indole-2-carboxamide |
| 1041 | 4-fluoro-N-((1R,3S)-3-((S)-3-(2-hydroxy-N-methylacetamido)pyrrolidin-1- |
| | yl)cyclohexyl)-7-methyl-1H-indole-2-carboxamide |
| 1042 | 4-fluoro-N-((1R,3S)-3-((R)-3-(2-methoxy-N-methylacetamido)pyrrolidin-1- |
| | yl)cyclohexyl)-7-methyl-1H-indole-2-carboxamide |
| 1043 | 4-fluoro-N-((1R,3S)-3-((S)-3-(2-methoxy-N-methylacetamido)pyrrolidin-1- |
| | yl)cyclohexyl)-7-methyl-1H-indole-2-carboxamide |
| 1044 | 4-fluoro-7-methyl-N-((1R,3S)-3-(6-oxooctahydro-2H-pyrido[1,2-a]pyrazin-2- |
| | yl)cyclohexyl)-1H-indole-2-carboxamide |
| 1045 | 4-fluoro-7-methyl-N-((1R,3S)-3-(4-oxohexahydropyrazino[2,1-c][1,4]oxazin- |
| | 8(1H)-yl)cyclohexyl)-1H-indole-2-carboxamide |
| 1046 | 4-fluoro-7-methyl-N-((1R,3S)-3-(8-methyl-6,9-dioxooctahydro-2H-pyrazino[1,2- |
| | a]pyrazin-2-yl)cyclohexyl)-1H-indole-2-carboxamide |
| 1047 | 4-fluoro-7-methyl-N-((1R,3S)-3-(6-oxooctahydro-2H-pyrazino[1,2-c]pyrimidin- |
| | 2-yl)cyclohexyl)-1H-indole-2-carboxamide |
| 1048 | 4-fluoro-7-methyl-N-((1R,3S)-3-(6-oxo-1,3,4,6-tetrahydro-2H-pyrido[1,2- |
| | a]pyrazin-2-yl)cyclohexyl)-1H-indole-2-carboxamide |
| 1049 | 4-fluoro-7-methyl-N-((1R,3S)-3-(4-oxo-4,6,7,9-tetrahydro-8H-pyrazino[1,2- |
| | a]pyrimidin-8-yl)cyclohexyl)-1H-indole-2-carboxamide |
| 1050 | 4-fluoro-7-methyl-N-((1R,3S)-3-(3-oxohexahydroimidazo[1,5-a]pyrazin-7(1H)- |
| | yl)cyclohexyl)-1H-indole-2-carboxamide |
| 1051 | 4-fluoro-7-methyl-N-((1R,3S)-3-(2-methyl-3-oxohexahydroimidazo[1,5- |
| | a]pyrazin-7(1H)-yl)cyclohexyl)-1H-indole-2-carboxamide |
| 1052 | 4-fluoro-N-((1R,3S)-3-(3-(2-hydroxy-N,2-dimethylpropanamido)pyrrolidin-1- |
| | yl)cyclohexyl)-7-methyl-1H-indole-2-carboxamide |
| 1053 | N-((1R,3S)-3-(3-(N-ethyl-2-hydroxyacetamido)pyrrolidin-1-yl)cyclohexyl)-4- |
| | fluoro-7-methyl-1H-indole-2-carboxamide |
| 1054 | N-((1R,3S)-3-(3-(N-ethylmethylsulfonamido)pyrrolidin-1-yl)cyclohexyl)-4- |
| | fluoro-7-methyl-1H-indole-2-carboxamide |
| 1055 | N-((1R,3S)-3-(4-acetyl-3-ethylpiperazin-1-yl)cyclohexyl)-4-fluoro-7-methyl-1H- |
| | indole-2-carboxamide |
| 1056 | 4-fluoro-7-methyl-N-((1R,3S)-3-(2-methyl-3-oxo-2,5,6,8-tetrahydro- |
| | [1,2,4]triazolo[4,3-a]pyrazin-7(3H)-yl)cyclohexyl)-1H-indole-2-carboxamide |
| 1057 | 4-fluoro-7-methyl-N-((1R,3S)-3-(2-methyl-3-oxo-2,5,6,8-tetrahydroimidazo[1,5- |
| | a]pyrazin-7(3H)-yl)cyclohexyl)-1H-indole-2-carboxamide |
| 1058 | 4-fluoro-7-methyl-N-((1R,3S)-3-(3-oxo-2,5,6,8-tetrahydro-[1,2,4]triazolo[4,3- |
| | a]pyrazin-7(3H)-yl)cyclohexyl)-1H-indole-2-carboxamide |
| 1059 | 4-fluoro-7-methyl-N-((1R,3S)-3-(3-oxo-2,5,6,8-tetrahydroimidazo[1,5-a]pyrazin- |
| | 7(3H)-yl)cyclohexyl)-1H-indole-2-carboxamide |
| 1060 | 4-fluoro-N-((1R,3S)-3-((S)-3-((1-hydroxy-N- |
| | methylmethyl)sulfonamido)pyrrolidin-1-yl)cyclohexyl)-7-methyl-1H-indole-2- |
| | carboxamide |
| 1061 | 4-fluoro-N-((1R,3S)-3-((S)-3-(N-(2-hydroxyethyl)methylsulfonamido)pyrrolidin- |
| | 1-yl)cyclohexyl)-7-methyl-1H-indole-2-carboxamide |
| 1062 | N-((1R,3S)-3-((3R,5R)-4-acetyl-3,5-dimethylpiperazin-1-yl)cyclohexyl)-4-fluoro- |
| | 7-methyl-1H-indole-2-carboxamide |
| 1063 | 4-fluoro-N-((1R,3R)-3-((S)-3-(2-methoxy-N-methylacetamido)pyrrolidin-1- |
| | yl)cyclohexyl)-7-methyl-1H-indole-2-carboxamide |
| 1064 | 4-fluoro-7-methyl-N-((1R,3R)-3-(pyrimidin-4-yl)cyclohexyl)-1H-indole-2- |
| | carboxamide |
| 1065 | 4-fluoro-7-methyl-N-((1S,3R)-3-(5-methyl-1,2,4-oxadiazol-3-yl)cyclohexyl)-1H- |
| | indole-2-carboxamide |
| 1066 | 4-fluoro-7-methyl-N-((1R,3R)-3-(2-(trifluoromethyl)-5,6-dihydro- |
| | [1,2,4]triazolo[1,5-a]pyrazin-7(8H)-yl)cyclohexyl)-1H-indole-2-carboxamide |
| 1067 | 4-fluoro-7-methyl-N-((1R,3R)-3-(4-oxohexahydropyrazino[2,1-c][1,4]oxazin- |
| | 8(1H)-yl)cyclohexyl)-1H-indole-2-carboxamide |
| 1068 | 4-fluoro-7-methyl-N-((1R,3S)-3-(6-oxo-1,6-dihydropyrimidin-4-yl)cyclohexyl)- |
| | 1H-indole-2-carboxamide |
| 1069 | N-((1R,3S)-3-((R)-4-acetyl-3-(trifluoromethyl)piperazin-1-yl)cyclohexyl)-4- |
| | fluoro-7-methyl-1H-indole-2-carboxamide |
| 1070 | N-((1R,3S)-3-((S)-4-acetyl-3-(trifluoromethyl)piperazin-1-yl)cyclohexyl)-4- |
| | fluoro-7-methyl-1H-indole-2-carboxamide |
| 1071 | N-((1R,3R)-3-((R)-4-acetyl-3-(trifluoromethyl)piperazin-1-yl)cyclohexyl)-4- |
| | fluoro-7-methyl-1H-indole-2-carboxamide |
| 1072 | N-((1R,3R)-3-((S)-4-acetyl-3-(trifluoromethyl)piperazin-1-yl)cyclohexyl)-4- |
| | fluoro-7-methyl-1H-indole-2-carboxamide |
| 1073 | N-((1R,3S)-3-((S)-4-acetyl-2-methylpiperazin-1-yl)cyclohexyl)-4-fluoro-7- |
| | methyl-1H-indole-2-carboxamide |
| 1074 | N-((1R,3R)-3-((S)-4-acetyl-2-methylpiperazin-1-yl)cyclohexyl)-4-fluoro-7- |
| | methyl-1H-indole-2-carboxamide |
| 1075 | N-((1R,3R)-3-((R)-4-acetyl-3-(hydroxymethyl)piperazin-1-yl)cyclohexyl)-4- |
| | fluoro-7-methyl-1H-indole-2-carboxamide |
| 1076 | N-((1R,3R)-3-((S)-4-acetyl-3-(hydroxymethyl)piperazin-1-yl)cyclohexyl)-4- |
| | fluoro-7-methyl-1H-indole-2-carboxamide |
| 1077 | N-((1S,3R)-3-((4-acetylpiperazin-1-yl)methyl)cyclohexyl)-4-fluoro-7-methyl-1H- |
| | indole-2-carboxamide |
| 1078 | 4-fluoro-7-methyl-N-((1R,3S)-3-((S)-6-oxooctahydro-2H-pyrido[1,2-a]pyrazin-2- |
| | yl)cyclohexyl)-1H-indole-2-carboxamide |
| 1079 | 4-fluoro-7-methyl-N-((1R,3R)-3-((R)-6-oxooctahydro-2H-pyrido[1,2-a]pyrazin- |
| | 2-yl)cyclohexyl)-1H-indole-2-carboxamide |
| 1080 | 4-fluoro-7-methyl-N-((1R,3R)-3-((S)-6-oxooctahydro-2H-pyrido[1,2-a]pyrazin-2- |
| | yl)cyclohexyl)-1H-indole-2-carboxamide |
| 1081 | 4-fluoro-7-methyl-N-((1R,3R)-3-((R)-3-oxohexahydroimidazo[1,5-a]pyrazin- |
| | 7(1H)-yl)cyclohexyl)-1H-indole-2-carboxamide |
| 1082 | 4-fluoro-7-methyl-N-((1R,3R)-3-((S)-3-oxohexahydroimidazo[1,5-a]pyrazin- |
| | 7(1H)-yl)cyclohexyl)-1H-indole-2-carboxamide |
| 1083 | 4-fluoro-7-methyl-N-((1R,3S)-3-((R)-3-oxohexahydroimidazo[1,5-a]pyrazin- |
| | 7(1H)-yl)cyclohexyl)-1H-indole-2-carboxamide |
| 1084 | 4-fluoro-7-methyl-N-((1R,3S)-3-((S)-3-oxohexahydroimidazo[1,5-a]pyrazin- |
| | 7(1H)-yl)cyclohexyl)-1H-indole-2-carboxamide |
| 1085 | N-((1R,3S)-3-((S)-4-acetyl-3-ethylpiperazin-1-yl)cyclohexyl)-4-fluoro-7-methyl- |
| | 1H-indole-2-carboxamide |
| 1086 | N-((1R,3R)-3-((S)-4-acetyl-3-ethylpiperazin-1-yl)cyclohexyl)-4-fluoro-7-methyl- |
| | 1H-indole-2-carboxamide |
| 1087 | 4-fluoro-7-methyl-N-((1R,3S)-3-((S)-3-(methylamino)pyrrolidin-1- |
| | yl)cyclohexyl)-1H-indole-2-carboxamide |
| 1088 | 4-fluoro-7-methyl-N-((1R,3R)-3-((S)-3-(methylamino)pyrrolidin-1- |
| | yl)cyclohexyl)-1H-indole-2-carboxamide |
| 1089 | N-((1R,3S)-3-((R)-3-(dimethylamino)-2-oxopyrrolidin-1-yl)cyclohexyl)-4-fluoro- |
| | 7-methyl-1H-indole-2-carboxamide |
| 1090 | N-((1R,3S)-3-((S)-3-(dimethylamino)-2-oxopyrrolidin-1-yl)cyclohexyl)-4-fluoro- |
| | 7-methyl-1H-indole-2-carboxamide |
| 1091 | N-((1R,3R)-3-((R)-4-acetyl-2-methylpiperazin-1-yl)cyclohexyl)-4-fluoro-7- |
| | methyl-1H-indole-2-carboxamide |
| 1092 | N-((1R,3S)-3-((R)-4-acetyl-2-methylpiperazin-1-yl)cyclohexyl)-4-fluoro-7- |
| | methyl-1-indole-2-carboxamide |
| 1093 | 4-fluoro-7-methyl-N-((1R,3S)-3-((R)-6-oxooctahydro-2H-pyrido[1,2-a]pyrazin-2- |
| | yl)cyclohexyl)-1H-indole-2-carboxamide |
| 1094 | N-((1R,3S)-3-((R)-4-acetyl-3-ethylpiperazin-1-yl)cyclohexyl)-4-fluoro-7-methyl- |
| | 1H-indole-2-carboxamide |
| 1095 | N-((1R,3R)-3-((R)-4-acetyl-3-ethylpiperazin-1-yl)cyclohexyl)-4-fluoro-7-methyl- |
| | 1H-indole-2-carboxamide |
| 1096 | N-((1R,3R)-3-((3R,5S)-4-acetyl-3,5-dimethylpiperazin-1-yl)cyclohexyl)-4-fluoro- |
| | 7-methyl-1H-indole-2-carboxamide |
| 1097 | N-((1R,3S)-3-((3R,5S)-4-acetyl-3,5-dimethylpiperazin-1-yl)cyclohexyl)-4-fluoro- |
| | 7-methyl-1H-indole-2-carboxamide |
| 1098 | N-((1R,3S)-3-((S)-4-acetyl-3-(hydroxymethyl)piperazin-1-yl)cyclohexyl)-4- |
| | fluoro-7-methyl-1H-indole-2-carboxamide |
| 1099 | N-((1R,3S)-3-((R)-4-acetyl-3-(hydroxymethyl)piperazin-1-yl)cyclohexyl)-4- |
| | fluoro-7-methyl-1H-indole-2-carboxamide |
| 1100 | N-((1R,3R)-3-(4-acetyl-4,7-diazaspiro[2.5]octan-7-yl)cyclohexyl)-4-fluoro-7- |
| | methyl-1H-indole-2-carboxamide |
| 1101 | 4-fluoro-7-methyl-N-((1R,3R)-3-((S)-8-methyl-6,9-dioxooctahydro-2H- |
| | pyrazino[1,2-a]pyrazin-2-yl)cyclohexyl)-1H-indole-2-carboxamide |
| 1102 | 4-fluoro-7-methyl-N-((1R,3R)-3-((R)-8-methyl-6,9-dioxooctahydro-2H- |
| | pyrazino[1,2-a]pyrazin-2-yl)cyclohexyl)-1H-indole-2-carboxamide |
| 1103 | 4-fluoro-7-methyl-N-((1R,3S)-3-((S)-8-methyl-6,9-dioxooctahydro-2H- |
| | pyrazino[1,2-a]pyrazin-2-yl)cyclohexyl)-1H-indole-2-carboxamide |
| 1104 | 4-fluoro-7-methyl-N-((1R,3S)-3-((R)-8-methyl-6,9-dioxooctahydro-2H- |
| | pyrazino[1,2-a]pyrazin-2-yl)cyclohexyl)-1H-indole-2-carboxamide |
| 1105 | 4-fluoro-7-methyl-N-((1R,3S)-3-(3-oxo-2,5,6,8-tetrahydro-[1,2,4]triazolo[4,3- |
| | a]pyrazin-7(3H)-yl)cyclohexyl)-1H-indole-2-carboxamide |
| 1106 | 4-fluoro-7-methyl-N-((1R,3R)-3-(3-oxo-2,5,6,8-tetrahydro-[1,2,4]triazolo[4,3- |
| | a]pyrazin-7(3H)-yl)cyclohexyl)-1H-indole-2-carboxamide |
| 1107 | N-((3S,5S)-5-(4-acetylpiperazin-1-yl)-1-methylpiperidin-3-yl)-4-fluoro-7-methyl- |
| | 1H-indole-2-carboxamide |
| 1108 | N-((3R,5R)-5-(4-acetylpiperazin-1-yl)-1-methylpiperidin-3-yl)-4-fluoro-7- |
| | methyl-1H-indole-2-carboxamide |
| 1109 | N-((3R,5S)-5-(4-acetylpiperazin-1-yl)-1-methylpiperidin-3-yl)-4-fluoro-7-methyl- |
| | 1H-indole-2-carboxamide |
| 1110 | N-((1S,3R)-3-(5-((dimethylamino)methyl)-1,2,4-oxadiazol-3-yl)cyclohexyl)-4- |
| | fluoro-7-methyl-1H-indole-2-carboxamide |
| 1111 | 4-fluoro-7-methyl-N-(3-(3-oxotetrahydro-3H-oxazolo[3,4-a]pyrazin-7(1H)- |
| | yl)cyclohexyl)-1H-indole-2-carboxamide |
| 1112 | 4-fluoro-N-((1R,3S)-3-(4-isobutyryl-3-(trifluoromethyl)piperazin-1- |
| | yl)cyclohexyl)-7-methyl-1H-indole-2-carboxamide |
| 1113 | 4-fluoro-7-methyl-N-((1R,3S)-3-(4-propionyl-3-(trifluoromethyl)piperazin-1- |
| | yl)cyclohexyl)-1H-indole-2-carboxamide |
| 1114 | N-((1R,3S)-3-(4-((E)-N′-cyano-N-methylcarbamimidoyl)piperazin-1- |
| | yl)cyclohexyl)-4-fluoro-7-methyl-1H-indole-2-carboxamide |
| 1115 | 4-fluoro-7-methyl-N-((1R,3S)-3-(4-((E)-1-(methylamino)-2-nitrovinyl)piperazin- |
| | 1-yl)cyclohexyl)-1H-indole-2-carboxamide |
| 1116 | N-((1R,3S)-3-(4-((E)-1-(cyanoimino)ethyl)piperazin-1-yl)cyclohexyl)-4-fluoro-7- |
| | methyl-1H-indole-2-carboxamide |
| 1117 | 4-fluoro-N-((1R,3S)-3-(4-(2-hydroxyacetyl)-3-(trifluoromethyl)piperazin-1- |
| | yl)cyclohexyl)-7-methyl-1H-indole-2-carboxamide |
| 1118 | N-((1R,3S)-3-(1-(4-acetylpiperazin-1-yl)ethyl)cyclohexyl)-4-fluoro-7-methyl-1H- |
| | indole-2-carboxamide |
| 1119 | N-((1R,3S)-3-((R)-3-(N-ethyl-2-hydroxyacetamido)pyrrolidin-1-yl)cyclohexyl)- |
| | 4-fluoro-7-methyl-1H-indole-2-carboxamide |
| 1120 | N-((1R,3R)-3-((R)-3-(N-ethyl-2-hydroxyacetamido)pyrrolidin-1-yl)cyclohexyl)- |
| | 4-fluoro-7-methyl-1H-indole-2-carboxamide |
| 1121 | N-((1R,3S)-3-((R)-3-(N-ethylmethylsulfonamido)pyrrolidin-1-yl)cyclohexyl)-4- |
| | fluoro-7-methyl-1H-indole-2-carboxamide |
| 1122 | N-((1R,3R)-3-((R)-3-(N-ethylmethylsulfonamido)pyrrolidin-1-yl)cyclohexyl)-4- |
| | fluoro-7-methyl-1H-indole-2-carboxamide |
| 1123 | N-((1R,3R)-3-((3R,5R)-4-acetyl-3,5-dimethylpiperazin-1-yl)cyclohexyl)-4- |
| | fluoro-7-methyl-1H-indole-2-carboxamide |
| 1124 | N-((1R,3R)-3-(1-acetylpiperidin-4-yl)cyclohexyl)-4-fluoro-7-methyl-1H-indole- |
| | 2-carboxamide |
| 1125 | 4-fluoro-7-methyl-N-((1R,3S)-3-((S)-2-methyl-3-oxohexahydroimidazo[1,5- |
| | a]pyrazin-7(1H)-yl)cyclohexyl)-1H-indole-2-carboxamide |
| 1126 | 4-fluoro-7-methyl-N-((1R,3S)-3-((R)-2-methyl-3-oxohexahydroimidazo[1,5- |
| | a]pyrazin-7(1H)-yl)cyclohexyl)-1H-indole-2-carboxamide |
| 1127 | 4-fluoro-7-methyl-N-((1R,3R)-3-((S)-2-methyl-3-oxohexahydroimidazo[1,5- |
| | a]pyrazin-7(1H)-yl)cyclohexyl)-1H-indole-2-carboxamide |
| 1128 | 4-fluoro-7-methyl-N-((1R,3R)-3-((R)-2-methyl-3-oxohexahydroimidazo[1,5- |
| | a]pyrazin-7(1H)-yl)cyclohexyl)-1H-indole-2-carboxamide |
| 1129 | N-((1R,3S)-3-((S)-3-(N-ethyl-2-hydroxyacetamido)pyrrolidin-1-yl)cyclohexyl)-4- |
| | fluoro-7-methyl-1H-indole-2-carboxamide |
| 1130 | N-((1R,3R)-3-((S)-3-(N-ethyl-2-hydroxyacetamido)pyrrolidin-1-yl)cyclohexyl)- |
| | 4-fluoro-7-methyl-1H-indole-2-carboxamide |
| 1131 | N-((1R,3S)-3-((S)-3-(N-ethylmethylsulfonamido)pyrrolidin-1-yl)cyclohexyl)-4- |
| | fluoro-7-methyl-1H-indole-2-carboxamide |
| 1132 | N-((1R,3R)-3-((S)-3-(N-ethylmethylsulfonamido)pyrrolidin-1-yl)cyclohexyl)-4- |
| | fluoro-7-methyl-1H-indole-2-carboxamide |
| 1133 | 4-fluoro-7-methyl-N-((1R,3S)-3-((R)-3-oxotetrahydro-3H-oxazolo[3,4-a]pyrazin- |
| | 7(1H)-yl)cyclohexyl)-1H-indole-2-carboxamide |
| 1134 | 4-fluoro-7-methyl-N-((1R,3R)-3-((R)-3-oxotetrahydro-3H-oxazolo[3,4-a]pyrazin- |
| | 7(1H)-yl)cyclohexyl)-1H-indole-2-carboxamide |
| 1135 | 4-fluoro-7-methyl-N-((1R,3R)-3-((S)-3-oxotetrahydro-3H-oxazolo[3,4-a]pyrazin- |
| | 7(1H)-yl)cyclohexyl)-1H-indole-2-carboxamide |
| 1136 | 4-fluoro-7-methyl-N-((1R,3S)-3-((S)-3-oxotetrahydro-3H-oxazolo[3,4-a]pyrazin- |
| | 7(1H)-yl)cyclohexyl)-1H-indole-2-carboxamide |
| 1137 | N-((1R,3R)-3-(7-acetyl-4,7-diazaspiro[2.5]octan-4-yl)cyclohexyl)-4-fluoro-7- |
| | methyl-1H-indole-2-carboxamide |
| 1138 | 4-fluoro-7-methyl-N-((1R,3S)-3-((S)-4-oxohexahydropyrazino[2,1-c][1,4]oxazin- |
| | 8(1H)-yl)cyclohexyl)-1H-indole-2-carboxamide |
| 1139 | 4-fluoro-7-methyl-N-((1R,3R)-3-((S)-4-oxohexahydropyrazino[2,1-c][1,4]oxazin- |
| | 8(1H)-yl)cyclohexyl)-1H-indole-2-carboxamide |
| 1140 | 4-fluoro-7-methyl-N-((1R,3S)-3-((R)-4-oxohexahydropyrazino[2,1-c][1,4]oxazin- |
| | 8(1H)-yl)cyclohexyl)-1H-indole-2-carboxamide |
| 1141 | 4-fluoro-7-methyl-N-((1R,3R)-3-((R)-4-oxohexahydropyrazino[2,1- |
| | c][1,4]oxazin-8(1H)-yl)cyclohexyl)-1H-indole-2-carboxamide |
| 1142 | 4-fluoro-7-methyl-N-((1R,3R)-3-(2-methyl-3-oxo-2,5,6,8-tetrahydro- |
| | [1,2,4]triazolo[4,3-a]pyrazin-7(3H)-yl)cyclohexyl)-1H-indole-2-carboxamide |
| 1143 | 4-fluoro-7-methyl-N-((1R,3R)-3-(6-oxo-1,3,4,6-tetrahydro-2H-pyrido[1,2- |
| | a]pyrazin-2-yl)cyclohexyl)-1H-indole-2-carboxamide |
| 1144 | 4-fluoro-N-((1R,3S)-3-((S)-3-(2-hydroxy-N,2-dimethylpropanamido)pyrrolidin- |
| | 1-yl)cyclohexyl)-7-methyl-1H-indole-2-carboxamide |
| 1145 | 4-fluoro-N-((1R,3R)-3-((S)-3-(2-hydroxy-N,2-dimethylpropanamido)pyrrolidin- |
| | 1-yl)cyclohexyl)-7-methyl-1H-indole-2-carboxamide |
| 1146 | 4-fluoro-N-((1R,3S)-3-((R)-3-(2-hydroxy-N,2-dimethylpropanamido)pyrrolidin- |
| | 1-yl)cyclohexyl)-7-methyl-1H-indole-2-carboxamide |
| 1147 | 4-fluoro-N-((1R,3R)-3-((R)-3-(2-hydroxy-N,2-dimethylpropanamido)pyrrolidin- |
| | 1-yl)cyclohexyl)-7-methyl-1H-indole-2-carboxamide |
| 1148 | N-((1R,3R)-3-(4-((E)-1-(cyanoimino)ethyl)piperazin-1-yl)cyclohexyl)-4-fluoro-7- |
| | methyl-1H-indole-2-carboxamide |
| 1149 | 4-fluoro-N-((1R,3S)-3-((R)-3-(N-(2-hydroxyethyl)methylsulfonamido)pyrrolidin- |
| | 1-yl)cyclohexyl)-7-methyl-1H-indole-2-carboxamide |
| 1150 | 4-fluoro-N-((1R,3R)-3-((R)-3-(N-(2-hydroxyethyl)methylsulfonamido)pyrrolidin- |
| | 1-yl)cyclohexyl)-7-methyl-1H-indole-2-carboxamide |
| 1151 | 4-fluoro-N-((1R,3R)-3-((S)-3-(N-(2-hydroxyethyl)methylsulfonamido)pyrrolidin- |
| | 1-yl)cyclohexyl)-7-methyl-1H-indole-2-carboxamide |
| 1152 | N-((1R,3R)-3-(4-((E)-N′-cyano-N-methylcarbamimidoyl)piperazin-1- |
| | yl)cyclohexyl)-4-fluoro-7-methyl-1H-indole-2-carboxamide |
| 1153 | N-(2-chlorobenzyl)-7-methyl-1H-indole-2-carboxamide |
| 1154 | (S)-N-(1-(2-chlorophenyl)ethyl)-7-methyl-1H-indole-2-carboxamide |
| 1155 | N-(1-(4-(1H-1,2,4-triazol-1-yl)phenyl)ethyl)-7-methyl-1H-indole-2-carboxamide |
| 1156 | N-(1-(2-chlorophenyl)ethyl)-7-methyl-1H-indole-2-carboxamide |
| 1157 | methyl 3-(2-chlorophenyl)-3-(7-methyl-1H-indole-2-carboxamido)propanoate |
| 1158 | N-(3-chloro-5-(4-(3-(pyrrolidin-1-yl)propyl)piperidin-1-yl)phenyl)-4-fluoro-7- |
| | methyl-1H-indole-2-carboxamide |
| 1159 | 4-fluoro-7-methyl-N-(1-methyl-5-(1-methylpiperidin-4-yl)-1H-imidazol-2-yl)- |
| | 1H-indole-2-carboxamide |
| 1160 | 4-fluoro-N-((1R,3R)-3-((R)-3-(2-methoxy-N-methylacetamido)pyrrolidin-1- |
| | yl)cyclohexyl)-7-methyl-1H-indole-2-carboxamide |
| 1161 | 4-fluoro-N-((1R,3S)-3-((R)-3-(N-(2-hydroxyethyl)methylsulfonamido)pyrrolidin- |
| | 1-yl)cyclohexyl)-7-methyl-1H-indene-2-carboxamide |
| 1162 | N-((1R,3S)-3-((3S,5S)-4-acetyl-3,5-dimethylpiperazin-1-yl)cyclohexyl)-4-fluoro- |
| | 7-methyl-1H-indole-2-carboxamide |
| 1163 | N-((1R,3R)-3-((3S,5S)-4-acetyl-3,5-dimethylpiperazin-1-yl)cyclohexyl)-4-fluoro- |
| | 7-methyl-1H-indole-2-carboxamide |
| 1164 | 4-fluoro-7-methyl-N-((1R,3R)-3-((R)-4-propionyl-3-(trifluoromethyl)piperazin-1- |
| | yl)cyclohexyl)-1H-indole-2-carboxamide |
| 1165 | 4-fluoro-7-methyl-N-((1R,3R)-3-(4-oxo-4,6,7,9-tetrahydro-8H-pyrazino[1,2- |
| | a]pyrimidin-8-yl)cyclohexyl)-1H-indole-2-carboxamide |
| 1166 | 4-fluoro-7-methyl-N-((1R,3R)-3-(3-oxo-2,5,6,8-tetrahydroimidazo[1,5-a]pyrazin- |
| | 7(3H)-yl)cyclohexyl)-1H-indole-2-carboxamide |
| 1167 | 4-fluoro-7-methyl-N-((1R,3S)-3-((S)-4-propionyl-3-(trifluoromethyl)piperazin-1- |
| | yl)cyclohexyl)-1H-indole-2-carboxamide |
| 1168 | 4-fluoro-7-methyl-N-((1R,3R)-3-((S)-4-propionyl-3-(trifluoromethyl)piperazin-1- |
| | yl)cyclohexyl)-1H-indole-2-carboxamide |
| 1169 | 4-fluoro-7-methyl-N-((1R,3R)-3-(4-((E)-1-(methylamino)-2-nitrovinyl)piperazin- |
| | 1-yl)cyclohexyl)-1H-indole-2-carboxamide |
| 1170 | 4-fluoro-7-methyl-N-((1R,3R)-3-(4-((tetrahydro-2H-pyran-4- |
| | yl)carbamoyl)piperazin-1-yl)cyclohexyl)-1H-indole-2-carboxamide |
| 1171 | 4-fluoro-N-((1R,3S)-3-((S)-4-isobutyryl-3-(trifluoromethyl)piperazin-1- |
| | yl)cyclohexyl)-7-methyl-1H-indole-2-carboxamide |
| 1172 | 4-fluoro-N-((1R,3R)-3-((S)-4-isobutyryl-3-(trifluoromethyl)piperazin-1- |
| | yl)cyclohexyl)-7-methyl-1H-indole-2-carboxamide |
| 1173 | 4-fluoro-N-((1R,3S)-3-((S)-4-(2-hydroxyacetyl)-3-(trifluoromethyl)piperazin-1- |
| | yl)cyclohexyl)-7-methyl-1H-indole-2-carboxamide |
| 1174 | 4-fluoro-N-((1R,3R)-3-((S)-4-(2-hydroxyacetyl)-3-(trifluoromethyl)piperazin-1- |
| | yl)cyclohexyl)-7-methyl-1H-indole-2-carboxamide |
| 1175 | 4-fluoro-N-((1R,3R)-3-((R)-4-isobutyryl-3-(trifluoromethyl)piperazin-1- |
| | yl)cyclohexyl)-7-methyl-1H-indole-2-carboxamide |
| 1176 | 4-fluoro-N-((1R,3R)-3-((R)-4-(2-hydroxyacetyl)-3-(trifluoromethyl)piperazin-1- |
| | yl)cyclohexyl)-7-methyl-1H-indole-2-carboxamide |
| 1177 | N-((1R,3S)-3-((3aR,6aS)-5-acetylhexahydropyrrolo[3,4-c]pyrrol-2(1H)- |
| | yl)cyclohexyl)-4-fluoro-7-methyl-1H-indole-2-carboxamide |
| 1178 | N-((1R,3R)-3-((3aR,6aS)-5-acetylhexahydropyrrolo[3,4-c]pyrrol-2(1H)- |
| | yl)cyclohexyl)-4-fluoro-7-methyl-1H-indole-2-carboxamide |
| 1179 | 4-fluoro-7-methyl-N-((1R,3R)-3-(2-methyl-3-oxo-2,5,6,8-tetrahydroimidazo[1,5- |
| | a]pyrazin-7(3H)-yl)cyclohexyl)-1H-indole-2-carboxamide |
| 1180 | N-((1R,3S)-3-((3aS,6aS)-1-acetylhexahydropyrrolo[3,4-b]pyrrol-5(1H)- |
| | yl)cyclohexyl)-4-fluoro-7-methyl-1H-indole-2-carboxamide |
| 1181 | N-((1R,3R)-3-((3aS,6aS)-1-acetylhexahydropyrrolo[3,4-b]pyrrol-5(1H)- |
| | yl)cyclohexyl)-4-fluoro-7-methyl-1H-indole-2-carboxamide |
| 1182 | N-((1R,3S)-3-((3aR,6aR)-1-acetylhexahydropyrrolo[3,4-b]pyrrol-5(1H)- |
| | yl)cyclohexyl)-4-fluoro-7-methyl-1H-indole-2-carboxamide |
| 1183 | N-((1R,3R)-3-((3aR,6aR)-1-acetylhexahydropyrrolo[3,4-b]pyrrol-5(1H)- |
| | yl)cyclohexyl)-4-fluoro-7-methyl-1H-indole-2-carboxamide |
| 1184 | N-((1R,3S)-3-((3aS,6aS)-4-acetylhexahydropyrrolo[3,2-b]pyrrol-1(2H)- |
| | yl)cyclohexyl)-4-fluoro-7-methyl-1H-indole-2-carboxamide |
| 1185 | N-((1R,3S)-3-((3aR,6aR)-4-acetylhexahydropyrrolo[3,2-b]pyrrol-1(2H)- |
| | yl)cyclohexyl)-4-fluoro-7-methyl-1H-indole-2-carboxamide |
| 1186 | N-((1R,3R)-3-((3aS,6aS)-4-acetylhexahydropyrrolo[3,2-b]pyrrol-1(2H)- |
| | yl)cyclohexyl)-4-fluoro-7-methyl-1H-indole-2-carboxamide |
| 1187 | N-((1R,3R)-3-((3aR,6aR)-4-acetylhexahydropyrrolo[3,2-b]pyrrol-1(2H)- |
| | yl)cyclohexyl)-4-fluoro-7-methyl-1H-indole-2-carboxamide |
| 1188 | 4-fluoro-7-methyl-N-((1S,3R)-3-(6-oxo-1,4,5,6-tetrahydro-1,2,4-triazin-3- |
| | yl)cyclohexyl)-1H-indole-2-carboxamide |
| 1189 | 4-fluoro-7-methyl-N-((1R,3S)-3-(3-(N-(oxetan-3-yl)acetamido)pyrrolidin-1- |
| | yl)cyclohexyl)-1H-indole-2-carboxamide |
| 1190 | 4-fluoro-7-methyl-N-((1R,3S)-3-(3-oxo-2,8-diazaspiro[4.5]decan-8- |
| | yl)cyclohexyl)-1H-indole-2-carboxamide |
| 1191 | 4-fluoro-7-methyl-N-(1′-methyl-2′-oxo-[1,4′-bipiperidin]-3-yl)-1H-indole-2- |
| | carboxamide |
| 1192 | N-((1R,3S)-3-((3R,5R)-4-acetyl-3,5-dimethylpiperazin-1-yl)cyclohexyl)-4-fluoro- |
| | 7-methyl-1H-indole-2-carboxamide |
| 1193 | N-((1R,3R)-3-((3R,5R)-4-acetyl-3,5-dimethylpiperazin-1-yl)cyclohexyl)-4- |
| | fluoro-7-methyl-1H-indole-2-carboxamide |
| 1194 | (R)-4-fluoro-N-(3-fluoro-5-(3-(N-methylacetamido)pyrrolidin-1-yl)phenyl)-7- |
| | methyl-1H-indole-2-carboxamide |
| 1195 | (S)-4-fluoro-N-(3-fluoro-5-(3-(N-methylacetamido)pyrrolidin-1-yl)phenyl)-7- |
| | methyl-1H-indole-2-carboxamide |
| 1196 | 4-fluoro-N-(3-fluoro-5-((S)-3-((R)-2-methyl-3-oxohexahydroimidazo[1,5- |
| | a]pyrazin-7(1H)-yl)pyrrolidin-1-yl)phenyl)-7-methyl-1H-indole-2-carboxamide |
| 1197 | 4-fluoro-N-(3-fluoro-5-((S)-3-((S)-2-methyl-3-oxohexahydroimidazo[1,5- |
| | a]pyrazin-7(1H)-yl)pyrrolidin-1-yl)phenyl)-7-methyl-1H-indole-2-carboxamide |
| 1198 | 4-fluoro-N-(3-fluoro-5-((R)-3-((S)-2-methyl-3-oxohexahydroimidazo[1,5- |
| | a]pyrazin-7(1H)-yl)pyrrolidin-1-yl)phenyl)-7-methyl-1H-indole-2-carboxamide |
| 1199 | 4-fluoro-N-(3-fluoro-5-((R)-3-((R)-2-methyl-3-oxohexahydroimidazo[1,5- |
| | a]pyrazin-7(1H)-yl)pyrrolidin-1-yl)phenyl)-7-methyl-1H-indole-2-carboxamide |
| 1200 | (R)-N-(3-(3-(dimethylamino)-2-oxopyrrolidin-1-yl)-5-fluorophenyl)-4-fluoro-7- |
| | methyl-1H-indole-2-carboxamide |
| 1201 | (S)-N-(3-(3-(dimethylamino)-2-oxopyrrolidin-1-yl)-5-fluorophenyl)-4-fluoro-7- |
| | methyl-1H-indole-2-carboxamide |
| 1202 | (R)-N-(3-(3-(4-(2-(dimethylamino)-2-oxoethyl)piperazin-1-yl)pyrrolidin-1-yl)-5- |
| | fluorophenyl)-4-fluoro-7-methyl-1H-indole-2-carboxamide |
| 1203 | (R)-N-(3-(3-(4-(2-(dimethylamino)-2-oxoethyl)piperazin-1-yl)pyrrolidin-1-yl)-5- |
| | fluorophenyl)-4-fluoro-7-methyl-1H-indole-2-carboxamide |
| 1204 | (S)-N-(3-(3-(4-(2-amino-2-oxoethyl)piperazin-1-yl)pyrrolidin-1-yl)-5- |
| | fluorophenyl)-4-fluoro-7-methyl-1H-indole-2-carboxamide |
| 1205 | (R)-N-(3-(3-(4-(2-amino-2-oxoethyl)piperazin-1-yl)pyrrolidin-1-yl)-5- |
| | fluorophenyl)-4-fluoro-7-methyl-1H-indole-2-carboxamide |
| 1206 | (S)-N-(3-(3-(1,1-dioxidothiomorpholino)pyrrolidin-1-yl)-5-fluorophenyl)-4- |
| | fluoro-7-methyl-1H-indole-2-carboxamide |
| 1207 | (R)-N-(3-(3-(1,1-dioxidothiomorpholino)pyrrolidin-1-yl)-5-fluorophenyl)-4- |
| | fluoro-7-methyl-1H-indole-2-carboxamide |
| 1208 | (R)-N-(3-(3-(4-acetylpiperazin-1-yl)pyrrolidin-1-yl)-5-fluorophenyl)-4-fluoro-7- |
| | methyl-1H-indole-2-carboxamide |
| 1209 | (S)-N-(3-(3-(4-acetylpiperazin-1-yl)pyrrolidin-1-yl)-5-fluorophenyl)-4-fluoro-7- |
| | methyl-1H-indole-2-carboxamide |
| 1210 | (S)-4-fluoro-N-(3-fluoro-5-(3-morpholinopyrrolidin-1-yl)phenyl)-7-methyl-1H- |
| | indole-2-carboxamide |
| 1211 | (R)-4-fluoro-N-(3-fluoro-5-(3-morpholinopyrrolidin-1-yl)phenyl)-7-methyl-1H- |
| | indole-2-carboxamide |
| 1212 | N-(3-((3aR,6aR)-4-acetylhexahydropyrrolo[3,2-b]pyrrol-1(2H)-yl)-5- |
| | fluorophenyl)-4-fluoro-7-methyl-1H-indole-2-carboxamide |
| 1213 | N-(3-((3aR,6aS)-4-acetylhexahydropyrrolo[3,2-b]pyrrol-1(2H)-yl)-5- |
| | fluorophenyl)-4-fluoro-7-methyl-1H-indole-2-carboxamide |
| 1214 | N-(3-((3aS,6aS)-4-acetylhexahydropyrrolo[3,2-b]pyrrol-1(2H)-yl)-5- |
| | fluorophenyl)-4-fluoro-7-methyl-1H-indole-2-carboxamide |
| 1215 | N-(3-((3aS,6aR)-4-acetylhexahydropyrrolo[3,2-b]pyrrol-1(2H)-yl)-5- |
| | fluorophenyl)-4-fluoro-7-methyl-1H-indole-2-carboxamide |
| 1216 | N-(3-((3aR,6aR)-1-acetylhexahydropyrrolo[3,4-b]pyrrol-5(1H)-yl)-5- |
| | fluorophenyl)-4-fluoro-7-methyl-1H-indole-2-carboxamide |
| 1217 | N-(3-((3aS,6aR)-1-acetylhexahydropyrrolo[3,4-b]pyrrol-5(1H)-yl)-5- |
| | fluorophenyl)-4-fluoro-7-methyl-1H-indole-2-carboxamide |
| 1218 | N-(3-((3aS,6aS)-1-acetylhexahydropyrrolo[3,4-b]pyrrol-5(1H)-yl)-5- |
| | fluorophenyl)-4-fluoro-7-methyl-1H-indole-2-carboxamide |
| 1219 | N-(3-((3aR,6aS)-1-acetylhexahydropyrrolo[3,4-b]pyrrol-5(1H)-yl)-5- |
| | fluorophenyl)-4-fluoro-7-methyl-1H-indole-2-carboxamide |
| 1220 | 4-fluoro-N-(3-fluoro-5-((S)-3-((S)-2-(hydroxymethyl)morpholino)pyrrolidin-1- |
| | yl)phenyl)-7-methyl-1H-indole-2-carboxamide |
| 1221 | 4-fluoro-N-(3-fluoro-5-((S)-3-((R)-2-(hydroxymethyl)morpholino)pyrrolidin-1- |
| | yl)phenyl)-7-methyl-1H-indole-2-carboxamide |
| 1222 | 4-fluoro-N-(3-fluoro-5-((R)-3-((R)-2-(hydroxymethyl)morpholino)pyrrolidin-1- |
| | yl)phenyl)-7-methyl-1H-indole-2-carboxamide |
| 1223 | 4-fluoro-N-(3-fluoro-5-((R)-3-((S)-2-(hydroxymethyl)morpholino)pyrrolidin-1- |
| | yl)phenyl)-7-methyl-1H-indole-2-carboxamide |
| 1224 | (R)-4-fluoro-N-(3-fluoro-5-(3-(4-methyl-3-oxopiperazin-1-yl)pyrrolidin-1- |
| | yl)phenyl)-7-methyl-1H-indole-2-carboxamide |
| 1225 | (S)-4-fluoro-N-(3-fluoro-5-(3-(4-methyl-3-oxopiperazin-1-yl)pyrrolidin-1- |
| | yl)phenyl)-7-methyl-1H-indole-2-carboxamide |
| 1226 | (S)-4-fluoro-N-(3-fluoro-5-(2-oxo-[1,3′-bipyrrolidin]-1′-yl)phenyl)-7-methyl-1H- |
| | indole-2-carboxamide |
| 1227 | (R)-4-fluoro-N-(3-fluoro-5-(2-oxo-[1,3′-bipyrrolidin]-1′-yl)phenyl)-7-methyl-1H- |
| | indole-2-carboxamide |
| 1228 | 4-fluoro-7-methyl-N-((1R,3S)-3-((3aS,6aS)-1-methyl-2- |
| | oxohexahydropyrrolo[3,4-b]pyrrol-5(1H)-yl)cyclohexyl)-1H-indole-2- |
| | carboxamide |
| 1229 | 4-fluoro-7-methyl-N-((1R,3R)-3-((3aS,6aS)-1-methyl-2- |
| | oxohexahydropyrrolo[3,4-b]pyrrol-5(1H)-yl)cyclohexyl)-1H-indole-2- |
| | carboxamide |
| 1230 | 4-fluoro-7-methyl-N-((1R,3S)-3-((3aR,6aR)-1-methyl-2- |
| | oxohexahydropyrrolo[3,4-b]pyrrol-5(1H)-yl)cyclohexyl)-1H-indole-2- |
| | carboxamide |
| 1231 | 4-fluoro-7-methyl-N-((1R,3R)-3-((3aR,6aR)-1-methyl-2- |
| | oxohexahydropyrrolo[3,4-b]pyrrol-5(1H)-yl)cyclohexyl)-1H-indole-2- |
| | carboxamide |
| 1232 | 4-fluoro-7-methyl-N-((1R,3S)-3-((3aS,6aS)-4-methyl-5- |
| | oxohexahydropyrrolo[3,2-b]pyrrol-1(2H)-yl)cyclohexyl)-1H-indole-2- |
| | carboxamide |
| 1233 | 4-fluoro-7-methyl-N-((1R,3S)-3-((3aR,6aR)-4-methyl-5- |
| | oxohexahydropyrrolo[3,2-b]pyrrol-1(2H)-yl)cyclohexyl)-1H-indole-2- |
| | carboxamide |
| 1234 | 4-fluoro-7-methyl-N-((1R,3R)-3-((3aS,6aS)-4-methyl-5- |
| | oxohexahydropyrrolo[3,2-b]pyrrol-1(2H)-yl)cyclohexyl)-1H-indole-2- |
| | carboxamide |
| 1235 | 4-fluoro-7-methyl-N-((1R,3R)-3-((3aR,6aR)-4-methyl-5- |
| | oxohexahydropyrrolo[3,2-b]pyrrol-1(2H)-yl)cyclohexyl)-1H-indole-2- |
| | carboxamide |

TABLE 1B

| Cpd. | | SETD2 | SETD2 |
|---|---|---|---|
| No. | STRUCTURE | (1434-1711) | A549 |
| 15 | 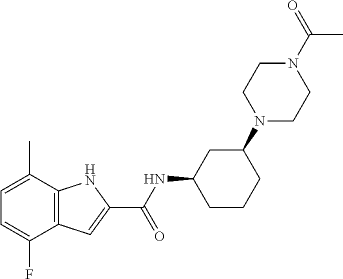 [SMILES: CC(=O)N1CCN([C@H]2CCC[C@@H](NC(=O)c3cc4c(F)ccc(C)c4[nH]3)C2)CC1] | 0.02 | 0.05 |
| 1228 | 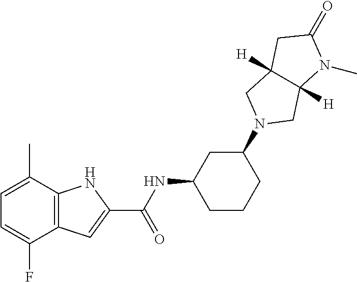 [SMILES: Cc1ccc(F)c2cc(C(=O)N[C@@H]3CCC[C@H](N4C[C@@H]5CC(=O)N(C)[C@@H]5C4)C3)[nH]c12] | 0.04 | 0.09 |
| 1229 | 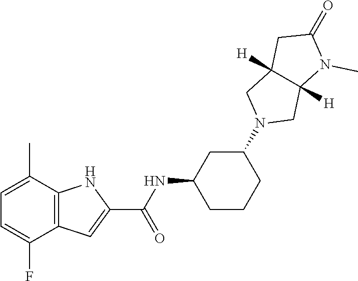 [SMILES: Cc1ccc(F)c2cc(C(=O)N[C@@H]3CCC[C@@H](N4C[C@@H]5CC(=O)N(C)[C@@H]5C4)C3)[nH]c12] | 8.8 | 2 |
| 1230 | 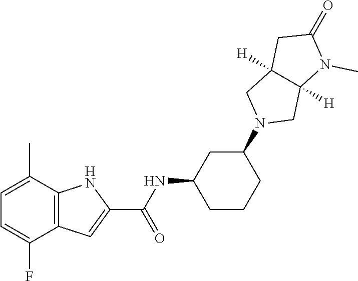 [SMILES: Cc1ccc(F)c2cc(C(=O)N[C@@H]3CCC[C@H](N4C[C@H]5CC(=O)N(C)[C@H]5C4)C3)[nH]c12] | 0.04 | 0.09 |
| 1231 | 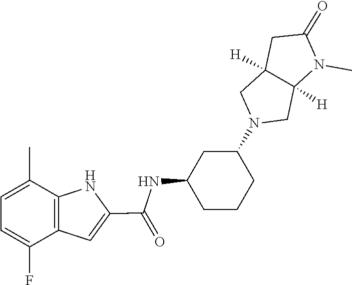 [SMILES: Cc1ccc(F)c2cc(C(=O)N[C@@H]3CCC[C@@H](N4C[C@H]5CC(=O)N(C)[C@H]5C4)C3)[nH]c12] | 9.8 | 2 |
| 1232 | 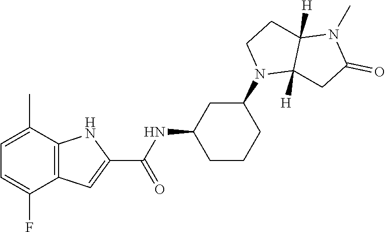 [SMILES: Cc1ccc(F)c2cc(C(=O)N[C@@H]3CCC[C@H](N4CC[C@H]5[C@@H]4CC(=O)N5C)C3)[nH]c12] | 0.008 | 0.01 |
| 1233 | 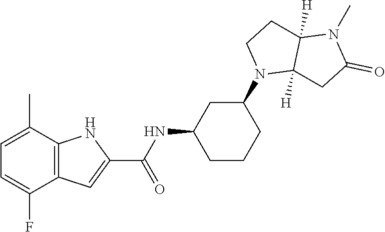 [SMILES: Cc1ccc(F)c2cc(C(=O)N[C@@H]3CCC[C@H](N4CC[C@@H]5[C@H]4CC(=O)N5C)C3)[nH]c12] | 0.04 | 0.04 |
| 1234 | 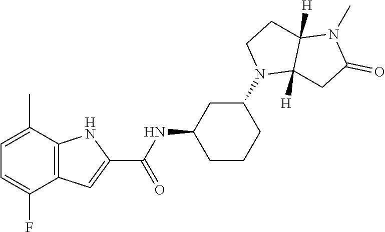 [SMILES: Cc1ccc(F)c2cc(C(=O)N[C@@H]3CCC[C@@H](N4CC[C@H]5[C@@H]4CC(=O)N5C)C3)[nH]c12] | 4.2 | 2 |
| 1235 | 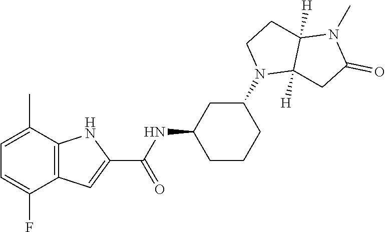 [SMILES: Cc1ccc(F)c2cc(C(=O)N[C@@H]3CCC[C@@H](N4CC[C@@H]5[C@H]4CC(=O)N5C)C3)[nH]c12] | 10 | 2 |

The present disclosure encompasses the preparation and use of salts of the Compounds of the Disclosure, including non-toxic pharmaceutically acceptable salts. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. The term “pharmaceutically acceptable salt” as used herein, refers to any salt, e.g., obtained by reaction with an acid or a base, of a Compound of the Disclosure that is physiologically tolerated in the target subject (e.g., a mammal, e.g., a human).

Acid addition salts can be formed by mixing a solution of the particular Compound of the Disclosure with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the compound of the present disclosure with a solution of a pharmaceutically acceptable non-toxic base such as sodium hydroxide, potassium hydroxide, choline hydroxide, sodium carbonate and the like.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, ethanol, and the like, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Second Therapeutic Agents

In one embodiment, the therapeutic methods, uses, compositions, and kits of the present disclosure comprise administering a therapeutically effective amount of a Compound of the Disclosure in combination with a therapeutically effective amount of a Second Therapeutic Agent to a subject in need thereof.

The term “Second Therapeutic Agent” as used herein comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

In one embodiment, the Second Therapeutic Agent comprises one compound from one drug class, i.e., one BTK inhibitor, one anti-CD20 monoclonal antibody, one alkylating agent, one topoisomerase II inhibitor, one vinca alkaloid, one platinum-based drug, one nucleoside anticancer agent, one PI3K inhibitor, one CDK4/6 inhibitor, one CARM1 inhibitor, inhibitor of an enzyme of DNA damage repair, one SYK inhibitor, or one MEK inhibitor.

In another embodiment, the Second Therapeutic Agent comprises ibrutinib, acalabrutinib, zanubrutinib, rituximab, mafosfamide, doxorubicin, vincristine, cytarabine, carboplatin, etoposide, gemcitabine, oxaliplatin, copanlisib, palbociclib, or EZM2302.

In another embodiment, the Second Therapeutic Agent comprises two different compounds from one drug class, e.g., two different BTK inhibitors, two different anti-CD20 monoclonal antibodies, two different alkylating agents, two different topoisomerase II inhibitors, two different vinca alkaloids, two different platinum-based drugs, two different nucleoside anticancer agents, two different PI3K inhibitors, two different CDK4/6 inhibitors, or two different CARM1 inhibitors.

In another embodiment, the Second Therapeutic Agent comprises three different compounds from one drug class, e.g., three different BTK inhibitors, three different anti-CD20 monoclonal antibodies, three different alkylating agents, three different topoisomerase II inhibitors, three different vinca alkaloids, three different platinum-based drugs, three different nucleoside anticancer agents, three different PI3K inhibitors, three different CDK4/6 inhibitors, or three different CARM1 inhibitors.

In another embodiment, the Second Therapeutic Agent comprises three different compounds from two drug classes, e.g., two different BTK inhibitors and one PI3Ki inhibitor; two different CDK4/6 inhibitors and one CARM1 inhibitor; and so on.

In another embodiment, the Second Therapeutic Agent comprises compounds from two different drug classes. For example, in one embodiment, the Second Therapeutic Agent comprises a first compound from a first drug class and second compound from a second drug class, wherein the first drug class and the second drug class are different. In a specific non-limiting example, the Second Therapeutic Agent comprises a BTK inhibitor, e.g., ibrutinib, and a PI3Ki inhibitor, e.g., copanlisib. Non-limiting examples of combinations of first and second drug classes of the Second Therapeutic Agent are provided in Table 4.

TABLE 4

| No. | Drug Class # 1 | Drug Class # 2 |
|---|---|---|
| 1 | BTK inhibitor | anti-CD20 mAb |
| 2 | BTK inhibitor | alkylating agent |
| 3 | BTK inhibitor | topoisomerase II inhibitor |
| 4 | BTK inhibitor | vinca alkaloid |
| 5 | BTK inhibitor | platinum-based drug |
| 6 | BTK inhibitor | nucleoside anticancer agent |
| 7 | BTK inhibitor | PI3K inhibitor |
| 8 | BTK inhibitor | CDK4/6 inhibitor |
| 9 | BTK inhibitor | CARM1 inhibitor |
| 10 | anti-CD20 mAb | alkylating agent |
| 11 | anti-CD20 mAb | topoisomerase II inhibitor |
| 12 | anti-CD20 mAb | vinca alkaloid |
| 13 | anti-CD20 mAb | platinum-based drug |
| 14 | anti-CD20 mAb | nucleoside anticancer agent |
| 15 | anti-CD20 mAb | PI3K inhibitor |
| 16 | anti-CD20 mAb | CDK4/6 inhibitor |
| 17 | anti-CD20 mAb | CARM1 inhibitor |
| 18 | alkylating agent | topoisomerase II inhibitor |
| 19 | alkylating agent | vinca alkaloid |
| 20 | alkylating agent | platinum-based drug |
| 21 | alkylating agent | nucleoside anticancer agent |
| 22 | alkylating agent | PI3K inhibitor |
| 23 | alkylating agent | CDK4/6 inhibitor |
| 24 | alkylating agent | CARM1 inhibitor |
| 25 | topoisomerase II inhibitor | vinca alkaloid |
| 26 | topoisomerase II inhibitor | platinum-based drug |
| 27 | topoisomerase II inhibitor | nucleoside anticancer agent |
| 28 | topoisomerase II inhibitor | PI3K inhibitor |
| 29 | topoisomerase II inhibitor | CDK4/6 inhibitor |
| 30 | topoisomerase II inhibitor | CARM1 inhibitor |
| 31 | vinca alkaloid | platinum-based drug |
| 32 | vinca alkaloid | nucleoside anticancer agent |
| 33 | vinca alkaloid | PI3K inhibitor |
| 34 | vinca alkaloid | CDK4/6 inhibitor |
| 35 | vinca alkaloid | CARM1 inhibitor |
| 36 | platinum-based drug | nucleoside anticancer agent |
| 37 | platinum-based drug | PI3K inhibitor |
| 38 | platinum-based drug | CDK4/6 inhibitor |
| 39 | platinum-based drug | CARM1 inhibitor |
| 40 | nucleoside anticancer agent | PI3K inhibitor |
| 41 | nucleoside anticancer agent | CDK4/6 inhibitor |
| 42 | nucleoside anticancer agent | CARM1 inhibitor |
| 43 | PI3K inhibitor | CDK4/6 inhibitor |
| 44 | PI3K inhibitor | CARM1 inhibitor |
| 45 | CDK4/6 inhibitor | CARM1 inhibitor |

In another embodiment, the Second Therapeutic Agent comprises compounds from three different drug classes. For example, in one embodiment, the Second Therapeutic Agent comprises a first compound from a first drug class, and a second compound from a second drug class, and a third compound from a third drug class, wherein the first drug class, the second drug class, and the third drug class are different. Non-limiting examples of combinations of first, second, and third drug classes of the Second Therapeutic Agent are provided in Table 5.

TABLE 5

| No. | Drug Class # 1 | Drug Class # 2 | Drug Class # 3 |
|---|---|---|---|
| 1 | BTK inhibitor | anti-CD20 mAb | alkylating agent |
| 2 | BTK inhibitor | anti-CD20 mAb | topoisomerase II |
| | | | inhibitor |
| 3 | BTK inhibitor | anti-CD20 mAb | vinca alkaloid |
| 4 | BTK inhibitor | anti-CD20 mAb | platinum-based drug |
| 5 | BTK inhibitor | anti-CD20 mAb | nucleoside |
| | | | anticancer agent |
| 6 | BTK inhibitor | anti-CD20 mAb | PI3K inhibitor |
| 7 | BTK inhibitor | anti-CD20 mAb | CDK4/6 inhibitor |
| 8 | BTK inhibitor | anti-CD20 mAb | CARM1 inhibitor |
| 9 | BTK inhibitor | alkylating agent | topoisomerase II |
| | | | inhibitor |
| 10 | BTK inhibitor | alkylating agent | vinca alkaloid |
| 11 | BTK inhibitor | alkylating agent | platinum-based drug |
| 12 | BTK inhibitor | alkylating agent | nucleoside |
| | | | anticancer agent |
| 13 | BTK inhibitor | alkylating agent | PI3K inhibitor |
| 14 | BTK inhibitor | alkylating agent | CDK4/6 inhibitor |
| 15 | BTK inhibitor | alkylating agent | CARM1 inhibitor |
| 16 | BTK inhibitor | topoisomerase II | vinca alkaloid |
| | | inhibitor | |
| 17 | BTK inhibitor | topoisomerase II | platinum-based drug |
| | | inhibitor | |
| 18 | BTK inhibitor | topoisomerase II | nucleoside |
| | | inhibitor | anticancer agent |
| 19 | BTK inhibitor | topoisomerase II | PI3K inhibitor |
| | | inhibitor | |
| 20 | BTK inhibitor | topoisomerase II | CDK4/6 inhibitor |
| | | inhibitor | |
| 21 | BTK inhibitor | topoisomerase II | CARM1 inhibitor |
| | | inhibitor | |
| 22 | BTK inhibitor | vinca alkaloid | platinum-based drug |
| 23 | BTK inhibitor | vinca alkaloid | nucleoside |
| | | | anticancer agent |
| 24 | BTK inhibitor | vinca alkaloid | PI3K inhibitor |
| 25 | BTK inhibitor | vinca alkaloid | CDK4/6 inhibitor |
| 26 | BTK inhibitor | vinca alkaloid | CARM1 inhibitor |
| 27 | BTK inhibitor | platinum-based drug | nucleoside |
| | | | anticancer agent |
| 28 | BTK inhibitor | platinum-based drug | PI3K inhibitor |
| 29 | BTK inhibitor | platinum-based drug | CDK4/6 inhibitor |
| 30 | BTK inhibitor | platinum-based drug | CARM1 inhibitor |
| 31 | BTK inhibitor | nucleoside | PI3K inhibitor |
| | | anticancer agent | |
| 32 | BTK inhibitor | nucleoside | CDK4/6 inhibitor |
| | | anticancer agent | |
| 33 | BTK inhibitor | nucleoside | CARM1 inhibitor |
| | | anticancer agent | |
| 34 | BTK inhibitor | PI3K inhibitor | CDK4/6 inhibitor |
| 35 | BTK inhibitor | PI3K inhibitor | CARM1 inhibitor |
| 36 | BTK inhibitor | CDK4/6 inhibitor | CARM1 inhibitor |
| 37 | anti-CD20 mAb | alkylating agent | topoisomerase II |
| | | | inhibitor |
| 38 | anti-CD20 mAb | alkylating agent | vinca alkaloid |
| 39 | anti-CD20 mAb | alkylating agent | platinum-based drug |
| 40 | anti-CD20 mAb | alkylating agent | nucleoside |
| | | | anticancer agent |
| 41 | anti-CD20 mAb | alkylating agent | PI3K inhibitor |
| 42 | anti-CD20 mAb | alkylating agent | CDK4/6 inhibitor |
| 43 | anti-CD20 mAb | alkylating agent | CARM1 inhibitor |
| 44 | anti-CD20 mAb | topoisomerase II | vinca alkaloid |
| | | inhibitor | |
| 45 | anti-CD20 mAb | topoisomerase II | platinum-based drug |
| | | inhibitor | |
| 46 | anti-CD20 mAb | topoisomerase II | nucleoside |
| | | inhibitor | anticancer agent |
| 47 | anti-CD20 mAb | topoisomerase II | PI3K inhibitor |
| | | inhibitor | |
| 48 | anti-CD20 mAb | topoisomerase II | CDK4/6 inhibitor |
| | | inhibitor | |
| 49 | anti-CD20 mAb | topoisomerase II | CARM1 inhibitor |
| | | inhibitor | |
| 50 | anti-CD20 mAb | vinca alkaloid | platinum-based drug |
| 51 | anti-CD20 mAb | vinca alkaloid | nucleoside |
| | | | anticancer agent |
| 52 | anti-CD20 mAb | vinca alkaloid | PI3K inhibitor |
| 53 | anti-CD20 mAb | vinca alkaloid | CDK4/6 inhibitor |
| 54 | anti-CD20 mAb | vinca alkaloid | CARM1 inhibitor |
| 55 | anti-CD20 mAb | platinum-based drug | nucleoside |
| | | | anticancer agent |
| 56 | anti-CD20 mAb | platinum-based drug | PI3K inhibitor |
| 57 | anti-CD20 mAb | platinum-based drug | CDK4/6 inhibitor |
| 58 | anti-CD20 mAb | platinum-based drug | CARM1 inhibitor |
| 59 | anti-CD20 mAb | nucleoside | PI3K inhibitor |
| | | anticancer agent | |
| 60 | anti-CD20 mAb | nucleoside | CDK4/6 inhibitor |
| | | anticancer agent | |
| 61 | anti-CD20 mAb | nucleoside | CARM1 inhibitor |
| | | anticancer agent | |
| 62 | anti-CD20 mAb | PI3K inhibitor | CDK4/6 inhibitor |
| 63 | anti-CD20 mAb | PI3K inhibitor | CARM1 inhibitor |
| 64 | anti-CD20 mAb | CDK4/6 inhibitor | CARM1 inhibitor |
| 65 | alkylating agent | topoisomerase II | vinca alkaloid |
| | | inhibitor | |
| 66 | alkylating agent | topoisomerase II | platinum-based drug |
| | | inhibitor | |
| 67 | alkylating agent | topoisomerase II | nucleoside |
| | | inhibitor | anticancer agent |
| 68 | alkylating agent | topoisomerase II | PI3K inhibitor |
| | | inhibitor | |
| 69 | alkylating agent | topoisomerase II | CDK4/6 inhibitor |
| | | inhibitor | |
| 70 | alkylating agent | topoisomerase II | CARM1 inhibitor |
| | | inhibitor | |
| 71 | alkylating agent | vinca alkaloid | platinum-based drug |
| 72 | alkylating agent | vinca alkaloid | nucleoside |
| | | | anticancer agent |
| 73 | alkylating agent | vinca alkaloid | PI3K inhibitor |
| 74 | alkylating agent | vinca alkaloid | CDK4/6 inhibitor |
| 75 | alkylating agent | vinca alkaloid | CARM1 inhibitor |
| 76 | alkylating agent | platinum-based drug | nucleoside |
| | | | anticancer agent |
| 77 | alkylating agent | platinum-based drug | PI3K inhibitor |
| 78 | alkylating agent | platinum-based drug | CDK4/6 inhibitor |
| 79 | alkylating agent | platinum-based drug | CARM1 inhibitor |
| 80 | alkylating agent | nucleoside | PI3K inhibitor |
| | | anticancer agent | |
| 81 | alkylating agent | nucleoside | CDK4/6 inhibitor |
| | | anticancer agent | |
| 82 | alkylating agent | nucleoside | CARMI inhibitor |
| | | anticancer agent | |
| 83 | alkylating agent | PI3K inhibitor | CDK4/6 inhibitor |
| 84 | alkylating agent | PI3K inhibitor | CARM1 inhibitor |
| 85 | alkylating agent | CDK4/6 inhibitor | CARM1 inhibitor |
| 86 | topoisomerase II | vinca alkaloid | platinum-based drug |
| | inhibitor | | |
| 87 | topoisomerase II | vinca alkaloid | nucleoside |
| | inhibitor | | anticancer agent |
| 88 | topoisomerase II | vinca alkaloid | PI3K inhibitor |
| | inhibitor | | |
| 89 | topoisomerase II | vinca alkaloid | CDK4/6 inhibitor |
| | inhibitor | | |
| 90 | topoisomerase II | vinca alkaloid | CARM1 inhibitor |
| | inhibitor | | |
| 91 | topoisomerase II | platinum-based drug | nucleoside |
| | inhibitor | | anticancer agent |
| 92 | topoisomerase II | platinum-based drug | PI3K inhibitor |
| | inhibitor | | |
| 93 | topoisomerase II | platinum-based drug | CDK4/6 inhibitor |
| | inhibitor | | |
| 94 | topoisomerase II | platinum-based drug | CARM1 inhibitor |
| | inhibitor | | |
| 95 | topoisomerase II | nucleoside | PI3K inhibitor |
| | inhibitor | anticancer agent | |
| 96 | topoisomerase II | nucleoside | CDK4/6 inhibitor |
| | inhibitor | anticancer agent | |
| 97 | topoisomerase II | nucleoside | CARM1 inhibitor |
| | inhibitor | anticancer agent | |
| 98 | topoisomerase II | PI3K inhibitor | CDK4/6 inhibitor |
| | inhibitor | | |
| 99 | topoisomerase II | PI3K inhibitor | CARM1 inhibitor |
| | inhibitor | | |
| 100 | topoisomerase II | CDK4/6 inhibitor | CARM1 inhibitor |
| | inhibitor | | |
| 101 | vinca alkaloid | platinum-based drug | nucleoside |
| | | | anticancer agent |
| 102 | vinca alkaloid | platinum-based drug | PI3K inhibitor |
| 103 | vinca alkaloid | platinum-based drug | CDK4/6 inhibitor |
| 104 | vinca alkaloid | platinum-based drug | CARM1 inhibitor |
| 105 | vinca alkaloid | nucleoside | PI3K inhibitor |
| | | anticancer agent | |
| 106 | vinca alkaloid | nucleoside | CDK4/6 inhibitor |
| | | anticancer agent | |
| 107 | vinca alkaloid | nucleoside | CARM1 inhibitor |
| | | anticancer agent | |
| 108 | vinca alkaloid | PI3K inhibitor | CDK4/6 inhibitor |
| 109 | vinca alkaloid | PI3K inhibitor | CARM1 inhibitor |
| 110 | vinca alkaloid | CDK4/6 inhibitor | CARM1 inhibitor |
| 111 | platinum-based drug | nucleoside | PI3K inhibitor |
| | | anticancer agent | |
| 112 | platinum-based drug | nucleoside | CDK4/6 inhibitor |
| | | anticancer agent | |
| 113 | platinum-based drug | nucleoside | CARM1 inhibitor |
| | | anticancer agent | |
| 114 | platinum-based drug | PI3K inhibitor | CDK4/6 inhibitor |
| 115 | platinum-based drug | PI3K inhibitor | CARM1 inhibitor |
| 116 | platinum-based drug | CDK4/6 inhibitor | CARM1 inhibitor |
| 117 | nucleoside | PI3K inhibitor | CDK4/6 inhibitor |
| | anticancer agent | | |
| 118 | nucleoside | PI3K inhibitor | CARM1 inhibitor |
| | anticancer agent | | |
| 119 | nucleoside | CDK4/6 inhibitor | CARM1 inhibitor |
| | anticancer agent | | |
| 120 | PI3K inhibitor | CDK 4/6 inhibitor | CARM1 inhibitor |

### III. Third Therapeutic Agents

In one embodiment, the therapeutic methods, uses, compositions, and kits of the present disclosure comprise administering a therapeutically effective amount of a Compound of the Disclosure in combination with a therapeutically effective amount of a Second Therapeutic Agent and a therapeutically effective amount of a Third Therapeutic Agent to a subject in need thereof.

The term “Third Therapeutic Agent” as used herein comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

In one embodiment, the Third Therapeutic Agent comprises one compound from one drug class, i.e., one glucocorticoid receptor agonist, one immunomodulatory drug, one proteasome inhibitor, one Bcl-2 inhibitor, one pleiotropic pathway modulator, one XPO1 inhibitor, one histone deacetylase inhibitor, or one EZH2 inhibitor.

In another embodiment, the Third Therapeutic Agent comprises two different compounds from one drug class, e.g., two different glucocorticoid receptor agonists, e.g., dexamethasone and prednisone, two different immunomodulatory drugs, two different proteasome inhibitors, two different Bcl-2 inhibitors, two different pleiotropic pathway modulators, two different XPO1 inhibitors, two different histone deacetylase inhibitors, or two different EZH2 inhibitors.

In another embodiment, the Third Therapeutic Agent comprises three different compounds from one drug class, e.g., three different glucocorticoid receptor agonists, e.g., dexamethasone, prednisone, and methylprednisolone, three different immunomodulatory drugs, three different proteasome inhibitors, three different Bcl-2 inhibitors, three different pleiotropic pathway modulators, three different XPO1 inhibitors, three different histone deacetylase inhibitors, or three different EZH2 inhibitors.

In another embodiment, the Third Therapeutic Agent comprises three different compounds from two drug classes, e.g., two different glucocorticoid receptor agonists, e.g., dexamethasone and prednisone, and one immunomodulatory drug; two different glucocorticoid receptor agonists and one proteasome inhibitor; and so on.

In another embodiment, the Third Therapeutic Agent comprises compounds from two different drug classes. For example, in one embodiment, the Second Therapeutic Agent comprises a first compound from a first drug class and second compound from a second drug class, wherein the first drug class and the second drug class are different. In a specific non-limiting example, the Third Therapeutic Agent comprises an EZH2 inhibitor, e.g., tazemetostat, and an immunomodulatory drug, e.g., lenoliminide. Non-limiting examples of combinations of first and second drug classes of the Third Therapeutic Agent are provided in Table 6.

TABLE 6

| No. | Drug Class # 1 | Drug Class # 2 |
|---|---|---|
| 1 | GR agonist | IMiD |
| 2 | GR agonist | Proteasome inhibitor |
| 3 | GR agonist | Bcl-2 inhibitor |
| 4 | GR agonist | Pleiotropic pathway modulator |
| 5 | GR agonist | XPO1 inhibitor |
| 6 | GR agonist | HDAC inhibitor |
| 7 | GR agonist | EZH2 inhibitor |
| 8 | IMiD | Proteasome inhibitor |
| 9 | IMiD | Bcl-2 inhibitor |
| 10 | IMiD | Pleiotropic pathway modulator |
| 11 | IMiD | XPO1 inhibitor |
| 12 | IMiD | HDAC inhibitor |
| 13 | IMiD | EZH2 inhibitor |
| 14 | Proteasome inhibitor | Bcl-2 inhibitor |
| 15 | Proteasome inhibitor | Pleiotropic pathway modulator |
| 16 | Proteasome inhibitor | XPO1 inhibitor |
| 17 | Proteasome inhibitor | HDAC inhibitor |
| 18 | Proteasome inhibitor | EZH2 inhibitor |
| 19 | Bcl-2 inhibitor | Pleiotropic pathway modulator |
| 20 | Bcl-2 inhibitor | XPO1 inhibitor |
| 21 | Bcl-2 inhibitor | HDAC inhibitor |
| 22 | Bcl-2 inhibitor | EZH2 inhibitor |
| 23 | Pleiotropic pathway modulator | XPO1 inhibitor |
| 24 | Pleiotropic pathway modulator | HDAC inhibitor |
| 25 | Pleiotropic pathway modulator | EZH2 inhibitor |
| 26 | XPO1 inhibitor | HDAC inhibitor |
| 27 | XPO1 inhibitor | EZH2 inhibitor |
| 28 | HDAC inhibitor | EZH2 inhibitor |

In another embodiment, the Third Therapeutic Agent comprises compounds from three different drug classes. For example, in one embodiment, the Second Therapeutic Agent comprises a first compound from a first drug class, and a second compound from a second drug class, and a third compound from a third drug class, wherein the first drug class, the second drug class, and the third drug class are different. Non-limiting examples of combinations of first, second, and third drug classes of the Third Therapeutic Agent are provided in Table 7.

TABLE 7

| No. | Drug Class # 1 | Drug Class # 2 | Drug Class # 3 |
|---|---|---|---|
| 1 | GR agonist | IMiD | Proteasome inhibitor |
| 2 | GR agonist | IMiD | Bcl-2 inhibitor |
| 3 | GR agonist | IMiD | Pleiotropic pathway |
| | | | modulator |
| 4 | GR agonist | IMiD | XPO1 inhibitor |
| 5 | GR agonist | IMiD | HDAC inhibitor |
| 6 | GR agonist | IMiD | EZH2 inhibitor |
| 7 | GR agonist | Proteasome inhibitor | Bcl-2 inhibitor |
| 8 | GR agonist | Proteasome inhibitor | Pleiotropic pathway |
| | | | modulator |
| 9 | GR agonist | Proteasome inhibitor | XPO1 inhibitor |
| 10 | GR agonist | Proteasome inhibitor | HDAC inhibitor |
| 11 | GR agonist | Proteasome inhibitor | EZH2 inhibitor |
| 12 | GR agonist | Bcl-2 inhibitor | Pleiotropic pathway |
| | | | modulator |
| 13 | GR agonist | Bcl-2 inhibitor | XPO1 inhibitor |
| 14 | GR agonist | Bcl-2 inhibitor | HDAC inhibitor |
| 15 | GR agonist | Bcl-2 inhibitor | EZH2 inhibitor |
| 16 | GR agonist | Pleiotropic pathway | XPO1 inhibitor |
| | | modulator | |
| 17 | GR agonist | Pleiotropic pathway | HDAC inhibitor |
| | | modulator | |
| 18 | GR agonist | Pleiotropic pathway | EZH2 inhibitor |
| | | modulator | |
| 19 | GR agonist | XPO1 inhibitor | HDAC inhibitor |
| 20 | GR agonist | XPO1 inhibitor | EZH2 inhibitor |
| 21 | GR agonist | HDAC inhibitor | EZH2 inhibitor |
| 22 | IMiD | Proteasome inhibitor | Bcl-2 inhibitor |
| 23 | IMiD | Proteasome inhibitor | Pleiotropic pathway |
| | | | modulator |
| 24 | IMiD | Proteasome inhibitor | XPO1 inhibitor |
| 25 | IMiD | Proteasome inhibitor | HDAC inhibitor |
| 26 | IMiD | Proteasome inhibitor | EZH2 inhibitor |
| 27 | IMiD | Bcl-2 inhibitor | Pleiotropic pathway |
| | | | modulator |
| 28 | IMiD | Bcl-2 inhibitor | XPO1 inhibitor |
| 29 | IMiD | Bcl-2 inhibitor | HDAC inhibitor |
| 30 | IMiD | Bcl-2 inhibitor | EZH2 inhibitor |
| 31 | IMiD | Pleiotropic pathway | XPO1 inhibitor |
| | | modulator | |
| 32 | IMiD | Pleiotropic pathway | HDAC inhibitor |
| | | modulator | |
| 33 | IMiD | Pleiotropic pathway | EZH2 inhibitor |
| | | modulator | |
| 34 | IMiD | XPO1 inhibitor | HDAC inhibitor |
| 35 | IMiD | XPO1 inhibitor | EZH2 inhibitor |
| 36 | IMiD | HDAC inhibitor | EZH2 inhibitor |
| 37 | Proteasome inhibitor | Bcl-2 inhibitor | Pleiotropic pathway |
| | | | modulator |
| 38 | Proteasome inhibitor | Bcl-2 inhibitor | XPO1 inhibitor |
| 39 | Proteasome inhibitor | Bcl-2 inhibitor | HDAC inhibitor |
| 40 | Proteasome inhibitor | Bcl-2 inhibitor | EZH2 inhibitor |
| 41 | Proteasome inhibitor | Pleiotropic pathway | XPO1 inhibitor |
| | | modulator | |
| 42 | Proteasome inhibitor | Pleiotropic pathway | HDAC inhibitor |
| | | modulator | |
| 43 | Proteasome inhibitor | Pleiotropic pathway | EZH2 inhibitor |
| | | modulator | |
| 44 | Proteasome inhibitor | XPO1 inhibitor | HDAC inhibitor |
| 45 | Proteasome inhibitor | XPO1 inhibitor | EZH2 inhibitor |
| 46 | Proteasome inhibitor | HDAC inhibitor | EZH2 inhibitor |
| 47 | Bcl-2 inhibitor | Pleiotropic pathway | XPO1 inhibitor |
| | | modulator | |
| 48 | Bcl-2 inhibitor | Pleiotropic pathway | HDAC inhibitor |
| | | modulator | |
| 49 | Bcl-2 inhibitor | Pleiotropic pathway | EZH2 inhibitor |
| | | modulator | |
| 50 | Bcl-2 inhibitor | XPO1 inhibitor | HDAC inhibitor |
| 51 | Bcl-2 inhibitor | XPO1 inhibitor | EZH2 inhibitor |
| 52 | Bcl-2 inhibitor | HDAC inhibitor | EZH2 inhibitor |
| 53 | Pleiotropic pathway | XPO1 inhibitor | HDAC inhibitor |
| | modulator | | |
| 54 | Pleiotropic pathway | XPO1 inhibitor | EZH2 inhibitor |
| | modulator | | |
| 55 | Pleiotropic pathway | HDAC inhibitor | EZH2 inhibitor |
| | modulator | | |
| 56 | XPO1 inhibitor | HDAC inhibitor | EZH2 inhibitor |

Non-limiting examples of combinations of a Second Therapeutic Agent and a Third Therapeutic Agent are provided in Table 8.

TABLE 8

| No. | Drug Class # 1 | Drug Class # 2 |
|---|---|---|
| 1 | BTK inhibitor | IMiD |
| 2 | BTK inhibitor | GR agonist |
| 3 | BTK inhibitor | Proteasome inhibitor |
| 4 | BTK inhibitor | Bcl-2 inhibitor |
| 5 | BTK inhibitor | Pleiotropic pathway modulator |
| 6 | BTK inhibitor | XPO1 inhibitor |
| 7 | BTK inhibitor | HDAC inhibitor |
| 8 | BTK inhibitor | EZH2 inhibitor |
| 9 | anti-CD20 mAb | IMiD |
| 10 | anti-CD20 mAb | GR agonist |
| 11 | anti-CD20 mAb | Proteasome inhibitor |
| 12 | anti-CD20 mAb | Bcl-2 inhibitor |
| 13 | anti-CD20 mAb | Pleiotropic pathway modulator |
| 14 | anti-CD20 mAb | XPO1 inhibitor |
| 15 | anti-CD20 mAb | HDAC inhibitor |
| 16 | anti-CD20 mAb | EZH2 inhibitor |
| 17 | alkylating agent | IMiD |
| 18 | alkylating agent | GR agonist |
| 19 | alkylating agent | Proteasome inhibitor |
| 20 | alkylating agent | Bcl-2 inhibitor |
| 21 | alkylating agent | Pleiotropic pathway modulator |
| 22 | alkylating agent | XPO1 inhibitor |
| 23 | alkylating agent | HDAC inhibitor |
| 24 | alkylating agent | EZH2 inhibitor |
| 25 | topoisomerase II inhibitor | IMiD |
| 26 | topoisomerase II inhibitor | GR agonist |
| 27 | topoisomerase II inhibitor | Proteasome inhibitor |
| 28 | topoisomerase II inhibitor | Bcl-2 inhibitor |
| 29 | topoisomerase II inhibitor | Pleiotropic pathway modulator |
| 30 | topoisomerase II inhibitor | XPO1 inhibitor |
| 31 | topoisomerase II inhibitor | HDAC inhibitor |
| 32 | topoisomerase II inhibitor | EZH2 inhibitor |
| 33 | vinca alkaloid | IMiD |
| 34 | vinca alkaloid | GR agonist |
| 35 | vinca alkaloid | Proteasome inhibitor |
| 36 | vinca alkaloid | Bc1-2 inhibitor |
| 37 | vinca alkaloid | Pleiotropic pathway modulator |
| 38 | vinca alkaloid | XPO1 inhibitor |
| 39 | vinca alkaloid | HDAC inhibitor |
| 40 | vinca alkaloid | EZH2 inhibitor |
| 41 | platinum-based drug | IMiD |
| 42 | platinum-based drug | GR agonist |
| 43 | platinum-based drug | Proteasome inhibitor |
| 44 | platinum-based drug | Bcl-2 inhibitor |
| 45 | platinum-based drug | Pleiotropic pathway modulator |
| 46 | platinum-based drug | XPO1 inhibitor |
| 47 | platinum-based drug | HDAC inhibitor |
| 48 | platinum-based drug | EZH2 inhibitor |
| 49 | nucleoside anticancer agents | IMiD |
| 50 | nucleoside anticancer agents | GR agonist |
| 51 | nucleoside anticancer agents | Proteasome inhibitor |
| 52 | nucleoside anticancer agents | Bcl-2 inhibitor |
| 53 | nucleoside anticancer agents | Pleiotropic pathway modulator |
| 54 | nucleoside anticancer agents | XPO1 inhibitor |
| 55 | nucleoside anticancer agents | HDAC inhibitor |
| 56 | nucleoside anticancer agents | EZH2 inhibitor |
| 57 | PI3K inhibitor | IMiD |
| 58 | PI3K inhibitor | GR agonist |
| 59 | PI3K inhibitor | Proteasome inhibitor |
| 60 | PI3K inhibitor | Bcl-2 inhibitor |
| 61 | PI3K inhibitor | Pleiotropic pathway modulator |
| 62 | PI3K inhibitor | XPO1 inhibitor |
| 63 | PI3K inhibitor | HDAC inhibitor |
| 64 | PI3K inhibitor | EZH2 inhibitor |
| 65 | CDK4/6 inhibitor | IMiD |
| 66 | CDK4/6 inhibitor | GR agonist |
| 67 | CDK4/6 inhibitor | Proteasome inhibitor |
| 68 | CDK4/6 inhibitor | Bcl-2 inhibitor |
| 69 | CDK4/6 inhibitor | Pleiotropic pathway modulator |
| 70 | CDK4/6 inhibitor | XPO1 inhibitor |
| 71 | CDK4/6 inhibitor | HDAC inhibitor |
| 72 | CDK4/6 inhibitor | EZH2 inhibitor |
| 73 | CARM1 inhibitor | IMiD |
| 74 | CARM1 inhibitor | GR agonist |
| 75 | CARM1 inhibitor | Proteasome inhibitor |
| 76 | CARM1 inhibitor | Bcl-2 inhibitor |
| 77 | CARM1 inhibitor | Pleiotropic pathway modulator |
| 78 | CARM1 inhibitor | XPO1 inhibitor |
| 79 | CARM1 inhibitor | HDAC inhibitor |
| 80 | CARM1 inhibitor | EZH2 inhibitor |

The term “drug class” as used herein refers to the grouping of biologically active molecules, i.e., drugs, based on their chemical nature, mechanism of action, e.g., binding to the same biological target, and/or mode of action to treat a disease, disorder, or condition in a subject. A Second Therapeutic Agent of the disclosure comprises one or more biologically active molecules from one or more drug classes. These drug classes include BTK inhibitors, anti-CD20 monoclonal antibodies, alkylating agents, topoisomerase II inhibitors, vinca alkaloids, platinum-based drugs, nucleoside anticancer agents, PI3K inhibitors, CDK4/6 inhibitors, CARM1 inhibitors, inhibitors of an enzyme of DNA damage repair, SYK inhibitors and MEK inhibitors. Likewise, a Third Therapeutic Agent of the disclosure comprises one or more biologically active molecules from one or more drug classes. These drug classes include glucocorticoid receptor agonists, immunomodulatory drugs, proteasome inhibitors, Bcl-2 inhibitors, pleiotropic pathway modulators, XPO1 inhibitors, histone deacetylase inhibitors, and EZH2 inhibitors.

The terms “BTK inhibitor” or “BTKi” as used herein refers to a compound that inhibits Bruton's tyrosine kinase, including wild-type BTK and mutant BTK. BTK inhibitors and methods of administering BTK inhibitors to a subject are known in the art. Exemplary BTK inhibitors include, but are not limited to, ibrutinib, evobrutinib, tirabrutinib, spebrutinib, poseltinib, pirtobrutinib (LOXO-305), acalabrutinib, and zanubrutinib.

The terms “anti-CD20 monoclonal antibody” or “anti CD20 mAb” as used herein refers to a compound that binds to CD20. Anti-CD20 monoclonal antibodies may include bispecific antibodies (BsAb). A non-limiting exemplary anti-CD20 bispecific antibody is BsAb CD20/CD3. CD20 is a surface antigen on B cells, whereas CD3 is an antigen on the surface of T-cells. Anti-CD20 monoclonal antibodies and methods of administering anti-CD20 monoclonal antibodies to a subject are known in the art. A non-limiting exemplary anti-CD20 monoclonal antibody is rituximab, obinutuzumab, ocaratuzumab, ibritumomab, tiuxetan, tositumomab, ofatumumab, ocrelizumab, and veltuzumab. Exemplary examples of a BsAb is mosunetuzumab, golimumab, and RGN1979.

The term “alkylating agent” as used herein refers to an alkylating agent for use in treating cancer that attaches an alkyl group to DNA. A non-limiting exemplary alkylating agent is mafosfamide.

The term “topoisomerase II inhibitor” as used herein refers to a compound for use in treating cancer that inhibits Type II topoisomerase. Exemplary topoisomerase II inhibitors include, but are not limited to, doxorubicin, etoposide, novobiocin, ciprofloxacin, teniposide, HU-331, ICRF-187, ICRF-193, and mitindomide.

The term “vinca alkaloid” as used herein refers to anti-mitotic and anti-microtubule alkaloid agents originally derived from vinca plants. Exemplary vinca alkaloids include, but are not limited to, vincristine, vinblastine, vindesine, vinorelbine, vincaminol, vineridine, vinburnine, vinpocetine, minovincine, methoxyminovincine, minovincinine, vincadifformine, desoxyvincaminol, and vincamajine.

The term “platinum-based drug” as used herein refers to platinum containing agents that coordinate to DNA to interfere with DNA repair. Exemplary platinum-based drugs include, but are not limited to, carboplatin, cisplatin, oxaliplatin, dicycloplatin, eptaplatin, lobaplatin, miriplatin, nedaplatin, picoplatin, satraplatin, and triplatin tetranitrate.

The term “nucleoside anticancer agent” as used herein refers to nucleoside analogs for treating cancer. Exemplary nucleoside anticancer agents include, but are not limited to, gemcitabine and cytarabine.

The terms “PI3K inhibitor” or “PIK3i” as used herein refers to a compound that inhibits phosphoinositide 3-kinase. PI3K inhibitors and methods of administering PI3K inhibitors to a subject are known in the art. Exemplary PI3K inhibitors include, but are not limited to, copanlisib, idelalisib, duvelisib, taselisib, buparlisib, alpelisib, umbralisib, dactolisib, and voxtalisib.

The term “CDK4/6 inhibitor” or “CDK4/6i” as used herein refers to a compound that inhibits two types of cyclin-dependent kinase—CDK4 and CDK6. CDK4/6 inhibitors and methods of administering CDK4/6 inhibitors to a subject are known in the art. Exemplary CDK4/6 inhibitors include but are not limited to, abemaciclib, ribociclib, and palbociclib.

The term “CARM1 inhibitor” or “CARM1i” as used herein refers to a compound that inhibits coactivator-associated arginine methyltransferase 1. CARM1 inhibitors and methods of administering CARM1 inhibitors to a subject are known in the art. A non-limiting exemplary CARM1 inhibitor is EZM2302.

The term “glucocorticoid receptor agonist” or “GR agonist” as used herein refers to a compound that activates the glucocorticoid receptor. Glucocorticoid receptor agonists and methods of administering glucocorticoid receptor agonists to a subject are known in the art. See, e.g., Pufall, M. A., Adv Exp Med Biol. 872:315-333 (2015). Exemplary glucocorticoid receptor agonists include, but are not limited to, dexamethasone, hydrocortisone, corticosterone, prednisolone, methylprednisolone, prednisone, triamcinolone, mapracorat, ciclesonide, and (20S)-protopanaxatriol. In one embodiment, the glucocorticoid receptor agonist is prednisone. In another embodiment, the glucocorticoid receptor agonist is dexamethasone.

The term “immunomodulatory drug” or “IMiD” as used herein refers to a compound that inhibits the production of tumour necrosis factor, interleukin 6, immunoglobulin G, and/or VEGF, and/or co-stimulates T cells and NK cells, and/or increases interferon gamma and interleukin 2 production. Immunomodulatory drugs and methods of administering immunomodulatory drugs to a subject are known in the art. Exemplary immunomodulatory drugs include, but art not limited to, thalidomide, lenalidomide, and pomalidomide. In one embodiment, the immunomodulatory drug is pomalidomide.

The term “proteasome inhibitor” as used herein refers to a compound that blocks the action of proteasomes and thus prevents the degredation of pro-apoptotic factors such as p53 protein. Proteasome inhibitors and methods of administering proteasome inhibitors to a subject are known in the art. Exemplary proteasome inhibitors include, but art not limited to, bortezomib, carfilzomib, and ixazomib. In one embodiment, the proteasome inhibitor is bortezomib.

The term “Bcl-2 inhibitor” as used herein refers to a compound that inhibits the anti-apoptotic Bcl-2 protein. Bcl-2 inhibitors and methods of administering Bcl-2 inhibitors to a subject are known in the art. Examplary Bcl-2 inhibitors include but are not limited to, navitoclax (ABT-263), ABT-737, Sabutoclax, AT-1019 (Gossypol), TW-37, venetoclax (ABT-199), obatoclax, HA14-1, A-1155463, A-1331852, and WEHI-539. In one embodiment, the Bcl-2 inhibitor is venetoclax.

The term “pleiotropic pathway modulator” as used herein refers to compound that binds to cereblon to promote protein degredation. Pleiotropic pathway modulators and methods of administering pleiotropic pathway modulators to a subject are known in the art are known in the art. See, e.g., Hagner et al., Blood 126:779-789 (2017). A non-limiting exemplary pleiotropic pathway modulator is CC-122.

The term “XPO1 inhibitor” as used herein refers to an inhibitor of exportin-1 (also known as chromosome region maintenance 1 protein homolog; CRM1). XPO1 inhibitors and methods of administering XPO1 inhibitors to a subject are known in the art. See, e.g., Wang and Liu, Stem Cell Invest 6:6 (2019). A non-limiting exemplary XPO1 inhibitor is selinexor.

The term “histone deacetylase inhibitor” or “HDAC inhibitor” as used herein refers to a compound that inhibits histone deactylase enzymes. Histone deacetylase inhibitors and methods of administering histone deacetylase inhibitors to a subject are known in the art. See, e.g., Eckschlager et al., Int. J. Mol. Sci. 18:1414 (2017) doi:10.3390/ijms18071414. Exemplary histone deacetylase inhibitors include, but are not limited to, romidepsin, belinostat, panobinostat, and vorinostate. In one embodiment, the histone deacetylase inhibitor is panobinostat.

The term “EZH2 inhibitor” as used herein refers to a compound that inhibits the enhancer of zeste homolog 2 enzyme. EZH2 inhibitors and methods of administering EZH2 inhibitors to a subject are known in the art. See, e.g., Lue and Amengual, Curr Hematol Malig Rep 13:369-382 (2018). Exemplary EZH2 inhibitors include, but are not limited to, tazemetostat (Tazverik©), EPZ011989, EPZ005687, GSK126, PF-06821497, and valemetostat. In one embodiment, the EZH2 inhibitor is tazemetostat.

The term “inhibitor of an enzyme of DNA damage repair” or “DNA repair enzyme inhibitor” refers to a compound that inhibits an enzyme that recognizes and corrects physical damage in DNA. Enzymes involved in DNA damage response pathways and frequently mutated in cancer include, but are not limited to, enzymes encoded by the genes ATM, ATR, PAXIP, BRCA1, BRCA2, RAD51, FRC, XRCC1, PCNA, PARP1, ERCC1, and MSH3.

The term “ATM inhibitor” as used herein refers to a compound that inhibits ataxia telangiectasia mutated kinase. ATM inhibitors and methods of administering ATM inhibitors to a subject are known in the art. Exemplary ATM inhibitors include, but are not limited to, AZD0156, dactolisib, KU-55933, CP-466722, and AZD1390.

The term “ATR inhibitor” as used herein refers to a compound that inhibits the ataxia telangiectasia and Rad3-related protein. ATR inhibitors and methods of administering ATR inhibitors to a subject are known in the art. Exemplary ATR inhibitors include, but are not limited to, AZD6738, VX-803, and elimusertib.

The term “Chk1 inhibitor” as used herein refers to a compound that inhibits the serine/threonine-specific protein kinase that, in humans, is encoded by the gene CHEKL. Chk1 inhibitors and methods of administering Chk1 inhibitors to a subject are known in the art. Exemplary Chk1 inhibitors include, but are not limited to, AZD7762, rabusertib, MK-8776, CHIR-124, and PF-477736.

The term “Wee1 inhibitor” as used herein refers to a compound that inhibits the tyrosine kinase belonging to the serine/threonine family of protein kinases, that in humans, is encoded by the gene Wee1. Wee1 inhibitors and methods of administering Wee1 inhibitors to a subject are known in the art. Exemplary Wee1 inhibitors include, but are not limited to, AZD1755.

The term “RAD51 inhibitor” as used herein refers to a compound that inhibits DNA repair protein RAD51 homolog 1 that, in humans, is encoded by the gene RAD51. RAD51 inhibitors and methods of administering RAD51 inhibitors to a subject are known in the art. Exemplary RAD51 inhibitors include, but are not limited to, B02 and RI-1.

The term “PARP inhibitor” as used herein refers to a compound that inhibits poly (ADP-ribose) polymerase protein(s). PARP inhibitors and methods of administering PARP inhibitors to a subject are known in the art. Exemplary PARP inhibitors include, but are not limited to, olaparib, niraparib, rucaparib, and talazoparib.

The term “AKT inhibitor” as used herein refers to a compound that inhibits serine/threonine-specific protein kinases that, in humans, are encoded by the genes AKT1, AKT2, and AKT3. AKT inhibitors and methods of administering AKT inhibitors to a subject are known in the art. Exemplary AKT inhibitors include, but are not limited to, MK2206.

The term “SYK inhibitor” as used herein refers to a compound that inhibits spleen tyrosine kinase that, in humans, is encoded by the gene SYK. SYK inhibitors and methods of administering SYK inhibitors to a subject are known in the art. Exemplary SYK inhibitors include, but are not limited to, tamatinib, fostamatinib, R406, MNS, lanraplenib, TAK-659, entospletinib, and BAY-61-3606.

The term “MEK inhibitor” as used herein refers to a compound that inhibits mitogen-activated protein kinase kinase enzymes. MEK inhibitors and methods of administering MEK inhibitors to a subject are known in the art. Exemplary MEK inhibitors include, but are not limited to, trametinib, selumetinib, and merdametinib.

### IV. Therapeutic Methods

In one embodiment, the present disclosure is directed to a method for treating a disease, condition, or disorder in a subject suffering from, or at risk of suffering from, the disease, condition, or disorder, the method comprising administering to the subject an effective amount of a Compound of the Disclosure and a Second Therapeutic Agent.

In another embodiment, present disclosure is directed to a method for treating a disease, condition, or disorder in a subject suffering from, or at risk of suffering from, the disease, condition, or disorder, the method comprising administering to the subject an effective amount of a Compound of the Disclosure, a Second Therapeutic Agent, and a Third Therapeutic Agent.

In one embodiment, the disease, condition, or disorder is responsive to or mediated by the inhibition of SETD2 protein by a Compound of the Disclosure.

In the therapeutic methods and uses provided herein, the Compound of the Disclosure, the Second Therapeutic Agent, and the optional Third Therapeutic Agent can be administered in combination under one or more of the following conditions: as separate pharmaceutical compositions, at different periodicities, e.g., simultaneously or sequentially, at different durations, at different concentrations, by different administration routes, etc. Other therapeutic, e.g., anticancer, agents may also be administered to the cancer patient.

In another embodiment, the present disclosure provides a method of treating a disease, disorder, or condition in a subject comprising administering a therapeutically effective amount of a Compound of the Disclosure in combination with a Second Therapeutic Agent.

In another embodiment, the present disclosure provides a method of treating a disease, disorder, or condition in a subject comprising administering a therapeutically effective amount of a Compound of the Disclosure in combination with a Second Therapeutic Agent to provide an additive effect.

In another embodiment, the present disclosure provides a method of treating a disease, disorder, or condition in a subject comprising administering a therapeutically effective amount of a Compound of the Disclosure in combination with a Second Therapeutic Agent to provide a synergistic effect, e.g., the combined therapeutic effects of the Compound of the Disclosure and the Second Therapeutic Agent have an effect that is more significant than each agent alone.

In another embodiment, the present disclosure provides a method of treating a disease, disorder, or condition in a subject comprising administering a therapeutically effective amount of a Compound of the Disclosure in combination with a Second Therapeutic Agent and a Third Therapeutic Agent.

In another embodiment, the present disclosure provides a method of treating a disease, disorder, or condition in a subject comprising administering a therapeutically effective amount of a Compound of the Disclosure in combination with a Second Therapeutic Agent and a Third Therapeutic Agent to provide an additive effect.

In another embodiment, the present disclosure provides a method of treating a disease, disorder, or condition in a subject comprising administering a therapeutically effective amount of a Compound of the Disclosure in combination with a Second Therapeutic Agent and a Third Therapeutic Agent to provide a synergistic effect.

In another aspect, the present disclosure provides a method of treating cancer in a subject comprising administering a therapeutically effective amount of a Compound of the Disclosure in combination with a Second Therapeutic Agent and, optionally, a Third Therapeutic Agent. While not being limited to a specific mechanism, in some embodiments, Compounds of the Disclosure treat cancer by inhibiting SETD2 protein. Examples of treatable cancers include, but are not limited to, the cancers listed in Table 2.

TABLE 2

| | |
|---|---|
| adrenal cancer | lymphoepithelioma |
| acinic cell carcinoma | lymphoma |
| acoustic neuroma | acute lymphocytic leukemia |
| acral lentigious melanoma | acute myelogeous leukemia |
| acrospiroma | chronic lymphocytic leukemia |
| acute eosinophilic leukemia | liver cancer |
| acute erythroid leukemia | small cell lung cancer |
| acute lymphoblastic leukemia | non-small cell lung cancer |
| acute megakaryoblastic leukemia | MALT lymphoma |
| acute monocytic leukemia | malignant fibrous histiocytoma |
| acute promyelocytic leukemia | malignant peripheral nerve sheath |
| | tumor |
| adenocarcinoma | malignant triton tumor |
| adenoid cystic carcinoma | mantle cell lymphoma |
| adenoma | marginal zone B-cell lymphoma |
| adenomatoid odontogenic tumor | mast cell leukemia |
| adenosquamous carcinoma | mediastinal germ cell tumor |
| adipose tissue neoplasm | medullary carcinoma of the breast |
| adrenocortical carcinoma | medullary thyroid cancer |
| adult T-cell leukemia/lymphoma | medulloblastoma |
| aggressive NK-cell leukemia | melanoma |
| AIDS-related lymphoma | meningioma |
| alveolar rhabdomyosarcoma | merkel cell cancer |
| alveolar soft part sarcoma | mesothelioma |
| ameloblastic fibroma | metastatic urothelial carcinoma |
| anaplastic large cell lymphoma | mixed Mullerian tumor |
| anaplastic thyroid cancer | mucinous tumor |
| angioimmunoblastic T-cell | multiple myeloma |
| lymphoma | |
| angiomyolipoma | muscle tissue neoplasm |
| angiosarcoma | mycosis fungoides |
| astrocytoma | myxoid liposarcoma |
| atypical teratoid rhabdoid tumor | myxoma |
| B-cell chronic lymphocytic | myxosarcoma |
| leukemia | |
| B-cell prolymphocytic leukemia | nasopharyngeal carcinoma |
| B-cell lymphoma | neurinoma |
| basal cell carcinoma | neuroblastoma |
| biliary tract cancer | neurofibroma |
| bladder cancer | neuroma |
| blastoma | nodular melanoma |
| bone cancer | ocular cancer |
| Brenner tumor | oligoastrocytoma |
| Brown tumor | oligodendroglioma |
| Burkitt's lymphoma | oncocytoma |
| breast cancer | optic nerve sheath meningioma |
| brain cancer | optic nerve tumor |
| carcinoma | oral cancer |
| carcinoma in situ | osteosarcoma |
| carcinosarcoma | ovarian cancer |
| cartilage tumor | Pancoast tumor |
| cementoma | papillary thyroid cancer |
| myeloid sarcoma | paraganglioma |
| chondroma | pinealoblastoma |
| chordoma | pineocytoma |
| choriocarcinoma | pituicytoma |
| choroid plexus papilloma | pituitary adenoma |
| clear-cell sarcoma of the kidney | pituitary tumor |
| craniopharyngioma | plasmacytoma |
| cutaneous T-cell lymphoma | polyembryoma |
| cervical cancer | precursor T-lymphoblastic lymphoma |
| colorectal cancer | primary central nervous system |
| | lymphoma |
| Degos disease | primary effusion lymphoma |
| desmoplastic small round cell | preimary peritoneal cancer |
| tumor | |
| diffuse large B-cell lymphoma | prostate cancer |
| dysembryoplastic neuroepithelial | pancreatic cancer |
| tumor | |
| dysgerminoma | pharyngeal cancer |
| embryonal carcinoma | pseudomyxoma periotonei |
| endocrine gland neoplasm | renal cell carcinoma |
| endodermal sinus tumor | renal medullary carcinoma |
| enteropathy-associated T-cell | retinoblastoma |
| lymphoma | |
| esophageal cancer | rhabdomyoma |
| fetus in fetu | rhabdomyosarcoma |
| fibroma | Richter's transformation |
| fibrosarcoma | rectal cancer |
| follicular lymphoma | sarcoma |
| follicular thyroid cancer | Schwannomatosis |
| ganglioneuroma | seminoma |
| gastrointestinal cancer | Sertoli cell tumor |
| germ cell tumor | sex cord-gonadal stromal tumor |
| gestational choriocarcinoma | signet ring cell carcinoma |
| giant cell fibroblastoma | skin cancer |
| giant cell tumor of the bone | small blue round cell tumors |
| glial tumor | small cell carcinoma |
| glioblastoma multiforme | soft tissue sarcoma |
| glioma | somatostatinoma |
| gliomatosis cerebri | soot wart |
| glucagonoma | spinal tumor |
| gonadoblastoma | splenic marginal zone lymphoma |
| granulosa cell tumor | squamous cell carcinoma |
| gynandroblastoma | synovial sarcoma |
| gallbladder cancer | Sezary's disease |
| gastric cancer | small intestine cancer |
| hairy cell leukemia | squamous carcinoma |
| hemangioblastoma | stomach cancer |
| head and neck cancer | T-cell lymphoma |
| hemangiopericytoma | testicular cancer |
| hematological malignancy | thecoma |
| hepatoblastoma | thyroid cancer |
| hepatosplenic T-cell lymphoma | transitional cell carcinoma |
| Hodgkin's lymphoma | throat cancer |
| non-Hodgkin's lymphoma | urachal cancer |
| invasive lobular carcinoma | urogenital cancer |
| intestinal cancer | urothelial carcinoma |
| kidney cancer | uveal melanoma |
| laryngeal cancer | uterine cancer |
| lentigo maligna | verrucous carcinoma |
| lethal midline carcinoma | visual pathway glioma |
| leukemia | vulvar cancer |
| leydig cell tumor | vaginal cancer |
| liposarcoma | Waldenstrom's macroglobulinemia |
| lung cancer | Warthin's tumor |
| lymphangioma | Wilms' tumor. |
| lymphangiosarcoma | |

In another embodiment, the cancer is pancreatic cancer or esophageal cancer.

In another embodiment, the cancer is selected from the group consisting of esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, prostate cancer, hematological cancer, pancreatic cancer, skin cancer, endometrial cancer, ovarian cancer, and colorectal cancer.

In another embodiment, the cancer or cancer cell is a hematological cancer. Exemplary hematological cancers include, but are not limited to, the cancers listed in Table 3.

TABLE 3

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In another embodiment, the cancer is multiple myeloma.

In another embodiment, the multiple myeloma is characterized as having chromosomal translocations involving the immunoglobulin heavy chain locus at 14q32. In another embodiment, the chromosomal translocation is a t(4;14) translocation, i.e., the multiple myeloma is t(4;14) multiple myeloma.

In another embodiment, the cancer is mantle cell lymphoma.

In another embodiment, the cancer is diffuse large B-cell lymphoma.

In another embodiment, the present disclosure provides a therapeutic method of modulating protein methylation, gene expression, cell proliferation, cell differentiation and/or apoptosis in vivo in the cancers mentioned above by administering a therapeutically effective amount of a Compound of the Disclosure to a subject in need of such therapy and a Second Therapeutic Agent and, optionally, a Third Therapeutic Agent.

The present disclosure also provides the following particular embodiments.

Embodiment 1. A method of treating a subject in need thereof, the method comprising administering to the subject a therapeutically effective amount of:
(a) compound of Formula I:

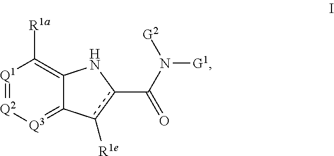

or a pharmaceutically acceptable salt or solvate thereof, wherein:
R^{1a} is selected from the group consisting of halogen, alkyl, alkoxy, cycloalkyl, (hydroxy)alkyl, and (cycloalkyl)alkyl;
Q¹ is selected from the group consisting of —C(R^{1b})═ and —N═;
Q² is selected from the group consisting of —C(R^{1c})═ and —N═;
Q³ is selected from the group consisting of —C(R^{1d})═ and —N═;
provided that at least one of Q¹, Q², or Q³ is —C(R^{1b})═, —C(R^{1c})═, or —C(R^{1d})═, respectively;
R^{1b}, R^{1c}, and R^{1d} are each independently selected from the group consisting of hydrogen, halogen, alkyl, alkenyl, (hydroxy)alkyl, and alkoxy;
R^{1e} is selected from the group consisting of hydrogen, halogen, alkyl, cycloalkyl, (hydroxy)alkyl, and (cycloalkyl)alkyl;
[symbol] is a single or double bond;
G¹ is selected from the group consisting of: optionally substituted aryl;
optionally substituted heteroaryl; optionally substituted heterocyclo; optionally substituted cycloalkyl; (aryl)alkyl; (heteroaryl)alkyl; (heterocyclo)alkyl; (amino)(aryl)alkyl; (heteroaryl)(aryl)alkyl; (heteroaryl)(heterocyclo)alkyl; (heteroaryl)(carboxamido)alkyl; (heteroaryl)(cycloalkyl)alkyl; (aryl)(alkoxycarbonyl)alkyl; (cycloalkyl)alkyl; (heteroaryl)(amino)alkyl; (cycloalkyl)(alkoxycarbonyl)alkyl; (heteroaryl)(alkoxycarbonyl)alkyl; (heterocyclo)(cycloalkyl)alkyl; (aryl)(cycloalkyl)alkyl; (aryl)(hydroxy)alkyl; (cycloalkyl)(hydroxy)alkyl; (hydroxy)alkyl; optionally substituted alkyl; (aryl)(haloalkyl)alkyl; (cycloalkyl)(haloalkyl)alkyl; (hydroxy)(haloalkyl)alkyl; and (alkoxycarbonyl)(haloalkyl)alkyl; and
G² is selected from the group consisting of hydrogen and alkyl; or
G¹ and G² taken together with the nitrogen atom to which they are attached form an optionally substituted heterocyclo; or
(b) a pharmaceutical composition comprising the compound of Formula I, a pharmaceutically acceptable salt or solvate thereof, or a pharmaceutical composition comprising a Compound of Formula I, or a pharmaceutically acceptable salt or solvate thereof, and a pharmaceutically acceptable carrier; and
(c) a Second Therapeutic Agent,
wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more DNA repair enzyme inhibitors, one or more SYK inhibitors, or one or more MEK inhibitors, or a combination thereof.

Embodiment 2. The method of Embodiment 1, wherein the Second Therapeutic Agent comprises a BTK inhibitor.

Embodiment 3. The method of Embodiment 2, wherein the BTK inhibitor is ibrutinib, acalabrutinib, or zanubrutinib.

Embodiment 4. The method of any one of Embodiments 1-3, wherein the Second Therapeutic Agent comprises an anti-CD20 monoclonal antibody.

Embodiment 5. The method of Embodiment 4, wherein the anti CD20 monoclonal antibody is rituximab.

Embodiment 6. The method of any one of Embodiments 1-5, wherein the Second Therapeutic Agent comprises a PI3K inhibitor.

Embodiment 7. The method of Embodiment 6, wherein the PI3K inhibitor is copanlisib.

Embodiment 8. The method of any one of Embodiments 1-7, wherein the Second Therapeutic Agent comprises a CDK4/6 inhibitor.

Embodiment 9. The method of Embodiment 8, wherein the CDK4/6 inhibitor is palbociclib.

Embodiment 10. The method of any one of Embodiments 1-9, wherein the Second Therapeutic Agent comprises a CARM1 inhibitor.

Embodiment 11. The method of Embodiment 10, wherein the CARM1 inhibitor is EZM2302.

Embodiment 12. The method of any one of Embodiments 1-11, wherein the Second Therapeutic Agent comprises an alkylating agent.

Embodiment 13. The method of Embodiment 12, wherein the alkylating agent is mafosfamide.

Embodiment 14. The method of any one of Embodiments 1-13, wherein the Second Therapeutic Agent comprises a topoisomerase II inhibitor.

Embodiment 15. The method of Embodiment 14, wherein the topoisomerase II inhibitor is doxorubicin and etoposide.

Embodiment 16. The method of any one of Embodiments 1-15, wherein the Second Therapeutic Agent comprises a vinca alkaloid.

Embodiment 17. The method of Embodiment 16, wherein the vinca alkaloid is vincristine.

Embodiment 18. The method of any one of Embodiments 1-17, wherein the Second Therapeutic Agent comprises a platinum-based drug.

Embodiment 19. The method of Embodiment 18, wherein the platinum-based drug is carboplatin or oxaliplatin.

Embodiment 20. The method of any one of Embodiments 1-19, wherein the Second Therapeutic Agent comprises a nucleoside anticancer agent.

Embodiment 21. The method of Embodiment 20, wherein the nucleoside anticancer agent is gemcitabine.

Embodiment 22. The method of any one of Embodiments 1-21 further comprising administering a therapeutically effective amount of a Third Therapeutic Agent to the subject, wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

Embodiment 23. The method of Embodiment 22, wherein the Third Therapeutic Agent comprises a glucocorticoid receptor agonist.

Embodiment 24. The method of Embodiment 23, wherein the glucocorticoid receptor agonist is dexamethasone or prednisolone.

Embodiment 25. The method of any one of Embodiments 22-24, wherein the Third Therapeutic Agent comprises an immunomodulatory drug.

Embodiment 26. The method of Embodiment 25, wherein the immunomodulatory drug is pomalidomide or lenalidomide.

Embodiment 27. The method of any one of Embodiments 22-26, wherein the Third Therapeutic Agent comprises a proteasome inhibitor.

Embodiment 28. The method of Embodiment 27, wherein the proteasome inhibitor is bortezomib.

Embodiment 29. The method of any one of Embodiments 22-29, wherein the Third Therapeutic Agent comprises a Bcl-2 inhibitor.

Embodiment 30. The method of Embodiment 29, wherein the Bcl-2 inhibitor is venetoclax.

Embodiment 31. The method of any one of Embodiments 22-30, wherein the Third Therapeutic Agent comprises a pleiotropic pathway modulator.

Embodiment 32. The method of Embodiment 31, wherein the pleiotropic pathway modulator is CC-122.

Embodiment 33. The method of any one of Embodiments 22-32, wherein the Third Therapeutic Agent comprises a XPO1 inhibitor.

Embodiment 34. The method of Embodiment 33, wherein the XPO1 inhibitor is selinexor.

Embodiment 35. The method of any one of Embodiments 22-34, wherein the Third Therapeutic Agent comprises a histone deacetylase inhibitor.

Embodiment 36. The method of Embodiment 35, wherein the histone deacetylase inhibitor is panobinostat.

Embodiment 37. The method of any one of Embodiments 22-36, wherein the Third Therapeutic Agent comprises an EZH2 inhibitor.

Embodiment 38. The method of Embodiment 37, wherein the EZH2 inhibitor is tazemetostat.

Embodiment 39. The method of Embodiment 1, wherein the Second Therapeutic Agent comprises a combination set forth in Table 4.

Embodiment 40. The method of Embodiment 1, wherein the Second Therapeutic Agent comprises a combination set forth in Table 5.

Embodiment 41. The method of Embodiment 22, wherein the Second Therapeutic Agent and Third Therapeutic Agent comprise a combination set forth in Table 8.

Embodiment 42. The method of any one of Embodiments 1-21, wherein the Compound of the Disclosure and the Second Therapeutic Agent are administered sequentially.

Embodiment 43. The method of any one of Embodiments 1-21, wherein the Compound of the Disclosure and the Second Therapeutic Agent are administered simultaneously.

Embodiment 44. The method of Embodiments 22-43, wherein the Compound of the Disclosure, the Second Therapeutic Agent, and the Third Therapeutic Agent are administered sequentially.

Embodiment 45. The method of any one of Embodiments 1-44, wherein the subject has cancer.

Embodiment 46. The method of Embodiment 45, wherein the cancer is any one or more of the cancers of Table 2.

Embodiment 47. The method of Embodiment 45, wherein the cancer is a hematological cancer.

Embodiment 48. The method of Embodiment 47, wherein the hematological cancer is any one or more of the cancers of Table 3.

Embodiment 49. The method of Embodiment 48, wherein the hematological cancer is multiple myeloma, mantle cell lymphoma, or diffuse large B-cell lymphoma.

Embodiment 50. The method of Embodiment 49, wherein the hematological cancer is t(4;14) multiple myeloma.

Embodiment 51. A compound of Formula I, or a pharmaceutically acceptable salt or solvate thereof, see Embodiment 1, or a pharmaceutical composition comprising a compound of Formula I, or a pharmaceutically acceptable salt or solvate thereof, and a pharmaceutically acceptable carrier for use in treating cancer in a subject, wherein the compound or composition is to be administered in combination with a Second Therapeutic Agent, and the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

Embodiment 52. The compound or composition for use of Embodiment 51, wherein the Second Therapeutic Agent comprises a BTK inhibitor.

Embodiment 53. The compound or composition for use of Embodiment 52, wherein the BTK inhibitor is ibrutinib, acalabrutinib, or zanubrutinib.

Embodiment 54. The compound or composition for use of any one of Embodiments 51-53, wherein the Second Therapeutic Agent comprises an anti-CD20 monoclonal antibody.

Embodiment 55. The compound or composition for use of Embodiment 54, wherein the anti CD20 monoclonal antibody is rituximab.

Embodiment 56. The compound or composition for use of any one of Embodiments 51-55, wherein the Second Therapeutic Agent comprises a PI3K inhibitor.

Embodiment 57. The compound or composition for use of Embodiment 56, wherein the PI3K inhibitor is copanlisib.

Embodiment 58. The compound or composition for use of any one of Embodiments 51-57, wherein the Second Therapeutic Agent comprises a CDK4/6 inhibitor.

Embodiment 59. The compound or composition for use of Embodiment 58, wherein the CDK4/6 inhibitor is palbociclib.

Embodiment 60. The compound or composition for use of any one of Embodiments 51-59, wherein the Second Therapeutic Agent comprises a CARM1 inhibitor.

Embodiment 61. The compound or composition for use of Embodiment 60, wherein the CARM1 inhibitor is EZM2302.

Embodiment 62. The compound or composition for use of any one of Embodiments 51-61, wherein the Second Therapeutic Agent comprises an alkylating agent.

Embodiment 63. The compound or composition for use of Embodiment 62, wherein the alkylating agent is mafosfamide.

Embodiment 64. The compound or composition for use of any one of Embodiments 51-63, wherein the Second Therapeutic Agent comprises a topoisomerase II inhibitor.

Embodiment 65. The compound or composition for use of Embodiment 64, wherein the topoisomerase II inhibitor is doxorubicin and etoposide.

Embodiment 66. The compound or composition for use of any one of Embodiments 51-65, wherein the Second Therapeutic Agent comprises a vinca alkaloid.

Embodiment 67. The compound or composition for use of Embodiment 66, wherein the vinca alkaloid is vincristine.

Embodiment 68. The compound or composition for use of any one of Embodiments 51-67, wherein the Second Therapeutic Agent comprises a platinum-based drug.

Embodiment 69. The compound or composition for use of Embodiment 68, wherein the platinum-based drug is carboplatin or oxaliplatin.

Embodiment 70. The compound or composition for use of any one of Embodiments 51-69, wherein the Second Therapeutic Agent comprises a nucleoside anticancer agent.

Embodiment 71. The compound or composition for use of Embodiment 70, wherein the nucleoside anticancer agent is gemcitabine.

Embodiment 72. The compound or composition for use of any one of Embodiments 51-71 further comprising a therapeutically effective amount of a Third Therapeutic Agent to be administered to the subject, wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

Embodiment 73. The compound or composition for use of Embodiment 72, wherein the Third Therapeutic Agent comprises a glucocorticoid receptor agonist.

Embodiment 74. The compound or composition for use of Embodiment 73, wherein the glucocorticoid receptor agonist is dexamethasone or prednisolone.

Embodiment 75. The compound or composition for use of any one of Embodiments 72-74, wherein the Third Therapeutic Agent comprises an immunomodulatory drug.

Embodiment 76. The compound or composition for use of Embodiment 75, wherein the immunomodulatory drug is pomalidomide or lenalidomide.

Embodiment 77. The compound or composition for use of any one of Embodiments 72-76, wherein the Third Therapeutic Agent comprises a proteasome inhibitor.

Embodiment 78. The compound or composition for use of Embodiment 77, wherein the proteasome inhibitor is bortezomib.

Embodiment 79. The compound or composition for use of any one of Embodiments 72-79, wherein the Third Therapeutic Agent comprises a Bcl-2 inhibitor.

Embodiment 80. The compound or composition for use of Embodiment 79, wherein the Bcl-2 inhibitor is venetoclax.

Embodiment 81. The compound or composition for use of any one of Embodiments 72-80, wherein the Third Therapeutic Agent comprises a pleiotropic pathway modulator.

Embodiment 82. The compound or composition for use of Embodiment 81, wherein the pleiotropic pathway modulator is CC-122.

Embodiment 83. The compound or composition for use of any one of Embodiments 72-82, wherein the Third Therapeutic Agent comprises a XPO1 inhibitor.

Embodiment 84. The compound or composition for use of Embodiment 73, wherein the XPO1 inhibitor is selinexor.

Embodiment 85. The compound or composition for use of any one of Embodiments 72-84, wherein the Third Therapeutic Agent comprises a histone deacetylase inhibitor.

Embodiment 86. The compound or composition for use of Embodiment 75, wherein the histone deacetylase inhibitor is panobinostat.

Embodiment 87. The compound or composition for use of any one of Embodiments 72-86, wherein the Third Therapeutic Agent comprises an EZH2 inhibitor.

Embodiment 88. The compound or composition for use of Embodiment 87, wherein the EZH2 inhibitor is tazemetostat.

Embodiment 89. The compound or composition for use of Embodiment 51, wherein the Second Therapeutic Agent comprises a combination set forth in Table 4.

Embodiment 90. The compound or composition for use of Embodiment 51, wherein the Second Therapeutic Agent comprises a combination set forth in Table 5.

Embodiment 91. The compound or composition for use of Embodiment 72, wherein the Second Therapeutic Agent and Third Therapeutic Agent comprise a combination set forth in Table 8.

Embodiment 92. The compound or composition for use of any one of Embodiments 51-71, wherein the Compound of the Disclosure and the Second Therapeutic Agent are to be administered sequentially.

Embodiment 93. The compound or composition for use of any one of Embodiments 51-71, wherein the Compound of the Disclosure and the Second Therapeutic Agent are to be administered simultaneously.

Embodiment 94. The compound or composition for use of Embodiments 72-93, wherein the Compound of the Disclosure, the Second Therapeutic Agent, and the Third Therapeutic Agent are to be administered sequentially.

Embodiment 95. The compound or composition for use of any one of Embodiments 51-94, wherein the subject has cancer.

Embodiment 96. The compound or composition for use of Embodiment 95, wherein the cancer is any one or more of the cancers of Table 2.

Embodiment 97. The compound or composition for use of Embodiment 95, wherein the cancer is a hematological cancer.

Embodiment 98. The compound or composition for use of Embodiment 97, wherein the hematological cancer is any one or more of the cancers of Table 3.

Embodiment 99. The compound or composition for use of Embodiment 98, wherein the hematological cancer is multiple myeloma, mantle cell lymphoma, or diffuse large B-cell lymphoma.

Embodiment 100. The compound or composition for use of Embodiment 99, wherein the hematological cancer is t(4;14) multiple myeloma.

Embodiment 101. Use of a compound of Formula I, or a pharmaceutically acceptable salt or solvate thereof, see Embodiment 1, or a pharmaceutical composition comprising a compound of Formula I, or a pharmaceutically acceptable salt or solvate thereof, and a pharmaceutically acceptable carrier for the manufacture of a medicament for treating cancer in a subject, wherein the compound or composition is to be administered in combination with a Second Therapeutic Agent and the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, or one or more CARM1 inhibitors, one or more inhibitors of enzymes of DNA damage repair, one or more SYK inhibitors, or one or more MEK inhibitors, or a combination thereof.

Embodiment 102. The use of Embodiment 101, wherein the Second Therapeutic Agent comprises a BTK inhibitor.

Embodiment 103. The use of Embodiment 102, wherein the BTK inhibitor is ibrutinib, acalabrutinib, or zanubrutinib.

Embodiment 104. The use of any one of Embodiments 101-103, wherein the Second Therapeutic Agent comprises an anti-CD20 monoclonal antibody.

Embodiment 105. The use of Embodiment 104, wherein the anti CD20 monoclonal antibody is rituximab.

Embodiment 106. The use of any one of Embodiments 101-105, wherein the Second Therapeutic Agent comprises a PI3K inhibitor.

Embodiment 107. The use of Embodiment 106, wherein the PI3K inhibitor is copanlisib.

Embodiment 108. The use of any one of Embodiments 101-107, wherein the Second Therapeutic Agent comprises a CDK4/6 inhibitor.

Embodiment 109. The use of Embodiment 108, wherein the CDK4/6 inhibitor is palbociclib.

Embodiment 110. The use of any one of Embodiments 101-109, wherein the Second Therapeutic Agent comprises a CARM1 inhibitor.

Embodiment 111. The use of Embodiment 110, wherein the CARM1 inhibitor is EZM2302.

Embodiment 112. The use of any one of Embodiments 101-111, wherein the Second Therapeutic Agent comprises an alkylating agent.

Embodiment 113. The use of Embodiment 112, wherein the alkylating agent is mafosfamide.

Embodiment 114. The use of any one of Embodiments 101-113, wherein the Second Therapeutic Agent comprises a topoisomerase II inhibitor.

Embodiment 115. The use of Embodiment 114, wherein the topoisomerase II inhibitor is doxorubicin and etoposide.

Embodiment 116. The use of any one of Embodiments 51-115, wherein the Second Therapeutic Agent comprises a vinca alkaloid.

Embodiment 117. The use of Embodiment 116, wherein the vinca alkaloid is vincristine.

Embodiment 118. The use of any one of Embodiments 101-117, wherein the Second Therapeutic Agent comprises a platinum-based drug.

Embodiment 119. The use of Embodiment 118, wherein the platinum-based drug is carboplatin or oxaliplatin.

Embodiment 120. The use of any one of Embodiments 101-119, wherein the Second Therapeutic Agent comprises a nucleoside anticancer agent.

Embodiment 121. The use of Embodiment 120, wherein the nucleoside anticancer agent is gemcitabine.

Embodiment 122. The use of any one of Embodiments 101-121 further comprising a therapeutically effective amount of a Third Therapeutic Agent to be administered to the subject, wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

Embodiment 123. The use of Embodiment 122, wherein the Third Therapeutic Agent comprises a glucocorticoid receptor agonist.

Embodiment 124. The use of Embodiment 123, wherein the glucocorticoid receptor agonist is dexamethasone or prednisolone.

Embodiment 125. The use of any one of Embodiments 122-124, wherein the Third Therapeutic Agent comprises an immunomodulatory drug.

Embodiment 126. The use of Embodiment 125, wherein the immunomodulatory drug is pomalidomide or lenalidomide.

Embodiment 127. The use of any one of Embodiments 122-126, wherein the Third Therapeutic Agent comprises a proteasome inhibitor.

Embodiment 128. The use of Embodiment 127, wherein the proteasome inhibitor is bortezomib.

Embodiment 129. The use of any one of Embodiments 122-129, wherein the Third Therapeutic Agent comprises a Bcl-2 inhibitor.

Embodiment 130. The use of Embodiment 129, wherein the Bcl-2 inhibitor is venetoclax.

Embodiment 131. The use of any one of Embodiments 122-130, wherein the Third Therapeutic Agent comprises a pleiotropic pathway modulator.

Embodiment 132. The use of Embodiment 131, wherein the pleiotropic pathway modulator is CC-122.

Embodiment 133. The use of any one of Embodiments 122-132, wherein the Third Therapeutic Agent comprises a XPO1 inhibitor.

Embodiment 134. The use of Embodiment 123, wherein the XPO1 inhibitor is selinexor.

Embodiment 135. The use of any one of Embodiments 122-134, wherein the Third Therapeutic Agent comprises a histone deacetylase inhibitor.

Embodiment 136. The use of Embodiment 125, wherein the histone deacetylase inhibitor is panobinostat.

Embodiment 137. The use of any one of Embodiments 122-136, wherein the Third Therapeutic Agent comprises an EZH2 inhibitor.

Embodiment 138. The use of Embodiment 137, wherein the EZH2 inhibitor is tazemetostat.

Embodiment 139. The use of Embodiment 101, wherein the Second Therapeutic Agent comprises a combination set forth in Table 4.

Embodiment 140. The use of Embodiment 101, wherein the Second Therapeutic Agent comprises a combination set forth in Table 5.

Embodiment 141. The use of Embodiment 122, wherein the Second Therapeutic Agent and Third Therapeutic Agent comprise a combination set forth in Table 8.

Embodiment 142. The use of any one of Embodiments 101-121, wherein the Compound of the Disclosure and the Second Therapeutic Agent are to be administered sequentially.

Embodiment 143. The use of any one of Embodiments 101-121, wherein the Compound of the Disclosure and the Second Therapeutic Agent are to be administered simultaneously.

Embodiment 144. The use of Embodiments 122-143, wherein the Compound of the Disclosure, the Second Therapeutic Agent, and the Third Therapeutic Agent are to be administered sequentially.

Embodiment 145. The use of any one of Embodiments 101-144, wherein the subject has cancer.

Embodiment 146. The use of Embodiment 145, wherein the cancer is any one or more of the cancers of Table 2.

Embodiment 147. The use of Embodiment 145, wherein the cancer is a hematological cancer.

Embodiment 148. The use of Embodiment 147, wherein the hematological cancer is any one or more of the cancers of Table 3.

Embodiment 149. The use of Embodiment 148, wherein the hematological cancer is multiple myeloma, mantle cell lymphoma, or diffuse large B-cell lymphoma.

Embodiment 150. The use of Embodiment 149, wherein the hematological cancer is t(4;14) multiple myeloma.

Embodiment 151. A kit for carrying out the method of any one of claims 1-50 or the use of any one of claims 51-150, the kit comprising: (a) a therapeutically effective amount compound of Formula I, or a pharmaceutically acceptable salt or solvate thereof, or (b) a pharmaceutical composition comprising a compound of Formula I, or a pharmaceutically acceptable salt or solvate thereof, and a pharmaceutically acceptable carrier; and (c) a therapeutically effective amount of a Second Therapeutic Agent, wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof and, optionally, (d) instructions for administering the Compound or Composition of the Disclosure and the Second Therapeutic Agent to the subject.

Embodiment 152. The kit of Embodiment 151, wherein the Second Therapeutic Agent comprises a BTK inhibitor.

Embodiment 153. The kit of Embodiment 152, wherein the BTK inhibitor is ibrutinib, acalabrutinib, or zanubrutinib.

Embodiment 154. The kit of any one of Embodiments 151-153, wherein the Second Therapeutic Agent comprises an anti-CD20 monoclonal antibody.

Embodiment 155. The kit of Embodiment 154, wherein the anti CD20 monoclonal antibody is rituximab.

Embodiment 156. The kit of any one of Embodiments 151-155, wherein the Second Therapeutic Agent comprises a PI3K inhibitor.

Embodiment 157. The kit of Embodiment 156, wherein the PI3K inhibitor is copanlisib.

Embodiment 158. The kit of any one of Embodiments 151-157, wherein the Second Therapeutic Agent comprises a CDK4/6 inhibitor.

Embodiment 159. The kit of Embodiment 158, wherein the CDK4/6 inhibitor is palbociclib.

Embodiment 160. The kit of any one of Embodiments 151-159, wherein the Second Therapeutic Agent comprises a CARM1 inhibitor.

Embodiment 161. The kit of Embodiment 160, wherein the CARM1 inhibitor is EZM2302.

Embodiment 162. The kit of any one of Embodiments 151-161, wherein the Second Therapeutic Agent comprises an alkylating agent.

Embodiment 163. The kit of Embodiment 162, wherein the alkylating agent is mafosfamide.

Embodiment 164. The kit of any one of Embodiments 151-163, wherein the Second Therapeutic Agent comprises a topoisomerase II inhibitor.

Embodiment 165. The kit of Embodiment 164, wherein the topoisomerase II inhibitor is doxorubicin and etoposide.

Embodiment 166. The kit of any one of Embodiments 51-165, wherein the Second Therapeutic Agent comprises a vinca alkaloid.

Embodiment 167. The kit of Embodiment 166, wherein the vinca alkaloid is vincristine.

Embodiment 168. The kit of any one of Embodiments 151-167, wherein the Second Therapeutic Agent comprises a platinum-based drug.

Embodiment 169. The kit of Embodiment 168, wherein the platinum-based drug is carboplatin or oxaliplatin.

Embodiment 170. The kit of any one of Embodiments 151-169, wherein the Second Therapeutic Agent comprises a nucleoside anticancer agent.

Embodiment 171. The kit of Embodiment 170, wherein the nucleoside anticancer agent is gemcitabine.

Embodiment 172. The kit of any one of Embodiments 151-171 further comprising a therapeutically effective amount of a Third Therapeutic Agent to be administered to the subject, wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

Embodiment 173. The kit of Embodiment 172, wherein the Third Therapeutic Agent comprises a glucocorticoid receptor agonist.

Embodiment 174. The kit of Embodiment 173, wherein the glucocorticoid receptor agonist is dexamethasone or prednisolone.

Embodiment 175. The kit of any one of Embodiments 172-174, wherein the Third Therapeutic Agent comprises an immunomodulatory drug.

Embodiment 176. The kit of Embodiment 175, wherein the immunomodulatory drug is pomalidomide or lenalidomide.

Embodiment 177. The kit of any one of Embodiments 172-176, wherein the Third Therapeutic Agent comprises a proteasome inhibitor.

Embodiment 178. The kit of Embodiment 177, wherein the proteasome inhibitor is bortezomib.

Embodiment 179. The kit of any one of Embodiments 172-179, wherein the Third Second Therapeutic Agent comprises a Bcl-2 inhibitor.

Embodiment 180. The kit of Embodiment 179, wherein the Bcl-2 inhibitor is venetoclax.

Embodiment 181. The kit of any one of Embodiments 172-180, wherein the Third Therapeutic Agent comprises a pleiotropic pathway modulator.

Embodiment 182. The kit of Embodiment 181, wherein the pleiotropic pathway modulator is CC-122.

Embodiment 183. The kit of any one of Embodiments 172-182, wherein the Third Therapeutic Agent comprises a XPO1 inhibitor.

Embodiment 184. The kit of Embodiment 173, wherein the XPO1 inhibitor is selinexor

Embodiment 185. The kit of any one of Embodiments 172-184, wherein the Third Therapeutic Agent comprises a histone deacetylase inhibitor.

Embodiment 186. The kit of Embodiment 185, wherein the histone deacetylase inhibitor is panobinostat.

Embodiment 187. The kit of any one of Embodiments 172-186, wherein the Third Therapeutic Agent comprises an EZH2 inhibitor.

Embodiment 188. The kit of Embodiment 187, wherein the EZH2 inhibitor is tazemetostat.

Embodiment 189. The kit of Embodiment 151, wherein the Second Therapeutic Agent comprises a combination set forth in Table 4.

Embodiment 190. The kit of Embodiment 151, wherein the Second Therapeutic Agent comprises a combination set forth in Table 5.

Embodiment 191. The kit of Embodiment 172, wherein the Second Therapeutic Agent and Third Therapeutic Agent comprise a combination set forth in Table 8.

Embodiment 192. The kit of any one of Embodiments 151-171, wherein the Compound of the Disclosure and the Second Therapeutic Agent are to be administered sequentially.

Embodiment 193. The kit of any one of Embodiments 151-171, wherein the Compound of the Disclosure and the Second Therapeutic Agent are to be administered simultaneously.

Embodiment 194. The kit of Embodiments 172-193, wherein the Compound of the Disclosure, the Second Therapeutic Agent, and the Third Therapeutic Agent are to be administered sequentially.

Embodiment 195. The kit of any one of Embodiments 151-194, wherein the subject has cancer.

Embodiment 196. The kit of Embodiment 195, wherein the cancer is any one or more of the cancers of Table 2.

Embodiment 197. The kit of Embodiment 195, wherein the cancer is a hematological cancer.

Embodiment 198. The kit of Embodiment 197, wherein the hematological cancer is any one or more of the cancers of Table 3.

Embodiment 199. The kit of Embodiment 198, wherein the hematological cancer is multiple myeloma, mantle cell lymphoma, or diffuse large B-cell lymphoma.

Embodiment 200. The kit of Embodiment 199, wherein the hematological cancer is t(4;14) multiple myeloma.

Embodiment 201. A kit comprising (a) a therapeutically effective amount of compound of Formula I, or a pharmaceutically acceptable salt or solvate thereof, see Embodiment 1, or (b) a pharmaceutical composition comprising a compound of Formula I, or a pharmaceutically acceptable salt or solvate thereof, and a pharmaceutically acceptable carrier; and (c) a therapeutically effective amount of a Second Therapeutic Agent, wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof and, optionally, (d) instructions for administering the Compound or Composition of the Disclosure and the Second Therapeutic Agent to a subject.

Embodiment 202. The kit of Embodiment 201, wherein the Second Therapeutic Agent comprises a BTK inhibitor.

Embodiment 203. The kit of Embodiment 202, wherein the BTK inhibitor is ibrutinib, acalabrutinib, or zanubrutinib.

Embodiment 204. The kit of any one of Embodiments 201-203, wherein the Second Therapeutic Agent comprises an anti-CD20 monoclonal antibody.

Embodiment 205. The kit of Embodiment 204, wherein the anti CD20 monoclonal antibody is rituximab.

Embodiment 206. The kit of any one of Embodiments 201-205, wherein the Second Therapeutic Agent comprises a PI3K inhibitor.

Embodiment 207. The kit of Embodiment 206, wherein the PI3K inhibitor is copanlisib.

Embodiment 208. The kit of any one of Embodiments 201-207, wherein the Second Therapeutic Agent comprises a CDK4/6 inhibitor.

Embodiment 209. The kit of Embodiment 208, wherein the CDK4/6 inhibitor is palbociclib.

Embodiment 210. The kit of any one of Embodiments 201-209, wherein the Second Therapeutic Agent comprises a CARM1 inhibitor.

Embodiment 211. The kit of Embodiment 210, wherein the CARM1 inhibitor is EZM2302.

Embodiment 212. The kit of any one of Embodiments 201-211, wherein the Second Therapeutic Agent comprises an alkylating agent.

Embodiment 213. The kit of Embodiment 212, wherein the alkylating agent is mafosfamide.

Embodiment 214. The kit of any one of Embodiments 201-213, wherein the Second Therapeutic Agent comprises a topoisomerase II inhibitor.

Embodiment 215. The kit of Embodiment 214, wherein the topoisomerase II inhibitor is doxorubicin and etoposide.

Embodiment 216. The kit of any one of Embodiments 51-215, wherein the Second Therapeutic Agent comprises a vinca alkaloid.

Embodiment 217. The kit of Embodiment 216, wherein the vinca alkaloid is vincristine.

Embodiment 218. The kit of any one of Embodiments 201-217, wherein the Second Therapeutic Agent comprises a platinum-based drug.

Embodiment 219. The kit of Embodiment 218, wherein the platinum-based drug is carboplatin or oxaliplatin.

Embodiment 220. The kit of any one of Embodiments 201-219, wherein the Second Therapeutic Agent comprises a nucleoside anticancer agent.

Embodiment 221. The kit of Embodiment 220, wherein the nucleoside anticancer agent is gemcitabine.

Embodiment 222. The kit of any one of Embodiments 201-221 further comprising a therapeutically effective amount of a Third Therapeutic Agent to be administered to the subject, wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

Embodiment 223. The kit of Embodiment 222, wherein the Third Therapeutic Agent comprises a glucocorticoid receptor agonist.

Embodiment 224. The kit of Embodiment 223, wherein the glucocorticoid receptor agonist is dexamethasone or prednisolone.

Embodiment 225. The kit of any one of Embodiments 222-224, wherein the Third Therapeutic Agent comprises an immunomodulatory drug.

Embodiment 226. The kit of Embodiment 225, wherein the immunomodulatory drug is pomalidomide or lenalidomide.

Embodiment 227. The kit of any one of Embodiments 222-226, wherein the Third Therapeutic Agent comprises a proteasome inhibitor.

Embodiment 228. The kit of Embodiment 227, wherein the proteasome inhibitor is bortezomib.

Embodiment 229. The kit of any one of Embodiments 222-229, wherein the Third Therapeutic Agent comprises a Bcl-2 inhibitor.

Embodiment 230. The kit of Embodiment 229, wherein the Bcl-2 inhibitor is venetoclax.

Embodiment 231. The kit of any one of Embodiments 222-230, wherein the Third Therapeutic Agent comprises a pleiotropic pathway modulator.

Embodiment 232. The kit of Embodiment 231, wherein the pleiotropic pathway modulator is CC-122.

Embodiment 233. The kit of any one of Embodiments 222-232, wherein the Third Therapeutic Agent comprises a XPO1 inhibitor.

Embodiment 234. The kit of Embodiment 223, wherein the XPO1 inhibitor is selinexor

Embodiment 235. The kit of any one of Embodiments 222-234, wherein the Third Therapeutic Agent comprises a histone deacetylase inhibitor.

Embodiment 236. The kit of Embodiment 235, wherein the histone deacetylase inhibitor is panobinostat.

Embodiment 237. The kit of any one of Embodiments 222-236, wherein the Third Therapeutic Agent comprises an EZH2 inhibitor.

Embodiment 238. The kit of Embodiment 237, wherein the EZH2 inhibitor is tazemetostat.

Embodiment 239. The kit of Embodiment 201, wherein the Second Therapeutic Agent comprises a combination set forth in Table 4.

Embodiment 240. The kit of Embodiment 201, wherein the Second Therapeutic Agent comprises a combination set forth in Table 5.

Embodiment 241. The kit of Embodiment 222, wherein the Second Therapeutic Agent and Third Therapeutic Agent comprise a combination set forth in Table 8.

Embodiment 242. The kit of any one of Embodiments 201-221, wherein the Compound of the Disclosure and the Second Therapeutic Agent are to be administered sequentially.

Embodiment 243. The kit of any one of Embodiments 201-221, wherein the Compound of the Disclosure and the Second Therapeutic Agent are to be administered simultaneously.

Embodiment 244. The kit of Embodiments 222-243, wherein the Compound of the Disclosure, the Second Therapeutic Agent, and the Third Therapeutic Agent are to be administered sequentially.

Embodiment 245. The kit of any one of Embodiments 201-244, wherein the subject has cancer.

Embodiment 246. The kit of Embodiment 245, wherein the cancer is any one or more of the cancers of Table 2.

Embodiment 247. The kit of Embodiment 245, wherein the cancer is a hematological cancer.

Embodiment 248. The kit of Embodiment 247, wherein the hematological cancer is any one or more of the cancers of Table 3.

Embodiment 249. The kit of Embodiment 248, wherein the hematological cancer is multiple myeloma, mantle cell lymphoma, or diffuse large B-cell lymphoma.

Embodiment 250. The kit of Embodiment 249, wherein the hematological cancer is t(4;14) multiple myeloma.

Embodiment 251. The method of any one of Embodiments 1-50, the compound for use of any one of claims 51-100, the use of any one of claims 101-150, or the kit of any one of Embodiments 151-250, wherein [symbol] is double bond.

Embodiment 252. The method, compound for use, use, or kit of Embodiment 251, wherein the compound is a compound of Formula II:

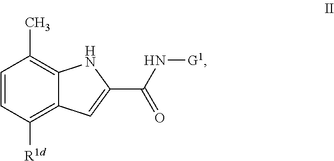

or a pharmaceutically acceptable salt or solvate thereof.

Embodiment 253. The method, compound for use, use, or kit of Embodiments 251 or 252, wherein G¹ is selected from the group consisting of: optionally substituted C₆-C₁₀ aryl; optionally substituted 5- to 9-membered heteroaryl; optionally substituted 3- to 10-membered heterocyclo; optionally substituted C₆-C₈ cycloalkyl; (5- to 9-membered heteroaryl)C₁-C₆ alkyl; (5- to 9-membered heteroaryl)(C₆₋₁₀ aryl)C₁-C₄ alkyl; (5- to 9-membered heteroaryl heteroaryl)(C₃-C₆ cycloalkyl)C₁-C₄ alkyl; and (C₃-C₆ cycloalkyl)C₁-C₄ alkyl, or a pharmaceutically acceptable salt or solvate thereof.

Embodiment 254. The method, compound for use, use, or kit of Embodiment 253, wherein the compound is a compound of Formula IV:

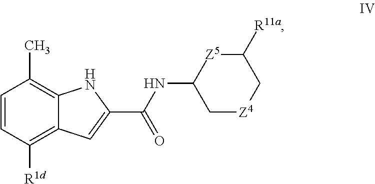

wherein:
Z⁴ is selected from the group consisting of —O—, —C(R^{28a})(R^{28b})—, and —N(R²³)—; or Z⁴ is absent;
Z⁵ is selected from the group consisting of —CH₂— and —CH₂CH₂—;
R^{11a} is selected from the group consisting of optionally substituted alkyl, optionally substituted heterocyclo, optionally substituted heteroaryl, and —N(R^{12b})C(═O)R^{13c};
R^{12b} is selected from the group consisting of hydrogen, alkyl, cycloalkyl, and heterocyclo, (C₁-C₄ alkoxy)C₁-C₄ alkyl, and (hydroxy)C₁-C₄ alkyl; and
R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, amino, (amino)alkyl, (C₃-C₆ cycloalkyl)oxy, and (4- to 8-membered heterocyclo)oxy;
R²³ is selected from the group consisting of hydrogen and C₁-C₄ alkyl; and
R^{28a} and R^{28b} are independently selected from the group consisting of hydrogen, alkyl, and halo;
or a pharmaceutically acceptable salt or solvate thereof.

Embodiment 255. The method, compound for use, use, or kit of Embodiment 254, wherein the compound is a compound of Formula IV-A:

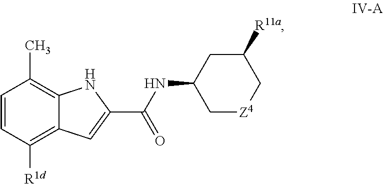

or a pharmaceutically acceptable salt or solvate thereof.

Embodiment 256. The method, compound for use, use, or kit of Embodiment 254, wherein the compound is a compound of Formula IV-B:

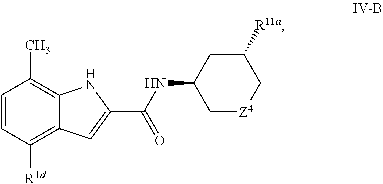

or a pharmaceutically acceptable salt or solvate thereof.

Embodiment 257. The method, compound for use, use, or kit of Embodiment 254, wherein the compound is a compound of Formula IV-C:

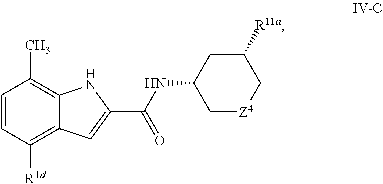

or a pharmaceutically acceptable salt or solvate thereof.

Embodiment 258. The method, compound for use, use, or kit of Embodiment 254, wherein the compound is a compound of Formula IV-D:

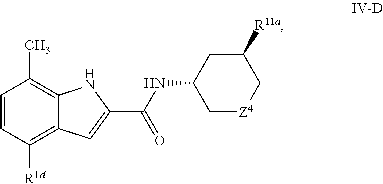

or a pharmaceutically acceptable salt or solvate thereof.

Embodiment 259. The method, compound for use, use, or kit of any one of Embodiments 254-258, wherein:
R^{11a} is selected from the group consisting of:
(A) unsubstituted 4- to 14-membered heterocyclo;
(B) substituted 4- to 14-membered heterocyclo having one, two or three substituents independently selected from the group consisting of:
(i) —N(R^{12a})C(═O)R^{13a}; (ii) —C(═O)R^{13b}; (iii) C₁-C₄ alkyl; (iv) (C₁-C₄ alkoxy)C₁-C₄ alkyl; (v) (hydroxy)C₁-C₄ alkyl; (vi) C₁-C₄ haloalkyl; (vii) amino; (vii) hydroxy; (viii) —N(R^{12a})S(═O)₂R²⁴; (ix) —S(═O)₂R²⁴; (x) unsubstituted C₃-C₆ cycloalkyl; (xi) substituted C₃-C₆ cycloalkyl having one or two substituents independently selected from the group consisting of halo, hydroxy, C₁-C₄ alkyl, amino, and (amino)C₁-C₄ alkyl; (xii) unsubstituted 4- to 14-membered heterocyclo; and substituted 4- to 14-membered heterocyclo having one or two substituents independently selected from the group consisting of amino, hydroxy, and C₁-C₄ alkyl; (xiii) —C(═N—R⁶⁰)R⁶¹; and (xiv) —C(═C—NO₂)R⁶⁴;
(C) unsubstituted 5- to 10-membered heteroaryl;
(D) substituted 5- or 6-membered heteroaryl having one, two, three, or four substituents independently selected from the group consisting of halo and C₁-C₄ alkyl;
(E) C₁-C₆ alkyl; and
(F) —N(R^{12b})C(═O)R^{13c};
R^{12a} and R^{12b} are each independently selected from the group consisting of hydrogen, C₁-C₄ alkyl, (C₁-C₄ alkoxy)C₁-C₄ alkyl, and (hydroxy)C₁-C₄ alkyl;
R^{13a}, R^{13b}, and R^{13c} are each independently selected from the group consisting of (A) C₁-C₆ alkyl; (B) C₁-C₆ haloalkyl; (C) unsubstituted C₃-C₆ cycloalkyl; (D) C₁-C₆ alkoxy; (E) (C₁-C₄ alkoxy)C₁-C₄ alkyl; (F) (hydroxy)C₁-C₄ alkyl; (G) (cyano)alkyl; (H) unsubstituted C₆-C₁₀ aryl; (I) substituted C₆-C₁₀ aryl, having one, two, three, or four substituents independently selected from the group consisting of halo, amino, hydroxy, and C₁-C₄ alkyl; (J) unsubstituted 5- or 6-membered heteroaryl; (K) substituted 5- or 6-membered heteroaryl having one, two, three, or four substituents independently selected from the group consisting of halo, amino, hydroxy, and C₁-C₄ alkyl; (L) unsubstituted 4- to 14-membered heterocyclo; (M) substituted 4- to 14-membered heterocyclo having one or two substituents independently selected from the group consisting of amino, hydroxy, and C₁-C₄ alkyl; (N) amino; (O) (amino)alkyl; (P) (C₃-C₆ cycloalkyl)oxy; and (Q) (4- to 8-membered heterocyclo)oxy; and
R²⁴ is selected from the group consisting of C₁-C₄ alkyl and (hydroxy)C₁-C₄ alkyl;
R⁶⁰ is selected from the group consisting of cyano, nitro, hydroxy, C₁-C₆ alkoxy, —C(═O)R⁶², and —S(═O)₂R⁶²;
R⁶¹ is selected from the group consisting of C₁-C₆ alkyl, C₃-C₆ cycloalkyl, and —NR^{63a}R^{63b};
R⁶² is selected from the group consisting of C₁-C₆ alkyl, C₃-C₆ cycloalkyl, and —NR^{63a}R^{63b};
R^{63a} is selected from the group consisting of hydrogen, C₁-C₆ alkyl, and C₃-C₆ cycloalkyl;
R^{63b} is selected from the group consisting of hydrogen, C₁-C₆ alkyl, and C₃-C₆ cycloalkyl; or
R^{63a} and R^{63b} taken together with the nitrogen atom to which they are attached form a 4- to 6-membered optionally substituted heterocyclo;
R⁶⁴ is selected from the group consisting of C₁-C₆ alkyl, C₃-C₆ cycloalkyl, and —NR^{63c}R^{63d};

R⁶³, is selected from the group consisting of hydrogen, C₁-C₆ alkyl, and C₃-C₆ cycloalkyl;
R^{63d} is selected from the group consisting of hydrogen, C₁-C₆ alkyl, and C₃-C₆ cycloalkyl; or
R^{63c} and R^{63d} taken together with the nitrogen atom to which they are attached form a 4- to 6-membered optionally substituted heterocyclo, or a pharmaceutically acceptable salt or solvate thereof.

Embodiment 260. The method, compound for use, use, or kit of Embodiment 259, wherein R^{11a} is a substituted 4- to 14-membered heterocyclo selected from the group consisting of:

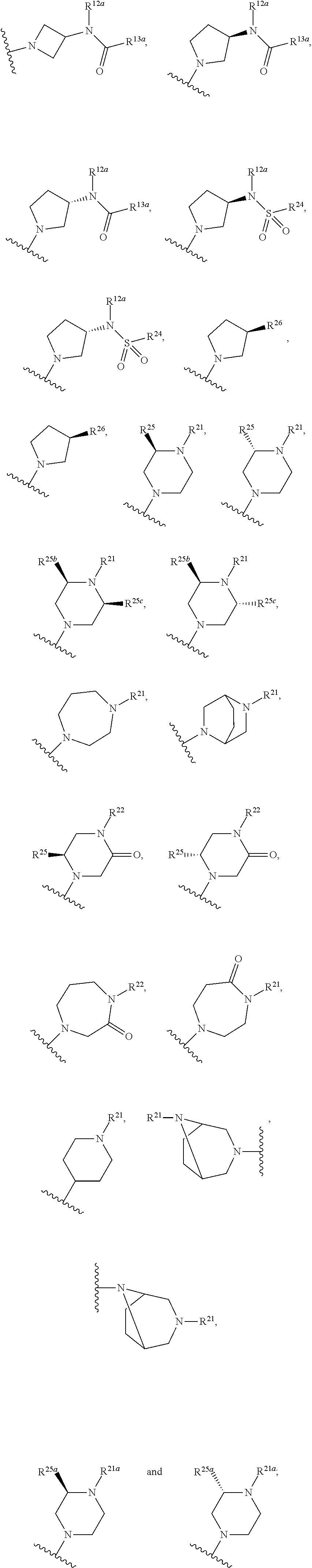

R^{12a} is selected from the group consisting of hydrogen, C₁-C₃ alkyl, (C₁-C₄ alkoxy)C₁-C₄ alkyl; and (hydroxy)C₁-C₄ alkyl;
R^{13a} is selected from the group consisting of C₁-C₄ alkyl; amino; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; (C₁-C₄ alkoxy)C₁-C₄ alkyl; (hydroxy)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;
R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;
R²¹ is selected from the group consisting of hydrogen, —C(═O)R^{13b}, C₁-C₄ alkyl, C₁-C₄ haloalkyl, unsubstituted 4- to 14-membered heterocyclo, and —S(═O)₂R²⁴;
R²² is C₁-C₄ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;
R²⁴ is selected from the group consisting of C₁-C₄ alkyl and (hydroxy)C₁-C₄ alkyl;
R²⁵ is selected from the group consisting of hydrogen, C₁-C₄ alkyl, and C₁-C₄ haloalkyl;
R^{25b} and R^{25c} are independently selected from the group consisting of C₁-C₄ alkyl and C₁-C₄ haloalkyl;
R²⁶ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl; and
R^{21a} and R^{25a} taken together with the atoms to which they are attached form an optionally substituted 4- to 8-membered heterocyclo, or a pharmaceutically acceptable salt or solvate thereof.

Embodiment 261. The method, compound for use, use, or kit of Embodiment 259, wherein R^{11a} is a substituted 4- to 14-membered heterocyclo selected from the group consisting of:

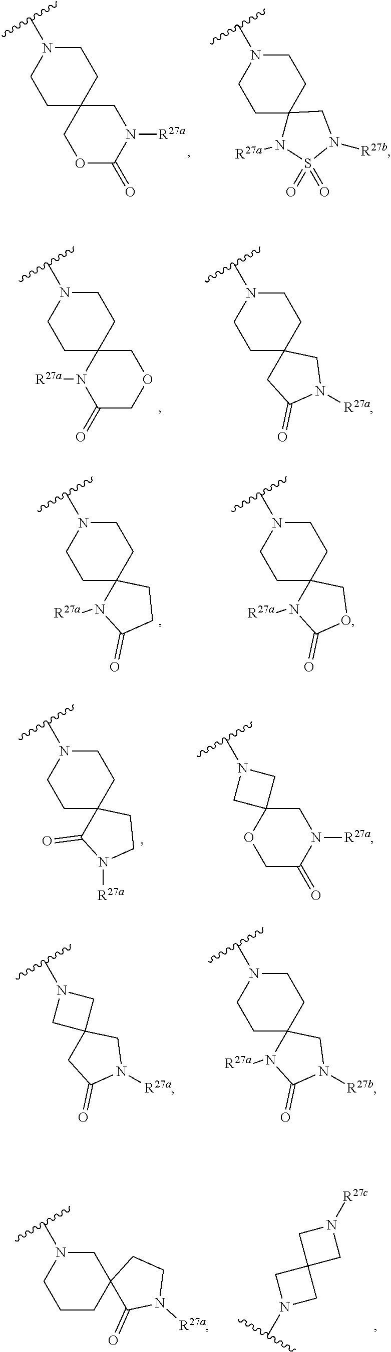

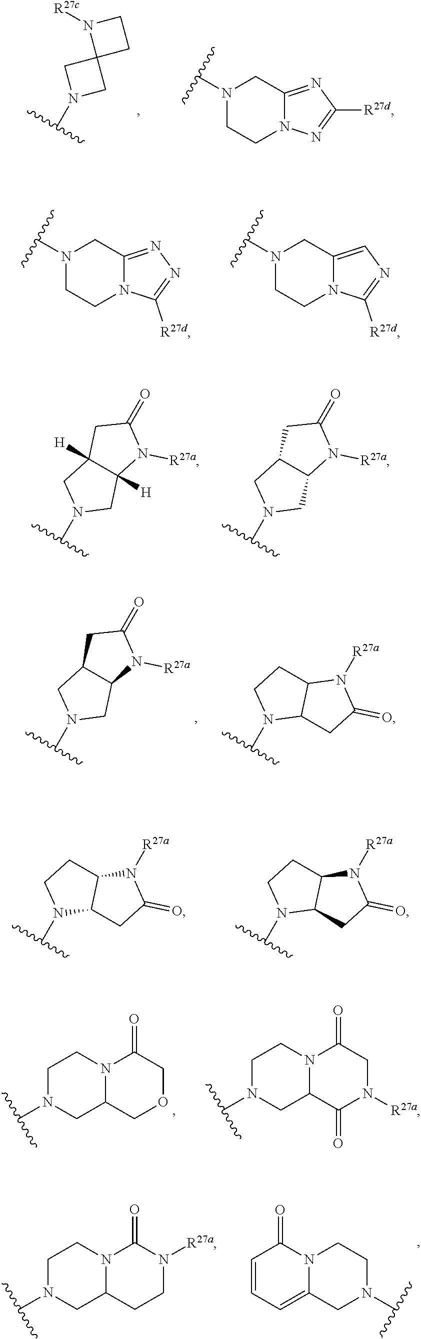

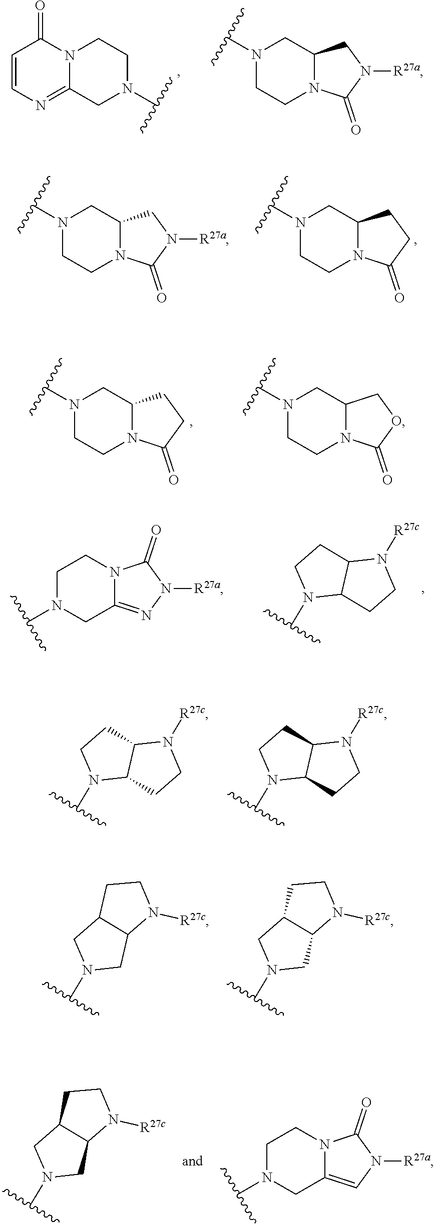

R^{27a} and R^{27b} are each independently selected from the group consisting of hydrogen, C₁-C₄ alkyl, C₁-C₄ haloalkyl, (C₁-C₄ alkoxy)C₁-C₄ alkyl; and (hydroxy)C₁-C₄ alkyl;
R^{27c} is selected from the group consisting of hydrogen; —C(═O)R^{13b}; C₁-C₄ alkyl; C₁-C₄ haloalkyl; unsubstituted 4- to 14-membered heterocyclo; and substituted 4- to 14-membered heterocyclo having one or two substituents independently selected from the group consisting of amino, hydroxy, and C₁-C₄ alkyl; and —S(═O)₂R²⁴;
R^{27d} is selected from the group consisting of hydrogen; C₁-C₄ alkyl; and C₁-C₄ haloalkyl;
R^{13b} is selected from the group consisting of C₁-C₄ alkyl; aminoC₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy; and
R²⁴ is selected from the group consisting of C₁-C₄ alkyl and (hydroxy)C₁-C₄ alkyl, or a pharmaceutically acceptable salt or solvate thereof.

Embodiment 262. The method, compound for use, use, or kit of Embodiment 261, wherein R^{11a} is a substituted 4- to 14-membered heterocyclo selected from the group consisting of:

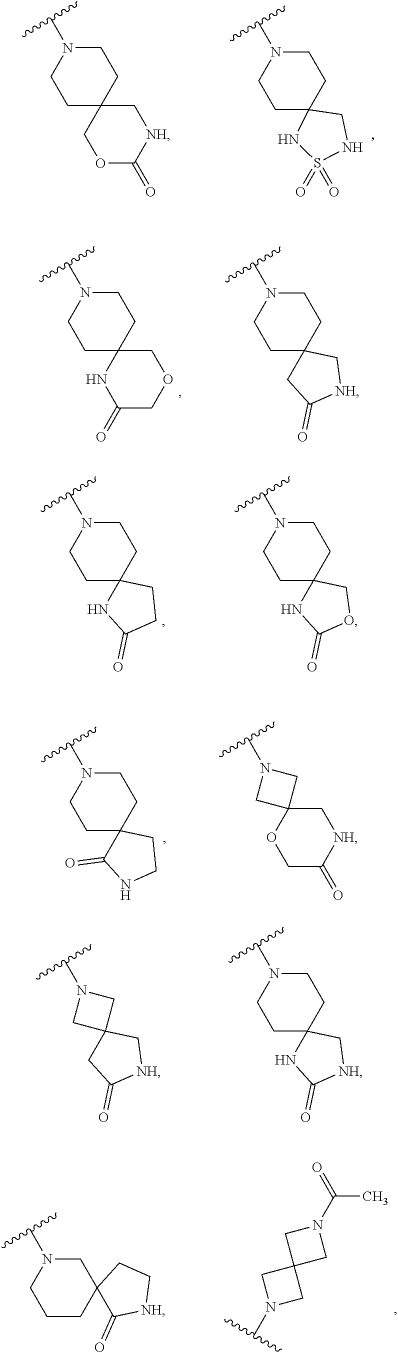

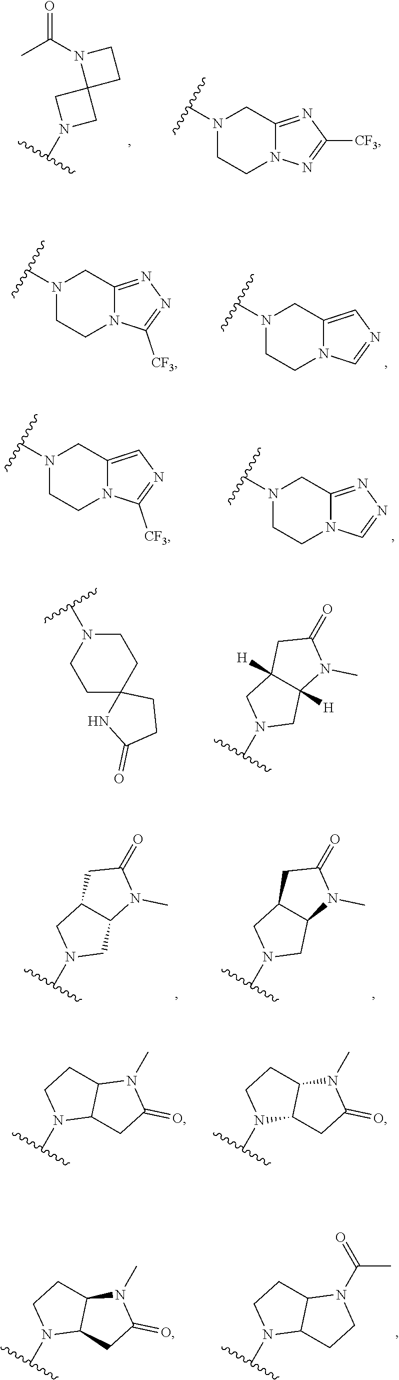

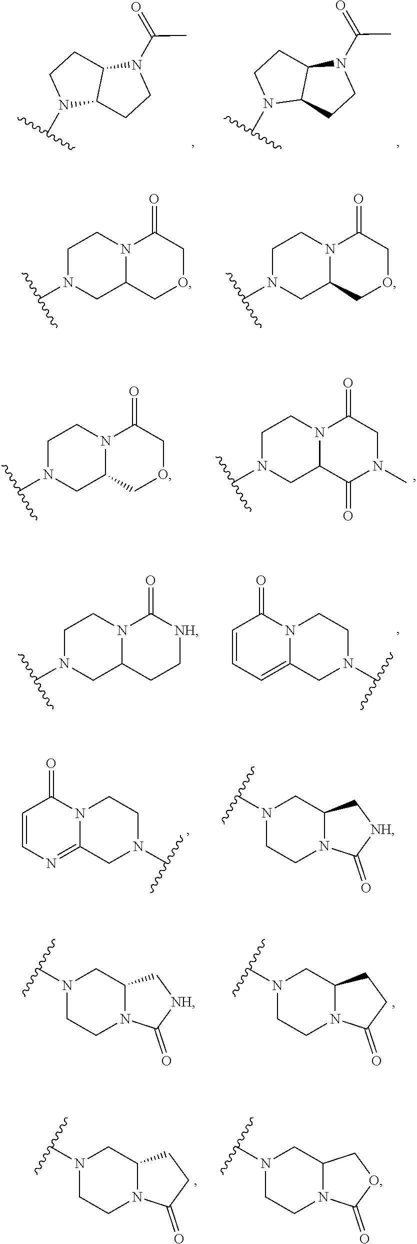

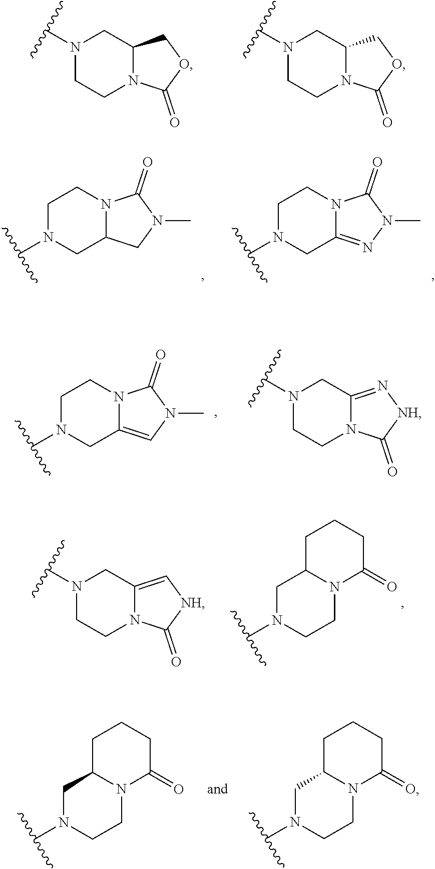

or a pharmaceutically acceptable salt or solvate thereof.

Embodiment 263. The method, compound for use, use, or kit of Embodiment 259, wherein R^{11a} is a substituted 4- to 14-membered heterocyclo selected from the group consisting of

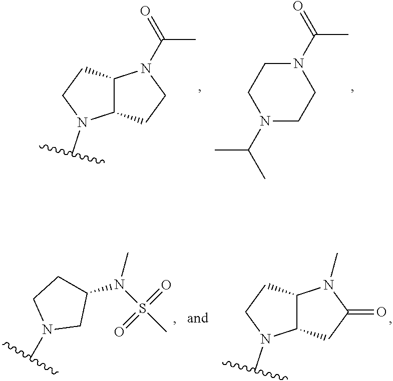

or a pharmaceutically acceptable salt or solvate thereof.

Embodiment 264. The method, compound for use, use, or kit of any one of Embodiments 254-263, wherein Z⁴ is —CH₂—, or a pharmaceutically acceptable salt or solvate thereof.

Embodiment 265. The method, compound for use, use, or kit of any one of Embodiments 251-264, wherein R^{1d} is fluoro, or a pharmaceutically acceptable salt or solvate thereof.

Embodiment 266. The method, compound for use, use, or kit of Embodiment 251, wherein the compound is a compound of Table 1, or a pharmaceutically acceptable salt or solvate thereof.

Embodiment 267. The method, compound for use, use, or kit of Embodiment 251, wherein the compound is a compound of Table 1B, or a pharmaceutically acceptable salt or solvate thereof.

Embodiment 268. The method of any one of Embodiments 1-50 or 251-267, the compound for use of any one of claims 51-100 or 251-267, the use of any one of Embodiments 101-150 or 251-267, or the kit of any one of Embodiments 151-250 or 251-267, wherein the Second Therapeutic Agent comprises a DNA repair enzyme inhibitor.

Embodiment 269. The method, compound for use, use, or kit of Embodiment 268, wherein the DNA repair enzyme inhibitor is an ATM inhibitor, ATR inhibitor, Chk1 inhibitor, Wee1 inhibitor, RAD51 inhibitor, PARP inhibitor, or AKT inhibitor.

Embodiment 270. The method of any one of Embodiments 1-50 or 251-269, the compound for use of any one of claims 51-100 or 251-269, the use of any one of Embodiments 101-150 or 251-269, or the kit of any one of Embodiments 151-250 or 251-269, wherein the Second Therapeutic Agent comprises a SYK inhibitor.

Embodiment 271. The method of any one of Embodiments 1-50 or 251-270, the compound for use of any one of claims 51-100 or 2251-270, the use of any one of Embodiments 101-150 or 251-270, or the kit of any one of Embodiments 151-250 or 251-270, wherein the Second Therapeutic Agent comprises a MEK inhibitor.

Compounds of the Disclosure can be administered to a subject in the form of a raw chemical without any other components present. Compounds of the Disclosure can also be administered to a subject as part of a pharmaceutical composition containing the compound combined with a suitable pharmaceutically acceptable carrier. Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995. Pharmaceutical compositions comprising a Compound of the Disclosure and a pharmaceutically acceptable carrier are collectively referred to as “Compositions of the Disclosure.”

Pharmaceutical compositions within the scope of the present disclosure include all compositions where a Compound of the Disclosure is combined with one or more pharmaceutically acceptable carriers. In one embodiment, the Compound of the Disclosure is present in the composition in an amount that is effective to achieve its intended therapeutic purpose. While individual needs may vary, a determination of optimal ranges of effective amounts of each compound is within the skill of the art. Typically, a Compound of the Disclosure can be administered to a mammal, e.g., a human, orally at a dose of from about 0.0025 to about 1500 mg per kg body weight of the mammal, or an equivalent amount of a pharmaceutically acceptable salt or solvate thereof, per day to treat the particular disorder. A useful oral dose of a Compound of the Disclosure administered to a mammal is from about 0.0025 to about 50 mg per kg body weight of the mammal, or an equivalent amount of the pharmaceutically acceptable salt or solvate thereof. For intramuscular injection, the dose is typically about one-half of the oral dose.

A unit oral dose may comprise from about 0.01 mg to about 1 g of the Compound of the Disclosure, e.g., about 0.01 mg to about 500 mg, about 0.01 mg to about 250 mg, about 0.01 mg to about 100 mg, 0.01 mg to about 50 mg, e.g., about 0.1 mg to about 10 mg, of the compound. The unit dose can be administered one or more times daily, e.g., as one or more tablets or capsules, each containing from about 0.01 mg to about 1 g of the compound, or an equivalent amount of a pharmaceutically acceptable salt or solvate thereof.

A Compound of Disclosure or pharmaceutical composition comprising a Compound of the Disclosure and, optionally a Second Therapeutic Agent can be administered to any subject, e.g., a cancer patient in need thereof, that may experience the beneficial effects of a Compound of the Disclosure. Foremost among such subject are mammals, e.g., humans and companion animals, although the disclosure is not intended to be so limited. In one embodiment, the subject is a human.

A pharmaceutical composition of the present disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

In one embodiment, a pharmaceutical composition of the present disclosure can be administered orally. In another embodiment, a pharmaceutical composition of the present disclosure can be administered orally and is formulated into tablets, dragees, capsules, or an oral liquid preparation. In one embodiment, the oral formulation comprises extruded multiparticulates comprising the Compound of the Disclosure.

Alternatively, a pharmaceutical composition of the present disclosure can be administered rectally, and is formulated in suppositories.

Alternatively, a pharmaceutical composition of the present disclosure can be administered by injection.

Alternatively, a pharmaceutical composition of the present disclosure can be administered transdermally.

Alternatively, a pharmaceutical composition of the present disclosure can be administered by inhalation or by intranasal or transmucosal administration.

Alternatively, a pharmaceutical composition of the present disclosure can be administered by the intravaginal route.

A pharmaceutical composition of the present disclosure can contain from about 0.01 to 99 percent by weight, e.g., from about 0.25 to 75 percent by weight, of a Compound of the Disclosure, e.g., about 1%, about 5%, about 10%, about 15%, about 20%, about 25%, about 30%, about 35%, about 40%, about 45%, about 50%, about 55%, about 60%, about 65%, about 70%, or about 75% by weight of a Compound of the Disclosure.

A pharmaceutical composition of the present disclosure is manufactured in a manner which itself will be known in view of the instant disclosure, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which can be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers can be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

In some embodiments, the Compound of the Disclosure, the Second Therapeutic Agent and, optionally, the Third Therapeutic Agent are administered in combination to a subject as part of a single pharmaceutical composition.

In some embodiments, the Compound of the Disclosure, the Second Therapeutic Agent and, optionally, the Third Therapeutic Agent are administered in combination to a subject separately, e.g., as two or more separate pharmaceutical compositions. For example, the Second Therapeutic Agent may comprise one of a BTK inhibitor, an anti-CD20 monoclonal antibody, an alkylating agent, a topoisomerase II inhibitor, a vinca alkaloid, a platinum-based drug, a nucleoside anticancer agent, a PI3K inhibitor, a CDK4/6 inhibitor, a CARM1 inhibitor, an inhibitor of an enzyme of DNA damage repair, a SYK inhibitor, or a MEK inhibitor. In this case, two separate pharmaceutical compositions—one comprising the Compound of the Disclosure and one comprising the Second Therapeutic Agent—are administered to a subject. The Second Therapeutic Agent may comprise a combination of two of a BTK inhibitor, an anti-CD20 monoclonal antibody, an alkylating agent, a topoisomerase II inhibitor, a vinca alkaloid, a platinum-based drug, a nucleoside anticancer agent, a PI3K inhibitor, a CDK4/6 inhibitor, a CARM1 inhibitor, an inhibitor of an enzyme of DNA damage repair, a SYK inhibitor, or a MEK inhibitor. In this case, three separate pharmaceutical compositions—one comprising the Compound of the Disclosure, one comprising the first Second Therapeutic Agent, and one comprising the second Second Therapeutic Agent—are administered to a subject. Likewise, if the Second Therapeutic Agent comprises a combination of, e.g., three or more of a BTK inhibitor, an anti-CD20 monoclonal antibody, a chemotherapeutic drug, a PI3K inhibitor, a CDK4/6 inhibitor, a CARM1 inhibitor, an inhibitor of an enzyme of DNA damage repair, a SYK inhibitor, or a MEK inhibitor, then three separate pharmaceutical compositions are administered to the subject. Separate pharmaceutical compositions can be administered to the subject, for example, at different periodicities, at different durations, and/or by different administration routes.

In some embodiments, a Compound of the Disclosure is administered to the patient prior to the Second Therapeutic Agent, e.g., 0.5, 1, 2, 3, 4, 5, 10, 12, or 18 hours, 1, 2, 3, 4, 5, or 6 days, or 1, 2, 3, or 4 weeks prior to the administration of the Second Therapeutic Agent.

In some embodiments, a Compound of the Disclosure is administered after the Second Therapeutic Agent, e.g., 0.5, 1, 2, 3, 4, 5, 10, 12, or 18 hours, 1, 2, 3, 4, 5, or 6 days, or 1, 2, 3, or 4 weeks after the administration of the Second Therapeutic Agent.

In some embodiments, a Compound of the Disclosure and the Second Therapeutic Agent are administered concurrently.

In some embodiments, a Compound of the Disclosure and the Second Therapeutic Agent are administered concurrently but on different schedules, e.g., a Compound of the Disclosure is administered daily while the Second Therapeutic Agent is administered, e.g., once a week, once every two weeks, once every three weeks, or once every four weeks.

A Compound of the Disclosure, a Second Therapeutic Agent and a Third Therapeutic Agent can be administered in any order to a subject. For example, the Compound of the Disclosure can be administered prior the Second Therapeutic Agent and Third Therapeutic Agent, the Compound of the Disclosure can be administered prior to the Second Therapeutic Agent and after the Third Therapeutic Agent, the Compound of the Disclosure can be administered after the Second Therapeutic Agent and Third Therapeutic Agent, and so on.

In practice, a physician determines the actual dosing regimen that is most suitable for an individual patient, which can vary with the age, weight, and response of the particular patient.

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration. The kit further can include a Second Therapeutic Agent. In some embodiments, the kit comprises a Compound of the Disclosure and a Second Therapeutic Agent as separate pharmaceutical compositions.

### V. Biomarkers

In another embodiment, present disclosure provides methods of treating a subject having cancer, e.g., multiple myeloma, comprising (a) determining whether a biomarker is present or absent in a biological sample taken from the subject; and (b) administering a therapeutically effective amount of a Compound of the Disclosure and a Second Therapeutic Agent to the subject if the biomarker is present in the biological sample. See, e.g., Goossens et al., Transl Cancer Res. 4:256-269 (2015); Kamel and Al-Amodi, Genomics Proteomics Bioinformatics 15:220-235 (2017); and Konikova and Kusenda, Neoplasma 50:31-40 (2003).

Biomarkers include, but are not limited to, chromosomal translocations in a cancer, e.g., mulitple myeloma, cell and WHSC1/NSD2/MMSET expression. In one embodiment, the measurable aspect of the biomarker is its expression status. In one embodiment, the measurable aspect of the biomarker is its mutation status.

In one embodiment, the biomarker is WHSC1/NSD2/MMSET expression which is differentially present in a subject of one phenotypic status, e.g., a subject having a hematological cancer, as compared with another phenotypic status, e.g., a normal undiseased subject or a patient having cancer without overexpression WHSC1/NSD2/MMSET. In one embodiment, the biomarker is overexpression of WHSC1/NSD2/MMSET.

Biomarker standards can be predetermined, determined concurrently, or determined after a biological sample is obtained from the subject. Biomarker standards for use with the methods described herein can, for example, include data from samples from subjects without cancer; data from samples from subjects with cancer, e.g., breast cancer, that is not metastatic; and data from samples from subjects with cancer, e.g., breast cancer, that metastatic. Comparisons can be made to establish predetermined threshold biomarker standards for different classes of subjects, e.g., diseased vs. non-diseased subjects. The standards can be run in the same assay or can be known standards from a previous assay.

A biomarker is differentially present between different phenotypic status groups if the mean or median expression or mutation levels of the biomarker is calculated to be different, i.e., higher or lower, between the groups. Thus, biomarkers provide an indication that a subject, e.g., a cancer patient, belongs to one phenotypic status or another.

The determination of the expression level or mutation status of a biomarker in a patient can be performed using any of the many methods known in the art. Any method known in the art for quantitating specific proteins and/or detecting WHSC1/NSD2/MMSET expression and/or chromosomal translocations, or the expression or mutation levels of any other biomarker in a patient or a biological sample may be used in the methods of the disclosure. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al. Cancer 83:1401 (1998); Hudlebusch et al., Clin Cancer Res 17:2919-2933 (2011). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, N Y, (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from the tumor tissue sample using RNAse free techniques. Such techniques are commonly known in the art.

In one embodiment of the disclosure, a biological sample is obtained from the patient and the biological sample is assayed for determination of a biomarker expression or mutation status.

In one embodiment, the present disclosure provides a method of treating a subject having cancer, e.g., multiple myeloma, the method comprising: (a) determining whether a chromosomal translocation is present or absent in a biological sample taken from the subject; and (b) administering a therapeutically effective amount of a Compound of the Disclosure, a Second Therapeutic Agent and, optionally, a Third Therapeutic Agent to the subject if a chromosomal translocation is present in the biological sample.

In another embodiment, the present disclosure provides a method of treating a subject having cancer, e.g., multiple myeloma, the method comprising administering a therapeutically effective amount of a Compound of the Disclosure a Second Therapeutic Agent and, optionally, a Third Therapeutic Agent to the subject having a chromosomal translocation.

In another embodiment, the present disclosure provides a method, comprising administering a therapeutically effective amount of a Compound of the Disclosure, a Second Therapeutic Agent and, optionally, a Third Therapeutic Agent, to a subject in need thereof, wherein: (a) the subject has multiple myeloma; and (b) the multiple myeloma is characterized as having a chromosomal translocation.

In any of the above embodiments, the chromosomal translocation is a t(4;14) translocation.

In one embodiment, the present disclosure provides a method of treating a subject having multiple myeloma, the method comprising: (a) determining whether an overexpression of WHSC1/NSD2/MMSET is present or absent in a biological sample taken from the subject; and (b) administering a therapeutically effective amount of a Compound of the Disclosure, a Second Therapeutic Agent and, optionally, a Third Therapeutic Agent, to the subject if an overexpression of WHSC1/NSD2/MMSET is present in the biological sample.

In one embodiment, the present disclosure provides a method of treating a subject having multiple myeloma, the method comprising administering a therapeutically effective amount of a Compound of the Disclosure, a Second Therapeutic Agent and, optionally, a Third Therapeutic Agent, to the subject if an overexpression of WHSC1/NSD2/MMSET is present in subject.

In another embodiment, the present disclosure provides a method, comprising administering a therapeutically effective amount of a Compound of the Disclosure, a Second Therapeutic Agent and, optionally, a Third Therapeutic Agent, to a subject in need thereof, wherein: (a) the subject has multiple myeloma; and (b) the multiple myeloma is characterized as having an overexpression of WHSC1/NSD2/MMSET.

### VI. Definitions

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R⁵⁶°)S(═O)₂R^{56d}, —C(═O)R⁵⁷, —S(═O)R^{56e}, —S(═O)₂R⁵⁸, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein:
R^{56a} is hydrogen or alkyl;
R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56c} is hydrogen or alkyl;
R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁶, is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁷ is haloalkyl, amino, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted heteroaryl, (C₃-C₆ cycloalkyl)oxy, or (4- to 8-membered heterocyclo)oxy;
R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl;
R⁶⁰ is selected from the group consisting of cyano, nitro, hydroxy, C₁-C₆ alkoxy, —C(═O)R⁶², and —S(═O)₂R⁶²;
R⁶¹ is selected from the group consisting of C₁-C₆ alkyl, C₃-C₆ cycloalkyl, and —NR^{63a}R^{63b};
R⁶² is selected from the group consisting of C₁-C₆ alkyl, C₃-C₆ cycloalkyl, and —NR^{63a}R^{63b}.

R^{63a} is selected from the group consisting of hydrogen, C₁-C₆ alkyl, and C₃-C₆ cycloalkyl;
R^{63b} is selected from the group consisting of hydrogen, C₁-C₆ alkyl, and C₃-C₆ cycloalkyl; or
R^{63a} and R^{63b} taken together with the nitrogen atom to which they are attached form a 4- to 6-membered optionally substituted heterocyclo; R⁶⁴ is selected from the group consisting of C₁-C₆ alkyl, C₃-C₆ cycloalkyl, and —NR^{63c}R^{63d}; and
R^{63c} is selected from the group consisting of hydrogen, C₁-C₆ alkyl, and C₃-C₆ cycloalkyl; R^{63d} is selected from the group consisting of hydrogen, C₁-C₆ alkyl, and C₃-C₆ cycloalkyl; or
R^{63c} and R^{63d} taken together with the nitrogen atom to which they are attached form a 4- to 6-membered optionally substituted heterocyclo.

In one embodiment, the optionally substituted alkyl is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸.

In another embodiment, the optionally substituted alkyl is substituted with two substituents. In another embodiment, the optionally substituted alkyl is substituted with one substituent. In another embodiment, the optionally substituted alkyl is an optionally substituted C₁-C₆ alkyl. In another embodiment, the optionally substituted alkyl is an optionally substituted C₁-C₄ alkyl. In one embodiment, the optionally substituted alkyl is an optionally substituted is a C₁ or C₂ alkyl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl has one carbon-to-carbon triple bond. In another embodiment, the alkynyl is a C₁-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —CH≡CHPh.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl. In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl is a C₁-C₆ alkyl. In another embodiment, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used herein by itself or part of another group refers to a stable straight or branched chain hydrocarbon radical containing 1 to 10 carbon atoms and at least two heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In one embodiment, the heteroalkyl contains two oxygen atoms. In another embodiment, the heteroalkyl contains one oxygen and one nitrogen atom. In another embodiment, the heteroalkyl contains two nitrogen atoms. Non-limiting exemplary heteroalkyl groups include —OCH₂CH₂NH₂, —NHCH₂CH₂OCH₃, and —OCH₂CH₂OCH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, —OR⁵⁹, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein R^{56a}, R^{56b}, R^{56c}, R^{56d}, R^{56e}, R⁵⁷, R⁵⁸, R⁶⁰, R⁶¹, and R⁶⁴ are as defined in connection with the term “optionally substituted alkyl” and R⁵⁹ is (hydroxy)alkyl or (amino)alkyl. Non-limiting exemplary optionally substituted cycloalkyl groups include 3-(4-acetylpiperazin-1-yl)cyclohexyl, 3-(3-(N-methylacetamido)pyrrolidin-1-yl)cyclohexyl, 3-morpholinocyclohexyl, and 3-(pyrimidin-5-yl)cyclohexyl. In one embodiment, the optionally substituted cycloalkyl is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, and —OR⁵⁹.

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

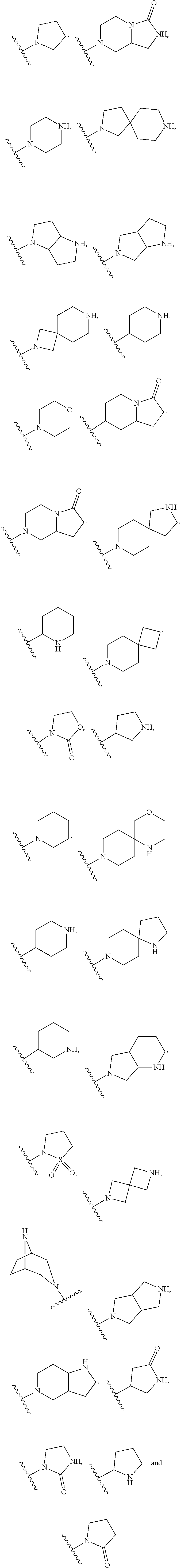

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkyl amino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, —OR⁵⁹, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein R^{56a}, R^{56b}, R^{56c}, R^{56d}, R^{56e}, R⁵⁷, R⁵⁸, R⁵⁹, R⁶⁰, R⁶¹, and R⁶⁴ are as defined in connection with the term “optionally substituted cycloalkyl.” Substitution may occur on any available carbon or nitrogen atom of the heterocyclo group. In one embodiment, the optionally substituted heterocyclo is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56c}, —S(═O)₂R⁵⁸, or —OR⁵⁹.

Non-limiting exemplary optionally substituted heterocyclo groups include:

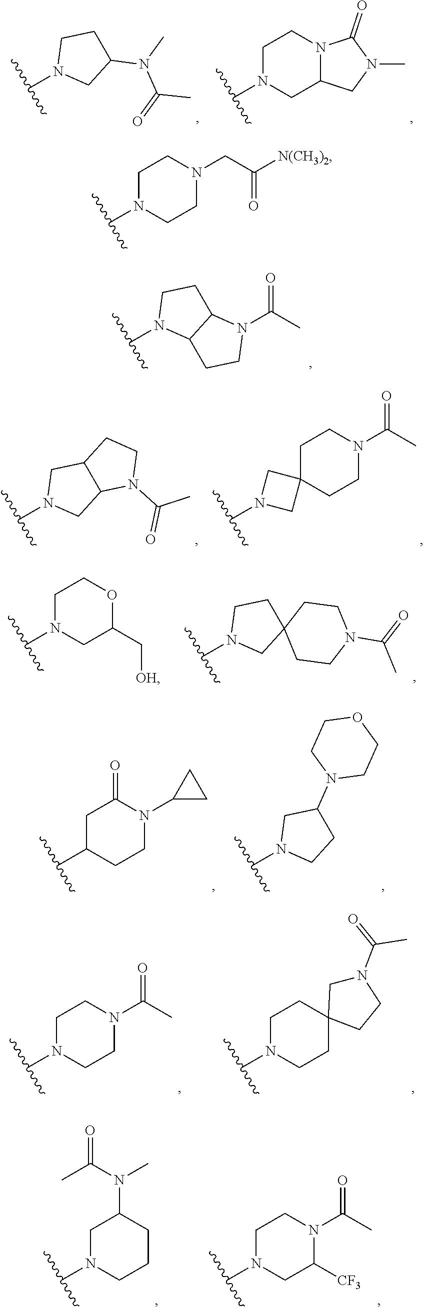

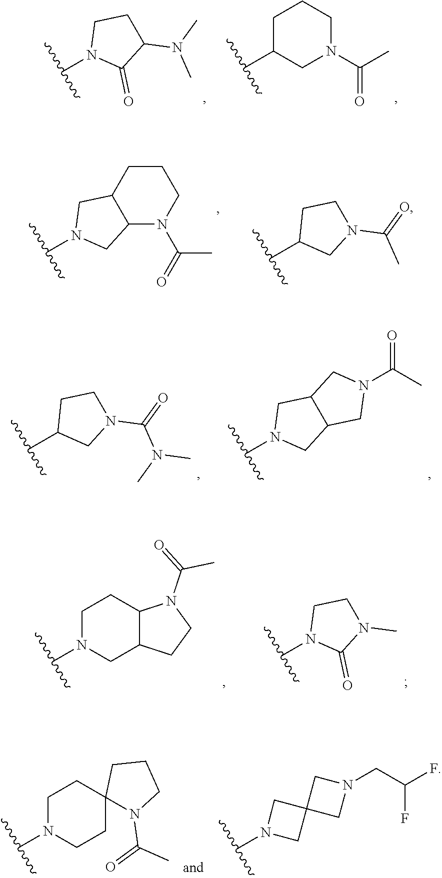

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R⁵⁶¹, —N(R^{56c})S(═O)₂R^{56d}, —C(═O)R⁵⁷, —S(═O)R^{56e}, —S(═O)₂R⁵⁸, —OR⁵⁹, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein R^{56a}, R^{56b}, R^{56c}, R^{56d}, R^{56e}, R⁵⁷, R⁵⁸, R⁵⁹, R⁶⁰, R⁶¹, and R⁶⁴ are as defined in connection with the term “optionally substituted cycloalkyl.” In one embodiment, the optionally substituted aryl is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹.

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, O-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, —OR⁵⁹, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein R^{56a}, R^{56b}, R^{56c}, R^{56d}, R^{56e}, R⁵⁷, R⁵⁸, R⁵⁹, R⁶⁰, R⁶¹, and R⁶⁴ are as defined in connection with the term “optionally substituted cycloalkyl.” In one embodiment, optionally substituted heteroaryl is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹.

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cycloalkyl)oxy” as used herein by itself or as part of another group refers to a cycloalkyl group attached to a terminal oxygen atom. A non-limiting exemplary cycloalkyloxy group is:

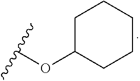 [SMILES: *OC1CCCCC1]

The term “(heterocyclo)oxy” as used herein by itself or as part of another group refers to a heterocyclo group attached to a terminal oxygen atom. A non-limiting exemplary (heterocyclo)oxy group is:

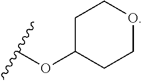 [SMILES: *OC1CCOCC1]

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one optionally substituted cycloalkyl group. In one embodiment, the cycloalkyl group is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (cycloalkyl)alkyl groups include:

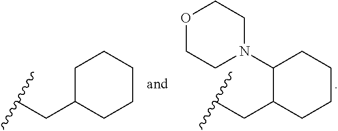 [SMILES: *CC1CCCCC1 ; *CC1CCCCC1N1CCOCC1]

The term “sulfonamido” as use herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

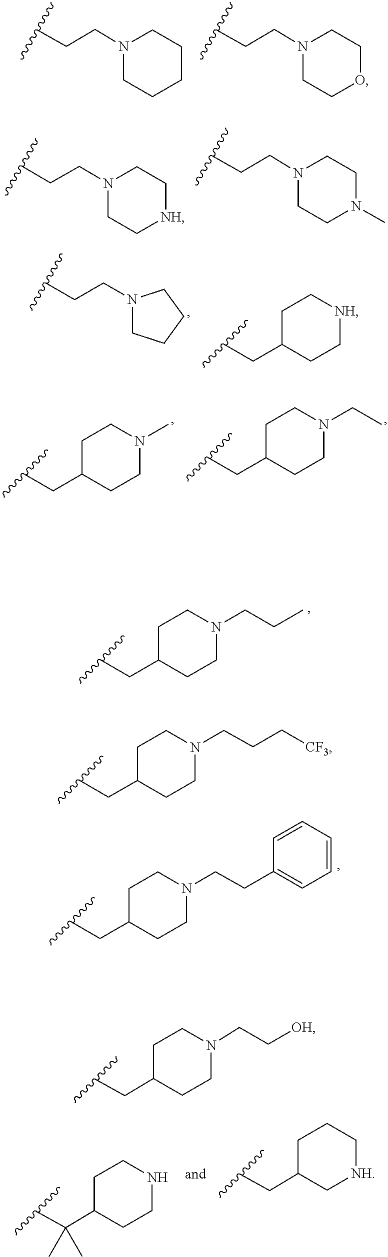

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

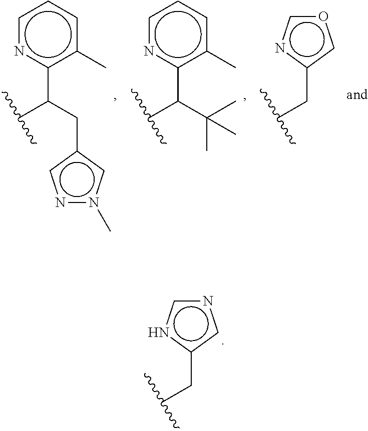

The term “(heteroaryl)(aryl)alkyl” as used herein by itself or as part of another group refers to an alkyl group substituted with one optionally substituted heteroaryl group and one optionally substituted aryl group. In one embodiment, the heteroaryl is an optionally substituted 5- to 9-membered heteroaryl group. In another embodiment, the heteroaryl is an optionally substituted 5- or 6-membered heteroaryl group. In one embodiment, the aryl is an optionally substituted phenyl group or optionally substituted naphthyl group. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (heteroaryl)(aryl)alkyl groups include:

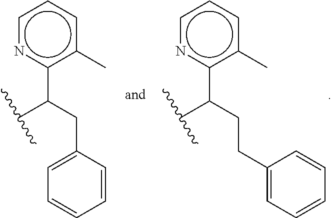

The term “(heteroaryl)(heterocyclo)alkyl” as used herein by itself or as part of another group refers to an alkyl group substituted with one optionally substituted heteroaryl group and one optionally substituted heterocyclo group. In one embodiment, the heteroaryl is an optionally substituted 5- to 9-membered heteroaryl group. In another embodiment, the heteroaryl is an optionally substituted 5- or 6-membered heteroaryl group. In one embodiment, the heterocyclo is an optionally substituted 5- to 8-membered heterocyclo. In another embodiment, the heterocyclo is an optionally substituted 5- or 6-membered heterocyclo. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (heteroaryl)(heterocyclo)alkyl group is:

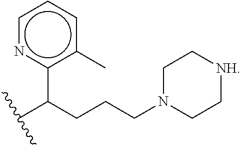

The term “(heteroaryl)(carboxamido)alkyl” as used herein by itself or as part of another group refers to an alkyl group substituted with one optionally substituted heteroaryl group and one carboxamido group. In one embodiment, the heteroaryl is an optionally substituted 5- to 9-membered heteroaryl group. In another embodiment, the heteroaryl is an optionally substituted 5- or 6-membered heteroaryl group. In one embodiment, the alkyl is a C₁-C₆ alkyl. In another embodiment, the alkyl is a C₁-C₄ alkyl. In another embodiment, the alkyl is a C₁-C₃ alkyl. Non-limiting exemplary (heteroaryl)(carboxamido)alkyl groups include:

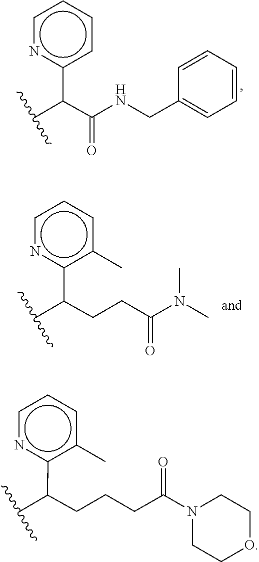

The term “carboxamido” as used herein by itself or as part of another group refers to a radical of formula —C(═O)NR^{55a}R^{55b}, wherein R^{55a} and R^{55b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{55a} and R^{55b} taken together with the nitrogen to which they are attached from a 4- to 8-membered optionally substituted heterocyclo group. Non-limiting exemplary carboxamido groups include: morpholine-4-carbonyl, N,N-dimethylaminocarbonyl, N-(1-methylpiperidin-4-yl)aminocarbonyl, 4-methylpiperazine-1-carbonyl, N-(3-aminocyclopentyl)aminocarbonyl, N-(pyridin-3-yl)aminocarbonyl, and N-(tetrahydrofuran-3-yl)aminocarbonyl.

The term “(heteroaryl)(cycloalkyl)alkyl” as used herein by itself or as part of another group refers to an alkyl group substituted with one optionally substituted heteroaryl group and one optionally substituted cycloalkyl group. In one embodiment, the heteroaryl is an optionally substituted 5- to 9-membered heteroaryl group. In another embodiment, the heteroaryl is an optionally substituted 5- or 6-membered heteroaryl group. In one embodiment, the cycloalkyl is an optionally substituted C₃-C₆ cycloalkyl. In one embodiment, the alkyl is a C₁-C₆ alkyl. In another embodiment, the alkyl is a C₁-C₄ alkyl. In another embodiment, the alkyl is a C₁-C₃ alkyl. A non-limiting exemplary (heteroaryl)(C₃-C₆ cycloalkyl) alkyl group is:

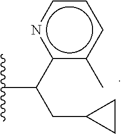

The term “(aryl)(alkoxycarbonyl)alkyl” as used herein by itself or as part of another group refers to an alkyl group substituted with one optionally substituted aryl group and one alkoxycarbonyl group. In one embodiment, the aryl is an optionally substituted phenyl group or optionally substituted naphthyl group. In another embodiment, the aryl is an optionally substituted phenyl group. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (aryl)(alkoxycarbonyl)alkyl group is:

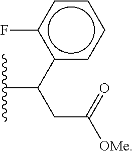

The term “alkoxycarbonyl” as used herein by itself or as part of another group refers to a carbonyl group, i.e., —C(═O)—, substituted by a C₁-C₆ alkoxy group. In one embodiment, the alkoxy group is a C₁-C₄ alkoxy. In another embodiment, the alkoxy group is a C₁-C₃ alkoxy. Non-limiting exemplary alkoxycarbonyl groups include —CO₂-Me and —CO₂Et.

The term “(heteroaryl)(amino)alkyl” as used herein by itself or as part of another group refers to an alkyl group substituted with one optionally substituted heteroaryl group and one amino group. In one embodiment, the heteroaryl is an optionally substituted 5- to 9-membered heteroaryl group. In another embodiment, the heteroaryl is an optionally substituted 5- or 6-membered heteroaryl group. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (heteroaryl)(amino)alkyl group is:

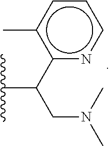

The term “(cycloalkyl)(alkoxycarbonyl)alkyl” as used herein by itself or as part of another group refers to an alkyl group substituted with one optionally substituted cycloalkyl group and one alkoxycarbonyl group. In one embodiment, the cycloalkyl is an optionally substituted C₃-C₆ cycloalkyl. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (cycloalkyl)(alkoxycarbonyl)alkyl group is:

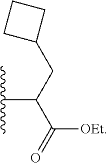 [SMILES: *C(CC1CCC1)C(=O)OCC]

The term “(heteroaryl)(alkoxycarbonyl)alkyl” as used herein by itself or as part of another group refers to an alkyl group substituted with one optionally substituted heteroaryl group and one alkoxycarbonyl group. In one embodiment, the heteroaryl is an optionally substituted 5- to 9-membered heteroaryl group. In another embodiment, the heteroaryl is an optionally substituted 5- or 6-membered heteroaryl group. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (heteroaryl)(alkoxycarbonyl)alkyl groups include:

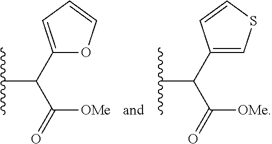 [SMILES: *C(C(=O)OC)c1ccco1 ; *C(C(=O)OC)c1ccsc1]

The term “(heterocyclo)(cycloalkyl)alkyl” as used herein by itself or as part of another group refers to an alkyl group substituted with one optionally substituted heterocyclo group and one optionally substituted cycloalkyl group. In one embodiment, the heterocyclo is an optionally substituted 5- to 8-membered heterocyclo. In another embodiment, the heterocyclo is an optionally substituted 5- or 6-membered heterocyclo. In one embodiment, the cycloalkyl is an optionally substituted C₃-C₆ cycloalkyl. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (heterocyclo)(cycloalkyl)alkyl group is:

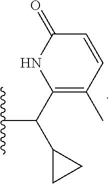 [SMILES: *C(c1[nH]c(=O)ccc1C)C1CC1]

The term “(aryl)(cycloalkyl)alkyl” as used herein by itself or as part of another group refers to an alkyl group substituted with one optionally substituted aryl group and one optionally substituted cycloalkyl group. In one embodiment, the aryl is an optionally substituted phenyl group or optionally substituted naphthyl group. In another embodiment, the aryl is an optionally substituted phenyl group. In one embodiment, the cycloalkyl is an optionally substituted C₃-C₆ cycloalkyl. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (aryl)(cycloalkyl)alkyl group is:

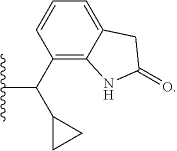 [SMILES: *C(c1cccc2c1NC(=O)C2)C1CC1]

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “(aryl)(hydroxy)alkyl” as used herein by itself or as part of another group refers to an alkyl substituted with one optionally substituted aryl group and one hydroxyl group. In one embodiment, the aryl is an optionally substituted phenyl group or optionally substituted naphthyl group. In another embodiment, the aryl is an optionally substituted phenyl group. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)(hydroxy)alkyl groups include:

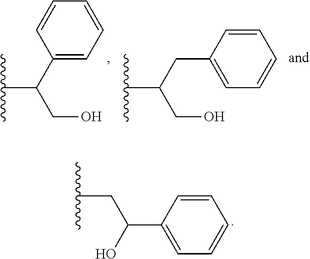 [SMILES: *C(CO)c1ccccc1 ; *C(CO)Cc1ccccc1 ; *CC(O)c1ccccc1]

The term “(cycloalkyl)(hydroxy)alkyl” as used herein by itself or as part of another group refers to an alkyl group substituted with one optionally substituted cycloalkyl group and one hydroxyl group. In one embodiment, the cycloalkyl group is an optionally substituted C₃-C₆ cycloalkyl group. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (cycloalkyl)(hydroxy)alkyl group is:

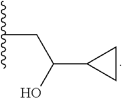 [SMILES: *CC(O)C1CC1]

The term “(alkoxycarbonyl)alkyl” as used herein by itself or as part of another group refers to an alkyl substituted with one or two alkoxycarbonyl groups. In one embodiment, the alkyl is a C₁-C₄ alkyl. In another embodiment, the alkyl is a C₁ or C₂ alkyl. A non-limiting exemplary (alkoxycarbonyl)alkyl groups is:

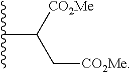 [SMILES: *C(CC(=O)OC)C(=O)OC]

The term “(aryl)(haloalkyl)alkyl” as used herein by itself or as part of another group refers to an alkyl substituted with one optionally substituted aryl group and one haloalkyl group. In one embodiment, the aryl is an optionally substituted group or optionally substituted naphthyl. In another embodiment, the aryl is an optionally substituted phenyl. In one embodiment, the haloalkyl is a C₁-C₄ haloalkyl. In one embodiment, the alkyl is a C₁-C₄ alkyl. In another embodiment, the alkyl is a C₁ or C₂ alkyl. A non-limiting exemplary (aryl)(haloalkyl)alkyl groups is:

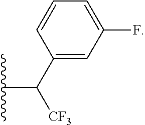 [SMILES: *C(c1cccc(F)c1)C(F)(F)F]

The term “(cycloalkyl)(haloalkyl)alkyl” as used herein by itself or as part of another group refers to an alkyl substituted with one optionally substituted cycloalkyl group and one haloalkyl group. In one embodiment, the cycloalkyl is an optionally substituted C₃-C₆ cycloalkyl. In one embodiment, the haloalkyl is a C₁-C₄ haloalkyl. In one embodiment, the alkyl is a C₁-C₄ alkyl. In another embodiment, the alkyl is a C₁ or C₂ alkyl. A non-limiting exemplary (cycloalkyl)(haloalkyl) alkyl groups is:

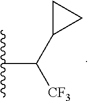 [SMILES: *C(C1CC1)C(F)(F)F]

The term “(hydroxy)(haloalkyl)alkyl” as used herein by itself or as part of another group refers to an alkyl substituted with one hydroxy group and one haloalkyl group. In one embodiment, the haloalkyl is a C₁-C₄ haloalkyl. In one embodiment, the alkyl is a C₁-C₄ alkyl. In another embodiment, the alkyl is a C₁ or C₂ alkyl. A non-limiting exemplary (hydroxy)(haloalkyl)alkyl groups is:

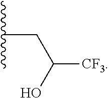 [SMILES: *CC(O)C(F)(F)F]

The term “(alkoxycarbonyl)(haloalkyl)alkyl” as used herein by itself or as part of another group refers to an alkyl substituted with one alkoxycarbonyl group and one haloalkyl group. In one embodiment, the haloalkyl is a C₁-C₄ haloalkyl. In one embodiment, the alkyl is a C₁-C₄ alkyl. In another embodiment, the alkyl is a C₁ or C₂ alkyl. A non-limiting exemplary (alkoxycarbonyl)(haloalkyl)alkyl groups is:

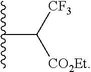 [SMILES: *C(C(=O)OCC)C(F)(F)F]

The term “(carboxamido)alkyl” as used herein by itself or as part of another group refers to an alkyl substituted with a carboxamido group. In one embodiment, the alkyl is a C₁-C₄ alkyl. In another embodiment, the alkyl is a C₁ or C₂ alkyl. Non-limiting exemplary (carboxamido)alkyl groups include —CH₂C(═O)NH₂, —C(H)(CH₃)C(═O)NH₂, —CH₂C(═O)N(H)CH₃, and —CH₂C(═O)N(CH₃)₂.

The term “(carboxy)alkyl” as used herein by itself or as part of another group refers to an alkyl substituted with —C(═O)OH. In one embodiment, the alkyl is a C₁-C₄ alkyl. In another embodiment, the alkyl is a C₁ or C₂ alkyl. A non-limiting exemplary (carboxy)alkyl group is —CH₂CO₂H.

The term “(amino)(hydroxy)alkyl” as used herein by itself or as part of another group refers to an alkyl substituted with one hydroxy group and one amino group. In one embodiment, the alkyl is a C₁-C₄ alkyl. A non-limiting exemplary “(amino)(hydroxy)alkyl group is:

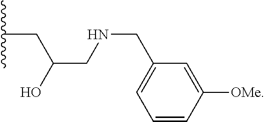 [SMILES: *CC(O)CNCc1cccc(OC)c1]

The term “(amino)(aryl)alkyl” as used herein by itself or as part of another group refers to an alkyl group substituted with one amino group and one optionally substituted aryl group. In one embodiment, the amino group is —NH₂, alkylamino, or dialkylamino. In one embodiment, the aryl group is an optionally substituted phenyl. In one embodiment, the alkyl is a C₁-C₆ alkyl. In another embodiment, the alkyl is a C₁-C₄ alkyl. Non-limiting exemplary (amino)(aryl)alkyl groups include:

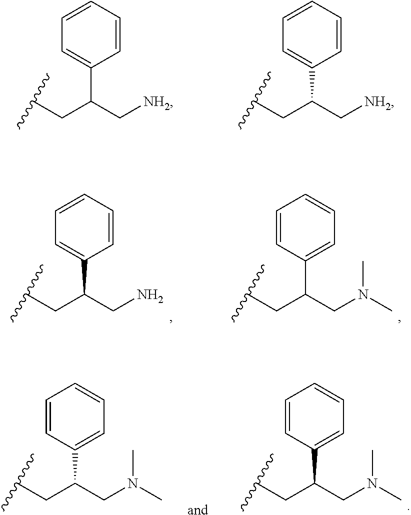

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In another embodiment, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

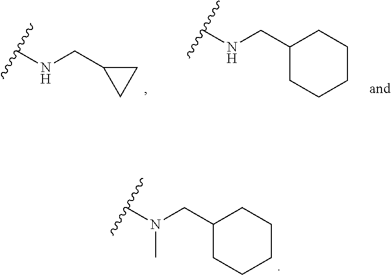

In another embodiment, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

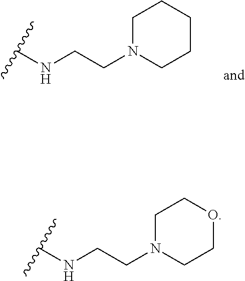 [SMILES: *NCCN1CCCCC1 ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure may contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

In one embodiment, Compounds of the Disclosure having one or more chiral centers are enantiomerically enriched, e.g., the ee is about 5% or more. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

The terms “a” and “an” refer to one or more.

The term “about,” as used herein, includes the recited number±10%. Thus, “about 10” means 9 to 11.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a Compound of the Disclosure to an individual in need of such treatment.

Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; modulate protein methylation in the target cells; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The term “disease” or “condition” or “disorder” denotes disturbances and/or anomalies that as a rule are regarded as being pathological conditions or functions, and that can manifest themselves in the form of particular signs, symptoms, and/or malfunctions. Compounds of the Disclosure inhibit SETD2 protein and can be used in treating diseases and conditions such as proliferative diseases, wherein inhibition of SETD2 protein provides a benefit. See, e.g., U.S. Provisional Appl. No. 62/545,353.

In some embodiments, the Compounds of the Disclosure can be used to treat a “SETD2 protein mediated disorder” A SETD2 protein mediated disorder is any pathological condition in which a SETD2 protein is known to play a role. In some embodiments, a SETD2 mediated disorder is a proliferative disease.

In some embodiments inhibiting SETD2 protein is the inhibition of the activity of one or more activities of SETD2 protein. In some embodiments, the activity of the SETD2 protein is the ability of the SETD2 protein to transfer a methyl group to a target protein, e.g., histone. It should be appreciated that the activity of SETD2 may be inhibited in vitro or in vivo. Exemplary levels of inhibition of the activity of SETD2 include at least 5% inhibition at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to about 100% inhibition.

The term “biological sample” as used herein refers any tissue or fluid from a subject that is suitable for detecting chromosomal translocations. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for chromosomal translocations using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (cIg-FISH). A biological sample can be obtained using techniques that are well within the scope of ordinary knowledge of a clinical practitioner. In one embodiment of the disclosure, the biological sample comprises blood cells.

The phrase “in combination” as used in connection with the administration of a Compound of the Disclosure and a Second Therapeutic Agent to a subject means that the Compound of the Disclosure and the Second Therapeutic Agent can be administered to the subject together, e.g., as part of a single pharmaceutical composition or formulation, or separately, e.g., as part of two or more separate pharmaceutical compositions or formulations. The phrase “in combination” as used in connection with the administration of a Compound of the Disclosure and a Second Therapeutic Agent to a subject is thus intended to embrace administration of a Compound of the Disclosure and a Second Therapeutic Agent in a sequential manner, wherein the Compound of the Disclosure and the Second Therapeutic Agent are administered to the subject at a different time, as well as administration concurrently, or in a substantially simultaneous manner. Simultaneous administration can be accomplished, for example, by administering to the subject a single capsule having a fixed ratio of each of the Compound of the Disclosure and the Second Therapeutic Agent or in multiple, single capsules for each of the Compound of the Disclosure and the Second Therapeutic Agent. Sequential or substantially simultaneous administration of the Compound of the Disclosure and the Second Therapeutic Agent agent can be accomplished by any appropriate route including, but not limited to, oral routes, intravenous routes, intramuscular routes, and direct absorption through mucous membrane tissues. The Compound of the Disclosure and the Second Therapeutic Agent can be administered by the same route or by different routes. For example, the Second Therapeutic Agent of the combination may be administered by intravenous injection while the Compound of the Disclosure of the combination may be administered orally. Alternatively, for example, both the Compound of the Disclosure and the Second Therapeutic Agent may be administered orally or both the Compound of the Disclosure and the Second Therapeutic Agent may be administered by intravenous injection. The Compound of the Disclosure and the Second Therapeutic Agent may also be administered in alternation. In one embodiment, the Compound of the Disclosure and the Second Therapeutic Agent are administered to a subject separately, e.g., as part of two or more separate pharmaceutical compositions or formulations. The same principles apply when a Compound of the Disclosure, a Second Therapeutic Agent, and a Third Therapeutic Agent are administered in combination to a subject. For example, the phrase in combination as used in connection with the administration of a Compound of the Disclosure, a Second Therapeutic Agent, and a Third Therapeutic Agent to a subject is intended to embrace administration of a Compound of the Disclosure, a Second Therapeutic Agent, and a Third Therapeutic agent in a sequential manner, wherein the Compound of the Disclosure, Second Therapeutic Agent, and Third Therapeutic Agent are administered to the subject at different times, as well as administration concurrently, or in a substantially simultaneous manner. In one embodiment, the Compound of the Disclosure, the Second Therapeutic Agent, and the Third Therapeutic Agent are each administered to a subject separately, e.g., as part of three or more separate pharmaceutical compositions or formulations.

### General Synthesis of Compounds

Compounds of the Disclosure are prepared using methods disclosed in WO 2020/037079, or by the illustrative methods shown in the General Schemes below. In the General Schemes, R^{1d}, R^{2b}, R^{2d}, R^{2e}, A¹, A², R^{11a}, R^{14a}, R^{14b}, R¹⁹, R²⁰, G, Z⁴, and q are as defined in connection with Formulae II, III, IV, V, or VI, unless otherwise indicated. In any of the General Schemes, suitable protecting groups can be employed in the synthesis, for example, when Z is (amino)alkyl or any other group that may group that may require protection, or when R⁸ is amino, (amino)alkyl, or any other group that may require protection. (See, Wuts, P. G. M.; Greene, T. W., “Greene's Protective Groups in Organic Synthesis”, 4th Ed., J. Wiley & Sons, N Y, 2007) unless otherwise indicated.

In General Scheme 1, the aryl hydrazine of Formula (1) is reacted with ethyl 2-oxopropanoate to give a compound of Formula (2). In step 2, the compound of Formula (2) is converted to the indole of Formula (3) under acidic conditions. In step 3, the compound of Formula (3) is hydrolyzed to give the indole-2-carboxylic acid of Formula (4). In step 4, a compound of Formula (4) is reacted with G¹NH₂ under standard coupling conditions to give a compound of Formula II.

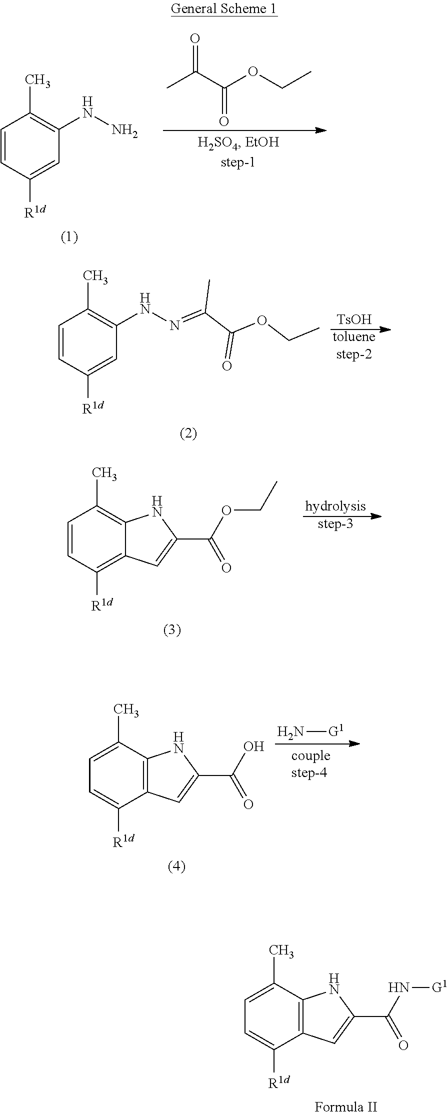

In General Scheme 2, a compound of Formula (5) is reacted with R^{2b}—H wherein R^{2b} is a heterocyclo, e.g., R^{2b}—H is piperidine, or an amine, e.g., R^{2b}—H is dimethyl amine, to give a compound of Formula (6). The nitro group of the compound of Formula (6) is reduced to give a compound of Formula (7). In step 3, the compound of Formula (7) is reacted with a compound of Formula (4), see General Scheme 1, under standard coupling conditions to give a compound of Formula III, wherein A¹ and A² are CH and R^{2b} is an optionally substituted heterocyclo or an amino group.

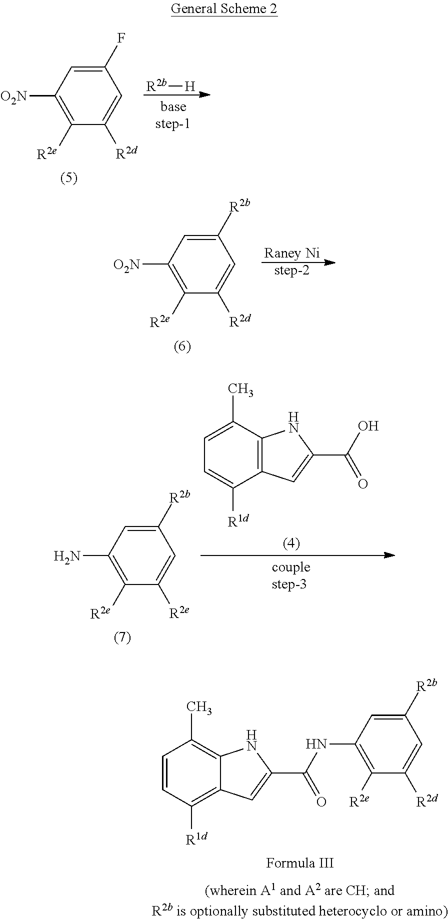

In General Scheme 3, a compound of Formula (8) is reacted with R^{2b}—H wherein R^{2b} is a heterocyclo, e.g., R^{2b}—H is piperidine, or an amine, e.g., R^{2b}—H is dimethyl amine, to give a compound of Formula (9). In step 2, the compound of Formula (9) is reacted with a compound of Formula (10) to give a compound of Formula III, wherein A¹ and/or A² are N and R^{2b} is an optionally substituted heterocyclo or an amino group.

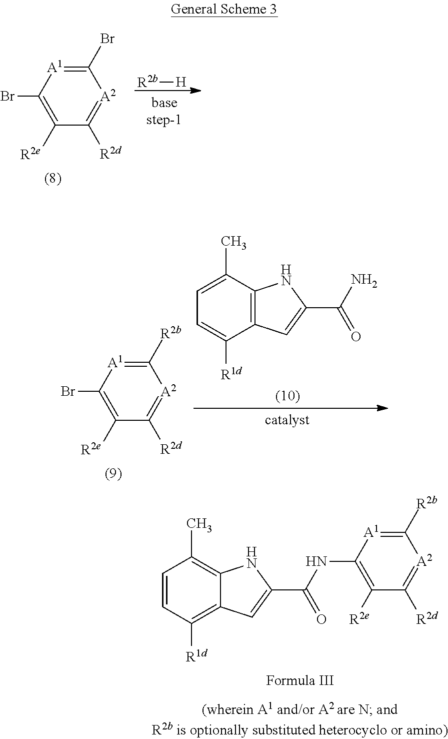

In General Scheme 4, a compound of Formula (11) is reacted with R^{11a}—H, wherein R^{11a} is a heterocyclo, e.g., R^{11a}—H is piperidine, to give a compound of Formula (12). In step 2, the Cbz group is removed to give a compound of Formula (13). The compound of Formula (13) is coupled with a compound of Formula (4) to give a compound of Formula IV, wherein R^{1la} is optionally substituted heterocyclo and Z⁵ is —CH₂—.

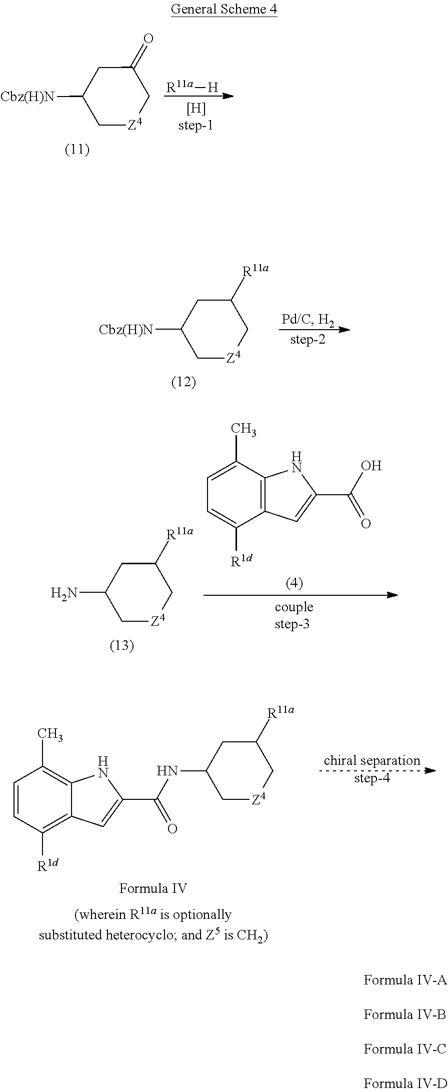

In step 1 of General Scheme 5, a nitrile of Formula (14) is reacted with a Grignard reagent (R^{14a}—MgBr) and the resulting product is reduced to give a compound of Formula (15). The compound of Formula (15) is coupled with a compound of Formula (4) to give a compound of Formula V, wherein p is 0.

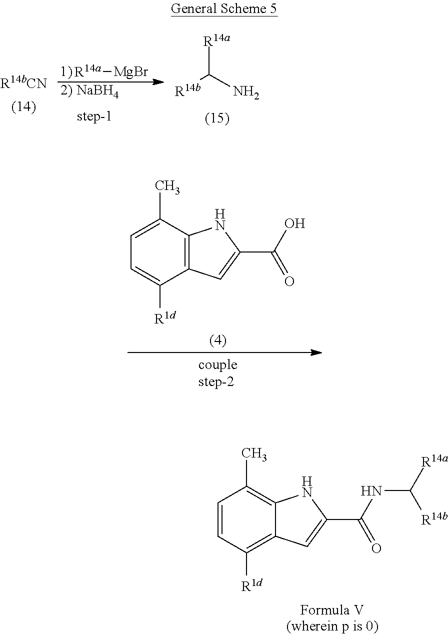

In General Scheme 6, an aldehyde of Formula (16) is reacted with an ester of Formula (17) to give a compound of Formula (18). In step 2, the compound of Formula (18) hydrolyzed to give a compound of Formula (19). In step 3, the compound of Formula (19) is converted to the isocyanate of Formula (20). The compound of Formula (20) is reacted with benzyl alcohol to give a compound of Formula (21). Hydrogenation of a compound of Formula (21) and removal of the Cbz groups gives an amine of Formula (23). Coupling a compound of Formula (23) with a compound of Formula (4) gives a compound of Formula V, wherein p is 1.

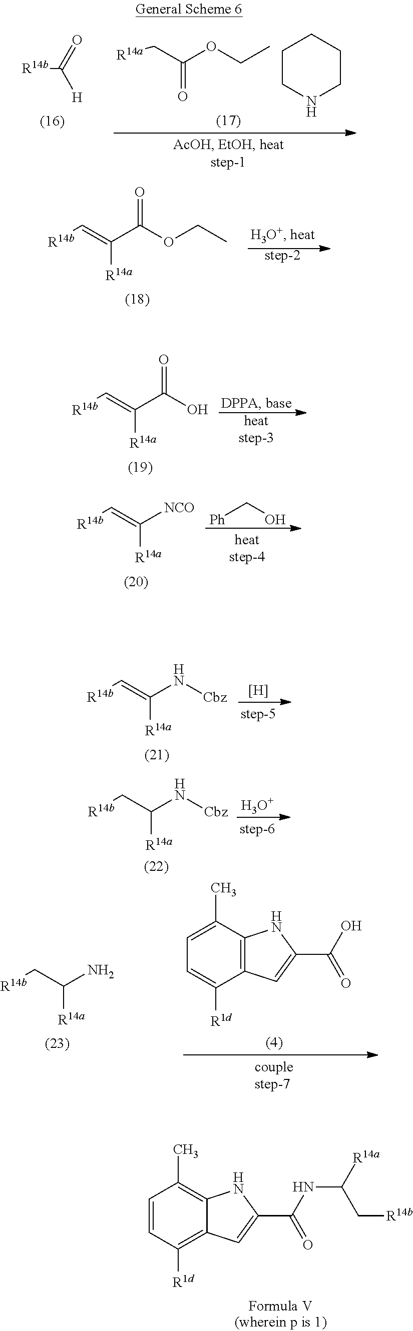

In General Scheme 7, the nitrile of Formula (24) is reduced to give an amine of Formula (25). The compound of Formula (25) is coupled with a compound of Formula (4) to give a compound of Formula VI.

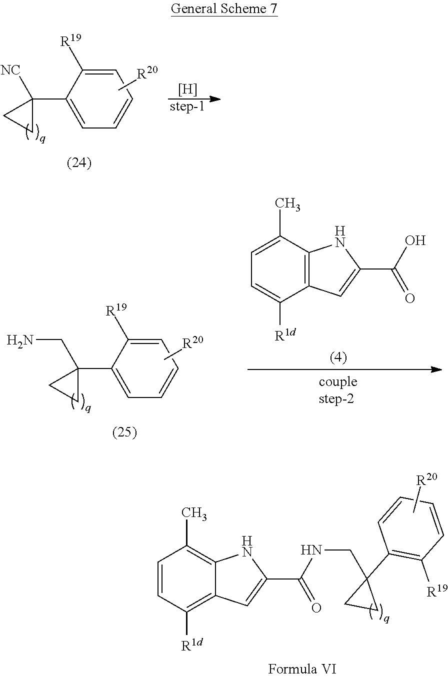

### EXAMPLES

### Example 1

### Synthesis of N-((1R,3S)-3-(4-acetylpiperazin-1-yl)cyclohexyl)-4-fluoro-7-methyl-1H-indole-2-carboxamide (Cpd. No. 15)

### Step 1. Synthesis of ethyl (2E)-2-[2-(5-fluoro-2-methylphenyl)hydrazin-1-ylidene]propanoate

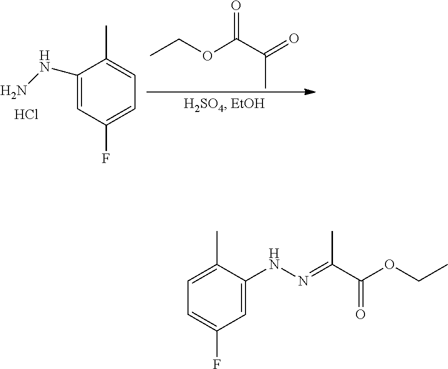

Into a 1000-mL round-bottom flask, was placed a solution of (5-fluoro-2-methylphenyl)hydrazine hydrochloride (100 g, 572.73 mmol, 1.00 equiv) in ethanol (400 mL), ethyl 2-oxopropanoate (66 g, 1.20 equiv), sulfuric acid (10 mL). The resulting solution was stirred for 2 h at 25° C. The reaction progress was monitored by LCMS. The resulting mixture was concentrated under vacuum. The solids were collected by filtration. This resulted in 120 g (yield=88%) of ethyl (2E)-2-[2-(5-fluoro-2-methylphenyl) hydrazin-1-ylidene]propanoate as a yellow solid. LCMS (Method A: ESI): RT=1.399 min, m/z=239.0 [M+H]⁺. ¹H NMR (400 MHz, DMSO-d₆) δ 11.96 (d, J=2.0 Hz, 1H), 7.15 (m, 2H), 6.62 (m, 1H), 4.25 (q, J=7.1 Hz, 2H), 2.12 (d, J=9.3 Hz, 6H), 1.29 (t, J=7.1 Hz, 3H) ppm.

### Step 2. Synthesis of ethyl 4-fluoro-7-methyl-1H-indole-2-carboxylate

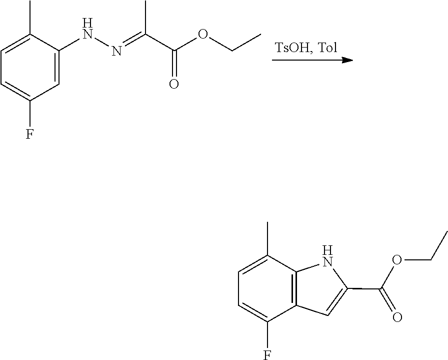 [SMILES: CCOC(=O)/C(C)=N/Nc1cc(F)ccc1C ; CCOC(=O)c1cc2c(F)ccc(C)c2[nH]1]

Into a 1000-mL round-bottom flask, was placed a solution of ethyl (2E)-2-[2-(5-fluoro-2-methylphenyl)hydrazin-1-ylidene]propanoate (40 g, 167.89 mmol, 1.00 equiv) in Toluene (400 mL), 4-methylbenzene-1-sulfonic acid (50 g, 290.36 mmol, 1.70 equiv). The resulting solution was stirred for 18 h at 100° C. The reaction progress was monitored by LCMS. The resulting solution was concentrated under vacuum, and the residue was dissolved by 100 ml of ethyl acetate. The resulting mixture was washed with 3×200 mL of saturated aqueous NaHCO₃. The residue was applied onto a silica gel column with ethyl acetate/petroleum ether (1:5). The resulting mixture was concentrated under vacuum. The solid was purified by recrystallization from ethanol. This resulted in 9.0 g (yield=24%) of ethyl 4-fluoro-7-methyl-1H-indole-2-carboxylate as a yellow solid. LCMS (Method A, ESI): RT=1.354 min: m/z=222.0 [M+H]⁺. ¹H NMR (400 MHz, DMSO-d₆) δ 12.07 (s, 1H), 7.17 (d, J=2.1 Hz, 1H), 7.00 (m, 1H), 6.77 (m, 7.8 Hz, 1H), 4.36 (q, J=7.1 Hz, 2H), 2.49 (d, J=1.0 Hz, 3H), 1.35 (t, J=7.1 Hz, 3H) ppm.

### Step 3. Synthesis of 4-fluoro-7-methyl-1H-indole-2-carboxylic acid

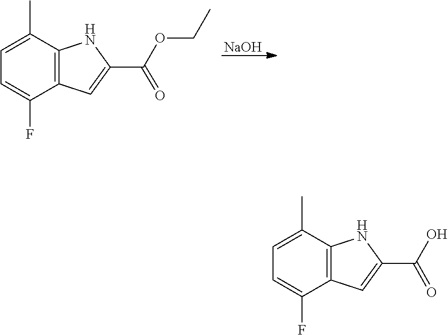

Into a 500-mL round-bottom flask, was placed a solution of ethyl 4-fluoro-7-methyl-1H-indole-2-carboxylate (9.1 g, 41.13 mmol, 1.00 equiv) in tetrahydrofuran (150 mL), sodium hydroxide (8 g, 200.00 mmol, 5.00 equiv), water (50 mL), methanol (2 mL). The resulting solution was stirred for 6 h at 25° C. The resulting mixture was concentrated under vacuum. The residue was diluted with water 50 ml, then adjusted to pH 5 with hydrogen chloride (3.0 mol/L). The resulting solution was extracted with 3×50 mL of ethyl acetate. The solid was collected by filtration. This resulted in 8.0 (yield=81%) g of 4-fluoro-7-methyl-1H-indole-2-carboxylic acid as a brown solid. LCMS (Method C, ESI): RT=0.989 min, m/z=192.0 [M−H]⁺. ¹H NMR (300 MHz, DMSO-d₆) δ 13.10 (s, 1H), 11.94 (s, 1H), 7.09 (d, J=2.1 Hz, 1H), 6.96 (m, 1H), 6.73 (m, 1H), 2.46 (d, J=1.1 Hz, 3H) ppm.

### Step 4. Synthesis of tert-butyl N-[3-(4-acetylpiperazin-1-yl)cyclohexyl]carbamate

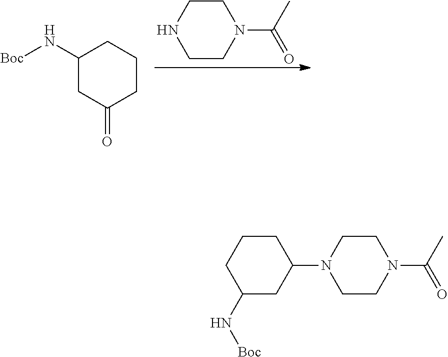

Into a 100-mL round-bottom flask, was placed tert-butyl N-(3-oxocyclohexyl)carbamate (800 mg, 3.75 mmol, 1.00 equiv), 1-(piperazin-1-yl)ethan-1-one (800 mg, 6.24 mmol, 1.66 equiv), methanol (10 mL), Pd/C (0.2 g), and to the above mixture, hydrogen was introduced. The resulting solution was stirred for 2 h at 25° C. The reaction progress was monitored by LCMS. The solids were filtered out. The resulting mixture was concentrated under vacuum. The crude product (900 mg) was purified by Flash-Prep-HPLC with the following conditions: Column, C18 silica gel; mobile phase; Detector, UV 254/220 nm. This resulted in 700 mg (yield=57%) of tert butyl N-[3-(4-acetylpiperazin-1-yl)cyclohexyl]carbamate as colorless oil. LCMS (Method A, ESI): RT=1.361 min, m/z=325.9 [M+H]⁺.

### Step 5. Synthesis of 1-[4-(3-aminocyclohexyl)piperazin-1-yl]ethan-1-one

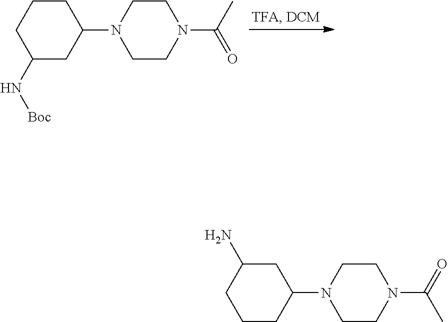 [SMILES: CC(=O)N1CCN(C2CCCC(NC(=O)OC(C)(C)C)C2)CC1 ; CC(=O)N1CCN(C2CCCC(N)C2)CC1]

Into a 100-mL round-bottom flask, was placed tert-butyl N-[3-(4-acetylpiperazin-1-yl)cyclohexyl]carbamate (700 mg, 2.15 mmol, 1.00 equiv), dichloromethane (3 mL), trifluoroacetic acid (2 mL) was added by dropwise. The resulting solution was stirred for 2 h at 25° C. The reaction progress was monitored by LCMS. The resulting mixture was concentrated under vacuum. This resulted in 700 mg of 1-[4-(3-aminocyclohexyl)piperazin-1-yl]ethan-1-one as a brown oil. LCMS (Method A, ES): RT=0.647 min. m/z=225.95 [M+H]⁺.

### Step 6. Synthesis of N-[(1R,3S)-3-(4-acetylpiperazin-1-yl)cyclohexyl]-4-fluoro-7-methyl-1H-indole-2-carboxamide (as the TFA salt)

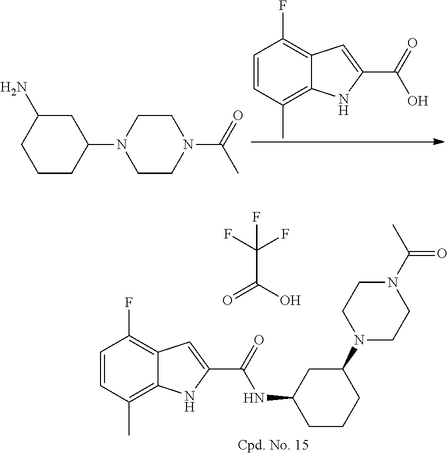

Into a 100-mL round-bottom flask, was placed 4-fluoro-7-methyl-1H-indole-2-carboxylic acid (100 mg, 0.52 mmol, 1.00 equiv), 1-[4-(3-aminocyclohexyl)piperazin-1-yl]ethan-1-one (110 mg, 0.49 mmol, 0.94 equiv), N,N-dimethylformamide (4 mL), DIEA (200 mg, 1.55 mmol, 2.99 equiv), HATU (260 mg, 0.68 mmol, 1.32 equiv) was added batchwise. The resulting solution was stirred for 2 h at 25° C. The reaction progress was monitored by LCMS, and the reaction solution was quenched by 10 ml of water. The resulting solution was extracted with 3×15 ml of ethyl acetate and the organic layers combined and concentrated under vacuum. The residue was applied onto a silica gel column with ethyl acetate/petroleum ether (1:1). The crude product was purified by Chiral-Prep-HPLC with the following conditions: Column, (R,R)-WHELK-014.6*50 mm, 3.5 μm:1-78220-30056749; mobile phase, Hexane (0.1% DEA):EtOH=85:15; Detector, UV 254 nm/220 nm. The product thus obtained was further purified by Prep-HPLC with the following conditions: Column, XBridge Prep Phenyl OBD Column, 5 μm, 19*150 mm; mobile phase, Water with 10 mmol TFA and MeCN (20.0% MeCN up to 30.0% in 10 min, up to 95.0% in 1 min, hold 95.0% in 1 min, down to 20.0% in 2 min); Detector, UV 254/220 nm. This resulted in 30.5 mg (yield=11%) of N-[(1R,3S)-3-(4-acetylpiperazin-1-yl)cyclohexyl]-4-fluoro-7-methyl-1H-indole-2-carboxamide trifluoroacetic acid salt as a white solid. LCMS (Method B, ES): RT=1.138 min, m/z=401.0 [M-TFA]+. ¹H NMR (300 MHz, Methanol-d4) δ 7.18 (s, 1H), 6.94-6.92 (m, 1H), 6.64-6.62 (m, 1H), 4.03-3.88 (m, 1H), 3.57-3.55 (m, 4H), 2.65 (t, J=16.4 Hz, 5H), 2.48 (t, J=1.0 Hz, 3H), 2.23 (d, J=12.0 Hz, 1H), 2.09 (s, 3H), 1.93 (d, J=12.2 Hz, 3H), 1.53-1.18 (m, 4H) ppm.

### Example 2

### Combination Studies

Mantle cell lymphoma (MCL) and diffuse large B-cell lymphoma (DLBCL) cell line cultures in log-linear growth rate were treated with combinations of Cpd. No. 15 and combination partners according to a co-treatment model. Assay-ready plates were prepared by dispensing the compounds with the HP-D300 nanoliter dispenser (Tecan, Mannedorf, Switzerland) onto 384-well white opaque plates (CulturPlate-384, White Opaque 384-well Microplate, Sterile and Tissue Culture Treated) to achieve either 2-fold or 3-fold serial dilutions in a concentration range bracketed around the IC₅₀ of Cpd. No. 15 and the combination partner. Concentrations were matrixed in an 8×9 array (8 concentrations of Cpd. No. 15 and 9 concentrations of its combination partner). Each combination was tested in quadruplicate wells. The final concentration of DMSO (vehicle) in the assay was 0.1% v/v. Fifty microliters of cell line suspension were directly dispensed to the assay-ready plates with an automated multichannel dispenser on to 384-well assay-ready plates. Assay plates were incubated for seven days unless otherwise indicated in a humidified atmosphere of 5% CO₂ at 37° C. Quantification of the effect of single agents or combinations on cell viability was performed through measurement of cellular adenosine triphosphate (ATP) using a CellTiter-Glo® (Promega, Madison, WI) Luminescent Cell Viability Assay. Luminescence was detected using a SpectraMax M5 microplate reader (Molecular Devices, Sunnyvale, CA). Concentration response plots were generated in GraphPad Prism version 7.0 for Windows, GraphPad Software, (La Jolla, California) and curves fitted to a four-parameter logistic model with variable slope. Percent of inhibition was calculated at each treatment concentration. Quantification of synergy was performed using the Loewe Additivity model and by calculating the Loewe Volume (VLoewe) with the CHALICE software (Horizon Discovery, Cambridge, UK) (Lehar 2007) (VLoewe>1: synergy, between 1 and −1: additivity, and <−1: antagonistic; if neither of the agents or the combination reached 50 percent inhibition of proliferation, it is deemed as “No Effect.” See Loewe, Arzneimittelforschung 3(6):285-290 (1953) and Lehar et al., Mol Syst Biol 3:80 (2007). The cell lines used in these studies were purchased from commercial suppliers. For example, NCI-H929, MM1.S, MINO, REC1, MAVERI, Z138, JEKO1, JVM2, and RPMI-8226 cell lines were purchased from the American Type Culture Collection (ATCC, Manassas VA); KMS-11, KMS34, and KMS-28-BM were purchased from the Japanese Collection of Research Bioresources (JCRB, Osaka, Japan); and L-363 and GRANTA519 were purchased from Leibniz Institute DSMZ-German Collection of Microorganisms and Cell Cultures.

The results of these combinations are summarized in Table A (mantle cell lymphoma cell lines) and Tables B-F (diffuse large B-cell lymphoma cell lines).

### Example 3

Diffuse large B-cell lymphoma (DLBCL) cell line cultures were tested with Cpd. No. 15 and combination partners according to a pre-treatment model. Cell lines in log-linear growth rate were seeded first into flasks and pre-treated with 5 concentrations of Compound 15 or DMSO for 7 days. On day 7 assay-ready plates were prepared by dispensing the compounds with the HP-D300 nanoliter dispenser (Tecan, Mannedorf, Switzerland) onto 384-well white opaque plates (CulturPlate-384, White Opaque 384-well Microplate, Sterile and Tissue Culture Treated) to achieve either 2-fold or 3-fold serial dilutions in a concentration range bracketed around the IC₅₀ of Cpd. No. 15 and the combination partner. Concentrations were matrixed in an 5×9 array (5 concentrations of Cpd. No. 15 and 9 concentrations of its combination partner). Each combination was tested in triplicate wells. The final concentration of DMSO (vehicle) in the assay was 0.1% v/v. Fifty microliters of cell line suspension were directly dispensed to the assay-ready plates with an automated multichannel dispenser on to 384-well assay-ready plates. Assay plates were incubated for seven days unless otherwise indicated in a humidified atmosphere of 5% CO₂ at 37° C. Quantification of the effect of single agents or combinations on cell viability was performed through measurement of cellular adenosine triphosphate (ATP) using a CellTiter-Glo® (Promega, Madison, WI) Luminescent Cell Viability Assay. Luminescence was detected using a SpectraMax M5 microplate reader (Molecular Devices, Sunnyvale, CA). Concentration response plots were generated in GraphPad Prism version 7.0 for Windows, GraphPad Software, (La Jolla, California) and curves fitted to a four-parameter logistic model with variable slope. Percent of inhibition was calculated at each treatment concentration. Quantification of synergy was performed using the Loewe Additivity model and by calculating the Loewe Volume (VLoewe) with the CHALICE software (Horizon Discovery, Cambridge, UK) (Lehar 2007) (VLoewe>1: synergy, between 1 and −1: additivity, and <−1: antagonistic; if neither of the agents or the combination reached 50 percent inhibition of proliferation, it is deemed as “No Effect.” See Loewe, Arzneimittelforschung 3(6):285-290 (1953) and Lehar et al., Mol Syst Biol 3:80 (2007). The cell lines used in these studies were purchased from commercial suppliers. The results of these combinations are summarized in Table G.

TABLE A

| | | MINO | REC1 | MAVER1 | Z138 | JEKO1 | JVM2 | GRANTA519 |
|---|---|---|---|---|---|---|---|---|
| | Combination | Cat. No. | Cat. No. | Cat. No. | Cat. No. | Cat. No. | Cat. No. | Cat. No. |
| Drug Class | Partner | CRL-3000 | CRL-3004 | CRL-3008 | CRL-3001 | CRL-3006 | CRL-3002 | ACC 342 |
| BTKi | Ibrutinib | Synergy | Additivity | Synergy | No Effect | Synergy | Additivity | Additivity |
| | Acalabrutinib | Synergy | Additivity | Synergy | No Effect | Synergy | Additivity | Additivity |
| | Zanubrutinib | Synergy | Additivity | Synergy | No Effect | Synergy | Additivity | Additivity |
| Anti CD20 Mab | Rituximab | No Effect | Additivity | Synergy | No Effect | Synergy | Additivity | Additivity |
| Alkylating agent | Mafosfamide | Additivity | Additivity | Additivity | Additivity | Additivity | Additivity | Additivity |
| Topoisomerase II | Doxorubicin | Additivity | Additivity | Additivity | Additivity | Additivity | Additivity | Additivity |
| inhibitor | | | | | | | | |
| Vinca alkaloid | Vincristine | Additivity | Synergy | Synergy | Additivity | Additivity | Additivity | Additivity |
| nucleoside | Cytarabine | Additivity | Additivity | Additivity | Additivity | Additivity | Additivity | Additivity |
| anticancer agents | | | | | | | | |
| PI3Ki | Copanlisib | Additivity | Additivity | Synergy | Additivity | Additivity | Additivity | Additivity |
| CDK4/6 | Palbociclib | Additivity | Additivity | Synergy | Additivity | Synergy | Additivity | Additivity |

TABLE B

| | | KARPAS422 | WSUDLCL2 | SUDHL6 | SUDHL10* |
|---|---|---|---|---|---|
| | Combination | Cat. No. | Cat. No. | Cat. No. | Cat. No. |
| Drug Class | Partner | 06101702-1VL | ACC 575 | CRL-2956 | ACC 576 |
| Anti CD20 Mab | Rituximab | No Effect | not tested | Synergy | Additivity |
| Alkylating agent | Mafosfamide | Additivity | Additivity | Synergy | Additivity |
| Topoisomerase II inhibitor | Doxorubicin | Additivity | Additivity | Synergy | Additivity |
| | Etoposide | Additivity | Additivity | Additivity | Additivity |
| Vinca alkaloid | Vincristine | Synergy | Synergy | Additivity | Additivity |
| platinum-based drug | Oxaliplatin | Additivity | Synergy | not tested | Additivity |
| | Carboplatin | Additivity | not tested | not tested | Additivity |
| nucleoside anticancer | Gemcitabine | Additivity | Additivity | Additivity | Additivity |
| agent | | | | | |
| BTKi | Ibrutinib | No Effect | Additivity | Synergy | No Effect |
| | Acalabrutinib | No Effect | Additivity | Synergy | No Effect |
| | Zanubrutinib | No Effect | Additivity | Synergy | No Effect |
| CARM1i | EPZ-2302 | Synergy | Synergy | Synergy | No Effect |

*SUDHL10: 5-Day Cotreatment

TABLE C

| | | SUDHL4 | RL** | DB | Pfeiffer |
|---|---|---|---|---|---|
| | Combination | Cat. No. | Cat. No. | Cat. No. | Cat. No. |
| Drug Class | Partner | ACC 495 | CRL-2261 | CRL-2289 | CRL-2632 |
| Anti CD20 Mab | Rituximab | not tested | not tested | No Effect | No Effect |
| Alkylating agent | Mafosfamide | Additivity | Synergy | Additivity | Additivity |
| Topoisomerase II inhibitor | Doxorubicin | Additivity | Additivity | Additivity | Additivity |
| | Etoposide | Additivity | Synergy | Additivity | Additivity |
| Vinca alkaloid | Vincristine | Additivity | Synergy | Additivity | Additivity |
| nucleoside anticancer | Gemcitabine | Additivity | Additivity | Additivity | Additivity |
| agent | | | | | |
| platinum-based drug | Carboplatin | not tested | not tested | Additivity | Additivity |
| | Oxaliplatin | not tested | not tested | Additivity | Additivity |
| BTKi | Ibrutinib | Synergy | Additivity | No Effect | No Effect |
| | Acalabrutinib | No Effect | No Effect | No Effect | No Effect |
| | Zanubrutinib | Synergy | No Effect | No Effect | No Effect |
| CARM1i | EPZ-2302 | Synergy | Synergy | No Effect | No Effect |

**RL: 6-Day Cotreatment

TABLE D

| | | DOHH2 | TOLEDO | HT | SUDHL5 |
|---|---|---|---|---|---|
| | Combination | Cat. No. | Cat. No. | Cat. No. | Cat. No. |
| Drug Class | Partner | ICLC HTL99022 | ACC 576 | CRL-2631 | ACC 571 |
| Anti CD20 Mab | Rituximab | Synergy | Additivity | Additivity | not tested |
| Alkylating agent | Mafosfamide | Additivity | Synergy | Additivity | Additivity |
| Topoisomerase II inhibitor | Doxorubicin | Additivity | Synergy | Additivity | Additivity |
| | Etoposide | Additivity | Synergy | Synergy | Additivity |
| Vinca alkaloid | Vincristine | Synergy | Synergy | Synergy | Synergy |
| nucleoside anticancer | Gemcitabine | Additivity | Additivity | Additivity | Additivity |
| agent | | | | | |
| platinum-based drug | Carboplatin | Additivity | Additivity | Additivity | Additivity |
| | Oxaliplatin | Additivity | Additivity | Additivity | Additivity |
| BTKi | Ibrutinib | Synergy | No Effect | No Effect | No Effect |
| | Acalabrutinib | No Effect | No Effect | No Effect | No Effect |
| | Zanubrutinib | Synergy | Additivity | No Effect | No Effect |
| CARM1i | EPZ-2302 | Synergy | Synergy | Synergy | Additivity |

TABLE E

| | | WSUNHL | SUDHL8 | WILL2 | SUDHL2 |
|---|---|---|---|---|---|
| | Combination | Cat. No. | Cat. No. | Cat. No. | Cat. No. |
| Drug Class | Partner | ACC 58 | CRL-2961 | ACC 652 | CRL-2260 |
| Anti CD20 Mab | Rituximab | Additivity | Additivity | Additivity | No Effect |
| Alkylating agent | Mafosfamide | Synergy | Additivity | Additivity | Additivity |
| Topoisomerase II inhibitor | Doxorubicin | Additivity | Additivity | Additivity | Additivity |
| | Etoposide | Synergy | Synergy | Additivity | Additivity |
| Vinca alkaloid | Vincristine | Synergy | Synergy | Additivity | Additivity |
| nucleoside anticancer | Gemcitabine | Additivity | Additivity | Additivity | Additivity |
| agent | | | | | |
| platinum-based drug | Carboplatin | Synergy | Synergy | Additivity | Additivity |
| | Oxaliplatin | Synergy | Additivity | Additivity | Additivity |
| BTKi | Ibrutinib | Additivity | No Effect | Additivity | No Effect |
| | Acalabrutinib | No Effect | No Effect | Additivity | No Effect |
| | Zanubrutinib | Additivity | No Effect | Additivity | No Effect |
| CARM1i | EPZ-022302-9 | Synergy | No Effect | Synergy | Synergy |

TABLE F

| | | TMD8 | Ri1 | WILL1 |
|---|---|---|---|---|
| | Combination | Cat. No. | Cat. No. | Cat. No. |
| Drug Class | Partner | M-097 | 96090512-1VL | ACC 651 |
| Anti CD20 Mab | Rituximab | No Effect | Additivity | No Effect |
| Alkylating agent | Mafosfamide | Additivity | Additivity | No Effect |
| Topoisomerase II inhibitor | Doxorubicin | Additivity | Synergy | Additivity |
| | Etoposide | Synergy | Synergy | Additivity |
| Vinca alkaloid | Vincristine | Additivity | Synergy | not tested |
| nucleoside anticancer | Gemcitabine | Additivity | Additivity | Additivity |
| agent | | | | |
| platinum-based drug | Oxaliplatin | Synergy | Additivity | Additivity |
| | Carboplatin | Additivity | Additivity | Additivity |
| BTKi | Ibrutinib | Synergy | Synergy | No Effect |
| | Acalabrutinib | Synergy | Synergy | No Effect |
| | Zanubrutinib | Synergy | Synergy | No Effect |
| CARM1i | EPZ-022302-9 | Synergy | Synergy | Synergy |

TABLE G

| | | Cell Lines | | | | |
|---|---|---|---|---|---|---|
| | | WILL2 | SUDHL4 | HT | Farage | OCILY7 |
| | | Cat. No. | Cat. No. | Cat. No. | Cat. No. | Cat. No. |
| Drug Class | Drug | ACC 652 | ACC 495 | CRL-2631 | CRL-2630 | ACC 688 |
| ATM inhibitor | AZD0156 | No Effect | Synergy | Synergy | Synergy | Synergy |
| ATR inhibitor | AZD6738 | No Effect | Synergy | Additivity | Synergy | Additivity |
| Chk1 inhibitor | AZD7762 | Additivity | Synergy | Additivity | Synergy | Additivity |
| Wee1 inhibitor | AZD1775 | Synergy | Synergy | Additivity | Synergy | Additivity |
| RAD51 inhibitor | B02 | Additivity | Synergy | Additivity | Synergy | Additivity |
| PARP inhibitor | Olaparib | Synergy | Synergy | Synergy | Synergy | Additivity |
| PARP inhibitor | Niraparib | Synergy | Synergy | Synergy | Synergy | Additivity |
| Alkylating agent | Mafosfamide (C) | not tested | Additivity | Additivity | Additivity | no effect |
| Topoisomerase II inhibitor | Doxorubicin (H) | Synergy | Synergy | Synergy | Synergy | Additivity |
| Topoisomerase II inhibitor | Etoposide | Synergy | Synergy | Synergy | Synergy | Additivity |
| BTK inhibitor | Ibrutinib | not tested | Synergy | no effect | Synergy | not tested |
| PI3Kdelta inhibitor | Idelalisib | not tested | Synergy | Synergy | Synergy | Additivity |
| AKT inhibitor | MK2206 | Synergy | Synergy | Synergy | Synergy | Synergy |
| platinum-based drug | Carboplatin | Synergy | | Synergy | Additivity | Additivity |
| Vinca alkaloid | Vincristine (O) | Synergy | Synergy | Synergy | Synergy | Additivity |
| Antimetabolite | Gemcitabine | Additivity | Additivity | Additivity | Additivity | Additivity |
| BTK inhibitor | Acalabrutinib | No Effect | No Effect | No Effect | Synergy | No Effect |
| BTK inhibitor | Zanubrutinib | not tested | Synergy | Synergy | Synergy | Synergy |
| SYK inhibitor | Tamatinib | Additivity | Synergy | Additivity | Synergy | Additivity |
| MEK inhibitor | Trametinib | not tested | Synergy | Synergy | Synergy | Synergy |

Having now fully described this invention, it will be understood by those of ordinary skill in the art that the same can be performed within a wide and equivalent range of conditions, formulations, and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, e.g., WO 2020/037079, WO 2021/168313, and publications cited herein are fully incorporated by reference herein in their entirety.

What is claimed is:

1. A method of treating a subject in need thereof, the method comprising administering to the subject a therapeutically effective amount of:
(a) compound of Formula I: 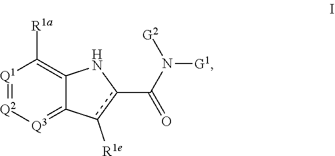
wherein:
R^{1a} is selected from the group consisting of halogen, alkyl, alkoxy, cycloalkyl, (hydroxy)alkyl, and (cycloalkyl)alkyl;
Q¹ is selected from the group consisting of —C(R^{1b})═ and —N═;
Q² is selected from the group consisting of —C(R^{1c})═ and —N═;
Q³ is selected from the group consisting of —C(R^{1d})═ and —N═;
provided that at least one of Q¹, Q², or Q³ is —C(R^{1b})═, —C(R^{1c})═, or —C(R^{1d})═, respectively;
R^{1b}, R^{1c}, and R^{1d} are each independently selected from the group consisting of hydrogen, halogen, alkyl, alkenyl, (hydroxy)alkyl, and alkoxy;
R^{1e} is selected from the group consisting of hydrogen, halogen, alkyl, cycloalkyl, (hydroxy)alkyl, and (cycloalkyl)alkyl; [symbol]
is a single or double bond;
G¹ is selected from the group consisting of: optionally substituted aryl; optionally substituted heteroaryl; optionally substituted heterocyclo; optionally substituted cycloalkyl; (aryl)alkyl; (heteroaryl)alkyl; (heterocyclo)alkyl; (amino)(aryl)alkyl; (heteroaryl)(aryl)alkyl; (heteroaryl)(heterocyclo)alkyl; (heteroaryl)(carboxamido)alkyl; (heteroaryl)(cycloalkyl)alkyl; (aryl)(alkoxycarbonyl)alkyl; (cycloalkyl)alkyl; (heteroaryl)(amino)alkyl; (cycloalkyl)(alkoxycarbonyl)alkyl; (heteroaryl)(alkoxycarbonyl)alkyl; (heterocyclo)(cycloalkyl)alkyl; (aryl)(cycloalkyl)alkyl; (aryl)(hydroxy)alkyl; (cycloalkyl)(hydroxy)alkyl; (hydroxy)alkyl; optionally substituted alkyl; (aryl)(haloalkyl)alkyl; (cycloalkyl)(haloalkyl)alkyl; (hydroxy)(haloalkyl)alkyl; and (alkoxycarbonyl)(haloalkyl)alkyl; and
G² is selected from the group consisting of hydrogen and alkyl; or
G¹ and G² taken together with the nitrogen atom to which they are attached form an optionally substituted heterocyclo, or a pharmaceutically acceptable salt or solvate thereof; and
(b) a Second Therapeutic Agent,
wherein:
the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof.

2. The method of claim 1, wherein the compound is a compound of Formula II: 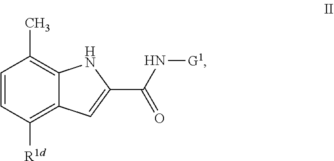
or a pharmaceutically acceptable salt or solvate thereof.

3. The method of claim 1 or 2, wherein G¹ is selected from the group consisting of: optionally substituted C₆-C₁₀ aryl; optionally substituted 5- to 9-membered heteroaryl; optionally substituted 3- to 10-membered heterocyclo; optionally substituted C₆-C₈ cycloalkyl; (5- to 9-membered heteroaryl)C₁-C₆ alkyl; (5- to 9-membered heteroaryl)(C₆₋₁₀ aryl)C₁-C₄ alkyl; (5- to 9-membered heteroaryl heteroaryl)(C₃-C₆ cycloalkyl)C₁-C₄ alkyl; and (C₃-C₆ cycloalkyl)C₁-C₄ alkyl, or a pharmaceutically acceptable salt or solvate thereof.

4. The method of claim 3, wherein the compound is a compound of Formula IV: 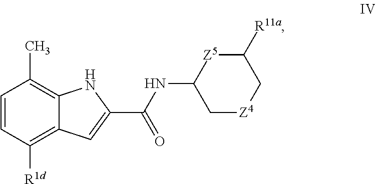
wherein:
Z⁴ is selected from the group consisting of —O—, —C(R^{28a})(R^{28b})—, and —N(R²³)—; or
Z⁴ is absent;
Z⁵ is selected from the group consisting of —CH₂— and —CH₂CH₂—;
R^{11a} is selected from the group consisting of optionally substituted alkyl, optionally substituted heterocyclo, optionally substituted heteroaryl, and —N(R^{12b})C(═O)R^{13c};
R^{12b} is selected from the group consisting of hydrogen, alkyl, cycloalkyl, and heterocyclo, (C₁-C₄ alkoxy)C₁-C₄ alkyl, and (hydroxy)C₁-C₄ alkyl; and
R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, amino, (amino)alkyl, (C₃-C₆ cycloalkyl)oxy, and (4- to 8-membered heterocyclo)oxy;
R²³ is selected from the group consisting of hydrogen and C₁-C₄ alkyl; and
R^{28a} and R^{28b} are independently selected from the group consisting of hydrogen, alkyl, and halo;
or a pharmaceutically acceptable salt or solvate thereof.

5. The method of claim 4, wherein the compound is a compound of Formula IV-A: 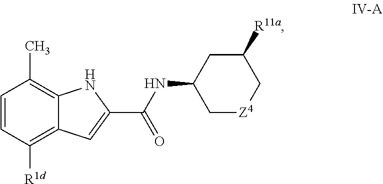
or a pharmaceutically acceptable salt or solvate thereof.

6. The method of claim 4, wherein the compound is a compound of Formula IV-B: 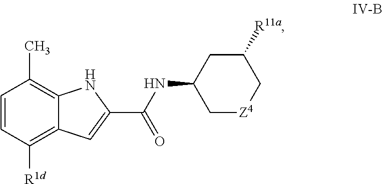
or a pharmaceutically acceptable salt or solvate thereof.

7. The method of claim 4, wherein the compound is a compound of Formula IV-C: 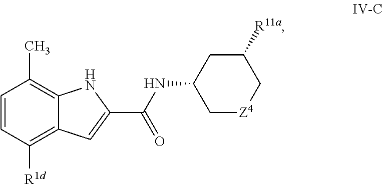
or a pharmaceutically acceptable salt or solvate thereof.

8. The method of claim 4, wherein the compound is a compound of Formula IV-D: 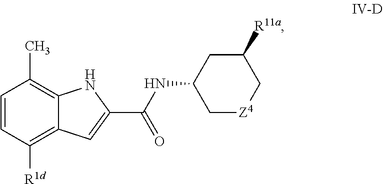
or a pharmaceutically acceptable salt or solvate thereof.

9. The method of any one of claims 4-8, wherein:
R^{11a} is selected from the group consisting of:
(A) unsubstituted 4- to 14-membered heterocyclo;
(B) substituted 4- to 14-membered heterocyclo having one, two or three substituents independently selected from the group consisting of:
(i) —N(R^{12a})C(═O)R^{13a}; (ii) —C(═O)R^{13b}; (iii) C₁-C₄ alkyl; (iv) (C₁-C₄ alkoxy)C₁-C₄ alkyl; (v) (hydroxy)C₁-C₄ alkyl; (vi) C₁-C₄ haloalkyl; (vii) amino; (vii) hydroxy; (viii) —N(R^{12a})S(═O)₂R²⁴; (ix) —S(═O)₂R²⁴; (x) unsubstituted C₃-C₆ cycloalkyl; (xi) substituted C₃-C₆ cycloalkyl having one or two substituents independently selected from the group consisting of halo, hydroxy, C₁-C₄ alkyl, amino, and (amino)C₁-C₄ alkyl; (xii) unsubstituted 4- to 14-membered heterocyclo; and substituted 4- to 14-membered heterocyclo having one or two substituents independently selected from the group consisting of amino, hydroxy, and C₁-C₄ alkyl; (xiii) —C(═N—R⁶⁰)R⁶¹; and (xiv) —C(═C—NO₂)R⁶⁴;
(C) unsubstituted 5- to 10-membered heteroaryl;
(D) substituted 5- or 6-membered heteroaryl having one, two, three, or four substituents independently selected from the group consisting of halo and C₁-C₄ alkyl;
(E) C₁-C₆ alkyl; and
(F) —N(R^{12b})C(═O)R^{13c};
R^{12a} and R^{12b} are each independently selected from the group consisting of hydrogen, C₁-C₄ alkyl, (C₁-C₄ alkoxy)C₁-C₄ alkyl, and (hydroxy)C₁-C₄ alkyl;
R^{13a}, R^{13b}, and R^{13c} are each independently selected from the group consisting of (A) C₁-C₆ alkyl; (B) C₁-C₆ haloalkyl; (C) unsubstituted C₃-C₆ cycloalkyl; (D) C₁-C₆ alkoxy; (E) (C₁-C₄ alkoxy)C₁-C₄ alkyl; (F) (hydroxy)C₁-C₄ alkyl; (G) (cyano)alkyl; (H) unsubstituted C₆-C₁₀ aryl; (I) substituted C₆-C₁₀ aryl, having one, two, three, or four substituents independently selected from the group consisting of halo, amino, hydroxy, and C₁-C₄ alkyl; (J) unsubstituted 5- or 6-membered heteroaryl; (K) substituted 5- or 6-membered heteroaryl having one, two, three, or four substituents independently selected from the group consisting of halo, amino, hydroxy, and C₁-C₄ alkyl; (L) unsubstituted 4- to 14-membered heterocyclo; (M) substituted 4- to 14-membered heterocyclo having one or two substituents independently selected from the group consisting of amino, hydroxy, and C₁-C₄ alkyl; (N) amino; (0) (amino)alkyl; (P) (C₃-C₆ cycloalkyl)oxy; and (Q) (4- to 8-membered heterocyclo)oxy; and
R²⁴ is selected from the group consisting of C₁-C₄ alkyl and (hydroxy)C₁-C₄ alkyl;
R⁶⁰ is selected from the group consisting of cyano, nitro, hydroxy, C₁-C₆ alkoxy, —C(═O)R⁶², and —S(═O)₂R⁶²;
R⁶¹ is selected from the group consisting of C₁-C₆ alkyl, C₃-C₆ cycloalkyl, and —NR^{63a}R^{63b};
R⁶² is selected from the group consisting of C₁-C₆ alkyl, C₃-C₆ cycloalkyl, and —NR^{63a}R^{63b};
R^{63a} is selected from the group consisting of hydrogen, C₁-C₆ alkyl, and C₃-C₆ cycloalkyl;
R^{63b} is selected from the group consisting of hydrogen, C₁-C₆ alkyl, and C₃-C₆ cycloalkyl; or
R^{63a} and R^{63b} taken together with the nitrogen atom to which they are attached form a 4- to 6-membered optionally substituted heterocyclo;
R⁶⁴ is selected from the group consisting of C₁-C₆ alkyl, C₃-C₆ cycloalkyl, and —NR^{63c}R^{63d};
R⁶³, is selected from the group consisting of hydrogen, C₁-C₆ alkyl, and C₃-C₆ cycloalkyl;
R^{63d} is selected from the group consisting of hydrogen, C₁-C₆ alkyl, and C₃-C₆ cycloalkyl; or
R^{63c} and R^{63d} taken together with the nitrogen atom to which they are attached form a 4- to 6-membered optionally substituted heterocyclo, or a pharmaceutically acceptable salt or solvate thereof.

10. The method of claim 9, wherein R^{11a} is a substituted 4- to 14-membered heterocyclo selected from the group consisting of: 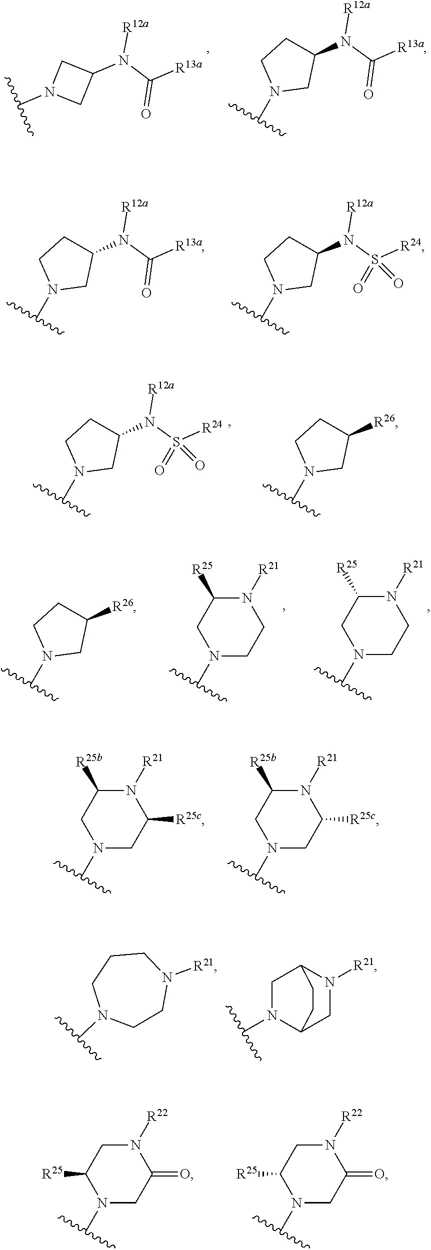 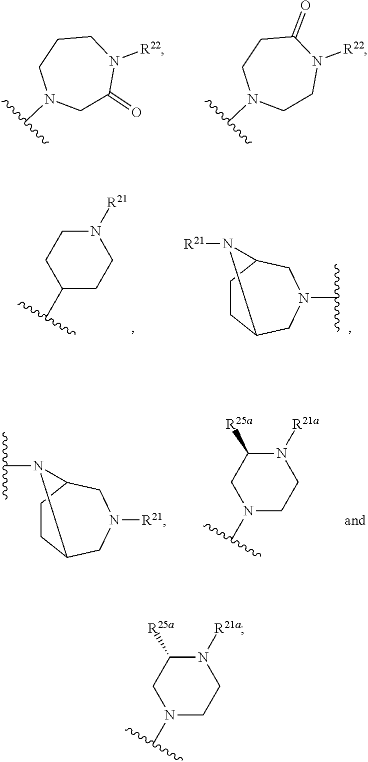
R^{12a} is selected from the group consisting of hydrogen, C₁-C₃ alkyl, (C₁-C₄ alkoxy)C₁-C₄ alkyl; and (hydroxy)C₁-C₄ alkyl;
R^{13a} is selected from the group consisting of C₁-C₄ alkyl; amino; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; (C₁-C₄ alkoxy)C₁-C₄ alkyl; (hydroxy)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;
R^{13b} is selected from the group consisting of C₁-C₄ alkyl; amino; C₁-C₄ haloalkyl;
C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;
R²¹ is selected from the group consisting of hydrogen, —C(═O)R^{13b}, C₁-C₄ alkyl, C₁-C₄ haloalkyl, unsubstituted 4- to 14-membered heterocyclo, and —S(═O)₂R²⁴;
R²² is C₁-C₄ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;
R²⁴ is selected from the group consisting of C₁-C₄ alkyl and (hydroxy)C₁-C₄ alkyl;
R²⁵ is selected from the group consisting of hydrogen, C₁-C₄ alkyl, and C₁-C₄ haloalkyl;
R^{25b} and R^{25c} are independently selected from the group consisting of C₁-C₄ alkyl and C₁-C₄ haloalkyl;
R²⁶ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl; and
R^{21a} and R^{25a} taken together with the atoms to which they are attached form an optionally substituted 4- to 8-membered heterocyclo, or a pharmaceutically acceptable salt or solvate thereof.

11. The method of claim 9, wherein R^{11a} is a substituted 4- to 14-membered heterocyclo selected from the group consisting of: 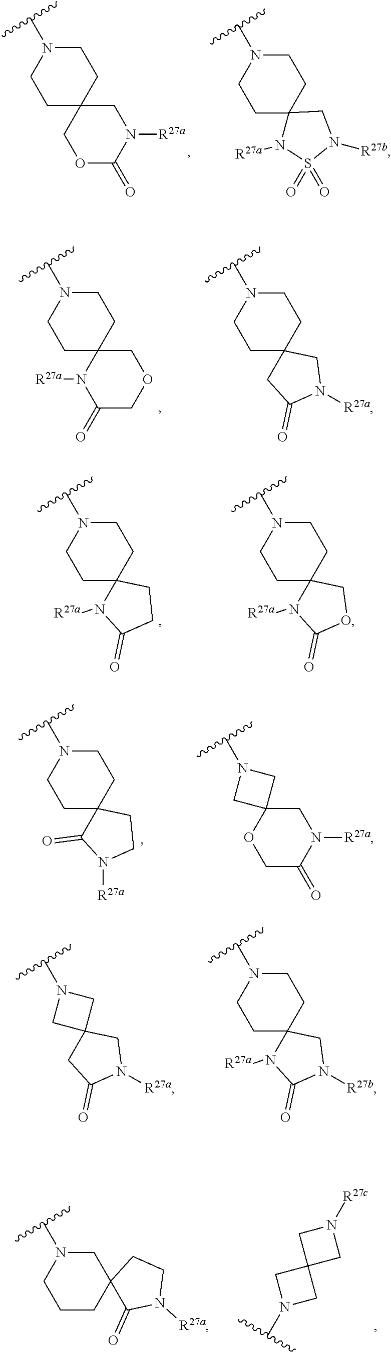 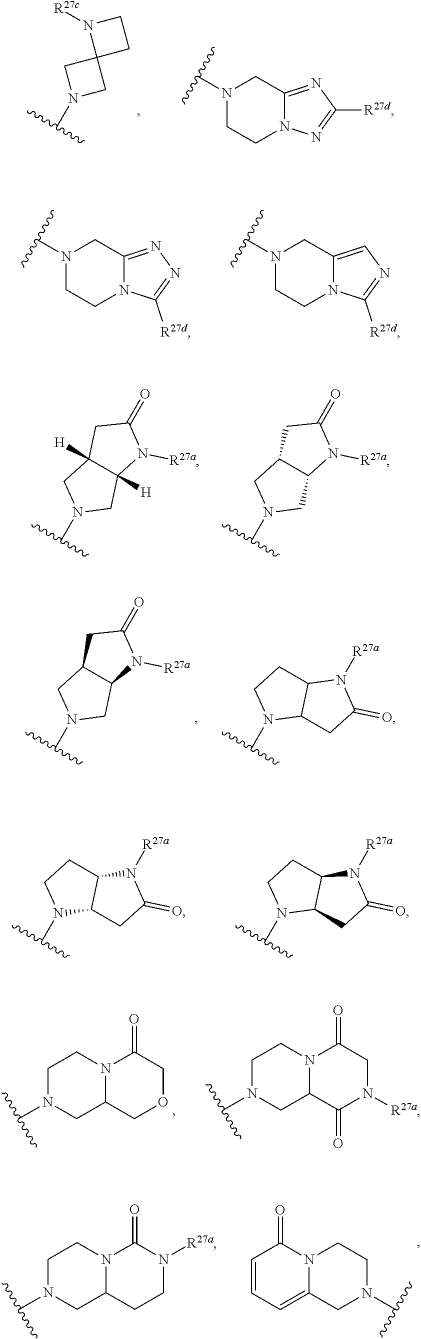 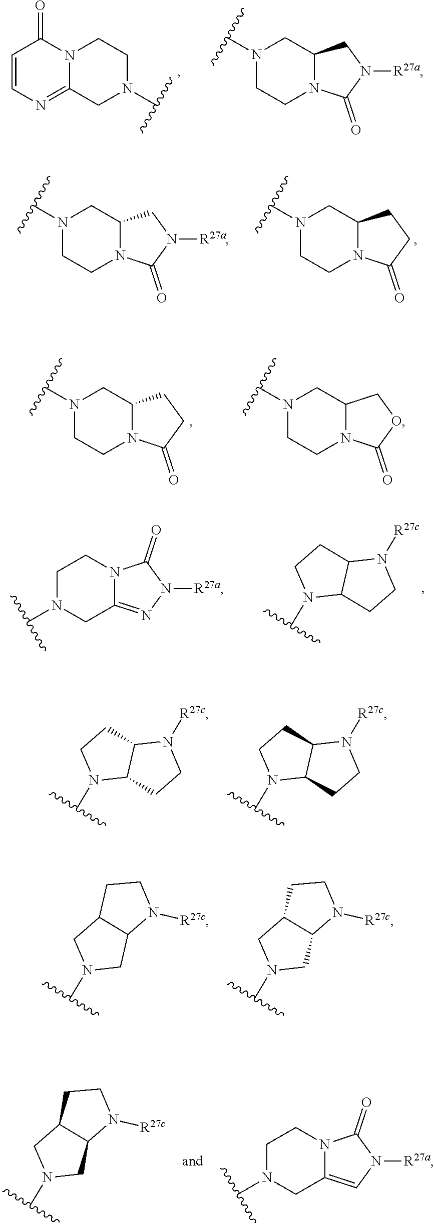
R^{27a} and R^{27b} are each independently selected from the group consisting of hydrogen, C₁-C₄ alkyl, C₁-C₄ haloalkyl, (C₁-C₄ alkoxy)C₁-C₄ alkyl; and (hydroxy)C₁-C₄ alkyl;
R^{27c} is selected from the group consisting of hydrogen; —C(═O)R^{13b}; C₁-C₄ alkyl; C₁-C₄ haloalkyl; unsubstituted 4- to 14-membered heterocyclo; and substituted 4- to 14-membered heterocyclo having one or two substituents independently selected from the group consisting of amino, hydroxy, and C₁-C₄ alkyl; and —S(═O)₂R²⁴;
R^{27d} is selected from the group consisting of hydrogen; C₁-C₄ alkyl; and C₁-C₄ haloalkyl;
R^{13b} is selected from the group consisting of C₁-C₄ alkyl; aminoC₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy; and
R²⁴ is selected from the group consisting of C₁-C₄ alkyl and (hydroxy)C₁-C₄ alkyl, or a pharmaceutically acceptable salt or solvate thereof.

12. The method of claim 11, wherein R^{11a} is a substituted 4- to 14-membered heterocyclo selected from the group consisting of: 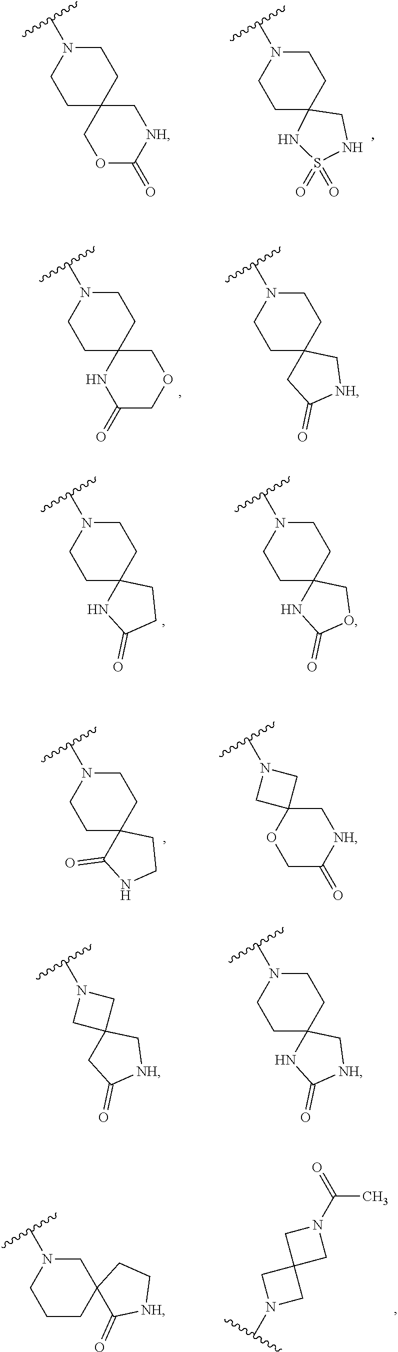 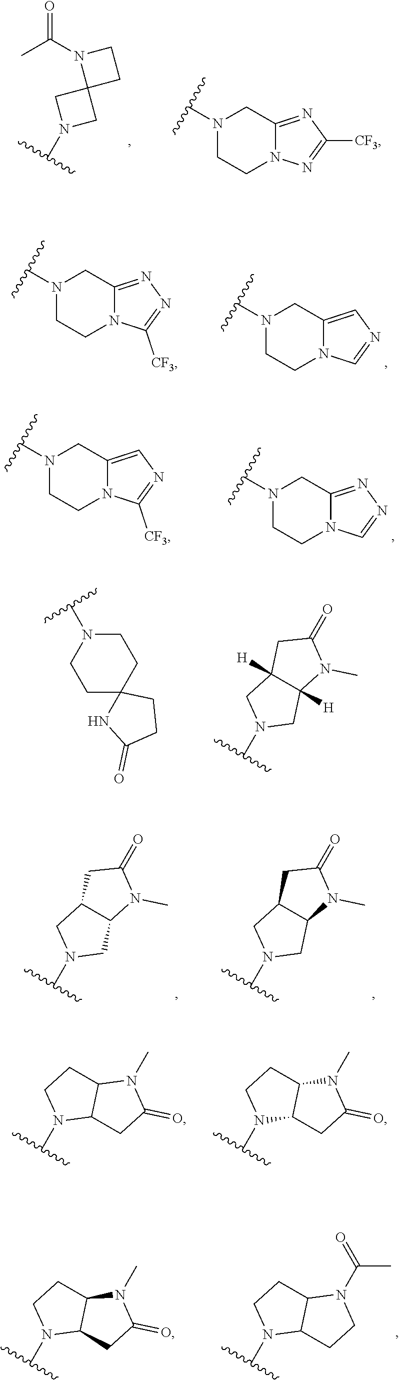 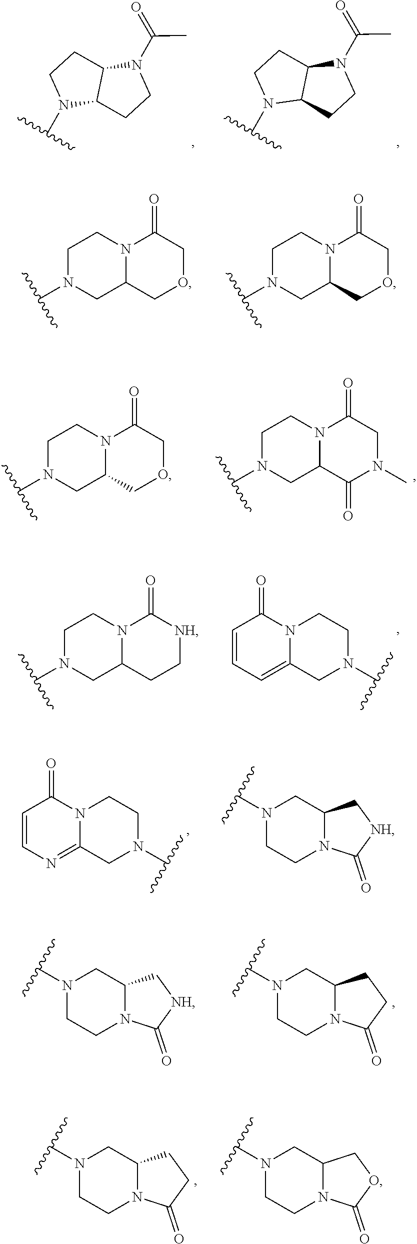 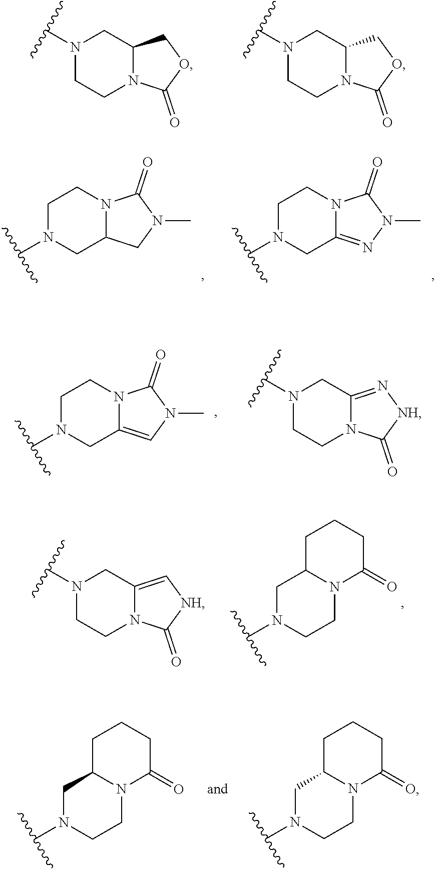
or a pharmaceutically acceptable salt or solvate thereof.

13. The method of claim 9, wherein R^{11a} is a substituted 4- to 14-membered heterocyclo selected from the group consisting of: 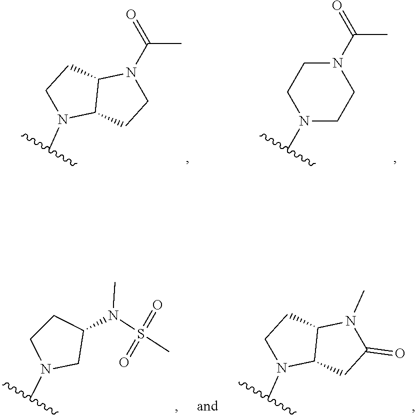
or a pharmaceutically acceptable salt or solvate thereof.

14. The method of claim 13, wherein R^{11a} is: 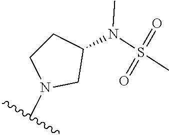 [SMILES: *N1CC[C@H](N(C)S(C)(=O)=O)C1]
or a pharmaceutically acceptable salt or solvate thereof.

15. The method of any one of claims 4-14, wherein Z⁴ is —CH₂—, or a pharmaceutically acceptable salt or solvate thereof.

16. The method of any one of claims 1-15, wherein R^{1d} is fluoro, or a pharmaceutically acceptable salt or solvate thereof.

17. The method of claim 1, wherein the compound is a compound of Table 1, or a pharmaceutically acceptable salt or solvate thereof.

18. The method of claim 1, wherein the compound is a compound of Table 1B, or a pharmaceutically acceptable salt or solvate thereof.

19. The method of any one of claims 1-18, wherein the Second Therapeutic Agent comprises a BTK inhibitor.

20. The method of claim 19, wherein the BTK inhibitor is ibrutinib, acalabrutinib, or zanubrutinib.

21. The method of any one of claims 1-20, wherein the Second Therapeutic Agent comprises an anti-CD20 monoclonal antibody.

22. The method of claim 21, wherein the anti CD20 monoclonal antibody is rituximab.

23. The method of any one of claims 1-22, wherein the Second Therapeutic Agent comprises a PI3K inhibitor.

24. The method of claim 23, wherein the PI3K inhibitor is copanlisib.

25. The method of any one of claims 1-24, wherein the Second Therapeutic Agent comprises a CDK4/6 inhibitor.

26. The method of claim 25, wherein the CDK4/6 inhibitor is palbociclib.

27. The method of any one of claims 1-26, wherein the Second Therapeutic Agent comprises a CARM1 inhibitor.

28. The method of claim 27, wherein the CARM1 inhibitor is EZM2302.

29. The method of any one of claims 1-28, wherein the Second Therapeutic Agent comprises an alkylating agent.

30. The method of claim 29, wherein the alkylating agent is mafosfamide.

31. The method of any one of claims 1-30, wherein the Second Therapeutic Agent comprises a topoisomerase II inhibitor.

32. The method of claim 29, wherein the topoisomerase II inhibitor is doxorubicin or etoposide.

33. The method of any one of claims 1-32, wherein the Second Therapeutic Agent comprises a vinca alkaloid.

34. The method of claim 33, wherein the vinca alkaloid is vincristine.

35. The method of any one of claims 1-34, wherein the Second Therapeutic Agent comprises a platinum-based drug.

36. The method of claim 33, wherein the platinum-based drug is carboplatin or oxaliplatin.

37. The method of any one of claims 1-36, wherein the Second Therapeutic Agent comprises a nucleoside anticancer agent.

38. The method of claim 37, wherein the nucleoside anticancer agent is gemcitabine.

39. The method of any one of claims 1-38, wherein the Second Therapeutic Agent comprises a DNA repair enzyme inhibitor.

40. The method of claim 39, wherein the DNA repair enzyme inhibitor is an ATM inhibitor, ATR inhibitor, Chk1 inhibitor, Wee1 inhibitor, RAD51 inhibitor, PARP inhibitor, or AKT inhibitor.

41. The method of claim 40, the ATM inhibitor is AZD0156, dactolisib, KU-55933, CP-466722, or AZD1390.

42. The method of claim 40, the ATR inhibitor is AZD6738 VX-803, or elimusertib.

43. The method of claim 40, the Chk1 inhibitor is AZD7762, rabusertib, MK-8776, CHIR-124, or PF-477736.

44. The method of claim 40, the Wee1 inhibitor is AZD1775.

45. The method of claim 40, the RAD51 inhibitor is B02 or RI-1.

46. The method of claim 40, the PARP inhibitor is olaparib, niraparib rucaparib, or talazoparib.

47. The method of claim 40, the AKT inhibitor is MK2206.

48. The method of any one of claims 1-47, wherein the Second Therapeutic Agent comprises a SYK inhibitor.

49. The method of claim 48, the SYK inhibitor is tamatinib, fostamatinib, R406, MNS, lanraplenib, TAK-659, entospletinib, or BAY-61-3606.

50. The method of any one of claims 1-49, wherein the Second Therapeutic Agent comprises a MEK inhibitor.

51. The method of claim 50, the MEK inhibitor is trametinib, selumetinib, or merdametinib.

52. The method of any one of claims 18-51, wherein the compound of Table 1B is Cpd. No. 15, or a pharmaceutically acceptable salt or solvate thereof.

53. The method of any one of claims 1-52 further comprising administering a therapeutically effective amount of a Third Therapeutic Agent to the subject, wherein the Third Therapeutic Agent comprises one or more glucocorticoid receptor agonists, one or more immunomodulatory drugs, one or more proteasome inhibitors, one or more Bcl-2 inhibitors, one or more pleiotropic pathway modulators, one or more XPO1 inhibitors, one or more histone deacetylase inhibitors, one or more EZH2 inhibitors, or a combination thereof.

54. The method claim 53, wherein the Third Therapeutic Agent comprises a glucocorticoid receptor agonist.

55. The method of claim 54, wherein the glucocorticoid receptor agonist is dexamethasone.

59. The method of any one of claims 53-55, wherein the Third Therapeutic Agent comprises an immunomodulatory drug.

60. The method of claim 59, wherein the immunomodulatory drug is pomalidomide or lenalidomide.

61. The method of any one of claims 53-60, wherein the Third Therapeutic Agent comprises a proteasome inhibitor.

62. The method of claim 61, wherein the proteasome inhibitor is bortezomib.

63. The method of any one of claims 53-62, wherein the Third Therapeutic Agent comprises a Bcl-2 inhibitor.

64. The method of claim 63, wherein the Bcl-2 inhibitor is venetoclax.

65. The method of any one of claims 53-64, wherein the Third Therapeutic Agent comprises a pleiotropic pathway modulator.

66. The method of claim 65, wherein the pleiotropic pathway modulator is CC-122.

67. The method of any one of claims 53-66, wherein the Third Therapeutic Agent comprises a XPO1 inhibitor.

68. The method of claim 67, wherein the XPO1 inhibitor is selinexor.

69. The method of any one of claims 53-68, wherein the Third Therapeutic Agent comprises a histone deacetylase inhibitor.

70. The method of claim 69, wherein the histone deacetylase inhibitor is panobinostat.

71. The method of any one of claims 53-70, wherein the Third Therapeutic Agent is an EZH2 inhibitor.

72. The method of claim 71, wherein the EZH2 inhibitor is tazemetostat.

73. The method of any one of claims 1-52, wherein the compound of Formula I, or a pharmaceutically acceptable salt or solvate thereof, and the Second Therapeutic Agent are administered to the subject separately.

74. The method of any one of claims 53-73, wherein the compound of Formula I, or a pharmaceutically acceptable salt or solvate thereof, the Second Therapeutic Agent, and the Third Therapeutic Agent are administered to the subject separately

75. The method of any one of claims 1-74, wherein the subject in need thereof has cancer.

76. The method of claim 75, wherein the cancer is any one or more of the cancers of Table 2.

77. The method of claim 75, wherein the cancer is a hematological cancer.

78. The method of claim 77, wherein the hematological cancer is any one or more of the cancers of Table 3.

79. The method of claim 78, wherein the hematological cancer is multiple myeloma, mantle cell lymphoma, or diffuse large B-cell lymphoma.

80. The method of claim 79, wherein the hematological cancer is mantle cell lymphoma.

81. The method of claim 79, wherein the hematological cancer is diffuse large B-cell lymphoma.

82. The method of claim 79, wherein the hematological cancer is multiple myeloma.

83. The method of claim 82, wherein the hematological cancer is t(4;14) multiple myeloma.

84. A kit for carrying out the method of any one of claims 1-83, the kit comprising:
(a) compound of Formula I: 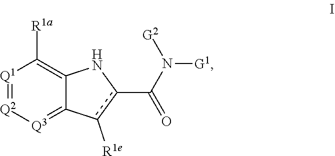
or a pharmaceutically acceptable salt or solvate thereof, wherein:
R^{1a} is selected from the group consisting of halogen, alkyl, alkoxy, cycloalkyl, (hydroxy)alkyl, and (cycloalkyl)alkyl;
Q¹ is selected from the group consisting of —C(R^{1b})═ and —N═;
Q² is selected from the group consisting of —C(R^{1c})═ and —N═;
Q³ is selected from the group consisting of —C(R^{1d})═ and —N═;
provided that at least one of Q¹, Q², or Q³ is —C(R^{1b})═, —C(R^{1c})═, or —C(R^{1d})═, respectively;
R^{1b}, R^{1c}, and R^{1d} are each independently selected from the group consisting of hydrogen, halogen, alkyl, alkenyl, (hydroxy)alkyl, and alkoxy;
R^{1e} is selected from the group consisting of hydrogen, halogen, alkyl, cycloalkyl, (hydroxy)alkyl, and (cycloalkyl)alkyl; [symbol]
is a single or double bond;
G¹ is selected from the group consisting of: optionally substituted aryl;
optionally substituted heteroaryl; optionally substituted heterocyclo; optionally substituted cycloalkyl; (aryl)alkyl; (heteroaryl)alkyl; (heterocyclo)alkyl; (amino)(aryl)alkyl; (heteroaryl)(aryl)alkyl; (heteroaryl)(heterocyclo)alkyl; (heteroaryl)(carboxamido)alkyl; (heteroaryl)(cycloalkyl)alkyl; (aryl)(alkoxycarbonyl)alkyl; (cycloalkyl)alkyl; (heteroaryl)(amino)alkyl; (cycloalkyl)(alkoxycarbonyl)alkyl; (heteroaryl)(alkoxycarbonyl)alkyl; (heterocyclo)(cycloalkyl)alkyl; (aryl)(cycloalkyl)alkyl; (aryl)(hydroxy)alkyl; (cycloalkyl)(hydroxy)alkyl; (hydroxy)alkyl; optionally substituted alkyl; (aryl)(haloalkyl)alkyl; (cycloalkyl)(haloalkyl)alkyl; (hydroxy)(haloalkyl)alkyl; and (alkoxycarbonyl)(haloalkyl)alkyl; and
G² is selected from the group consisting of hydrogen and alkyl; or
G¹ and G² taken together with the nitrogen atom to which they are attached form an optionally substituted heterocyclo,
wherein:
the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof; and
(c) instructions for administering the compound of Formula I, or a pharmaceutically acceptable salt or solvate thereof, and the Second Therapeutic Agent to the subject.

85. A kit comprising:
(a) compound of Formula I: 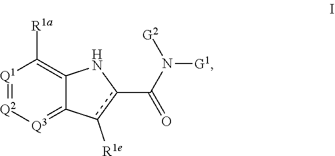
or a pharmaceutically acceptable salt or solvate thereof, wherein:
R^{1a} is selected from the group consisting of halogen, alkyl, alkoxy, cycloalkyl, (hydroxy)alkyl, and (cycloalkyl)alkyl;
Q¹ is selected from the group consisting of —C(R^{1b})═ and —N═;
Q² is selected from the group consisting of —C(R^{1c})═ and —N═;
Q³ is selected from the group consisting of —C(R^{1d})═ and —N═;
provided that at least one of Q¹, Q², or Q³ is —C(R^{1b})═, —C(R^{1c})═, or —C(R^{1d})═, respectively;
R^{1b}, R^{1c}, and R^{1d} are each independently selected from the group consisting of hydrogen, halogen, alkyl, alkenyl, (hydroxy)alkyl, and alkoxy;
R^{1e} is selected from the group consisting of hydrogen, halogen, alkyl, cycloalkyl, (hydroxy)alkyl, and (cycloalkyl)alkyl; [symbol]
is a single or double bond;
G¹ is selected from the group consisting of: optionally substituted aryl;
optionally substituted heteroaryl; optionally substituted heterocyclo; optionally substituted cycloalkyl; (aryl)alkyl; (heteroaryl)alkyl; (heterocyclo)alkyl; (amino)(aryl)alkyl; (heteroaryl)(aryl)alkyl; (heteroaryl)(heterocyclo)alkyl; (heteroaryl)(carboxamido)alkyl; (heteroaryl)(cycloalkyl)alkyl; (aryl)(alkoxycarbonyl)alkyl; (cycloalkyl)alkyl; (heteroaryl)(amino)alkyl; (cycloalkyl)(alkoxycarbonyl)alkyl; (heteroaryl)(alkoxycarbonyl)alkyl; (heterocyclo)(cycloalkyl)alkyl; (aryl)(cycloalkyl)alkyl; (aryl)(hydroxy)alkyl; (cycloalkyl)(hydroxy)alkyl; (hydroxy)alkyl; optionally substituted alkyl; (aryl)(haloalkyl)alkyl; (cycloalkyl)(haloalkyl)alkyl; (hydroxy)(haloalkyl)alkyl; and (alkoxycarbonyl)(haloalkyl)alkyl; and
G² is selected from the group consisting of hydrogen and alkyl; or
G¹ and G² taken together with the nitrogen atom to which they are attached form an optionally substituted heterocyclo,
(b) a Second Therapeutic Agent,
wherein the Second Therapeutic Agent comprises one or more BTK inhibitors, one or more anti-CD20 monoclonal antibodies, one or more alkylating agents, one or more topoisomerase II inhibitors, one or more vinca alkaloids, one or more platinum-based drugs, one or more nucleoside anticancer agents, one or more PI3K inhibitors, one or more CDK4/6 inhibitors, one or more CARM1 inhibitors, one or more inhibitors of enzymes of DNA damage repair, one or more SYK inhibitors, or one or more MEK inhibitors, or a combination thereof; and
(c) instructions for administering the compound of Formula I, or a pharmaceutically acceptable salt or solvate thereof, and the Second Therapeutic Agent to a subject.
